(19) United States
(12) Patent Application Publication
(10) Pub. No.: US 2024/0189315 A1
(43) Pub. Date: Jun. 13, 2024

(54) Cyclic Pyridine Derivatives as cGAS Inhibitors

(71) Applicant: Boehringer Ingelheim International GmbH, Ingelheim am Rhein (DE)

(72) Inventors: Annekatrin Charlotte HEIMANN, Biberach an der Riss (DE); Christian GNAMM, Biberach an der Riss (DE); Cédrickx GODBOUT, Schemmerhofen (DE); Sandra Ruth HANDSCHUH, Biberach an der Riss (DE); Christoph HOENKE, Biberach an der Riss (DE); Joerg Thomas KLEY, Mittelbiberach (DE); Christian Andreas KUTTRUFF, Donaueschingen (DE); Jun LI, Danbury, CT (US); Dirk REINERT, Bad Duerkheim (DE); Raphael STUBER, Schwendi (DE); Theodor THEIS, Biberach an der Riss (DE)

(21) Appl. No.: 18/501,509

(22) Filed: Nov. 3, 2023

Related U.S. Application Data
(60) Provisional application No. 63/382,946, filed on Nov. 9, 2022.

(30) Foreign Application Priority Data
Dec. 9, 2022 (EP) 22212488.5

(51) Int. Cl. A61K 31/529; A61K 31/4418; A61K 31/496; A61K 39/395; A61K 45/06; C07D 498/22

(52) U.S. Cl. CPC A61K 31/529; A61K 31/4418; A61K 31/496; A61K 39/3955; A61K 45/06; C07D 498/22

(57) ABSTRACT

The invention relates to compounds of formula I

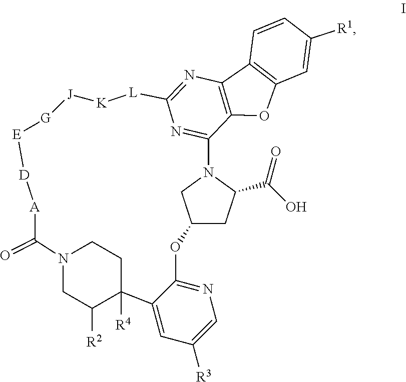

wherein R¹, R², R³, R⁴, A, D, E, G, J, K and L are defined as in claim 1, and prodrugs, deuterated analogues and pharmaceutical acceptable salts thereof, for the treatment of diseases such as systemic lupus erythematosus, systemic sclerosis (SSc), interferonopathies, non-alcoholic steatohepatitis (NASH), interstitial lung disease (ILD) and idiopathic pulmonary fibrosis (IPF).

### 1. BACKGROUND OF THE INVENTION

1.1 cGAS Inhibitors

Innate immunity is considered a first line cellular stress response defending the host cell against invading pathogens and initiating signaling to the adaptive immune system. These processes are triggered by conserved pathogen-associated molecular patterns (PAMPs) through sensing by diverse pattern recognition receptors (PRRs) and subsequent activation of cytokine and type I interferon gene expression. The major antigen-presenting cells, such as monocytes, macrophages, and dendritic cells produce type I interferons and are critical for eliciting adaptive T- and B-cell immune system responses. The major PRRs detect aberrant, i.e. mislocalized, immature or unmodified nucleic acids on either the cell surface, the inside of lysosomal membranes or within other cellular compartments (Barbalat et al., Annu. Rev. Immunol. 29, 185-214 (2011)).

“Cyclic GMP-AMP Synthase” (cGAS UniProtKB-Q8N884)) is the predominant sensor for aberrant double-stranded DNA (dsDNA) originating from pathogens or mislocalization or misprocessing of nuclear or mitochondrial cellular dsDNA (Sun et al., Science 339, 786-791 (2013); Wu et al., Science 339, 826-830 (2013); Ablasser et al., Nature 498, 380-384 (2013)). Binding of dsDNA to cGAS activates the reaction of GTP and ATP to form the cyclic dinucleotide GMP-AMP (referred to as cGAMP). cGAMP then travels to and activates the endoplasmatic reticulum membrane-anchored adaptor protein, “Stimulator of Interferon Genes” (STING). Activated STING recruits and activates TANK-binding kinase 1(TBK1) which in turn phosporylates the transcription factor family of Interferon regulatory factors (IRFs) inducing cytokine and type I interferon mRNA expression.

The critical role of cGAS in dsDNA sensing has been established in different pathogenic bacteria (Hansen et al., EMBOJ. 33, 1654 (2014)), viruses (Ma et al., PNAS 112, E4306 (2015)) and retroviruses (Gao et al., Science 341, 903-906 (2013)). Additionally, cGAS is essential in various other biological processes such as cellular senescence (Yang et al., PNAS 114, E4612 (2017), Glück et al., Nat. Cell Biol. 19, 1061-1070 (2017)) and recognition of ruptured micronuclei in the surveillance of potential cancer cells (Mackenzie et al., Nature 548, 461-465 (2017); Harding et al., Nature 548, 466-470 (2017)).

While the cGAS pathway is important for host defense against invading pathogens, cellular stress and genetic factors may also cause production of aberrant cellular dsDNA, e.g. by nuclear or mitochondrial leakage, and thereby trigger autoinflammatory responses. Aicardi-Goutieres syndrome (AGS; Crow et al., Nat. Genet. 38, 917-920 (2006))—a lupus-like severe autoinflammatory immune-mediated disorder—arises from loss-of-function mutations in TREX1, a primary DNA exonuclease responsible for degrading aberrant DNA in cytosol. Knock-out of cGAS in TREX1-deficient mice prevented otherwise lethal autoimmune responses, supporting cGAS as driver of interferonopathies (Gray et al., J. Immunol. 195, 1939-1943 (2015); Gao et al., PNAS 112, E5699-E5705 (2015)). Likewise, embryonic lethality caused by deficiency of DNAse2, an endonuclease responsible for degradation of excessive DNA in lysosomes during endocytosis, was completely rescued by additional knock-out of cGAS (Gao et. al, PNAS 112, E5699-E5705 (2015)) or STING (Ahn et al., PNAS 109, 19386-19391 (2012)). These observations support cGAS as a drug target and inhibition of cGAS may provide a therapeutic strategy for preventing autoinflammation and treating diseases such as systemic lupus erythematosus (SLE) with involvement of anti-dsDNA antibodies (Pisetsky et al., Nat. Rev. Rheumatol. 12, 102-110 (2016)).

1.2 Prior Art

Due to the observation that inhibition of the cGAS-pathway may provide a therapeutic strategy for preventing autoinflammation and for treating e.g. autoimmune diseases many efforts to develop cGAS inhibitors have been undertaken.

In WO 2019/241787 for example, methyl 4-amino-6-(phenylamino)-1,3,5-triazine-2-carboxylates such as CU-32 and CU-76 have been disclosed as cGAS-inhibitors with “in vitro hcGAS IC₅₀-values” slightly below 1 μM (IC₅₀(CU-32)=0.66 μM and IC₅₀(CU-76=0.27 μM).

In Hall et al., PLoS ONE 12(9); e0184843 (2017), compound PF-06928215 has been published as an inhibitor of cGAS with an “in vitro hcGAS IC₅₀-value” of 0.049 μM as measured by a fluorescence polarization assay. However, compound PF-06928215 showed no acceptable cellular activity as a cGAS inhibitor.

In WO 2020/142729 and in WO2022/174012, (benzofuro[3,2-d]pyrimidin-4-yl)pyrrolidine-2-carboxylic acid derivatives have been disclosed as cGAS inhibitors for the therapy of autoimmune disorders such as Aicardi-Goutieres Syndrome (AGS), lupus erythematosus, scleroderma, inflammatory bowel disease and non-alcoholic steatohepatitis (NASH). However, the compounds of this invention differ from the (benzofuro[3,2-d]pyrimidin-4-yl)pyrrolidine-2-carboxylic acid derivatives of WO 2020/142729 in their completely different substitution pattern in the 4-position of the pyrrolidine ring.

Recently provided cGAS inhibitors, such as the ones in WO 2020/142729 or in WO 2022/174012, usually show an insufficient cellular cGAS inhibitory potency (with IC₅₀-values regarding inhibition of the cGAS/STING pathway as measured in cellular assays of usually larger than 1 μM, often of larger than 5 μM). However, it is crucial to provide therapeutic cGAS inhibitors that do not only show a satisfying biochemical (in vitro) inhibitory potency (“hcGAS IC₅₀”), but also a satisfying cellular inhibitory potency (for example by showing inhibition of IFN induction in virus-stimulated THP-1 cells (THP1₍ᵥᵢᵣ₎ IC₅₀)) in order to ensure that the compound is able to show a therapeutic effect in a patient. Other important properties that may be predictive for successful development of a cGAS inhibitor as a therapeutic agent are satisfying cGAS-selectivity (versus off-target activity) and acceptable inhibitory potency in human whole blood.

Surprisingly it has now been found that the compounds of formula I or II show at the same time the following three properties:
a satisfying “biochemical (in vitro) IC₅₀-value regarding cGAS inhibition” (with a hcGAS IC₅₀ of ≤100 nM, preferably of ≤50 nM, in particular of ≤10 nM),
a satisfying “inhibition of IFN induction in virus-stimulated THP-1 cells (with a THP1|C₅₀₍ᵥᵢᵣ₎ of ≤1 μM, preferably of ≤500 nM, more preferably of 5100 nM, in particular of ≤50 nM) and
a satisfying selectivity for cGAS-inhibition (with a ratio THP1 IC_{50(cGAMP)}/THP1 IC₅₀₍ᵥᵢᵣ₎ of ≥10, more preferably ≥50, more preferably ≥500, in particular ≥1000).

Additionally the compounds of formula I or II also show acceptable IC₅₀-values with regard to inhibition of IFN induction in dsDNA-stimulated human whole blood assays, preferably with human whole blood IC₅₀-values with regard to cGAS inhibition (hWB IC₅₀) of ≤5000 nM, more preferably of ≤1000 nM, in particular of ≤100 nM.

The cGAS inhibitors of the invention with this particular pharmacological profile which combines an excellent in vitro inhibitory potency and an excellent cellular inhibitory potency with a high selectivity for cGAS inhibition have a high probability to also exhibit a good therapeutic effect in the patient. Due to their high cellular inhibitory potency compounds with this particular pharmacological profile should be able to pass the cell membrane barrier and therefore reach their intracellular target location and due to their selectivity to exclusively inhibit cGAS activity, these compounds should not show unwanted off target effects, for example side effects somewhere within the signaling pathway downstream of cGAS or cytotoxic effects.

### 2. DESCRIPTION OF THE INVENTION

The invention relates to a compound of formula I

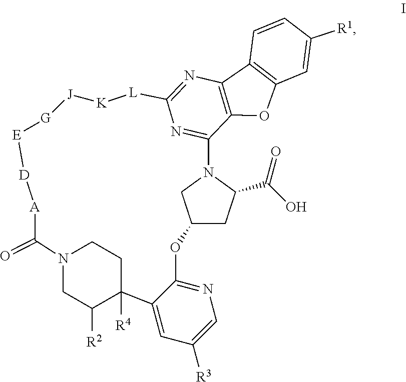

wherein
R¹ is selected from the group consisting of hydrogen, halogen, methyl, ethyl, —CF₃, —CHF₂, —CFH₂ and methoxy,
R² is selected from the group consisting of hydrogen and methyl;
R³ is selected from the group consisting of hydrogen, methyl, halogen, ethinyl, propinyl, —CO—(C₁₋₃-alkyl), —CO—NH₂, —CO—NHCH₃, —CO—N(CH₃)₂ and a 5- or 6-membered heteroaryl ring with 1 or 2 heteroatoms each independently selected from N, S or O, whereby this heteroaryl ring may optionally be further substituted by one or two further substituents each independently selected from the group consisting of F, Cl, Br, —O—CH₃, methyl, —CF₃, —CHF₂ and CH₂F;
R⁴ is selected from the group consisting of hydrogen, —OH and F;
and wherein
A is selected from the group consisting of —CH₂—, —O—, —CF₂— and —CHCH₃—; D is selected from the group consisting of —CH₂—, —O—, —CF₂— and —CHCH₃—; E is selected from the group consisting of —CH₂—, —CO—, —O—, —CF₂— and —CHCH₃—;
G is selected from the group consisting of —NH—, —NCH₃—, —CH₂—, —O—, —CF₂— and —CHCH₃—;
J is selected from the group consisting of —CO—, —CH₂—, —O—, —CF₂— and —CHCH₃—;
K is selected either from the group consisting of —CH₂—, —CF₂— and —O— or is absent;
L is selected either from the group consisting of —CH₂—, —CHCH₃— and —CF₂— or is absent; and prodrugs, deuterated analogues and pharmaceutical acceptable salts thereof.

Hereby variables A, D, E, G, J, K and L are preferably selected in such a way that two or more heteroatoms may not follow directly upon each other.

In a preferred embodiment the invention concerns the compound of formula II, wherein

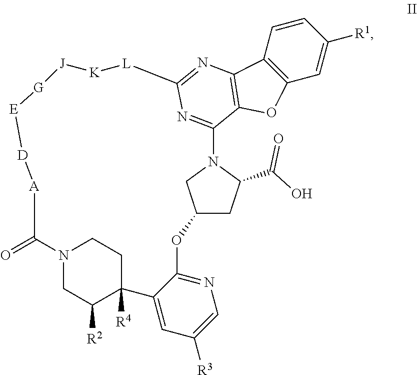

R¹ is either hydrogen and or is a halogen which is preferably selected from the group consisting of F and Cl;
R² is selected from the group consisting of hydrogen and methyl;
R³ is selected from the group consisting of hydrogen, Cl, Br, ethinyl, propinyl, —CO—(CH₃) and a 5- or 6-membered heteroaryl ring with 1 or 2 heteroatoms each independently selected from N, S or O, whereby this heteroaryl ring may optionally be further substituted by one or two further substituents each independently selected from the group consisting of F, —O—CH₃ and methyl;
R⁴ is F;
and wherein
A is selected from the group consisting of —CH₂—, —O—, —CF₂— and —CHCH₃—;
D is selected from the group consisting of —CH₂— and —O—;
E is selected from the group consisting of —CH₂—, —CO— and —O—;
G is selected from the group consisting of —NH—, —NCH₃—, —CH₂— and —O—;
J is selected from the group consisting of —CO—, —CH₂— and —O—;
K is either selected from the group consisting of —CH₂—, —CF₂—and —O— or is absent;
L is either —CH₂— or is absent;
and prodrugs, deuterated analogues and pharmaceutical acceptable salts thereof.

Hereby variables A, D, E, G, J, K and L are preferably selected in such a way that two or more heteroatoms may not follow directly upon each other.

In a further preferred embodiment the invention relates to the above-mentioned compound of formula I or to the above-mentioned compound of formula II,
wherein L is absent,
and prodrugs, deuterated analogues and pharmaceutical acceptable salts thereof.

In a further preferred embodiment the invention concerns the above-mentioned compound of formula I or the above-mentioned compound of formula II,
wherein L and K are absent,
and prodrugs, deuterated analogues and pharmaceutical acceptable salts thereof.

In a further preferred embodiment the invention relates to the above-mentioned compound of formula I or to the above-mentioned compound of formula II,
wherein L is absent and wherein K is —CF₂—,
and prodrugs, deuterated analogues and pharmaceutical acceptable salts thereof.

In another preferred embodiment the invention concerns the above-mentioned compound of formula I or the above-mentioned compound of formula II,
wherein L is absent and wherein A is selected from the group consisting of —CH₂— and —CF₂—,
and prodrugs, deuterated analogues and pharmaceutical acceptable salts thereof.

In a further preferred embodiment the invention relates to the above-mentioned compound of formula I or o the above-mentioned compound of formula II,
wherein L is absent and whereby A is —O—,
and prodrugs, deuterated analogues and pharmaceutical acceptable salts thereof.

In another preferred embodiment the invention concerns the above-mentioned compound of formula I or the above-mentioned compound of formula II,
wherein L is absent and wherein D is —O—,
and prodrugs, deuterated analogues and pharmaceutical acceptable salts thereof.

In a further preferred embodiment the invention relates to the above-mentioned compound of formula I or to the above-mentioned compound of formula II,
wherein L is absent and
wherein R³ is selected from the group consisting of Cl, Br, ethinyl, propinyl and a 5- or 6-membered heteroaryl ring selected from the group consisting of pyridinyl and pyrazolyl, whereby this heteroaryl ring may optionally be further substituted by one or two further substituents each independently selected from the group consisting of F, —O—CH₃ and methyl;
and prodrugs, deuterated analogues and pharmaceutical acceptable salts thereof.

In another preferred embodiment the invention concerns the above-mentioned compound of formula I or the above-mentioned compound of formula II,
wherein R¹ is selected from the group consisting of F and Cl,
and prodrugs, deuterated analogues and pharmaceutical acceptable salts thereof.

In a further preferred embodiment the invention relates to the above-mentioned compound of formula I or to the above-mentioned compound of formula II,
wherein R¹ is hydrogen,
and prodrugs, deuterated analogues and pharmaceutical acceptable salts thereof.

In another particularly preferred embodiment the invention concerns the above-mentioned compound of formula I or the above-mentioned compound of formula II, which is selected from the group consisting of

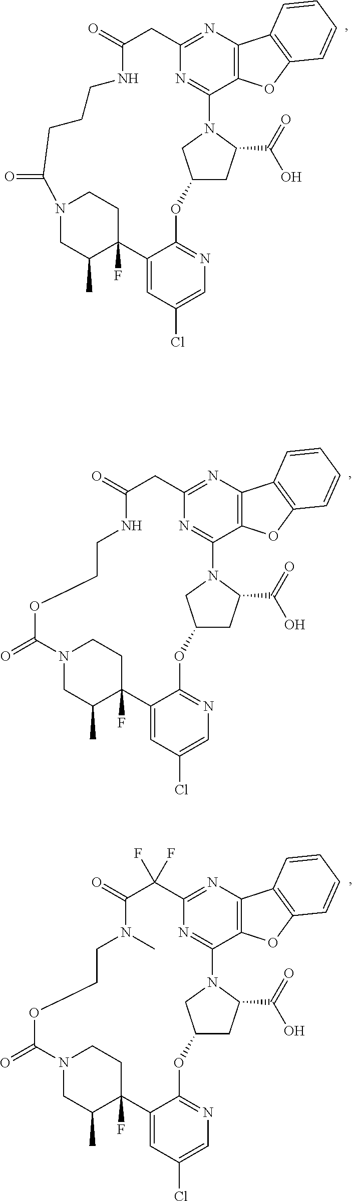 [SMILES: C[C@H]1CN2CC[C@]1(F)c1cc(Cl)cnc1O[C@H]1C[C@@H](C(=O)O)N(C1)c1nc(nc3c1oc1ccccc13)CC(=O)NCCCC2=O ; C[C@H]1CN2CC[C@]1(F)c1cc(Cl)cnc1O[C@H]1C[C@@H](C(=O)O)N(C1)c1nc(nc3c1oc1ccccc13)CC(=O)NCCCOC2=O ; C[C@H]1CN2CC[C@]1(F)c1cc(Cl)cnc1O[C@H]1C[C@@H](C(=O)O)N(C1)c1nc(nc3c1oc1ccccc13)C(F)(F)C(=O)N(C)CCCOC2=O]

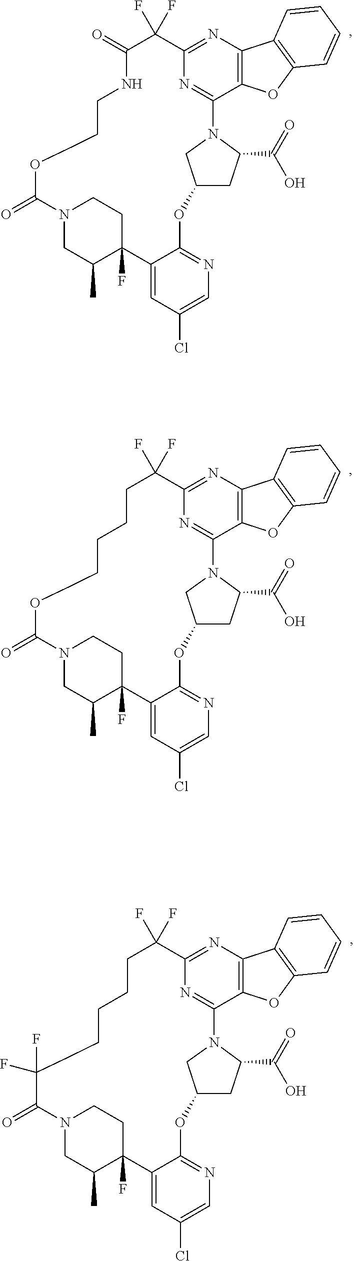 [SMILES: C[C@H]1CN2CC[C@]1(F)c1cc(Cl)cnc1O[C@H]1C[C@@H](C(=O)O)N(C1)c1nc(nc3c1oc1ccccc13)C(F)(F)C(=O)NCCCOC2=O ; C[C@H]1CN2CC[C@]1(F)c1cc(Cl)cnc1O[C@H]1C[C@@H](C(=O)O)N(C1)c1nc(nc3c1oc1ccccc13)C(F)(F)CCCCCOC2=O ; C[C@H]1CN2CC[C@]1(F)c1cc(Cl)cnc1O[C@H]1C[C@@H](C(=O)O)N(C1)c1nc(nc3c1oc1ccccc13)C(F)(F)CCCCCC(F)(F)C2=O]

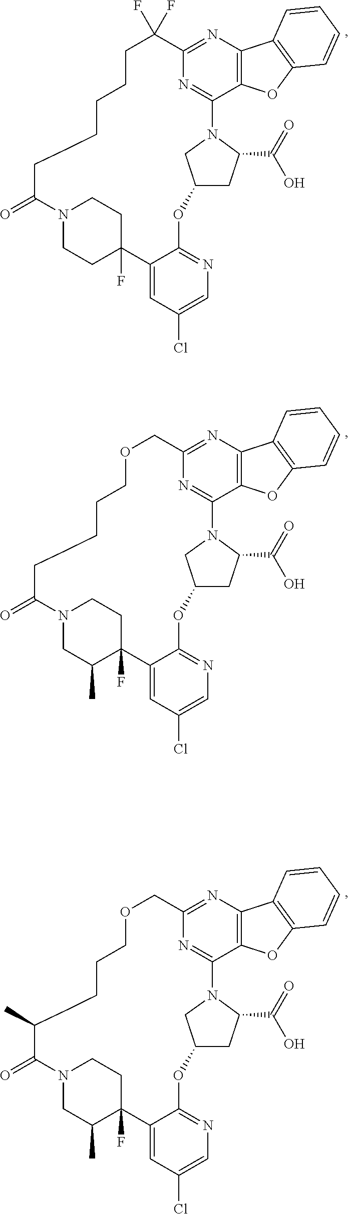 [SMILES: O=C(O)[C@@H]1C[C@H]2CN1c1nc(nc3c1oc1ccccc13)C(F)(F)CCCCCCC(=O)N1CCC(F)(CC1)c1cc(Cl)cnc1O2 ; C[C@H]1CN2CC[C@]1(F)c1cc(Cl)cnc1O[C@H]1C[C@@H](C(=O)O)N(C1)c1nc(nc3c1oc1ccccc13)COCCCCCC2=O ; C[C@H]1CCCCOCc2nc(c3oc4ccccc4c3n2)N2C[C@H](C[C@H]2C(=O)O)Oc2ncc(Cl)cc2[C@@]2(F)CCN(C[C@@H]2C)C1=O]

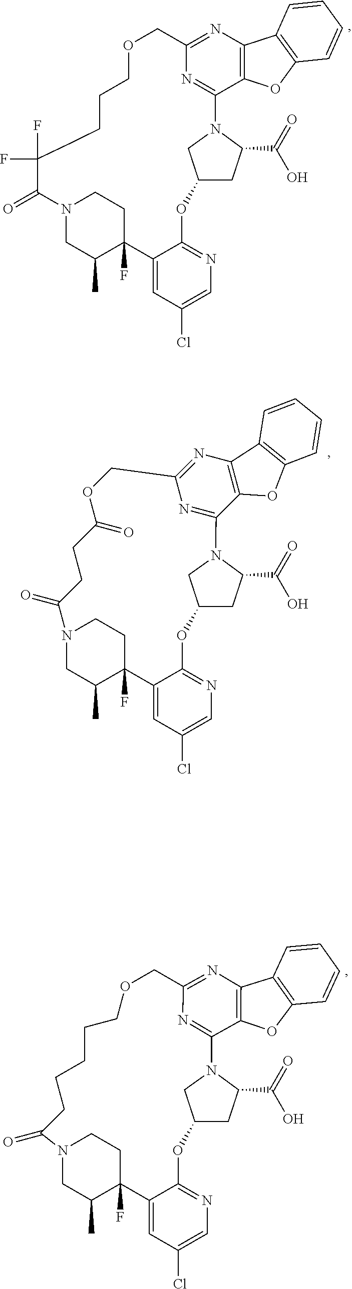 [SMILES: C[C@H]1CN2CC[C@]1(F)c1cc(Cl)cnc1O[C@H]1C[C@@H](C(=O)O)N(C1)c1nc(nc3c1oc1ccccc13)COCCCCC(F)(F)C2=O ; C[C@H]1CN2CC[C@]1(F)c1cc(Cl)cnc1O[C@H]1C[C@@H](C(=O)O)N(C1)c1nc(nc3c1oc1ccccc13)COC(=O)CCC2=O ; C[C@H]1CN2CC[C@]1(F)c1cc(Cl)cnc1O[C@H]1C[C@@H](C(=O)O)N(C1)c1nc(nc3c1oc1ccccc13)COCCCCCC2=O]

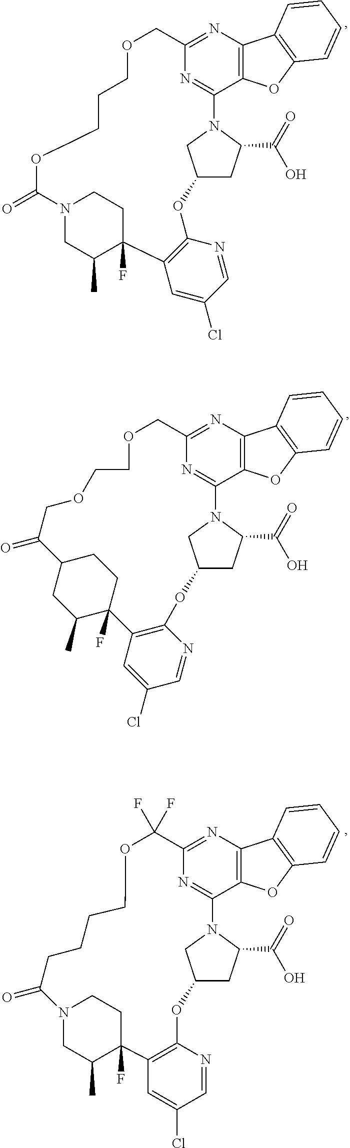 [SMILES: C[C@H]1CN2CC[C@]1(F)c1cc(Cl)cnc1O[C@H]1C[C@@H](C(=O)O)N(C1)c1nc(nc3c1oc1ccccc13)COCCCCOC2=O ; C[C@H]1CC2CC[C@]1(F)c1cc(Cl)cnc1O[C@H]1C[C@@H](C(=O)O)N(C1)c1nc(nc3c1oc1ccccc13)COCCOCC2=O ; C[C@H]1CN2CC[C@]1(F)c1cc(Cl)cnc1O[C@H]1C[C@@H](C(=O)O)N(C1)c1nc(nc3c1oc1ccccc13)C(F)(F)OCCCCC2=O]

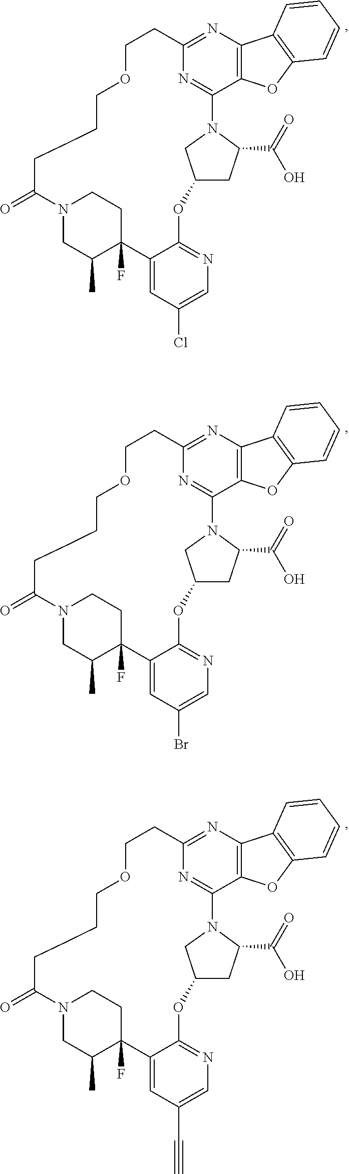 [SMILES: C[C@H]1CN2CC[C@]1(F)c1cc(Cl)cnc1O[C@H]1C[C@@H](C(=O)O)N(C1)c1nc(nc3c1oc1ccccc13)CCOCCCCC2=O ; C[C@H]1CN2CC[C@]1(F)c1cc(Br)cnc1O[C@H]1C[C@@H](C(=O)O)N(C1)c1nc(nc3c1oc1ccccc13)CCOCCCCC2=O ; C#Cc1cnc2c(c1)[C@@]1(F)CCN(C[C@@H]1C)C(=O)CCCCOCCc1nc(c3oc4ccccc4c3n1)N1C[C@H](C[C@H]1C(=O)O)O2]

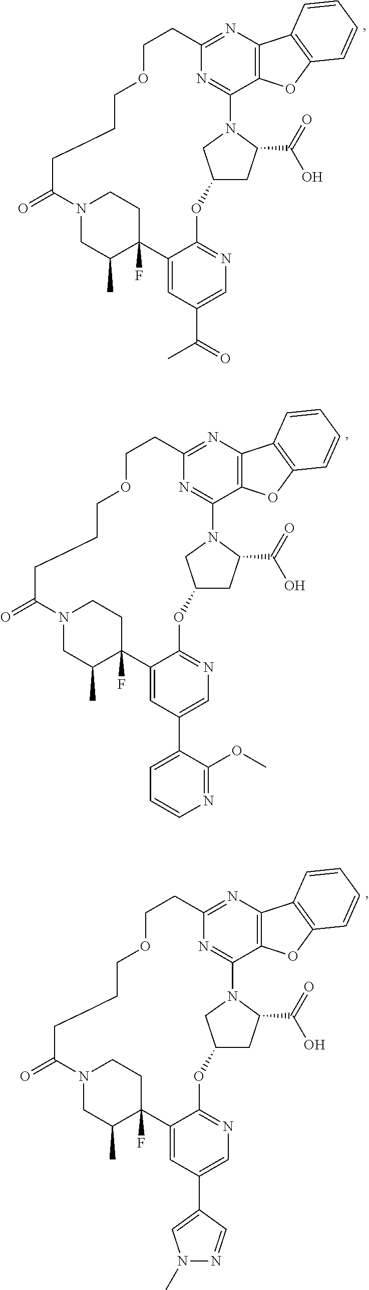 [SMILES: CC(=O)c1cnc2c(c1)[C@@]1(F)CCN(C[C@@H]1C)C(=O)CCCCOCCc1nc(c3oc4ccccc4c3n1)N1C[C@H](C[C@H]1C(=O)O)O2 ; COc1ncccc1-c1cnc2c(c1)[C@@]1(F)CCN(C[C@@H]1C)C(=O)CCCCOCCc1nc(c3oc4ccccc4c3n1)N1C[C@H](C[C@H]1C(=O)O)O2 ; C[C@H]1CN2CC[C@]1(F)c1cc(-c3cnn(C)c3)cnc1O[C@H]1C[C@@H](C(=O)O)N(C1)c1nc(nc3c1oc1ccccc13)CCOCCCCC2=O]

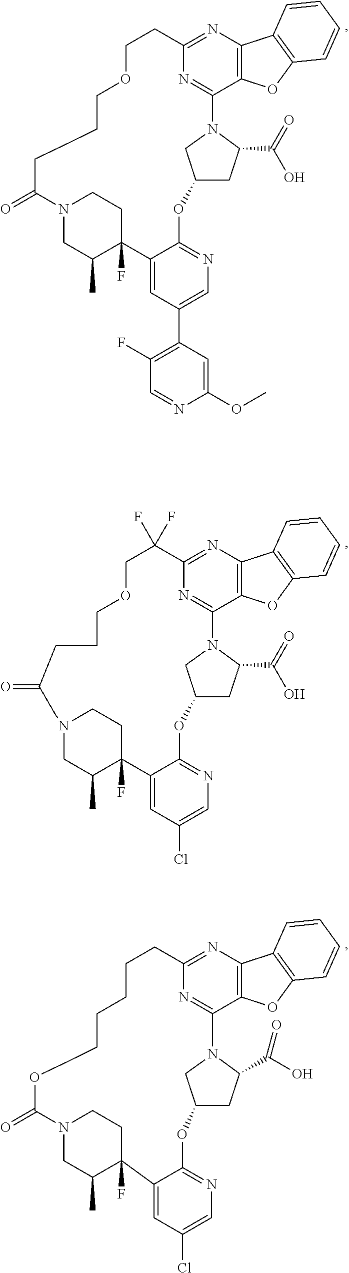 [SMILES: COc1cc(-c2cnc3c(c2)[C@@]2(F)CCN(C[C@@H]2C)C(=O)CCCCOCCc2nc(c4oc5ccccc5c4n2)N2C[C@H](C[C@H]2C(=O)O)O3)c(F)cn1 ; C[C@H]1CN2CC[C@]1(F)c1cc(Cl)cnc1O[C@H]1C[C@@H](C(=O)O)N(C1)c1nc(nc3c1oc1ccccc13)C(F)(F)COCCCC(=O)C2 ; C[C@H]1CN2CC[C@]1(F)c1cc(Cl)cnc1O[C@H]1C[C@@H](C(=O)O)N(C1)c1nc(nc3c1oc1ccccc13)CCCCCCOC2=O]

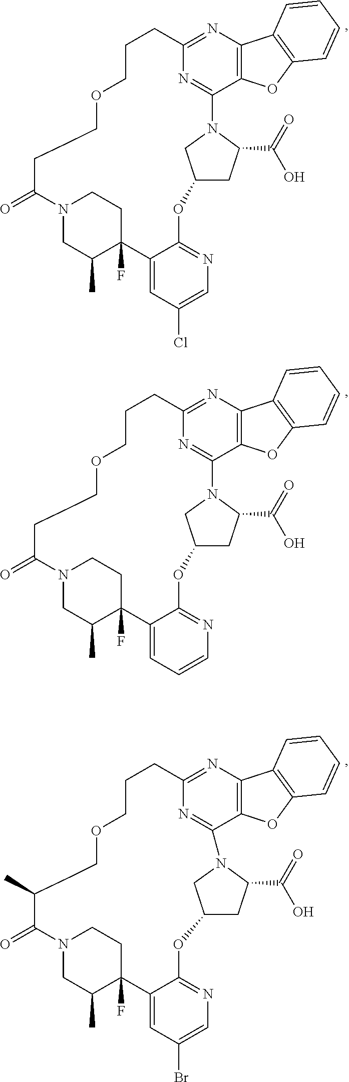 [SMILES: C[C@H]1CN2CC[C@]1(F)c1cc(Cl)cnc1O[C@H]1C[C@@H](C(=O)O)N(C1)c1nc(nc3c1oc1ccccc13)CCCOCCCC2=O ; C[C@H]1CN2CC[C@]1(F)c1cccnc1O[C@H]1C[C@@H](C(=O)O)N(C1)c1nc(nc3c1oc1ccccc13)CCCOCCCC2=O ; C[C@H]1CCOCCCc2nc(c3oc4ccccc4c3n2)N2C[C@H](C[C@H]2C(=O)O)Oc2ncc(Br)cc2[C@@]2(F)CCN(C[C@@H]2C)C1=O]

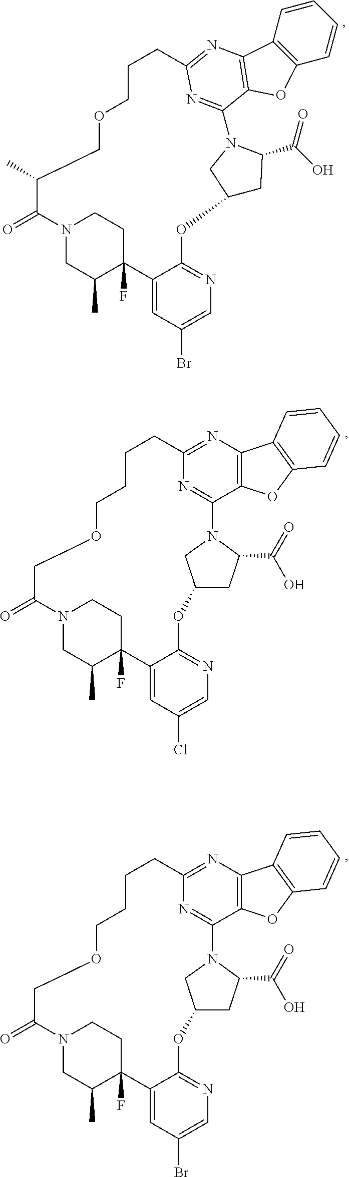 [SMILES: C[C@@H]1CCOCCCc2nc(c3oc4ccccc4c3n2)N2C[C@H](C[C@H]2C(=O)O)Oc2ncc(Br)cc2[C@@]2(F)CCN(C[C@@H]2C)C1=O ; C[C@H]1CN2CC[C@]1(F)c1cc(Cl)cnc1O[C@H]1C[C@@H](C(=O)O)N(C1)c1nc(nc3c1oc1ccccc13)CCCCOCCC2=O ; C[C@H]1CN2CC[C@]1(F)c1cc(Br)cnc1O[C@H]1C[C@@H](C(=O)O)N(C1)c1nc(nc3c1oc1ccccc13)CCCCOCCC2=O]

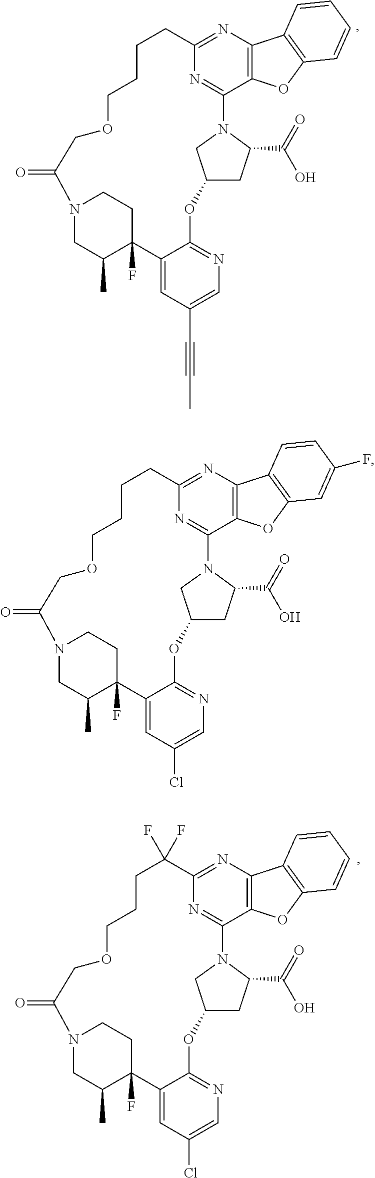 [SMILES: CC#Cc1cnc2c(c1)[C@@]1(F)CCN(C[C@@H]1C)C(=O)COCCCCc1nc(c3oc4ccccc4c3n1)N1C[C@H](C[C@H]1C(=O)O)O2 ; C[C@H]1CN2CC[C@]1(F)c1cc(Cl)cnc1O[C@H]1C[C@@H](C(=O)O)N(C1)c1nc(nc3c1oc1cc(F)ccc13)CCCCOCC2=O ; C[C@H]1CN2CC[C@]1(F)c1cc(Cl)cnc1O[C@H]1C[C@@H](C(=O)O)N(C1)c1nc(nc3c1oc1ccccc13)C(F)(F)CCCOCC2=O]

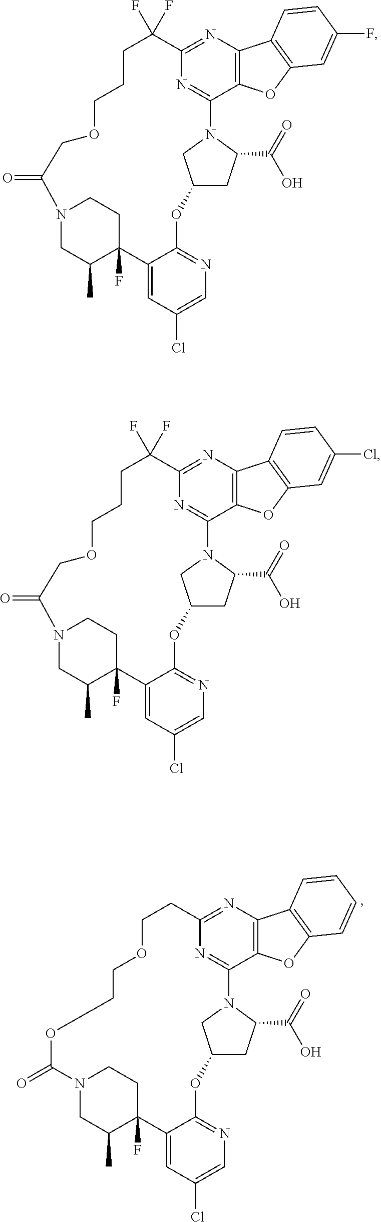 [SMILES: C[C@H]1CN2CC[C@]1(F)c1cc(Cl)cnc1O[C@H]1C[C@@H](C(=O)O)N(C1)c1nc(nc3c1oc1cc(F)ccc13)C(F)(F)CCCOCC2=O ; C[C@H]1CN2CC[C@]1(F)c1cc(Cl)cnc1O[C@H]1C[C@@H](C(=O)O)N(C1)c1nc(nc3c1oc1cc(Cl)ccc13)C(F)(F)CCCOCC2=O ; C[C@H]1CN2CC[C@]1(F)c1cc(Cl)cnc1O[C@H]1C[C@@H](C(=O)O)N(C1)c1nc(nc3c1oc1ccccc13)CCOCCOC2=O]

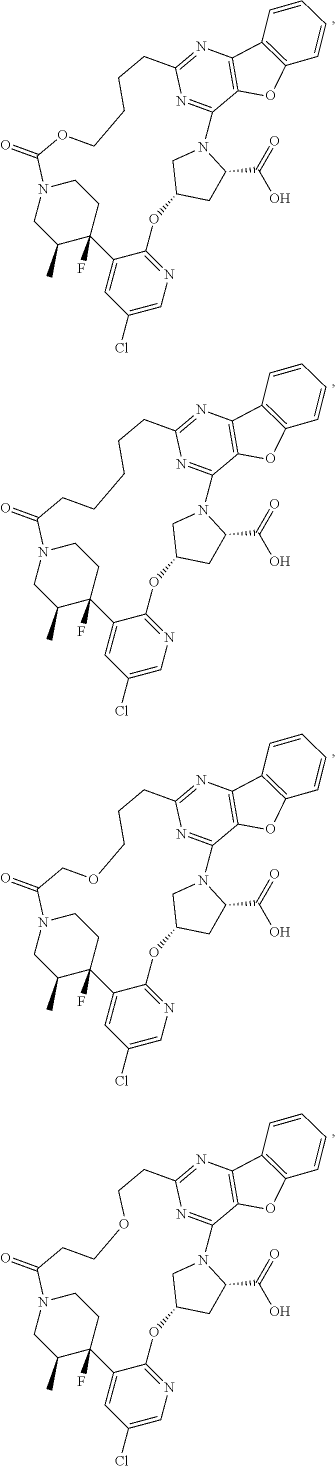 [SMILES: C[C@H]1CN2CC[C@]1(F)c1cc(Cl)cnc1O[C@H]1C[C@@H](C(=O)O)N(C1)c1nc(nc3c1oc1ccccc13)CCCCOC2=O ; C[C@H]1CN2CC[C@]1(F)c1cc(Cl)cnc1O[C@H]1C[C@@H](C(=O)O)N(C1)c1nc(nc3c1oc1ccccc13)CCCCCC2=O ; C[C@H]1CN2CC[C@]1(F)c1cc(Cl)cnc1O[C@H]1C[C@@H](C(=O)O)N(C1)c1nc(nc3c1oc1ccccc13)CCCOCC2=O ; C[C@H]1CN2CC[C@]1(F)c1cc(Cl)cnc1O[C@H]1C[C@@H](C(=O)O)N(C1)c1nc(nc3c1oc1ccccc13)CCOCCC2=O]

and prodrugs, deuterated analogues and pharmaceutical acceptable salts thereof.

In a further preferred embodiment the invention relates to the above-mentioned compound of formula I or to the above-mentioned compound of formula II, wherein
A is selected from the group consisting of —CH₂-and —O—;
D is selected from the group consisting of —CH₂-and —O—;
E is selected from the group consisting of-CH₂— and —O—;
G is selected from the group consisting of —CH₂-and —O—;
J is selected from the group consisting of —CH₂— and —O—;
K is either selected from the group consisting of —CH₂— and —CF₂—;
L is absent;
and prodrugs, deuterated analogues and pharmaceutical acceptable salts thereof.

In another particularly preferred embodiment the invention concerns the above-mentioned compound of formula II, which is selected from the group consisting of

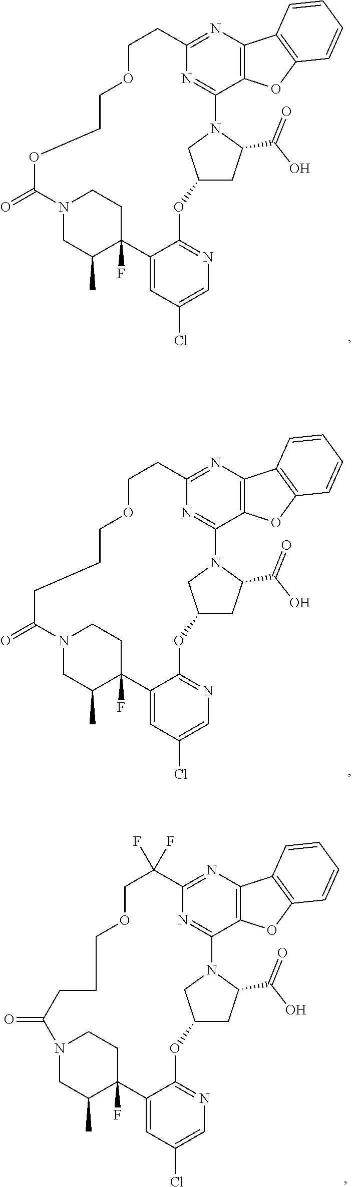 [SMILES: C[C@H]1CN2CC[C@]1(F)c1cc(Cl)cnc1O[C@H]1C[C@@H](C(=O)O)N(C1)c1nc(nc3c1oc1ccccc13)CCOCCOC2=O ; C[C@H]1CN2CC[C@]1(F)c1cc(Cl)cnc1O[C@H]1C[C@@H](C(=O)O)N(C1)c1nc(nc3c1oc1ccccc13)CCOCCCC2=O ; C[C@H]1CN2CC[C@]1(F)c1cc(Cl)cnc1O[C@H]1C[C@@H](C(=O)O)N(C1)c1nc(nc3c1oc1ccccc13)C(F)(F)COCCCC2=O]

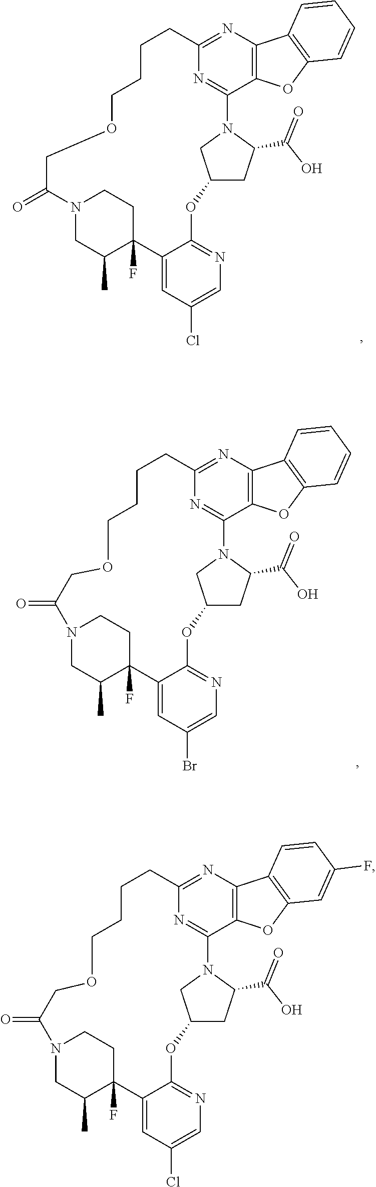 [SMILES: C[C@H]1CN2CC[C@]1(F)c1cc(Cl)cnc1O[C@H]1C[C@@H](C(=O)O)N(C1)c1nc(nc3c1oc1ccccc13)CCCCOCC2=O ; C[C@H]1CN2CC[C@]1(F)c1cc(Br)cnc1O[C@H]1C[C@@H](C(=O)O)N(C1)c1nc(nc3c1oc1ccccc13)CCCCOCC2=O ; C[C@H]1CN2CC[C@]1(F)c1cc(Cl)cnc1O[C@H]1C[C@@H](C(=O)O)N(C1)c1nc(nc3c1oc1cc(F)ccc13)CCCCOCC2=O]

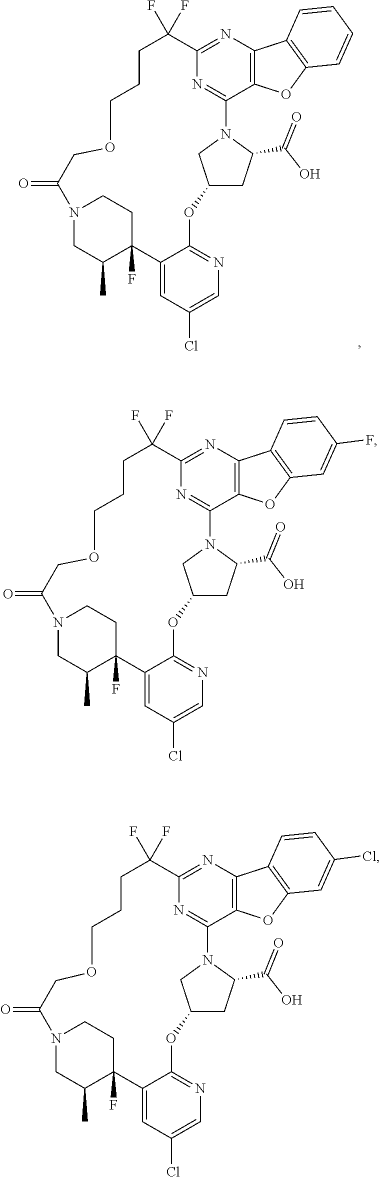 [SMILES: C[C@H]1CN2CC[C@]1(F)c1cc(Cl)cnc1O[C@H]1C[C@@H](C(=O)O)N(C1)c1nc(nc3c1oc1ccccc13)C(F)(F)CCCOCC2=O ; C[C@H]1CN2CC[C@]1(F)c1cc(Cl)cnc1O[C@H]1C[C@@H](C(=O)O)N(C1)c1nc(nc3c1oc1cc(F)ccc13)C(F)(F)CCCOCC2=O ; C[C@H]1CN2CC[C@]1(F)c1cc(Cl)cnc1O[C@H]1C[C@@H](C(=O)O)N(C1)c1nc(nc3c1oc1cc(Cl)ccc13)C(F)(F)CCCOCC2=O]

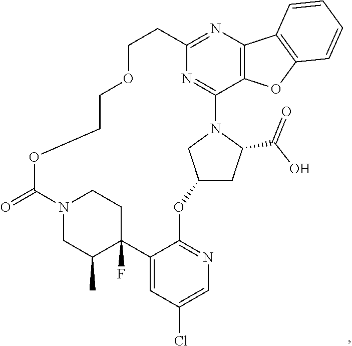 [SMILES: C[C@H]1CN2CC[C@]1(F)c1cc(Cl)cnc1O[C@H]1C[C@@H](C(=O)O)N(C1)c1nc(nc3c1oc1ccccc13)CCOCCOC2=O]

and prodrugs, deuterated analogues and pharmaceutical acceptable salts thereof.

In another particularly preferred embodiment the invention concerns the above-mentioned compound of formula I or the above-mentioned compound of formula II, wherein
A is selected from the group consisting of —CH₂-and —O—;
D is selected from the group consisting of —CH₂-and —O—;
E is —CH₂—;
G is selected from the group consisting of —CH₂-and —O—;
J is —CH₂—;
K is either selected from the group consisting of —CH₂— and —CF₂—;
L is absent;
and prodrugs, deuterated analogues and pharmaceutical acceptable salts thereof.

In another particularly preferred embodiment the invention concerns the above-mentioned compound of formula II, which is selected from the group consisting of

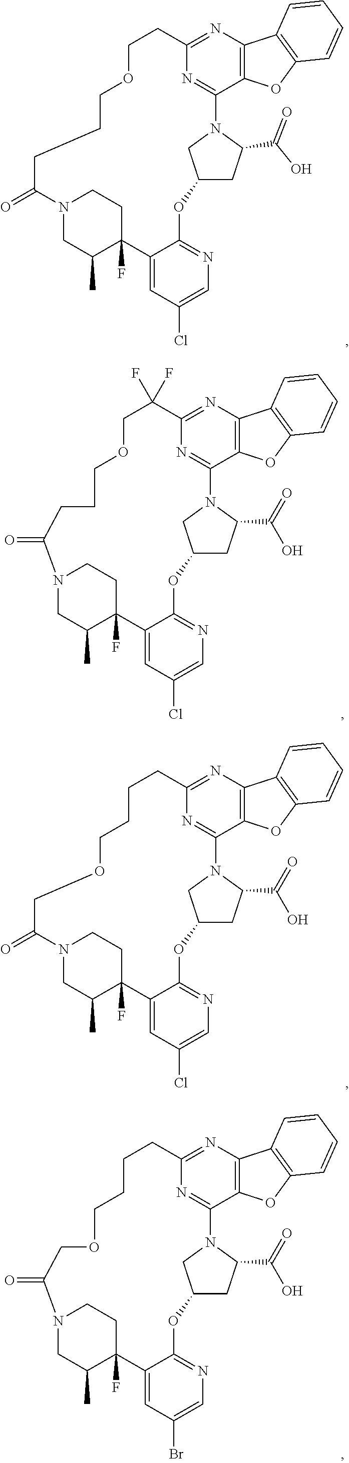

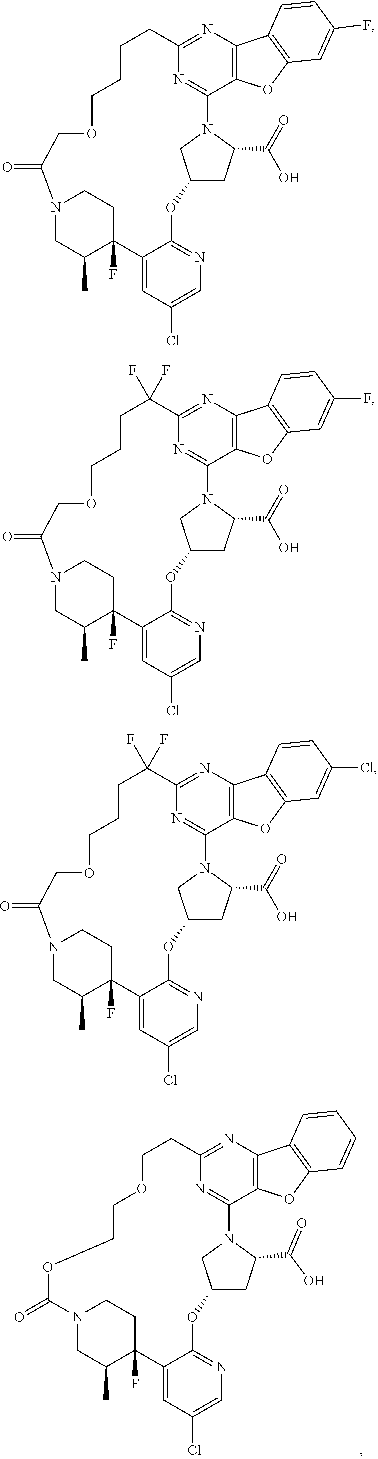

and prodrugs, deuterated analogues and pharmaceutical acceptable salts thereof.

Prodrugs of the compounds of formula I are preferably compounds of formula Ia

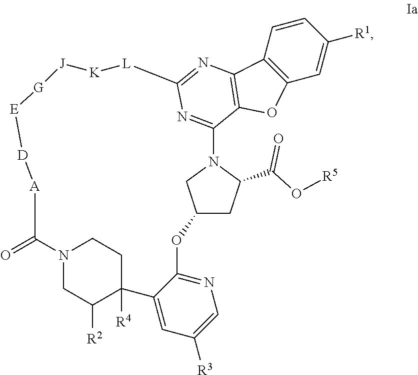

wherein variables R¹, R², R³, R⁴, A, D, E, G, J, K and L are defined as aforementioned and
wherein R³ is C₁₋₄-alkyl, aryl, —CH₂-aryl, NH—SO₂—C₁₋₃-alkyl.

Particularly preferred are the prodrugs of formula Ia, wherein variables R¹, R², R³, R⁴, A, D, E, G, J, K and L are defined as aforementioned and wherein R⁵ is methyl.

Prodrugs of the compounds of formula II are preferably compounds of formula IIa

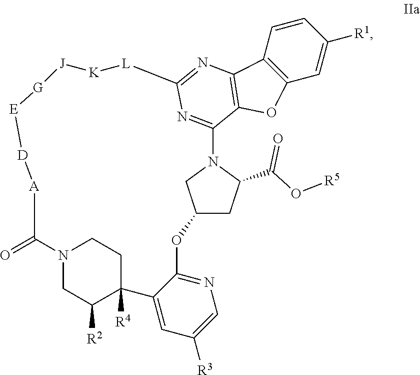

wherein variables R¹, R², R³, R⁴, A, D, E, G, J, K and L are defined as aforementioned and
wherein R³ is C₁₋₄-alkyl, aryl, —CH₂-aryl, NH—SO₂—C₁₋₃-alkyl.

Particularly preferred are the prodrugs of formula Ia, wherein variables R¹, R², R³, R⁴, A, D, E, G, J, K and L are defined as aforementioned and wherein R⁵ is methyl.

In another preferred embodiment the invention relates to
a) intermediate compounds of formula (A-I)

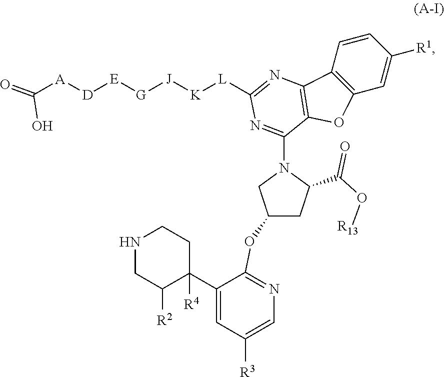

wherein R¹, R², R³, R⁴, A, D, E, G, J, K and L are defined as above-mentioned and wherein R₁₃ is selected from the group consisting of hydrogen, methyl, ethyl and tert-butyl,
b) intermediate compounds of formula (A-II)

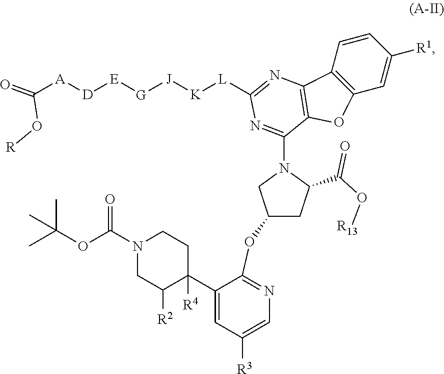

wherein R¹, R², R³, R⁴, A, D, E, G, J, K and L are defined as above-mentioned,
wherein R₁₃ is selected from the group consisting of hydrogen, methyl, ethyl and tert-butyl,
and wherein R is either hydrogen or a protecting group selected from the group consisting of tert-butyl, methyl, ethyl and benzyl,
c) intermediate compounds of formula (B-I)

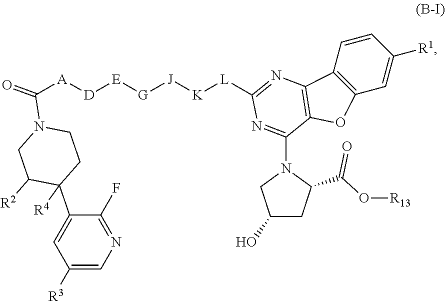

wherein R¹, R², R³, R⁴, A, D, E, G, J, K and L are defined as above-mentioned and wherein R₁ is selected from the group consisting of hydrogen, methyl, ethyl and tert-butyl,
d) intermediate compounds of formula (C-I)

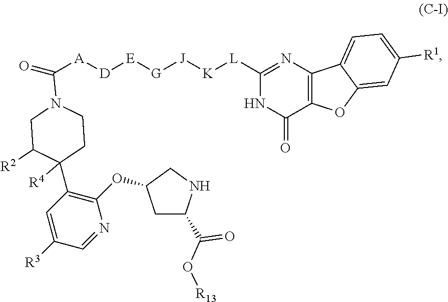

wherein R¹, R², R³, R⁴, A, D, E, G, J, K and L are defined as above-mentioned and wherein R₁ is selected from the group consisting of hydrogen, methyl, ethyl and tert-butyl,
   or
e) formula (C-II)

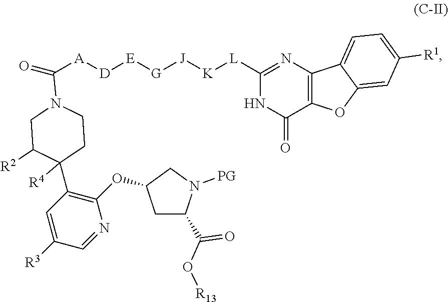

wherein R¹, R², R³, R⁴, A, D, E, G, J, K and L are defined as above-mentioned and wherein R₁ is selected from the group consisting of hydrogen, methy, ethyl and tert-butyl,
and wherein PG is selected from the group consisting of tert-butoxycarbonyl (Boc), allyloxycarbonyl (Alloc), benzyloxycarbonyl (Cbz) and fluorenylmethoxycarbonyl (Fmoc).

In a further preferred embodiment the invention concerns an above-mentioned compound of formula I or an above-mentioned compound of formula II or a prodrug of formula Ia or IIa, deuterated analogues and pharmaceutical acceptable salts thereof, for use in the treatment of a disease that can be treated by the inhibition of cGAS.

In a further preferred embodiment the invention relates to an above-mentioned compound of formula I or II or a prodrug of formula Ia or IIa, deuterated analogues and pharmaceutical acceptable salts thereof, for use in the treatment of a disease selected from the group consisting of systemic lupus erythematosus (SLE), interferonopathies, Aicardi-Goutières syndrome (AGS), COPA syndrome, familial chilblain lupus, age-related macular degeneration (AMD), retinopathy, glaucoma, amyotrophic lateral sclerosis (ALS), diabetes, obesity, inflammatory bowel disease (IBD), chronic obstructive pulmonary disease (COPD), Bloom's syndrome, Sjogren's syndrome, Parkinsons disease, heart failure and cancer, systemic sclerosis (SSc), dermatomyositis, non-alcoholic steatotic hepatitis (NASH), interstitial lung disease (ILD), preferably progressive fibrosing interstitial lung disease (PF-ILD), in particular idiopathic pulmonary fibrosis (IPF). Aging, muscle disorders, sepsis, rheumatoid arthritis, osteoarthritis and COVID-19.

In another preferred embodiment the invention relates to an above-mentioned compound of formula I or II or a prodrug of formula Ia or IIa, deuterated analogues and pharmaceutical acceptable salts thereof, for use in the treatment of a disease selected from the group consisting of systemic lupus erythematosus (SLE), interferonopathies, Aicardi-Goutières syndrome (AGS), COPA syndrome, familial chilblain lupus, dermatomyositis, age-related macular degeneration (AMD), amyotrophic lateral sclerosis (ALS), inflammatory bowel disease (IBD), chronic obstructive pulmonary disease (COPD), Bloom's syndrome, Sjogren's syndrome, rheumatoid arthritis and Parkinsons disease.

In a further preferred embodiment the invention relates to an above-mentioned compound of formula I or II or a prodrug of formula Ia or IIa, deuterated analogues and pharmaceutical acceptable salts thereof, for use in the treatment of a disease selected from the group consisting of systemic sclerosis (SSc), non-alcoholic steatohepatitis (NASH), interferonopathies, interstitial lung disease (ILD), preferably progressive fibrosing interstitial lung disease (PF-ILD), in particular idiopathic pulmonary fibrosis (IPF).

In another preferred embodiment the invention relates to an above-mentioned compound of formula I or II or a prodrug of formula Ia or IIa, deuterated analogues and pharmaceutical acceptable salts thereof, for use in the treatment of a disease selected from the group consisting of age-related macular degeneration (AMD), retinopathy, glaucoma, diabetes, obesity, aging, muscle disorders, sepsis, osteoarthritis, heart failure, COVID-19/SARS-CoV-2 infection, renal inflammation, renal fibrosis, dysmetabolism, vascular diseases, cardiovascular diseases and cancer.

In another preferred embodiment the invention relates to a pharmaceutical composition comprising an above-mentioned compound of formula I or II or a prodrug of formula Ia or IIa, deuterated analogues and pharmaceutical acceptable salts thereof, and optionally one or more pharmaceutically acceptable carriers and/or excipients.

In another preferred embodiment the invention relates to a pharmaceutical composition comprising an above-mentioned compound of formula I or II or a prodrug of formula Ia or IIa, deuterated analogues and pharmaceutical acceptable salts thereof, in combination with one or more active agents selected from the group consisting of anti-inflammatory agents, anti-fibrotic agents, anti-allergic agents/anti-histamines, bronchodilators, beta 2 agonists/betamimetics, adrenergic agonists, anticholinergic agents, methotrexate, mycophenolate mofetil, leukotriene modulators, JAK inhibitors, anti-interleukin antibodies, non-specific immunotherapeutics such as interferons or other cytokines/chemokines, cytokine/chemokine receptor modulators, toll-like receptor agonists, immune checkpoint regulators, an anti-TNF antibody such as Humira™, an anti-BAFF antibody such as Belimumab and Etanercept, and optionally one or more pharmaceutically acceptable carriers and/or excipients.

In a further preferred embodiment the invention relates to a pharmaceutical composition comprising an above-mentioned compound of formula I or II or a prodrug of formula Ia or IIa, deuterated analogues and pharmaceutical acceptable salts thereof, and one or more anti-fibrotic agents selected from the group consisting of Pirfenidon and Nintedanib and optionally one or more pharmaceutically acceptable carriers and/or excipients.

In another preferred embodiment the invention relates to a pharmaceutical composition comprising an above-mentioned compound of formula I or II or a prodrug of formula Ia or IIa, deuterated analogues and pharmaceutical acceptable salts thereof, and one or more anti-inflammatory agents selected from the group consisting of NSAIDs and corticosteroids and optionally one or more pharmaceutically acceptable carriers and/or excipients.

In a further preferred embodiment the invention relates to a pharmaceutical composition comprising an above-mentioned compound of formula I or II or a prodrug of formula Ia or IIa, deuterated analogues and pharmaceutical acceptable salts thereof, and one or more active agents selected from the group consisting of bronchodilators, beta 2 agonists/betamimetics, adrenergic agonists and anticholinergic agents and optionally one or more pharmaceutically acceptable carriers and/or excipients.

In another preferred embodiment the invention relates to a pharmaceutical combination comprising an above-mentioned compound of formula I or II or a prodrug of formula Ia or IIa, deuterated analogues and pharmaceutical acceptable salts thereof, and one or more anti-interleukin antibodies selected from the group consisting of anti-IL-23 such as Risankizumab, anti-IL-17 antibodies, anti-IL-1 antibodies, anti-IL-4 antibodies, anti-IL-13 antibodies, anti-IL-5 antibodies, anti-IL-6 antibodies such as Actemra™, anti-IL-12 antibodies and anti-IL-15 antibodies.

### 3. TERMS AND DEFINITIONS USED

Unless stated otherwise, all the substituents are independent of one another. If for example a number of C₁₋₆-alkyl groups are possible substituents at a group, in the case of three substituents, for example, C₁₋₆-alkyl could represent, independently of one another, a methyl, a n-propyl and a tert-butyl.

A crossed bond like the middle bond in following butyl-molecule

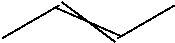

represents a double bond of unknown configuration (either cis, trans or a mixture thereof).

By the term “C₁₋₆-alkyl” (including those which are part of other groups) are meant branched and unbranched alkyl groups with 1 to 6 carbon atoms and by the term “C₁₋₃-alkyl” are meant branched and unbranched alkyl groups with 1 to 3 carbon atoms. “C₁₋₄-alkyl” accordingly denotes branched and unbranched alkyl groups with 1 to 4 carbon atoms. Alkyl groups with 1 to 4 carbon atoms are preferred. Examples of these include methyl, ethyl, n-propyl, iso-propyl, n-butyl, iso-butyl, sec-butyl, tert-butyl, n-pentyl, iso-pentyl, neo-pentyl and hexyl. The abbreviations Me, Et, n-Pr, i-Pr, n-Bu, i-Bu, t-Bu, etc., may also optionally be used for the above-mentioned groups. Unless stated otherwise, the definitions propyl, butyl, pentyl and hexyl include all the possible isomeric forms of the groups in question. Thus, for example, propyl includes n-propyl and iso-propyl, butyl includes iso-butyl, sec-butyl and tert-butyl etc.

By the term “C₁₋₆-alkylene” (including those which are part of other groups) are meant branched and unbranched alkylene groups with 1 to 6 carbon atoms and by the term “C₁₋₄-alkylene” are meant branched and unbranched alkylene groups with 1 to 4 carbon atoms. Alkylene groups with 1 to 4 carbon atoms are preferred. Examples of these include methylene, ethylene, propylene, 1-methylethylene, butylene, 1-methylpropylene, 1,1-dimethylethylene, 1,2-dimethylethylene, pentylene, 1,1-dimethylpropylene, 2,2-dimethylpropylene, 1,2-dimethylpropylene, 1,3-dimethylpropylene and hexylene. Unless stated otherwise, the definitions propylene, butylene, pentylene and hexylene include all the possible isomeric forms of the groups in question with the same number of carbons. Thus, for example, propyl includes also 1-methylethylene and butylene includes 1-methylpropylene, 1,1-dimethylethylene, 1,2-dimethylethylene etc.

If the carbon chain is substituted by a group which together with one or two carbon atoms of the alkylene chain forms a carbocyclic ring with 3, 5 or 6 carbon atoms, this includes, inter alia, the following examples of the rings:

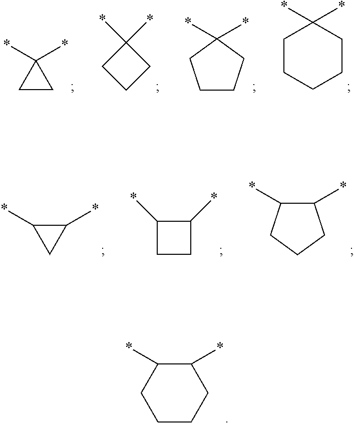

By the term “C₂₋₆-alkenyl” (including those which are part of other groups) are meant branched and unbranched alkenyl groups with 2 to 6 carbon atoms and by the term “C₂₋₄-alkenyl” are meant branched and unbranched alkenyl groups with 2 to 4 carbon atoms, provided that they have at least one double bond. Alkenyl groups with 2 to 4 carbon atoms are preferred. Examples include: ethenyl or vinyl, propenyl, butenyl, pentenyl or hexenyl. Unless stated otherwise, the definitions propenyl, butenyl, pentenyl and hexenyl include all the possible isomeric forms of the groups in question. Thus, for example, propenyl includes 1-propenyl and 2-propenyl, butenyl includes 1-, 2- and 3-butenyl, 1-methyl-1-propenyl, 1-methyl-2-propenyl etc.

By the term “C₂₋₅-alkynyl” (including those which are part of other groups) are meant branched and unbranched alkynyl groups with 2 to 5 carbon atoms and by the term “C₂₋₄-alkynyl” are meant branched and unbranched alkynyl groups with 2 to 4 carbon atoms, provided that they have at least one triple bond. Alkynyl groups with 2 to 4 carbon atoms are preferred.

By the term “C₂₋₆-alkenylene” (including those which are part of other groups) are meant branched and unbranched alkenylene groups with 2 to 6 carbon atoms and by the term “C₂₋₄-alkenylene” are meant branched and unbranched alkylene groups with 2 to 4 carbon atoms. Alkenylene groups with 2 to 4 carbon atoms are preferred. Examples of these include: ethenylene, propenylene, 1-methylethenylene, butenylene, 1-methylpropenylene, 1,1-dimethylethenylene, 1,2-dimethylethenylene, pentenylene, 1,1-dimethylpropenylene, 2,2-dimethylpropenylene, 1,2-dimethylpropenylene, 1,3-dimethylpropenylene and hexenylene. Unless stated otherwise, the definitions propenylene, butenylene, pentenylene and hexenylene include all the possible isomeric forms of the groups in question with the same number of carbons. Thus, for example, propenyl also includes 1-methylethenylene and butenylene includes 1-methylpropenylene, 1,1-dimethylethenylene, 1, 2-dimethylethenylene.

By the term “aryl” (including those which are part of other groups) are meant aromatic ring systems with 6 or 10 carbon atoms. Examples include phenyl or naphthyl, the preferred aryl group being phenyl. Unless otherwise stated, the aromatic groups may be substituted by one or more groups selected from among methyl, ethyl, iso-propyl, tert-butyl, hydroxy, fluorine, chlorine, bromine and iodine.

By the term “aryl-C₁₋₆-alkylene” (including those which are part of other groups) are meant branched and unbranched alkylene groups with 1 to 6 carbon atoms, which are substituted by an aromatic ring system with 6 or 10 carbon atoms. Examples include benzyl, 1- or 2-phenylethyl and 1- or 2-naphthylethyl. Unless otherwise stated, the aromatic groups may be substituted by one or more groups selected from among methyl, ethyl, iso-propyl, tert-butyl, hydroxy, fluorine, chlorine, bromine and iodine.

By the term “heteroaryl-C₁₋₆-alkylene” (including those which are part of other groups) are meant—even though they are already included under “aryl-C₁₋₆-alkylene”-branched and unbranched alkylene groups with 1 to 6 carbon atoms, which are substituted by a heteroaryl.

If not specifically defined otherwise, a heteroaryl of this kind includes five- or six-membered heterocyclic aromatic groups or 5-10-membered, bicyclic heteroaryl rings which may contain one, two, three or four heteroatoms selected from among oxygen, sulfur and nitrogen, and contain so many conjugated double bonds that an aromatic system is formed. The following are examples of five- or six-membered heterocyclic aromatic groups and bicyclic heteroaryl rings:

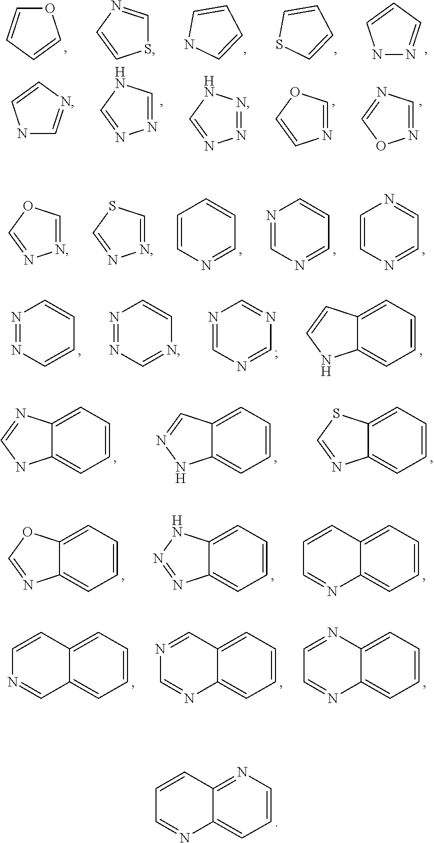

Unless otherwise stated, these heteroaryls may be substituted by one or more groups selected from among methyl, ethyl, iso-propyl, tert-butyl, hydroxy, amino, nitro, alkoxy, fluorine, chlorine, bromine and iodine.

The following are examples of heteroaryl-C₁₋₆-alkylenes:

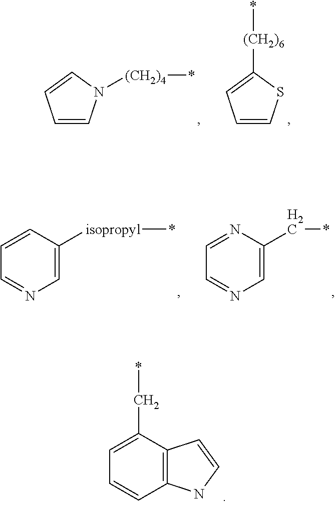

By the term “C₁₋₆-haloalkyl” (including those which are part of other groups) are meant branched and unbranched alkyl groups with 1 to 6 carbon atoms, which are substituted by one or more halogen atoms. By the term “C₁₋₄-haloalkyl” are meant branched and unbranched alkyl groups with 1 to 4 carbon atoms, which are substituted by one or more halogen atoms. Alkyl groups with 1 to 4 carbon atoms are preferred. Examples include: CF₃, CHF₂, CH₂F, CH₂CF₃.

By the term “C₃₋₇-cycloalkyl” (including those which are part of other groups) are meant cyclic alkyl groups with 3 to 7 carbon atoms, if not specifically defined otherwise. Examples include: cyclopropyl, cyclobutyl, cyclopentyl, cyclohexyl and cycloheptyl. Unless otherwise stated, the cyclic alkyl groups may be substituted by one or more groups selected from among methyl, ethyl, iso-propyl, tert-butyl, hydroxy, fluorine, chlorine, bromine and iodine.

If not specifically defined otherwise, by the term “C₃₋₁₀-cycloalkyl” are also meant monocyclic alkyl groups with 3 to 7 carbon atoms and also bicyclic alkyl groups with 7 to 10 carbon atoms, or monocyclic alkyl groups which are bridged by at least one C₁₋₃-carbon bridge.

By the term “heterocyclic rings” or “heterocycle” are meant, unless stated otherwise, five-, six- or seven-membered, saturated, partially saturated or unsaturated heterocyclic rings which may contain one, two or three heteroatoms selected from among oxygen, sulfur and nitrogen, while the ring may be linked to the molecule through a carbon atom or through a nitrogen atom, if there is one. Although included by the term “heterocyclic rings” or “heterocycles”, the term “saturated heterocyclic ring” refers to five-, six- or seven-membered saturated rings. Examples include:

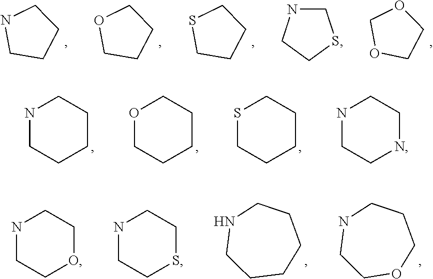

Although included by the term “heterocyclic rings” or “heterocyclic group”, the term “partially saturated heterocyclic group” refers to five-, six- or seven-membered partially saturated rings which contain one or two double bonds, without so many double bonds being produced that an aromatic system is formed, unless specifically defined otherwise. Examples include:

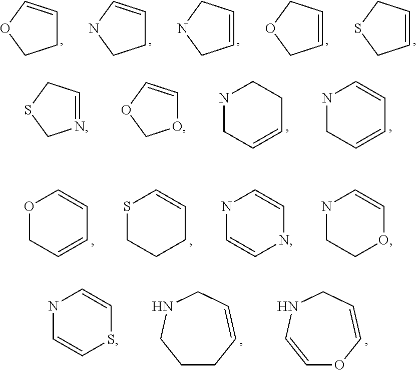

Although included by the term “heterocyclic rings” or “heterocycles”, the term “heterocyclic aromatic rings”, “unsaturated heterocyclic group” or “heteroaryl” refers to five- or six-membered heterocyclic aromatic groups or 5-10-membered, bicyclic heteroaryl rings which may contain one, two, three or four heteroatoms selected from among oxygen, sulfur and nitrogen, and contain so many conjugated double bonds that an aromatic system is formed, unless not specifically defined otherwise. Examples of five- or six-membered heterocyclic aromatic groups include:

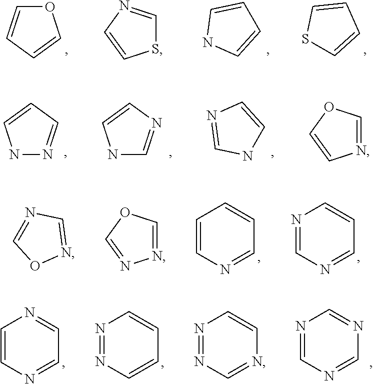

Unless otherwise mentioned, a hetercyclic ring (or heterocycle) may be provided with a keto group. Examples include:

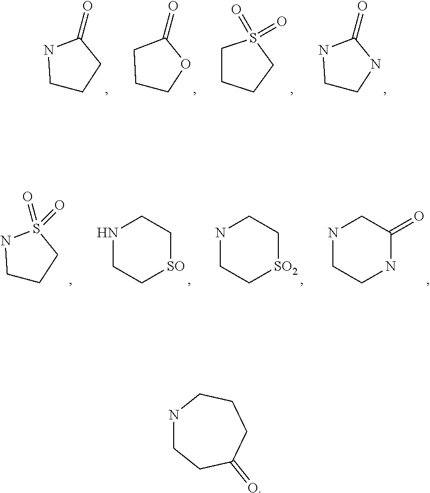

Although covered by the term “cycloalkyl”, the term “bicyclic cycloalkyls” generally denotes eight-, nine- or ten-membered bicyclic carbon rings. Examples include:

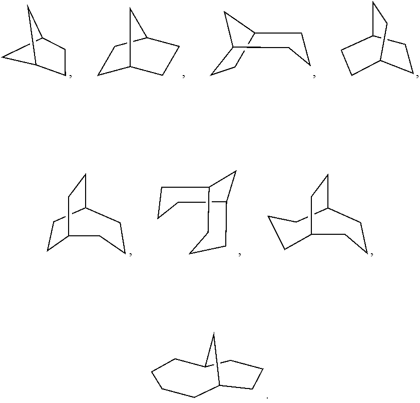

Although already included by the term “heterocycle”, the term “bicyclic heterocycles” generally denotes eight-, nine- or ten-membered bicyclic rings which may contain one or more heteroatoms, preferably 1-4, more preferably 1-3, even more preferably 1-2, particularly one heteroatom, selected from among oxygen, sulfur and nitrogen, unless not specifically defined otherwise. The ring may be linked to the molecule through a carbon atom of the ring or through a nitrogen atom of the ring, if there is one. Examples include:

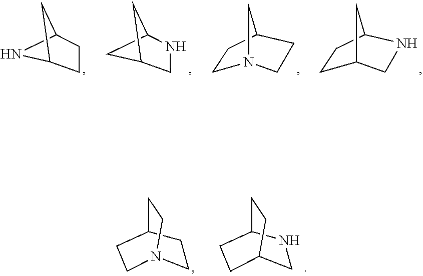

Although already included by the term “aryl”, the term “bicyclic aryl” denotes a 5-10 membered, bicyclic aryl ring which contains sufficient conjugated double bonds to form an aromatic system. One example of a bicyclic aryl is naphthyl.

Although already included under “heteroaryl”, the term “bicyclic heteroaryl” denotes a 5-10 membered, bicyclic heteroaryl ring which may contain one, two, three or four heteroatoms, selected from among oxygen, sulfur and nitrogen, and contains sufficient conjugated double bonds to form an aromatic system, unless specifically defined otherwise.

Although included by the term “bicyclic cycloalkyls” or “bicyclic aryl”, the term “fused cycloalkyl” or “fused aryl” denotes bicyclic rings wherein the bridge separating the rings denotes a direct single bond. The following are examples of a fused, bicyclic cycloalkyl:

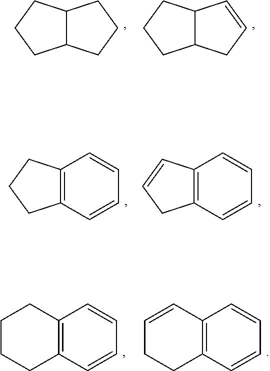

Although included by the term “bicyclic heterocycles” or “bicyclic heteroaryls”, the term “fused bicyclic heterocycles” or “fused bicyclic heteroaryls” denotes bicyclic 5-10 membered heterorings which contain one, two, three or four heteroatoms, selected from among oxygen, sulfur and nitrogen and wherein the bridge separating the rings denotes a direct single bond. The “fused bicyclic heteroaryls” moreover contain sufficient conjugated double bonds to form an aromatic system. Examples include pyrrolizine, indole, indolizine, isoindole, indazole, purine, quinoline, isoquinoline, benzimidazole, benzofuran, benzopyran, benzothiazole, benzothiazole, benzoisothiazole, pyridopyrimidine, pteridine, pyrimidopyrimidine,

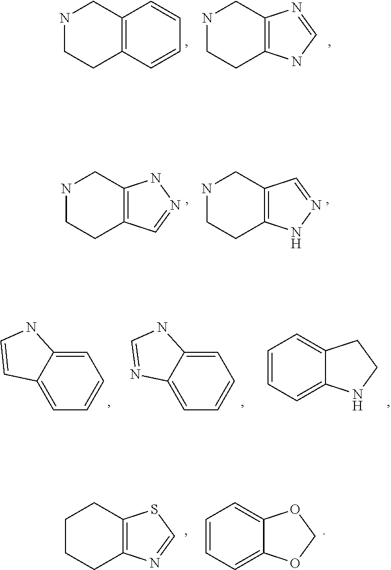

“Halogen” within the scope of the present invention denotes fluorine, chlorine, bromine or iodine. Unless stated to the contrary, fluorine, chlorine and bromine are regarded as preferred halogens.

As mentioned previously, the compounds of formulas I or II may be converted into the salts thereof, particularly for pharmaceutical use into the physiologically and pharmacologically acceptable salts thereof. The phrase “pharmaceutically acceptable” is employed herein to refer to those compounds, materials, compositions, and/or dosage forms which are, within the scope of sound medical judgement, suitable for use in contact with the tissue of human beings and animals without excessive toxicity, irritation, allergic response, or other problem or complication, and commensurate with a reasonable benefit/risk ratio. These salts may be present on the one hand as physiologically and pharmacologically acceptable acid addition salts of the compounds of formulas I or II with inorganic or organic acids. On the other hand, the compound of formulas I or II may be converted by reaction with inorganic bases into physiologically and pharmacologically acceptable salts with alkali or alkaline earth metal cations as counter-ion. The acid addition salts may be prepared for example using hydrochloric acid, hydrobromic acid, sulfuric acid, phosphoric acid, methanesulphonic acid, p-toluenesulfonic acid, acetic acid, fumaric acid, succinic acid, lactic acid, citric acid, tartaric acid or maleic acid. It is also possible to use mixtures of the above-mentioned acids. To prepare the alkali and alkaline earth metal salts of the compounds of formulas I or II it is preferable to use the alkali and alkaline earth metal hydroxides and hydrides, of which the hydroxides and hydrides of the alkali metals, particularly sodium, potassium, magnesium, calcium, zinc and diethanolamine, are preferred, while sodium and potassium hydroxide are particularly preferred.

The invention relates to the compounds in question, optionally in the form of the individual optical isomers, diastereomers, mixtures of diastereomers, mixtures of the individual enantiomers or racemates, in the form of the tautomers as well as in the form of the free bases or the corresponding acid addition salts with pharmacologically acceptable acids—such as for example acid addition salts with hydrohalic acids—for example hydrochloric or hydrobromic acid—or organic acids—such as for example oxalic, fumaric, diglycolic or methanesulfonic acid.

The compounds of formula I or II according to the invention may optionally be present as mixtures of diastereomeric isomers but may also be obtained as pure diastereoisomers. Preferred are the compounds with the specific stereochemistry of formula II or III, in particular the compounds of formula II.

### 4. METHODS OF SYNTHESIS

General Procedure

The following methods are suitable for preparing compounds of general formulas I or II. The compounds according to the invention may be obtained using methods of synthesis which are known to the one skilled in the art and described in the literature of organic synthesis. General methods for functional group protection and deprotection steps are described e.g. in: Greene, T. W. and Wuts, P.G.M. (eds.): Protective Groups in Organic Synthesis, third edition 1999; John Wiley and Sons, Inc. Preferably the compounds are obtained analogously to the methods of preparation explained more fully hereinafter, in particular as described in the experimental section. Compounds of general formula (I) may be prepared using several alternative synthetic routes, among which the following routes shall serve as examples.

Route A:

Compounds of general formulas I and II, especially with A representing —CH₂— or substituted —CH₂—, can be accessed from compounds of formula (A-I) through standard amidation procedures. R13 may thereby represent hydrogen or a protecting group like e.g. tert-butyl or methyl, which can be removed by standard deprotection methods. R may represent hydrogen or a protecting group like e.g. tert-butyl, benzyl, methyl or ethyl, which can be removed by standard deprotection methods. Compounds of formula (A-I) can be prepared from compounds of formula (A-II) applying standard deprotection methods. Compounds of formula (A-II) can be prepared by reacting compounds of formula (A-Ill) with compounds of formula (A-IV) applying a strong base like e.g. sodium hydride. Methods applicable for the preparation of compounds (A-Ill) become obvious to one skilled in the art from consulting routes C and D described below and examples described in the experimental section. Compounds of formula (A-IV) can be prepared through methods described hereinafter for the syntheses of intermediates P.

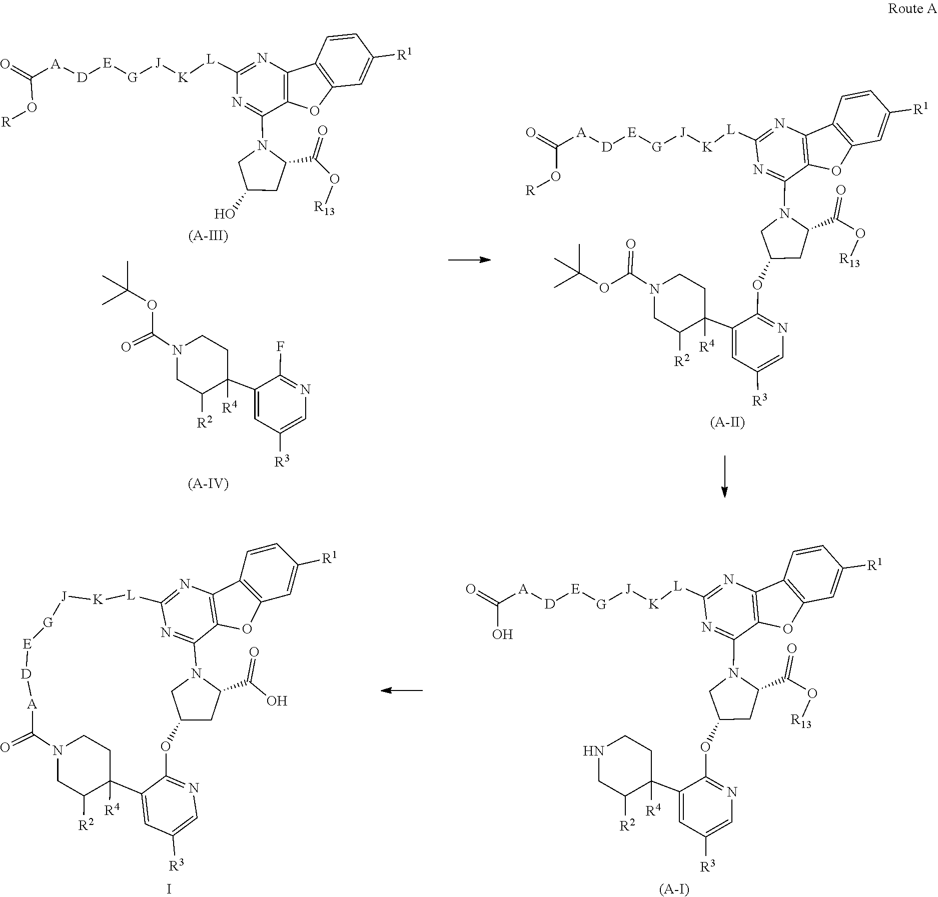

Route B:

Compounds of general formulas I and II can be prepared by reacting compounds of general formula (B-I) in the presence of a strong base like e.g. sodium hydride. R13 may thereby represent hydrogen or a protecting group like e.g. tert-butyl which can be removed by standard deprotection methods. Compounds (B-I) can be prepared by reacting compounds of the general formula (B-II) with compounds of the general formula (B-Ill) applying standard amidation conditions. Methods applicable for the preparation of compounds (B-Ill) become obvious to one skilled in the art from consulting the synthesis of intermediates P described in the experimental section. Alternatively, compounds of the general formula (B-I) can be prepared by reacting compounds of the general formula (B-IV) with compounds of the general formula (B-V) applying standard reaction conditions for transition metal catalyzed coupling reactions (as e.g. Heck reaction) followed by hydrogenation of the resulting alkene under standard hydrogenation conditions. Compounds of formula (B-V) can be prepared through methods described hereinafter for the syntheses of intermediates P.

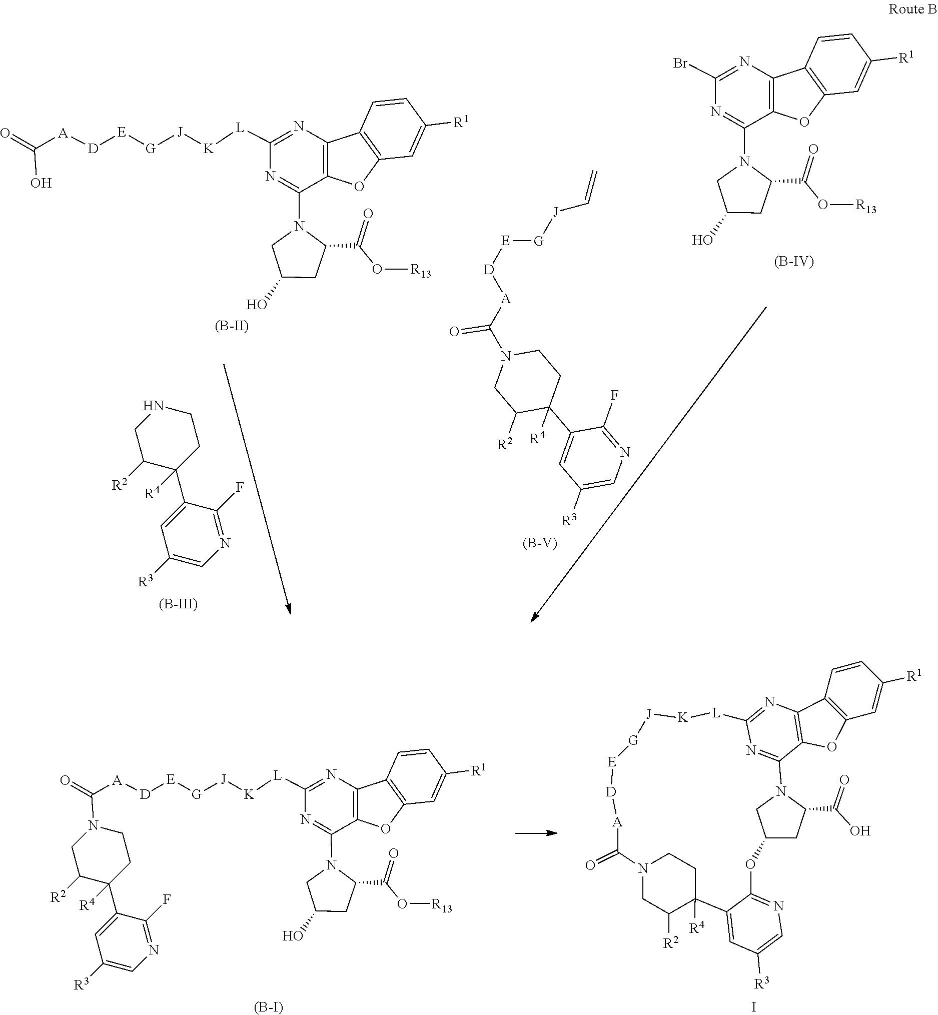

Route C:

Compounds of general formulas I and II can be prepared by reacting compounds of general formula (C-I) with an activating reagent like BOP (((1H-Benzo[d][1,2,3]triazol-1yl)oxy)tris(dimethylamino)-phosphonium hexafluorophosphate(V)) in the presence of a base, i.e. DBU. (C-I) is generated from (C-II) by removal of the protection group PG under standard deprotection conditions known to one skilled in the art. PG in formula (C-II) may represent a e.g. tert-butoxycarbonyl (Boc), allyloxycarbonyl (Alloc), benzyloxycarbonyl (Cbz). Compounds of the general formula (C-II) can be prepared from compounds of the general formula (C-III) with the respective enantiopure hydroxyproline (optionally bearing a protecting group R13), optionally in the presence of a base. Compounds of the general formula (C-III) are accessible from compounds of general formula (C-IV) applying alkaline conditions, e.g., triethylamine/TMSCI at elevated temperature. Compounds of the general formula (C-IV) can be synthesized from carboxylic acids of the general formula (C-VI), by reaction with amino-benzofurans of the general formula (C-V) under standard amidation conditions.

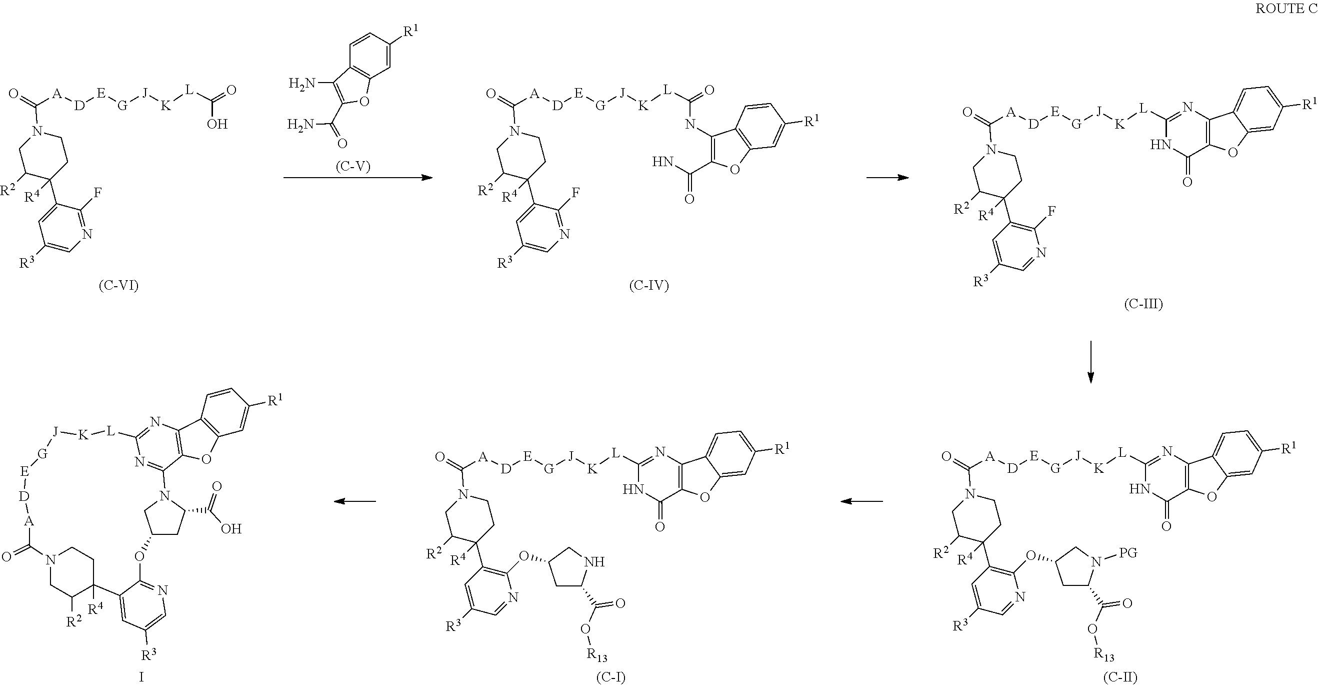

Route D:

Compounds of general formula I and II can be prepared from compounds of general formula (D-I) by various types of ring closing reactions, as exemplified by but not restricted to: Heck type coupling reactions (with R11 representing a bromine or iodine atom and R12 containing a terminal alkene), ring closing metathesis reaction (with both R11 and R12 containing a terminal alkene) followed by hydrogenation of the resulting alkene, amidation (with R11 bearing a carboxylic acid and R12 bearing a primary or secondary amino group or vice versa). R13 may thereby represent hydrogen or a protecting group such as e.g. tert-butyl which can be removed by standard deprotection methods. Methods for the synthesis of compounds (D-I) become obvious to one skilled in the art from consulting the above routes A and B and examples described in the experimental section.

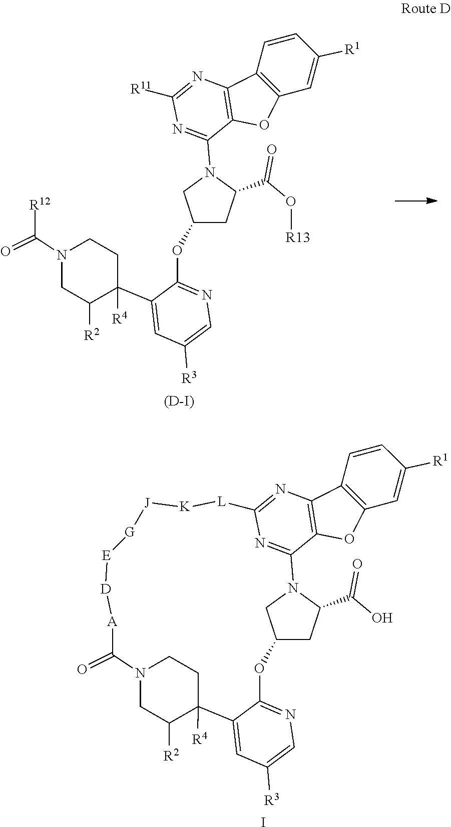

### SYNTHESES OF INTERMEDIATES

The syntheses described hereinafter were in part carried out according to the following general procedures indicated.

General procedure Int-A: Hydrogenation with Pd/C catalyst (see: Intermediate N-09, step 4)

General procedure Int-B: Ring closure under basic conditions (see: Intermediate N-09, step 5)

General procedure Int-C: Chlorination applying phosphoryl trichloride (see: Intermediate N-09, step 7)

General procedure Int-D: S_{N}Ar with hydroxyproline ester (see: Intermediate N-09, step 8)

General procedure Int-E: Ester cleavage applying lithium hydroxide (see: Intermediate N-04, step 2)

General procedure Int-F: Amidation applying PFTU (see: Intermediate N-04, step 3)

General procedure Int-G: Heck type coupling (see: Intermediate N-17, step 1)

General procedure A: Amidation (TBTU/NMP) (see: EX-01 step 1)

General procedure B: S_{N}Ar (NaH/DMA) (see: EX-01-step 2)

General procedure C: ester hydrolysis (LiOH/THF) (see: EX-01 step 4)

General procedure D: Amidation (HATU/DMF) (see: EX-01 step 5)

General procedure E: amidation (HATU/DMA) (see: EX-02 step 5)

General procedure F: BOC deprotection applying PTSA (see: EX-03, step 2)

General procedure G: Macrocyclization (HATU/DMA) (see: EX-03 step 3)

General procedure H: tBu deprotection (TFA/DCM) (see: EX-04 step 6)

General procedure I: cyclization to pyridine (NaH/NMP) (see: EX-05 step 2)

General procedure J: Amidation applying DCC (see: EX-08 step 3)

General procedure K: Olefin metathesis (see: EX-08 step 4)

General procedure L: Hydrogenation using Raney-Ni (see: EX-08 step 5)

General procedure M: Heck coupling (see: EX-22 step 1)

General procedure N: Hydrogenation using Pd/C (see: EX15 step 3)

General procedure O: Suzuki coupling (see: EX-20.01 step 1)

### INTERMEDIATE N-01

### tert-Butyl (2S,4S)-1-[4-(2-ethoxy-2-oxoethyl)-8-oxa-3,5-diazatricyclo[7.4.0.0^{2,7}]trideca-1(9),2(7),3,5,10,12-hexaen-6-yl]-4-hydroxypyrrolidine-2-carboxylate

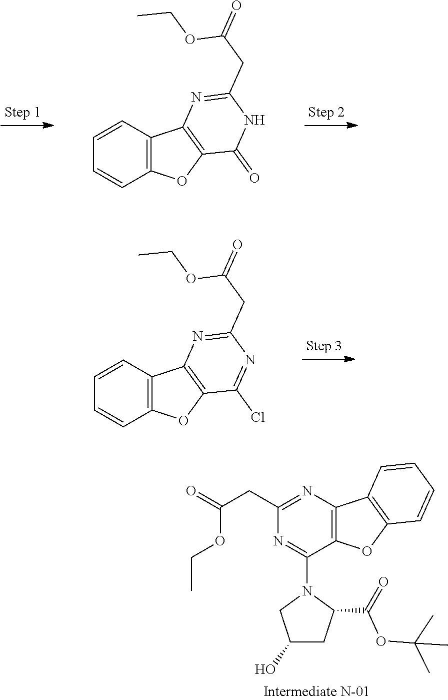 [SMILES: CCOC(=O)Cc1nc2c(oc3ccccc32)c(=O)[nH]1 ; CCOC(=O)Cc1nc(Cl)c2oc3ccccc3c2n1 ; CCOC(=O)Cc1nc(N2C[C@@H](O)C[C@H]2C(=O)OC(C)(C)C)c2oc3ccccc3c2n1]

Step 1: Ethyl 2-{6-oxo-8-oxa-3,5-diazatricyclo[7.4.0.0^{2,7}]trideca-1(9),2(7),3,10,12-pentaen-4-yl}acetate

To a mixture of ethyl-3-aminobenzofuran-2-carboxylate (5.00 g; 24.4 mmol) in 4M HCl in (50.00 mL; 200 mmol) was added ethyl cyanoacetate (5.19 mL; 48.7 mmol) at RT. The mixture was heated at 100° C. for 4 h. After cooling to RT, another 2.6 mL (24.4 mmol) of ethyl cyanoacetate were added and heating was continued for 48 h at 100° C. The solvent was evaporated, and the crude residue was diluted with 100 mL MeOH, filtered and dried. The crude product was taken directly to the next step.

ESI-MS: 273.0 [M+H]⁺; Rₜ (HPLC): 0.63 min (Method B)

### Step 2: Ethyl 2-{6-chloro-8-oxa-3,5-diazatricyclo[7.4.0.0^{2,7}]trideca-1(9),2(7),3,5,10,12-hexaen-4-yl}acetate

A mixture of Ethyl 2-{6-oxo-8-oxa-3,5-diazatricyclo[7.4.0.0^{2,7}]trideca-1(9),2(7),3,10,12-pentaen-4-yl}acetate (3.35 g; 12.3 mmol) in POCl₃ (50.0 mL; 547 mmol) was heated at 110° C. for 1.5 h. The reaction mixture was cooled to RT and added dropwise to an ice bath (500 mL) under stirring over 30 min. Ethyl acetate was added and the layers were separated. To the organic layer, sat. NaHCO₃ sol. was added slowly, and the phases were separated. The org. layer was washed with water and brine, dried over sodium sulfate, filtered, and concentrated in vacuo.

ESI-MS: 291.0 [M+H]⁺; Rₜ (HPLC): 0.43 min (Method A)

### Step 3: tert-Butyl (2S,4S)-1-[4-(2-ethoxy-2-oxoethyl)-8-oxa-3,5-diazatricyclo[7.4.0.0^{2,7}]trideca-1(9),2(7),3,5,10,12-hexaen-6-yl]-4-hydroxypyrrolidine-2-carboxylate

To ethyl 2-{6-chloro-8-oxa-3,5-diazatricyclo[7.4.0.0^{2,7}]trideca-1(9),2(7),3,5,10,12-hexaen-4-yl}acetate (8.17 mmol; 1.00 eq., 2.50 g) in 30 mL NMP was added tert-butyl (2S,4S)-4-hydroxypyrrolidine-2-carboxylate hydrochloride (8.99 mmol; 2.01 g, preparation described in WO2005/35525) and DIPEA (27.0 mmol; 4.64 mL), and this mixture was stirred at 70° C. for 1.5 h. The reaction mixture was cooled to RT and added slowly to 300 mL of ice water. The precipitate was filtered, washed several times with water and dried.

ESI-MS: 442.0 [M+H]⁺; Rₜ (HPLC): 0.67 min (Method B)

### INTERMEDIATE N-02

### tert-Butyl (2S,4S)-1-{4-bromo-8-oxa-3,5-diazatricyclo[7.4.0.0^{2,7}]trideca-1(13),2(7),3,5,9,11-hexaen-6-yl}-4-hydroxypyrrolidine-2-carboxylate

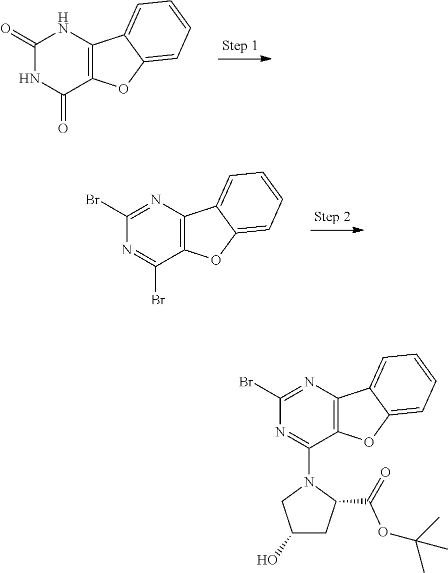 [SMILES: O=c1[nH]c(=O)c2oc3ccccc3c2[nH]1 ; Brc1nc(Br)c2oc3ccccc3c2n1 ; CC(C)(C)OC(=O)[C@@H]1C[C@H](O)CN1c1nc(Br)nc2c1oc1ccccc12]

### Step 1: 4,6-Dibromo-8-oxa-3,5-diazatricyclo[7.4.0.0^{2,7}]trideca-1(13),2(7),3,5,9,11-hexaene

A mixture of 1H-Benzo[4,5]furo[3,2-d]pyrimidine-2,4-dione (11.6 g; 0.0573 mol, preparation described in KR2015/84657) and POBr₃ (40.8 mL, 0.402 mol) was heated at 150° C. for 3 h. The mixture was cooled to RT, the pH was adjusted to pH=7 using aq. sat. NaHCO₃ solution while cooling at 0° C., and the mixture was extracted with EtOAc (3×50 mL). The organic layers were combined, dried over sodium sulfate, and evaporated under reduced pressure. The remaining residue was purified by dissolving in a mixture of DCM (3 V to the weight of the crude) and EtOAc (3 V to the weight of the crude) while stirring. After stirring at RT for 30 min, the mixture was filtered, and the supernatant was evaporated under reduced pressure.

ESI-MS: 327/329/331 [M+H]⁺(2Br); Rₜ (HPLC): 0.67 min (Method A)

### Step 2: tert-Butyl (2S,4S)-1-{4-bromo-8-oxa-3,5-diazatricyclo[7.4.0.0^{2,7}]trideca-1(13),2(7),3,5,9,11-hexaen-6-yl}-4-hydroxypyrrolidine-2-carboxylate

To a mixture of 4,6-Dibromo-8-oxa-3,5-diazatricyclo[7.4.0.0^{2,7}]trideca-1(13),2(7),3,5,9,11-hexaene (5.56 g, 16.9 mmol) in 93 mL DMF was added tert-butyl (2S,4S)-4-hydroxypyrrolidine-2-carboxylate hydrochloride (4.17 g, 16.6 mmol, described in WO2005/35525) and K₂CO₃ (7.03 g, 50.8 mmol). After stirring at RT over-night, the reaction mixture was poured into water and neutralized with 4M aq. HCl solution. The precipitate was collected by filtration and dried under vacuum.

ESI-MS: 434/436 [M+H]⁺ (2Br); Rₜ (HPLC): 0.64 min (Method A)

### Intermediate N-03

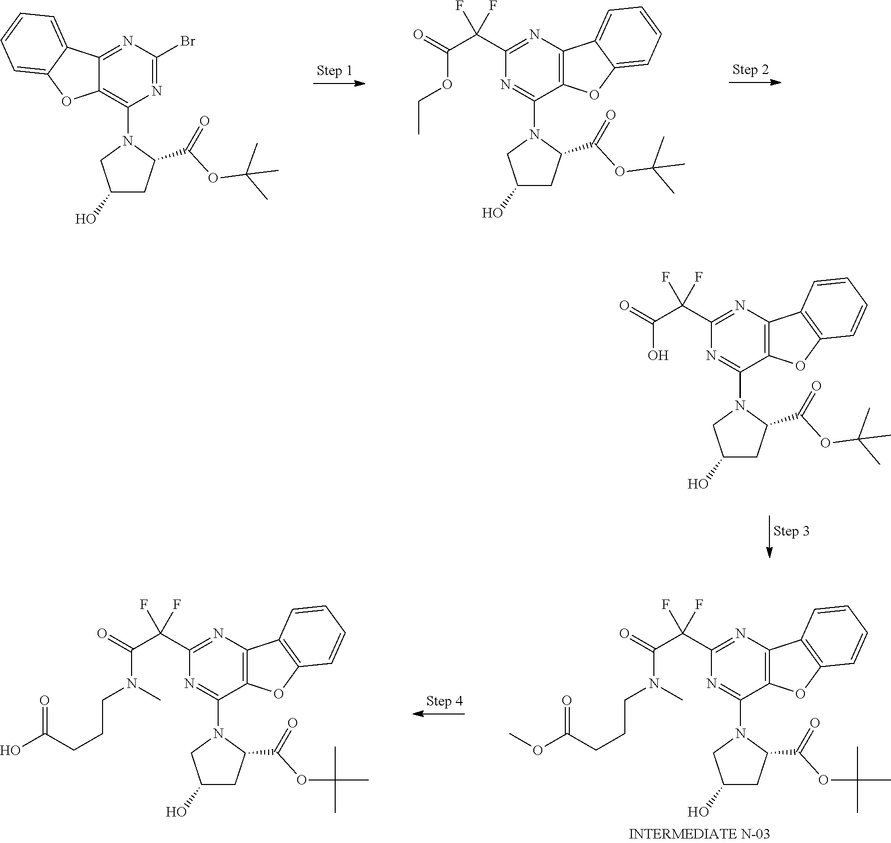 [SMILES: CC(C)(C)OC(=O)[C@@H]1C[C@H](O)CN1c1nc(Br)nc2c1oc1ccccc12 ; CCOC(=O)C(F)(F)c1nc(N2C[C@@H](O)C[C@H]2C(=O)OC(C)(C)C)c2oc3ccccc3c2n1 ; CC(C)(C)OC(=O)[C@@H]1C[C@H](O)CN1c1nc(C(F)(F)C(=O)O)nc2c1oc1ccccc12 ; CN(CCCC(=O)O)C(=O)C(F)(F)c1nc(N2C[C@@H](O)C[C@H]2C(=O)OC(C)(C)C)c2oc3ccccc3c2n1 ; COC(=O)CCCN(C)C(=O)C(F)(F)c1nc(N2C[C@@H](O)C[C@H]2C(=O)OC(C)(C)C)c2oc3ccccc3c2n1]

### N-03 Step 1: tert-Butyl (2S,4S)-1-[4-(2-ethoxy-1,1-difluoro-2-oxoethyl)-8-oxa-3,5-diazatricyclo-[7.4.0.0^{2,7}]trideca-1(13),2(7),3,5,9,11-hexaen-6-yl]-4-hydroxypyrrolidine-2-carboxylate

Copper bronze powder (508 mg, 8.00 mmol) was added to a mixture of ethylbromodifluoroacetate (533 μL, 4.00 mmol) and Intermediate N-02 (914 mg, 2.00 mmol) in 19.4 mL DMSO. The mixture was heated to 70° C. and stirred for 3 h. Then, an additional amount of ethylbromodifluoroacetate (533 μL, 2.00 eq) and copper bronze powder (508 mg, 4.00 eq.) were added and stirring was continued at 70° C. for 1 h. The reaction mixture was diluted with ACN/H₂O, acidified with TFA, filtered through a pad of celite and purified by means of prep. HPLC (P09; XBridge C18; ACN/H₂O/TFA).

ESI-MS: 478 [M+H]⁺; Rₜ (HPLC): 0.67 min (Method A)

### N-03 Step 2: 2-{6-[(2S,4S)-2-[(tert-butoxy)carbonyl]-4-hydroxypyrrolidin-1-yl]-8-oxa-3,5-diazatricyclo[7.4.0.0^{2,7}]trideca-1(9),2(7),3,5,10,12-hexaen-4-yl}-2,2-difluoroacetic acid

The product (ethyl ester) obtained in the previous step (217 mg, 0.432 mmol) in 4.34 mL THF was treated with aq. 2M LiOH solution (432 μL; 2.00 eq, 0.864 mmol) at RT until the starting material was completely consumed (1 h). The reaction mixture was diluted with 10 mL water, acidified with 1 mL 1N HCl and extracted with DCM. The combined organic phase was dried over sodium sulfate, filtered, and evaporated. The residue was taken up in ACN/H₂O and lyophilized.

ESI-MS: 450 [M+H]⁺; Rₜ (HPLC): 0.53 min (Method A)

### N-03 Step 3: tert-butyl (2S,4S)-1-(4-{difluoro[(4-methoxy-4-oxobutyl)(methyl)carbamoyl]methyl}-8-oxa-3,5-diazatricyclo[7.4.0.0^{2,7}]trideca-1(9),2(7),3,5,10,12-hexaen-6-yl)-4-hydroxypyrrolidine-2-carboxylate

At RT, HATU (250 mg, 0.624 mmol) was added to a mixture of 2-{6-[(2S,4S)-2-[(tert-butoxy)carbonyl]-4-hydroxypyrrolidin-1-yl]-8-oxa-3,5-diazatricyclo[7.4.0.0^{2,7}]trideca-1(9),2(7),3,5,10,12-hexaen-4-yl}-2,2-difluoroacetic acid (422 mg, 0.939 mmol), methyl 4-(methylamino)butanoate hydrochloride (110 mg; 0.624 mmol, 1.0 eq, preparation described in CN114173824) and DIPEA (231 μL, 1.34 mmol) in 3.17 mL DMF. The reaction mixture was stirred at RT until the starting material was completely consumed (2.5 h), then diluted with ACN/H₂O, acidified with TFA, and purified by means of prep. HPLC (XBridge C18; ACN/H₂O/TFA).

ESI-MS: 563 [M+H]⁺; Rₜ (HPLC): 0.61 min (Method A)

### N-03 Step 4: 4-(2-{6-[(2S,4S)-2-[(tert-butoxy)carbonyl]-4-hydroxypyrrolidin-1-yl]-8-oxa-3,5-diazatricyclo[7.4.0.0^{2,7}]trideca-1(9),2(7),3,5,10,12-hexaen-4-yl}-2,2-difluoro-N-methylacetamido)-butanoic acid

The product obtained in the previous step was reacted according to General procedure Int-E to yield intermediate N-03 as the final product.

ESI-MS: 450 [M+H]⁺; Rₜ (HPLC): 0.53 min (Method A)

### Intermediate N-04

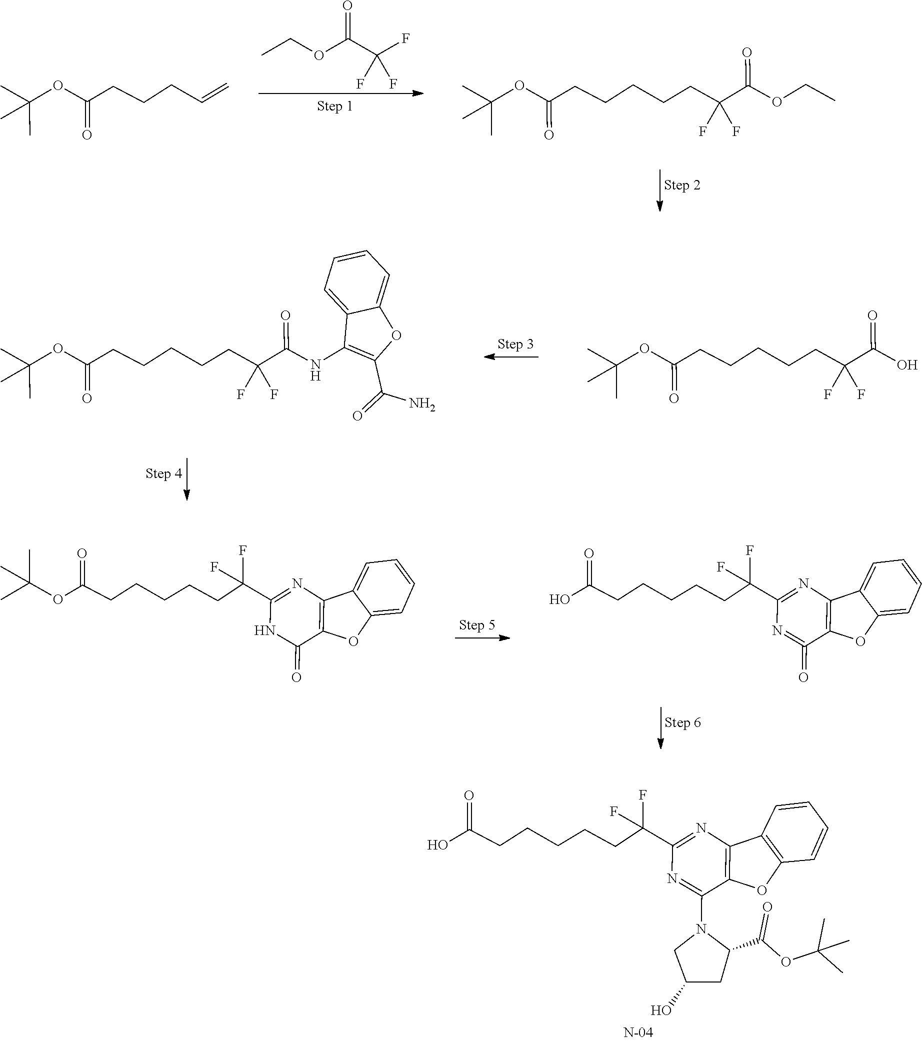

N-04 Step 1:

The zinc powder used was washed with 2% aq. hydrochloric acid, water, and acetone prior to use. The purified powder was dried in high vacuum and stored under argon. A flask was charged with zinc powder (2.44 g; 36.9 mmol), nickel(II)chloride hexahydrate (0.754 g; 3.14 mmol), THF (35.0 mL) and 3 drops of water and the mixture was stirred for 10 min at RT. Then, tert-butyl hex-5-enoate (4.63 g; 18.5 mmol; prepared as described in WO2010/15447) was added in one portion and ethyl iodofluoroacetate (2.80 mL; 18.5 mmol) was added dropwise (exothermic reaction during addition) while the temperature was kept below 30° C. After completed addition, the reaction mixture was stirred for 4 h at 60° C. The reaction mixture was poured into a mixture of sat. ammonium chloride solution (100 mL) and diethyl ether (100 mL) and stirred for 10 min. Then, the mixture was filtered through a pad of celite and after phase separation, the aqueous phase was extracted with diethyl ether. The combined organic layers were washed with water, dried with sodium sulfate, filtered, and evaporated. The crude product was purified by FC (silica gel; CH/DCM 10%->100%).

ESI-MS:312 [M+NH₄]⁺ Rₜ (HPLC): 0.78 min (method A)

N-04 Step 2:

General Procedure Int-E:

Lithium hydroxide (276 mg; 11.0 mmol) was added to a solution of the product from step 1 in 2:1 THF/H₂O and the reaction mixture was stirred at RT until reaction control by RP HPLC indicated consumption of the starting material (here: 2.5 h). Volatiles were removed in vacuo; the residue was acidified to pH=1 by addition of 5.50 mL of 1N aq. hydrochloric acid and the mixture was extracted with EtOAc thrice. The combined organic phase was dried with sodium sulfate, filtered, and evaporated.

ESI-MS:211 [M-isobutene+H]⁺ Rₜ (HPLC): 0.59 min (method A)

N-04 Step 3:

General Procedure Int-F:

At RT, PFTU (2.40 g; 5.60 mmol) was added to a stirred solution of the product from step 2 (1.40 g; 5.09 mmol) and DIPEA (970 μL; 5.60 mmol) in DMF (21.0 mL) and the mixture was stirred for 30 min. Then, 3-aminobenzofuran-2-carboxamide (1.01 g; 5.60 mmol) and further DIPEA (970 μL; 5.60 mmol) were added and the mixture was stirred for 10 min at RT. Then, the reaction mixture was heated to 50° C. and stirred for 16 h at this temperature. As reaction control by RP HPLC indicated incomplete consumption of starting materials, additional DIPEA (441 μL; 2.80 mmol) and PFTU (0.86 g; 2.04 mmol) were added and stirring at 50° C. was continued for an additional 2.5 h. The reaction mixture was diluted with water, acidified with TFA, filtered, and purified by means of RP HPLC (XBridge C18; ACN/water; modifier: TFA).

ESI-MS:425 [M+H]⁺ Rₜ (HPLC): 0.72 min (method A)

N-04 Step 4:

At RT, chlorotrimethylsilane (4.05 mL; 30.3 mmol) was added slowly to a solution of the product from step 3 (950 mg; 2.13 mmol and triethylamine (13.0 mL; 92.4 mmol) in 1,2-dichloroethane (28.5 mL). After completed addition, the reaction mixture was heated to 85° C. and stirred for 24 h at this temperature. The reaction mixture was poured into 30 mL 4 M hydrochloric acid (pH=1) and extracted with DCM twice. The combined organic phase was washed with water, dried with sodium sulfate, filtered and evaporated to afford tert-butyl 7,7-difluoro-7-{6-oxo-8-oxa-3,5-diazatricyclo[7.4.0.0^{2,7}]trideca-1(13),2(7),3,9,11-pentaen-4-yl}heptanoate.

ESI-MS:405 [M−H]⁻ Rₜ (HPLC): 0.44 min (method D)

N-04 Step 5:

The product from step 4 was converted to the chlorinated product applying general procedure Int-C, followed by purification by RP-HPLC (XBridge C18; ACN/water; modifier: TFA)

ESI-MS:367 [M−H]⁻ Rₜ (HPLC): 0.64 min (method A)

N-04 Step 6:

The product from step 6 was reacted according to general procedure Int-D to yield the title compound.

ESI-MS:520 [M+H]⁺ Rₜ (HPLC): 0.61 min (method A)

### Intermediate N-05

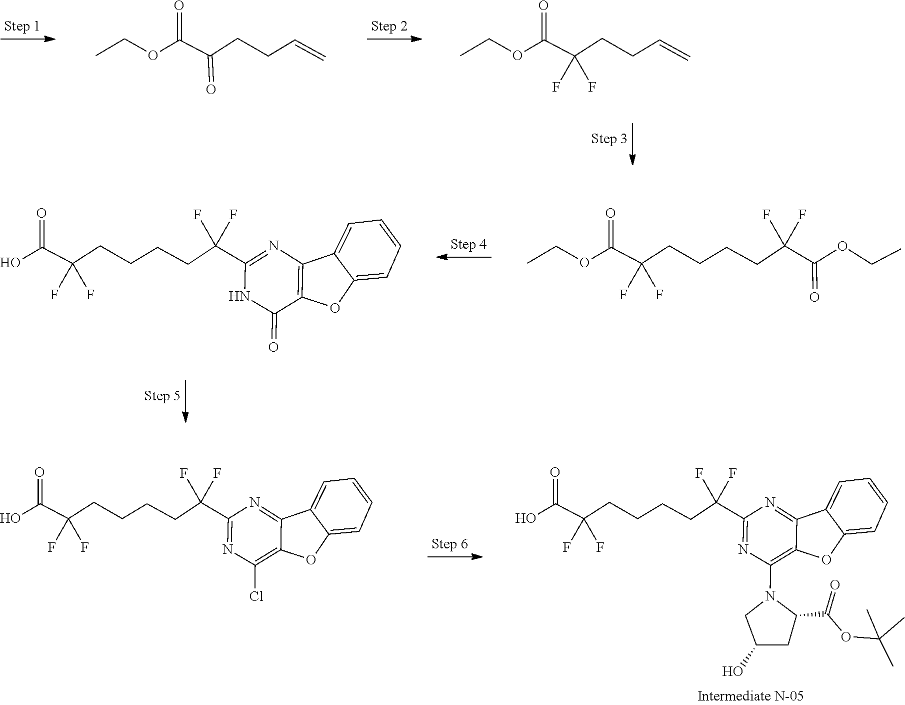

N-05 Step 1:

To a solution of magnesium powder (45.0 g; 1.85 mol) in THF (285 mL), was added Iodine (1.00 g; 3.94 mmol), followed by dropwise addition of a solution of 1-bromo-3-butene (111 g; 821 mmol) in THF (850 mL). The temperature was thereby kept below 50° C. The resulting mixture was cooled to −75° C. and a solution of diethyl oxalate (100 g; 684 mmol; 93.5 mL) in THF (1.89 L) was added dropwise. The mixture was stirred at −75° C. for further 4 h and then quenched by addition of saturated aq. ammonium chloride solution (900 mL) at 0° C. The pH was adjusted to pH 3 by addition of aq. hydrochloric acid (1 M). The mixture was extracted three times with EtOAc (500 mL). The combined organic layers were washed with brine (900 mL), dried over sodium sulfate, filtered, and concentrated under reduced pressure to give a residue which was purified by FC (silica gel; Petroleum ether/Ethyl acetate 0%->100%).

¹H NMR: (400 MHz, CDCl₃) δ=5.85-5.72 (m, 1H), 5.11-4.92 (m, 2H), 4.37-4.24 (m, 2H), 2.92 (t, J=7.3 Hz, 2H), 2.36 (q, J=7.1 Hz, 2H), 1.24-1.21 (m, 3H)

N-05 Step 2:

To a solution the product from step 1 (50.0 g, 320 mmol) in DCM (1000 mL) was added Bis-(2-methoxyethyl)aminosulfur trifluoride (Deoxofluor) (120 g, 544 mmol, 119 mL) and ethanol (2.95 g, 64.0 mmol) at 0° C. The mixture was stirred at 25° C. for 12 h. The reaction mixture was quenched by addition of 500 mL saturated aq. sodium bicarbonate and then extracted three times with DCM (500 mL). The combined organic layers were washed with aq. hydrochloric acid (1 M; 200 mL) and brine (200 mL), dried over sodium sulfate, filtered, and concentrated under reduced pressure to give a residue. The crude product was distilled in vacuo (30° C., 0.09 MPa/oil pump).

¹H NMR: (400 MHz, CDCl₃): δ=5.80 (br dd, J=10.3, 16.9 Hz, 1H), 5.13-5.00 (m, 2H), 4.39-4.28 (m, 2H), 2.30-2.12 (m, 4H), 1.43-1.34 (m, 3H)

N-05 Step 3:

To a mixture of zinc powder (8.79 g, 134 mmol) and THF (30.0 mL) was added nickel(II)chloride hexahydrate (804 mg, 3.38 mmol). The mixture was stirred at −65° C. for 5 min. Then was added dropwise ethyl difluoroiodoacetate (12.0 g, 48.0 mmol) and the product from step 2 (6.00 g, 33.7 mmol) at −65° C. The mixture was stirred at 25° C. for 12 h. The reaction mixture was quenched by addition saturated aq. ammonium chloride (60.0 mL) at 0° C., and then extracted three times with DCM (60.0 mL). The combined organic layers were washed with brine (60.0 mL), dried over sodium sulfate, filtered and concentrated under reduced pressure.

¹H NMR: (400 MHz, chloroform-d): 6=4.33 (q, J=7.2 Hz, 4H), 2.18-1.98 (m, 4H), 1.56 (td, J=3.8, 8.0 Hz, 4H), 1.36 (t, J=7.2 Hz, 6H)

N-05 Step 4:

To a solution of the product from step 3 (11.0 g, 36.4 mmol) in dioxane (30.0 mL) was added 1,5,7-triazabicyclo[4.4.0]dec-5-ene (14.2 g, 102 mmol) and 3-amino-1-benzofuran-2-carboxamide (4.50 g, 25.5 mmol). The mixture was stirred at 110° C. for 2 h and then was diluted with water (30.0 mL) and extracted three times with EtOAc (30.0 mL). The combined organic layers were washed with brine (30.0 mL), dried over sodium sulfate, filtered and concentrated under reduced pressure. The crude product was purified by RP HPLC (ACN/water, modifier: TFA).

¹H NMR: (400 MHz, DMSO-d): 6=8.09 (d, J=7.7 Hz, 1H), 7.88 (d, J=8.4 Hz, 1H), 7.72 (t, J=7.8 Hz, 1H), 7.53 (t, J=7.5 Hz, 1H), 2.40-2.37 (m, 2H), 2.12-2.07 (m, 2H), 1.54-1.48 (m, 4H).

N-05 Step 5:

The product from step 4 was chlorinated applying general procedure Int-C.

N-05 Step 6:

The product from step 5 was reacted applying general procedure Int-D to yield the title compound.

ESI-MS: 556 [M+H]⁺

Rₜ (HPLC): 0.64 min (method A)

### Intermediate N-06

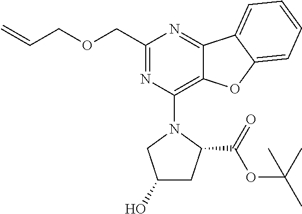 [SMILES: C=CCOCc1nc(N2C[C@@H](O)C[C@H]2C(=O)OC(C)(C)C)c2oc3ccccc3c2n1]

N-06 Step 1:

2-Allyloxyacetic acid (9.02 g; 73.8 mmol) was dissolved in DCM with a drop of DMF and cooled to 0 T. Oxalyl chloride (23.4 g; 184 mmol) was added dropwise. The reaction mixture was stirred at 0° C. for 2 h. Then the solvent was evaporated under reduced pressure. The residue was dissolved in DMF and added to a stirred solution of 3-aminobenzofuran-2-carboxamide (13.0 g; 73.8 mmol) in DMF. After 2 h, the mixture was poured into 100 mL of water and stirred for 5 min. The solid formed was collected, dissolved in DCM and dried over magnesium sulfate. Volatiles were evaporated and the solid was triturated with tert-butyl methyl ether to yield 3-[2-(prop-2-en-1-yloxy)acetamido]-1-benzofuran-2-carboxamide.

N-06 Step 2:

A suspension of the product from step 1 in 4 M aq. sodium hydroxide was stirred for 2 h at 70 T. The mixture was acidified by addition of aq. hydrochloric acid and the formed precipitate was collected and dried to afford 4-[(prop-2-en-1-yloxy)methyl]-8-oxa-3,5-diazatricyclo[7.4.0.0²⁷]trideca-1(9),2(7),3,10,12-pentaen-6-one.

The product from step 2 was further reacted in a 2-step sequence according to general procedure Int-C and then Int-D to yield the title compound.

ESI-MS:426 [M+H]⁺ Rₜ (HPLC): 0.49 min (method A)

### Intermediate N-07

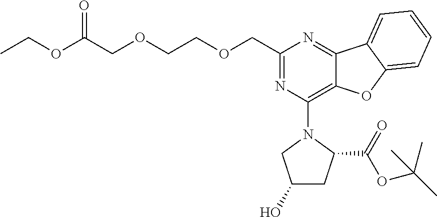 [SMILES: CCOC(=O)COCCOCc1nc(N2C[C@@H](O)C[C@H]2C(=O)OC(C)(C)C)c2oc3ccccc3c2n1]

N-07 Step 1:

A mixture of 3-aminobenzofuran-2-carboxamide (2.00 g; 11.4 mmol), ethyl 2-[2-(2-ethoxy-2-oxoethoxy)ethoxy]acetate (5.32 g; 22.7 mmol) and 1H,2H,3H,4H,6H,7H,8H-[1,3]diazino[1,2-a]-pyrimidine (6.45 g; 45.4 mmol) was heated to 120° C. for 150 min. After cooling to RT, aq. hydrochloric acid (1M; 70 mL) was added. The precipitate was filtered off with suction, washed with water and dried in vacuo at 60° C. to yield a mixture of ethyl 2-[2-({6-oxo-8-oxa-3,5-diazatricyclo[7.4.0.0^{2,7}]trideca-1(9),2(7),3,10,12-pentaen-4-yl}methoxy)ethoxy]acetate and the respective free acid which was taken to the next step.

N-07 Step 2:

For re-esterification of the acid, the mixture from step 1 (2.8 g) was dissolved in DCM (200 mL). 2 Drops of DMF were added, followed by the addition of oxalyl chloride (392 μL, 4.57 mmol). The mixture was stirred overnight, then ethanol (10 mL) was added, and the mixture stirred for a further 2 h. The mixture was concentrated under reduced pressure. Methyl tert-butyl ether was added and the precipitate formed was washed with methyl tert-butyl ether and dried at 50° C. to yield ethyl 2-[2-({6-oxo-8-oxa-3,5-diazatricyclo[7.4.0.0^{2,7}]trideca-1(9),2(7),3,10,12-pentaen-4-yl}methoxy)ethoxy]acetate.

The product from step 2 was further reacted in a 2-step sequence according to first general procedure Int-C and then Int-D to yield the title compound.

ESI-MS:516 [M+H]⁺ Rₜ (HPLC): 0.80 min (method E)

### Intermediate N-08

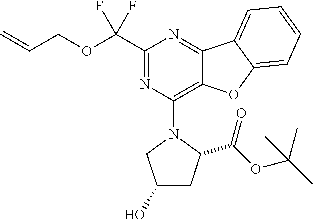 [SMILES: C=CCOC(F)(F)c1nc(N2C[C@@H](O)C[C@H]2C(=O)OC(C)(C)C)c2oc3ccccc3c2n1]

N-08 Step 1:

Sodium hydride (60% in mineral oil; 1.73 g, 44.2 mmol) was added portion wise to sodium 2-chloro-2,2-difluoroacetate (4.5 g, 29.5 mmol) and prop-2-en-1-ol (2.14 g, 36.8 mmol) in THF (30 mL) at 0° C. over a period of 2 minutes under nitrogen. The resulting suspension was stirred at 65° C. for 16 h.

The reaction mixture was cooled and diluted with aq. hydrochloric acid (2 M) until pH=5-6 was reached and then the aqueous layer was extracted twice with DCM (30 mL). The combined organic layers were washed with brine, dried with sodium sulfate, filtered and evaporated to afford crude product. The crude product was purified by FC (silica gel; petrol ether/EtOAc 0%->30% to afford 2-(allyloxy)-2,2-difluoroacetic acid.

¹H NMR (400 MHz, CDCl₃) δ 9.34 (s, 1H), 6.01-5.91 (m, 1H), 5.45-5.36 (m, 1H), 5.33-5.26 (m, 1H), 4.50 (dt, J=5.8, 1.2 Hz, 2H)

N-08 Step 2:

To a solution of the product from step 1 (5 g, 33 mmol) in pyridine (100 mL) was added 3-amino-1-benzofuran-2-carboxamide (4.63 g, 26 mmol), followed by the dropwise addition of phosphoryl trichloride (15.3 g, 0.1 mol) at 0° C. under nitrogen atmosphere. The resulting mixture was stirred overnight at RT. The mixture was diluted with water (200 mL) and extracted three times with EtOAc (200 mL). The combined organic layers were washed with brine, dried over sodium sulfate, and concentrated. The residue was purified by FC (silica gel; petrol ether/EtOAc 15%) to afford 3-(2-(allyloxy)-2,2-difluoroacetamido)benzofuran-2-carboxamide.

ESI-MS: 311 [M+H]⁺

N-08 Step 3:

The product from step 2 (3 g, 9.7 mmol) was added into aq. sodium hydroxide (14.5 mL, 4 M, 58.2 mmol), then THF (1.5 mL) was added. The reaction mixture was stirred for 4 h at 70° C. The cooled (RT) reaction mixture was acidified with aq. hydrochloric acid (2 M) to pH=5-6 and the formed precipitate was collected and dried to afford crude 2-((allyloxy)difluoromethyl)benzofuro[3,2-d]pyrimidin-4(3H)-one which was taken to the next step without further purification.

N-08 Step 4:

A 0.5 L 3-neck flask was charged with DMF (1.36 g, 18.6 mmol) and DCM (250 mL) equipped with a thermometer and a nitrogen balloon. The flask was cooled to 0° C. then was added dropwise a solution of oxalyl chloride (3.54 g, 27.9 mmol) in DCM (5 mL) over 5 min, while the temperature was maintained at 0 to 5° C. The mixture was stirred at ambient temperature for 0.5 h. The reaction was cooled in an ice-water bath to 0° C. and the product from step 3 (1.8 g, 6.2 mmol) was added portion wise. The reaction was stirred at RT for 15 min, then at 40° C. for 2 h. After cooling to RT, the reaction was poured into ice, neutralized with aq. sodium bicarbonate, extracted twice with DCM (100 mL). The combined organic phase was washed with water, dried with sodium sulfate and concentrated. The crude product was purified by FC (silica gel; petrol ether/EtOAc 2%->10%) to afford the title compound 2-((allyloxy)difluoromethyl)-4-chlorobenzofuro[3,2-d]pyrimidine.

¹H NMR (400 MHz, DMSO-d6) δ 8.41-8.31 (m, 1H), 7.79 (m, 2H), 7.60-7.54 (m, 1H), 7.26 (s, 1H), 6.06 (dq, J=10.8, 6.0 Hz, 1H), 5.45 (dd, J=17.2, 1.2 Hz, 1H), 5.30 (dd, J=10.4, 1.0 Hz, 1H), 4.70 (d, J=6.0 Hz, 2H)

N-08 Step 5:

The product from step 4 was reacted according to general procedure Int-D to yield the title compound.

ESI-MS: 462 [M+H]⁺

Rₜ (HPLC): 0.68 min (method A)

### Intermediate N-09

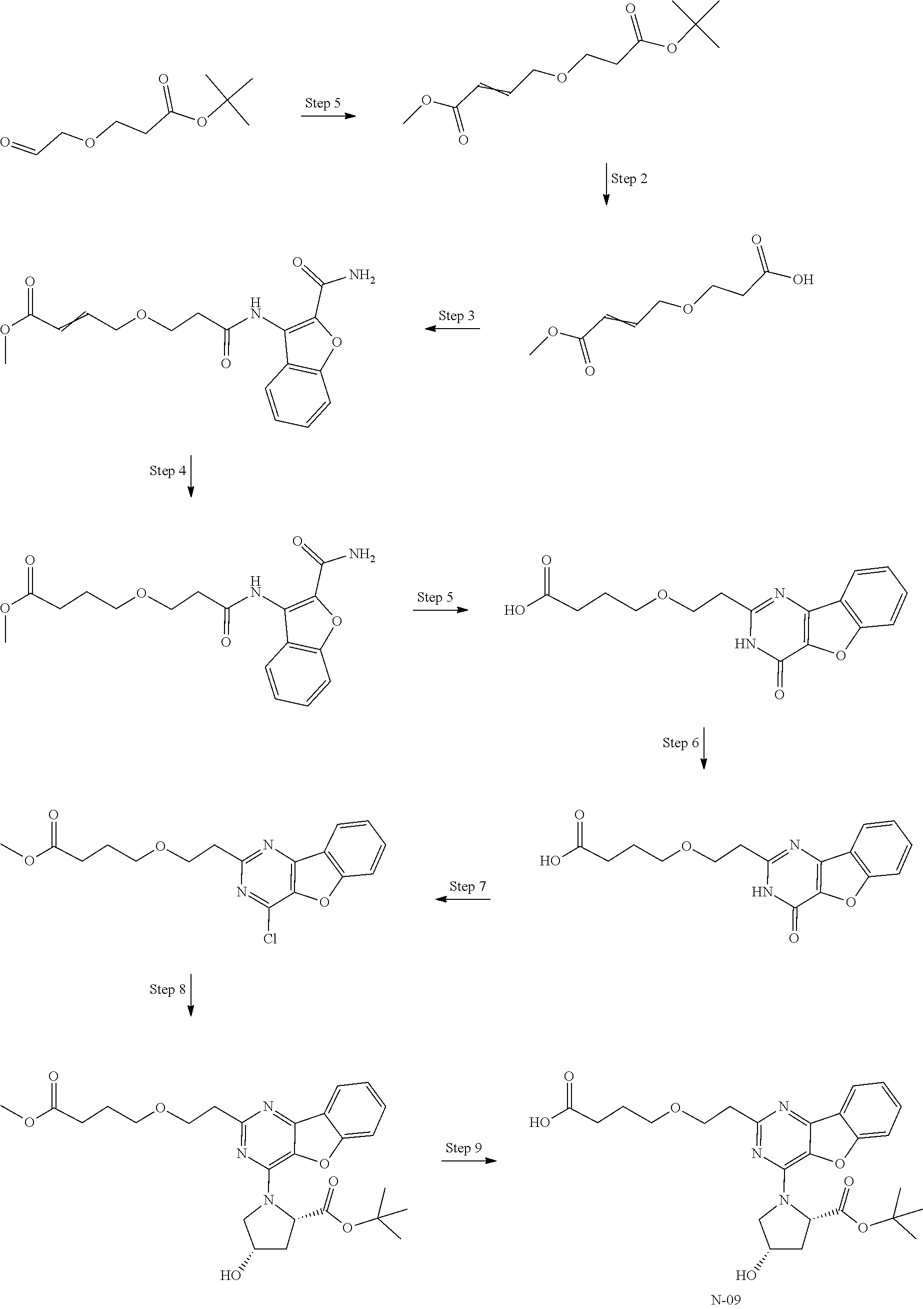

N-09 Step 1:

A mixture of tert-butyl 3-(2-oxoethoxy)propanoate (7.25 g; 38.5 mmol) which was prepared as described in EP2409977, and methyl (triphenylphosphoranylidene)acetate (13.1 g; 38.5 mmol) in DCM (200 mL) was stirred at RT overnight. The mixture was evaporated under reduced pressure and taken up in CH/EtOAc (3:1). Insolubles were removed by filtration and the filtrate was evaporated. The crude product was purified by FC (silica gel; CH/EtOAc 10%->45%) to yield the product as a mixture of cis- and trans-isomers.

N-09 Step 2:

The product from step 1 (1.50 g; 6.14 mmol) was stirred in a mixture of DCM (15 mL) and TFA (10 mL) over night. The mixture was evaporated and taken up in methanol (10 mL). Polymer bound tetraalkylammoniumcarbonate (2 weight equivalents) was added and the mixture was stirred for 90 min. Insolubles were filtered off and the filtrate was evaporated.

N-09 Step 3:

To a solution of the product from step 2 (5.00 g; 21.3 mmol) in ACN (140 mL) was added 1-chloro-N,N,2-trimethylpropenylamine (4.45 mL; 33.6 mmol). The mixture was stirred at RT for 10 min, then pyridine (5.10 mL; 63.0 mmol) and 3-aminobenzofuran-2-carboxamide (3.70 g; 21.0 mmol) was added. The mixture was stirred over night at RT, then water was added. The mixture was extracted with DCM and the organic layer was separated and evaporated. The crude product was purified first by FC (silica gel; petrol ether/EtOAc 40%->80%) and second by RP HPLC (Sunfire C18, ACN/water, modifier: TFA).

ESI-MS:347 [M+H]⁺ Rₜ (HPLC): 0.80 min (method C)

N-09 Step 4: General Procedure Int-A:

A mixture of the product from step 3 (3.40 g; 9.73 mmol), palladium on charcoal (10%; 350 mg) and ethanol (500 mL) was shaken under hydrogen pressure (50 psi) until RP HPLC indicated conversion of the starting material (here: 90 min). The catalyst was filtered off and the filtrate was evaporated to dryness.

ESI-MS:349 [M+H]⁺ Rₜ (HPLC): 0.81 min (method C)

N-09 Step 5:

General Procedure Int-B:

A mixture of the product from step 4 (4.55 g; 13.1 mmol) and aq. sodium hydroxide (4 M; 100 mL; 400 mmol) was stirred at 60° C. until reaction control by RP HPLC indicated conversion of the starting material (here: 60 min). The mixture was allowed to cool to RT and was then acidified by addition of aq. hydrochloric acid (4 M). The precipitated was collected and dried at 60° C.

ESI-MS:317 [M+H]⁺ Rₜ (HPLC): 0.73 min (method C)

N-09 Step 6:

To a mixture of the product from step 5 (3.53 g; 11.2 mmol), DCM (60 mL), and a few drops of DMF was added at RT oxalyl chloride (1.24 mL; 14.5 mmol). The mixture was stirred for 3 h at RT, then methanol was added and stirring was continued for another 60 min. The mixture was extracted with water and the organic layer was separated and evaporated to dryness.

ESI-MS:331 [M+H]⁺ Rₜ (HPLC): 0.82 min (method C)

N-09 Step 7:

General Procedure Int-C:

A mixture of the product from step 6 (3.70 g; 11.2 mmol) and phosphoryl trichloride (70 mL) was stirred at 90° C. for 4 h. Surplus phosphoryl trichloride was removed by distillation and water was added carefully. The resulting mixture was extracted with EtOAc, the organic layer was separated and evaporated. The crude product was taken to the next step.

ESI-MS:349 [M+H]⁺ Rₜ (HPLC): 1.02 min (method C)

N-09 Step 8:

General Procedure Int-D:

A mixture of the product from step 7 (300 mg; 0.896 mmol), tert-butyl (2S,4S)-4-hydroxypyrrolidine-2-carboxylate hydrochloride (253 mg; 1.08 mmol), potassium carbonate (300 mg; 2.06 mmol) and DMF (7.0 mL) was stirred at RT overnight. Water was added and the mixture was acidified by addition of aq. hydrochloric acid (1 M). The mixture was extracted with EtOAc, the organic layer was evaporated, and the crude product was purified by FC (silica gel; petrol ether/EtOAc 40%->75%)

ESI-MS:500 [M+H]⁺ Rₜ (HPLC): 0.75 min (method C)

N-09 Step 9:

The product from step 8 was reacted according to the general procedure Int-E to afford the ester cleaved title compound.

ESI-MS:486 [M+H]⁺ Rₜ (HPLC): 0.70 min (method C)

### Intermediate N-10

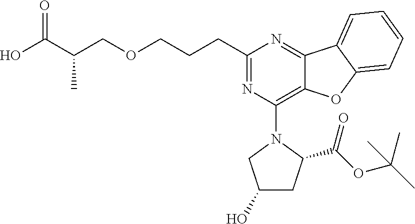 [SMILES: C[C@@H](COCCCc1nc(N2C[C@@H](O)C[C@H]2C(=O)OC(C)(C)C)c2oc3ccccc3c2n1)C(=O)O]

Preparation was performed from intermediate N-02 and methyl (2S)-3-allyloxy-2-methyl-propanoate analogously to the 3-step sequence described for the synthesis of N-17.

ESI-MS:500.3 [M+H]⁺ Rₜ (HPLC): 2.44 min (method H)

### Intermediate N-11

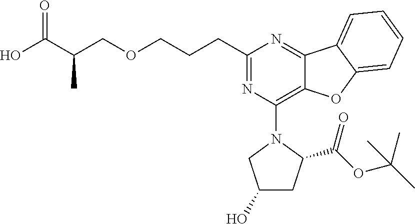 [SMILES: C[C@H](COCCCc1nc(N2C[C@@H](O)C[C@H]2C(=O)OC(C)(C)C)c2oc3ccccc3c2n1)C(=O)O]

Preparation was performed from intermediate N-02 and methyl (2R)-3-allyloxy-2-methyl-propanoate analogously to the 3-step sequence described for the synthesis of N-17.

ESI-MS:500.6 [M+H]⁺ Rₜ (HPLC): 2.44 min (method H)

### Intermediate N-12

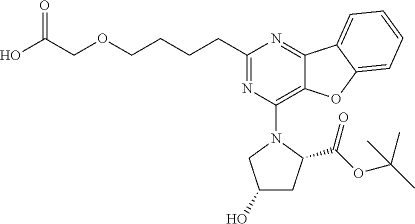 [SMILES: CC(C)(C)OC(=O)[C@@H]1C[C@H](O)CN1c1nc(CCCCOCC(=O)O)nc2c1oc1ccccc12]

Preparation was performed from intermediate N-02 and 2-(but-3-en-1-yloxy)acetic acid applying a two-step sequence:
Step 1: According to general procedure Int-G
Step 2: According to general procedure Int-A to yield the title compound.

ESI-MS:486 [M+H]⁺ Rₜ (HPLC): 0.54 min (method B)

### Intermediate N-13

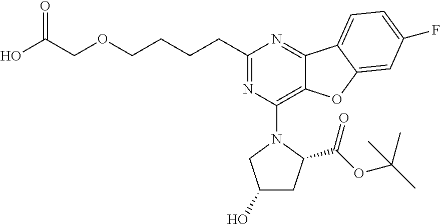 [SMILES: CC(C)(C)OC(=O)[C@@H]1C[C@H](O)CN1c1nc(CCCCOCC(=O)O)nc2c1oc1cc(F)ccc12]

Preparation was performed from intermediate N-18 and 2-(but-3-en-1-yloxy)acetic acid applying a two-step sequence:
N-13 Step 1: according to general procedure Int-G
N-13 Step 2: according to general procedure Int-A to yield the title compound.

ESI-MS:504 [M+H]⁺ Rₜ (HPLC): 0.45 min (method A)

### Intermediate N-14: 2-(4-{6-[(2S,4S)-2-[(tert-butoxy)carbonyl]-4-hydroxypyrrolidin-1-yl]-8-oxa-3,5-diazatricyclo[7.4.0.0^{2,7}]trideca-1(13),2,4,6,9,11-hexaen-4-yl}-4,4-difluorobutoxy)acetic acid

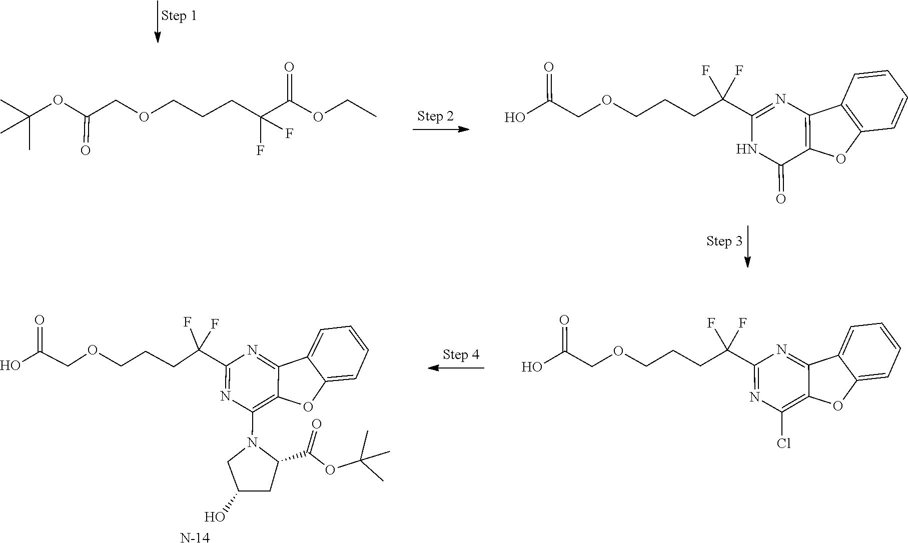

N-14 Step 1:

A dry reaction vessel was equipped with a magnetic stir bar and charged with tert-butyl 2-allyloxyacetate (1.00 g; 5.81 mmol), anhydrous nickel(II)chloride (0.038 g; 0.290 mmol) and sodium carbonate (0.61 g; 5.81 mmol). The reaction vessel was then briefly evacuated and backfilled with argon (this sequence was repeated a total of three times). Anhydrous DMF (40 mL), ethyl bromodifluoroacetate (1.5 mL, 11.6 mmol) and phenylsilane (2.9 mL; 23.2 mmol) were added to the reaction vessel via syringe sequentially. The vessel was heated at 70° C. in an oil-bath and stirred until TLC monitoring indicated consumption of the starting material (here: over night). The reaction mixture was diluted with 30 mL of EtOAc, and the organic layer was washed with 80 mL of saturated aqueous sodium chloride solution. After that, the organic layer was dried over sulfate and concentrated under reduced pressure and purified further by FC (silica gel; hexane/EtOAc) to yield ethyl 5-(2-tert-butoxy-2-oxo-ethoxy)-2,2-difluoro-pentanoate.

N-14 Step 2:

At RT, 1,5,7-Triazabicyclo[4.4.0]dec-5-ene (0.79 g; 5.68 mmol) was added to a solution of 3-aminobenzofuran-2-carboxamide (0.25 g; 1.42 mmol) in 1,4-dioxane (1 mL). Then, the product from step 1 (0.84 g; 2.84 mmol) was added, the temperature was increased to 120° C. and stirring was continued until TLC indicated almost complete conversion (here: 18 h). The reaction mixture was diluted with water (15 mL) and extracted with DCM (3×7 mL). The pH of the resulting aq. layer was adjusted to between 4 and 5 and the precipitated solid filtered off.

ESI-MS:353 [M+H]⁺ Rₜ (HPLC): 1.61 min (method F)

N-14 Step 3:

The product from step 2 was reacted according to general procedure Int-C.

ESI-MS:371 [M+H]⁺ Rₜ (HPLC): 1.82 min (method F)

N-14 Step 4:

The product from step 3 was reacted according to general procedure Int-D to yield the title compound.

ESI-MS:522.6 [M+H]⁺ Rₜ (HPLC): 2.96 min (method E)

### Intermediate N-15

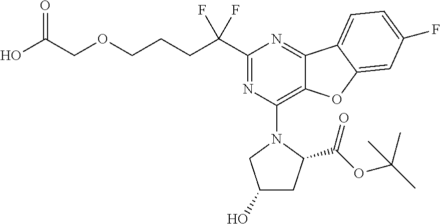 [SMILES: CC(C)(C)OC(=O)[C@@H]1C[C@H](O)CN1c1nc(C(F)(F)CCCOCC(=O)O)nc2c1oc1cc(F)ccc12]

Preparation was performed analogously to the procedure described for the synthesis of intermediate N-14 applying 3-amino-6-fluoro-1-benzofuran-2-carboxamide as starting material in step 2.

The starting material 3-amino-6-fluoro-1-benzofuran-2-carboxamide was prepared as follows:

4-fluoro-2-hydroxybenzonitrile (2.06 g; 14.3 mmol) was dissolved in ethanol (80 mL). Potassium carbonate (3.02 g; 21.8 mmol) and 2-bromoacetamide (2.40 g; 17.4 mmol) were added and the mixture was heated to 78° C. for 1 h. Potassium hydroxide (powdered; 1.91 g; 28.97 mmol) was added, stirring and heating was continued overnight. The mixture was allowed to cool to RT, water was added, ethanol evaporated, and the precipitate was filtered, washed with water and dried on air.

### Intermediate N-16

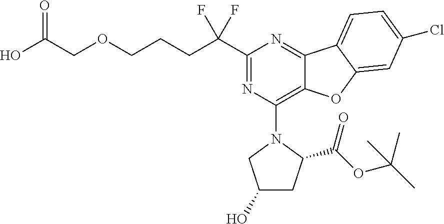 [SMILES: CC(C)(C)OC(=O)[C@@H]1C[C@H](O)CN1c1nc(C(F)(F)CCCOCC(=O)O)nc2c1oc1cc(Cl)ccc12]

Preparation was performed analogously to the procedure described for the synthesis of intermediate N-14 applying 3-amino-6-chloro-1-benzofuran-2-carboxamide (prepared as described in EP1710233) as starting material in step 2.

### Intermediate N-17

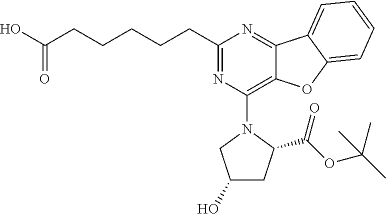 [SMILES: CC(C)(C)OC(=O)[C@@H]1C[C@H](O)CN1c1nc(CCCCCC(=O)O)nc2c1oc1ccccc12]

Preparation was performed from intermediate N-02 applying the following 3-step sequence:

N-17 Step 1:

General Procedure Int-G:

Intermediate N-02 (300 mg; 0.69 mmol) and 5-hexenoic acid methyl ester (298 μL; 2.07 mmol) were dissolved in DMF (10 mL; 123 mmol). Triethylamine (0.39 mL; 2.76 mmol) was added and the mixture was degassed with argon. Palladium(II)-acetate (31 mg; 0.14 mmol) and tri-o-tolylphosphine (84 mg; 0.28 mmol) were added under argon. The sealed vial was stirred at 95° C. overnight. The mixture was diluted with ACN/water, acidified with TFA, filtered over a syringe-filter. Purification by means of prep. RP HPLC (Sunfire C18, ACN/water; modifier: TFA) yielded tert-butyl (2S,4S)-4-hydroxy-1-[4-[6-methoxy-6-oxohex-1-en-1-yl]-8-oxa-3,5-diazatricyclo[7.4.0.0^{2,7}]trideca-1(13),2,4,6,9,11-hexaen-6-yl]pyrrolidine-2-carboxylate.

N-17 Step 2:

The product from step 1 was hydrogenated according to general procedure Int-A to yield tert-butyl (2S,4S)-4-hydroxy-1-[4-(6-methoxy-6-oxohexyl)-8-oxa-3,5-diazatricyclo[7.4.0.0^{2,7}]trideca-1(13),2,4,6,9,11-hexaen-6-yl]pyrrolidine-2-carboxylate.

ESI-MS:484 [M+H]⁺ Rₜ (HPLC): 0.65 min (method B)

N-17 Step 3:

The product from step 2 was reacted according to general procedure Int-E to afford the ester cleaved title compound.

### Intermediate N-18: tert-butyl (2S,4S)-1-{4-bromo-11-fluoro-8-oxa-3,5-diazatricyclo-[7.4.0.0^{2,7}]trideca-1(13),2,4,6,9,11-hexaen-6-yl}-4-hydroxypyrrolidine-2-carboxylate

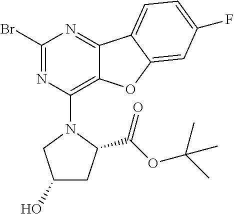 [SMILES: CC(C)(C)OC(=O)[C@@H]1C[C@H](O)CN1c1nc(Br)nc2c1oc1cc(F)ccc12]

Preparation from 11-fluoro-8-oxa-3,5-diazatricyclo[7.4.0.0^{2,7}]trideca-1(13),2(7),9,11-tetraene-4,6-dione analogously to the reaction sequence described for the synthesis of intermediate N-02. The starting material 11-fluoro-8-oxa-3,5-diazatricyclo[7.4.0.0^{2,7}]trideca-1(13),2(7),9,11-tetraene-4,6-dione was prepared analogously to the synthesis of 11-chloro-8-oxa-3,5-diazatricyclo[7.4.0.0^{2,7}]-trideca-1(13),2(7),9,11-tetraene-4,6-dione described in WO2019059577.

### Intermediate P-01

### tert-Butyl (3S,4R)-4-(5-chloro-2-fluoropyridin-3-yl)-4-fluoro-3-methylpiperidine-1-carboxylate

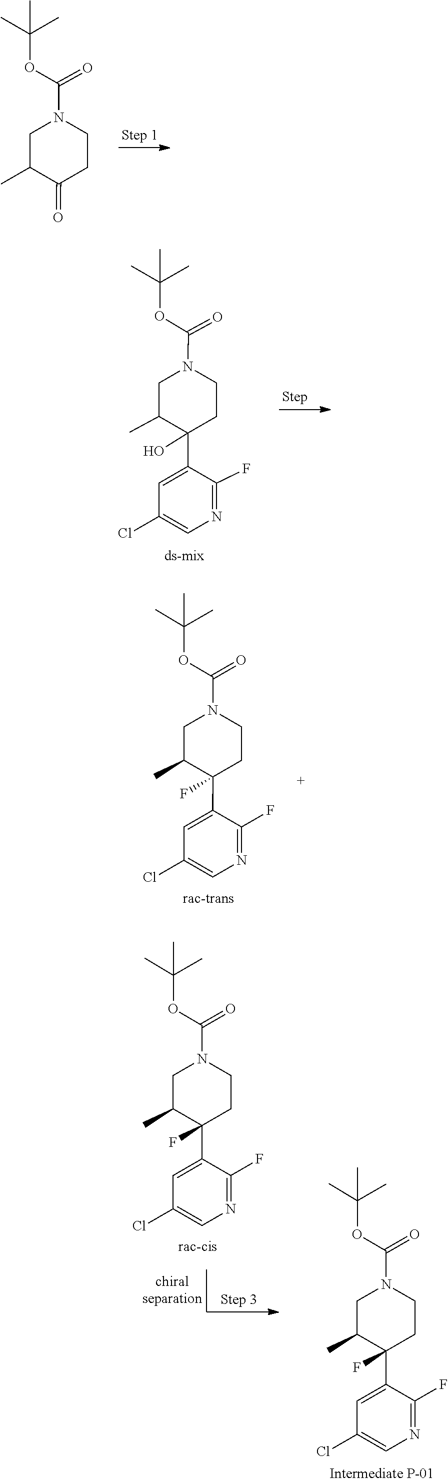 [SMILES: CC1CN(C(=O)OC(C)(C)C)CCC1=O ; CC1CN(C(=O)OC(C)(C)C)CCC1(O)c1cc(Cl)cnc1F ; "Step" ; C[C@H]1CN(C(=O)OC(C)(C)C)CC[C@@]1(F)c1cc(Cl)cnc1F ; C[C@H]1CN(C(=O)OC(C)(C)C)CC[C@]1(F)c1cc(Cl)cnc1F ; C[C@H]1CN(C(=O)OC(C)(C)C)CC[C@]1(F)c1cc(Cl)cnc1F]

P-01 Step 1: tert-Butyl 4-(5-chloro-2-fluoropyridin-3-yl)-4-hydroxy-3-methylpiperidine-1-carboxylate

Under an argon atmosphere, a solution of 3-bromo-5-chloro-2-fluoropyridine (10.0 g, 48.0 mmol, commercially available at Activate, MFCD04972409) in THF (150 mL; 15.0 V) was cooled to −78° C. Then, isopropylmagnesium chloride lithium chloride complex (1.3 M in THF, 38.4 mL, 50.0 mmol) was added dropwise while the temperature was kept below −65° C. Then, the reaction mixture was stirred for an additional 50 min at −78° C. Then, a pre-mixed solution of tert-butyl 3-methyl-4-oxopiperidine-1-carboxylate (11.6 g; 55.0 mmol, preparation described in WO2011/159852) and lanthanum trichloride lithium chloride complex (0.6 M in THF, 7.92 mL, 5.00 mmol) in THF (20. 0 mL; 2.00 V) was added dropwise while the temperature was kept below −65° C., and after completed addition, the reaction mixture was allowed to reach room temperature overnight.

The reaction mixture was quenched with 20 mL sat. NH₄Cl solution and the THF was removed in vacuum. The mixture was acidified with 6 mL 1N HCl and was extracted with EtOAc thrice, the combined organic phase was dried with sodium sulfate and concentrated in vacuum. The residue was co-evaporated with 15 mL toluene thrice. The crude product was used in the next step without further purification.

ESI-MS: 289.0/291.0 [M+H]⁺; Rₜ (HPLC): 0.64/0.68 min (Method A)

1H NMR: (400 MHz, CDCl3): δ ppm 8.02-8.11 (m, 2H) 3.80-4.20 (m, 2H) 3.01-3.20 (m, 1H) 2.68-2.91 (m, 1H) 2.25-2.49 (m, 2H) 1.99 (s, 1H) 1.48 (s, 9H) 0.65 (d, J=6.80 Hz, 3H).

P-01 Step 2: tert-Butyl 4-(5-chloro-2-fluoropyridin-3-yl)-4-fluoro-3-methylpiperidine-1-carboxylate

To the solution of tert-Butyl 4-(5-chloro-2-fluoropyridin-3-yl)-4-hydroxy-3-methylpiperidine-1-carboxylate (5.00 g, 15.0 mmol) in anhydrous DCM (70 mL, 14.0 V), DAST (3.83 mL 29.0 mmol) was added dropwise while cooling the reaction mixture in an ice-MeOH bath. Reaction was maintained in ice-MeOH bath under stirring for 3.5 h, quenched by addition of saturated NaHCO₃ solution and extracted with DCM thrice. The combined organic layer was dried over sodium sulfate, filtered and evaporated, the crude was further purified by flash column chromatography (n-heptane/EA=50/1 to 5/1).

ESI-MS: 291.0/291.0 [M-isobutene]+, chlorine isotope pattern, Rₜ (HPLC): 0.75 min (rac-trans) and 0.80 min (rac-cis) (Method A)

P-01 Step 3: tert-Butyl (3S,4R)-4-(5-chloro-2-fluoropyridin-3-yl)-4-fluoro-3-methylpiperidine-1-carboxylate

The cis-racemate obtained in step 2 was purified by prep-HPLC (neutral condition) and then further separated by chiral SFC (column: REGIS (s,s) WHELK-01 (250 mm*50 mm, 10 um); mobile phase: [0.1% N H3H2O IPA]; B %: 11%-11%, 2.3 min).

Intermediate P-01 (3S,4R) was the first eluting peak under these conditions.

1H NMR (400 MHz, CDCl3): δ ppm 8.10-8.14 (m, 1H) 7.93 (dd, J=8.19, 2.56 Hz, 1H) 4.08 (br s, 2H) 3.11 (br s, 1H) 2.82 (br s, 1H) 2.18-2.49 (m, 2H) 1.83 (br t, J=12.19 Hz, 1H) 1.50 (s, 9H) 0.71 (d, J=6.75 Hz, 3H). −19F NMR (400 MHz, CDCl3): δ ppm −69.5, −179.4.

The absolute configuration of this intermediate was confirmed via a single crystal X-ray diffraction.

### Intermediate P-02

### 5-Chloro-2-fluoro-3-[(3S,4R)-4-fluoro-3-methylpiperidin-4-yl]pyridine hydrochloride

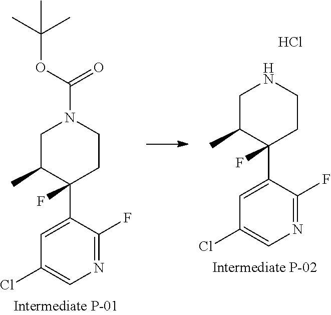 [SMILES: C[C@H]1CN(C(=O)OC(C)(C)C)CC[C@]1(F)c1cc(Cl)cnc1F ; C[C@H]1CNCC[C@]1(F)c1cc(Cl)cnc1F ; Cl]

tert-Butyl (3S,4R)-4-(5-chloro-2-fluoropyridin-3-yl)-4-fluoro-3-methylpiperidine-1-carboxylate (8.27 mmol, 3.02 g) was dissolved in 4N HCl in 1,4-dioxane (10.3 mL, 41.4 mmol) and the reaction mixture was stirred at rt for 1 h. The reaction mixture was diluted with diethyl ether, the formed precipitate was collected by filtration, washed with diethyl ether and dried.

ESI-MS: 247/249 (1Cl) [M+H]⁺; Rₜ (HPLC): 0.33 min (Method A)

Intermediate P-03:

### tert-Butyl 4-(5-chloro-2-fluoropyridin-3-yl)-4-fluoropiperidine-1-carboxylate

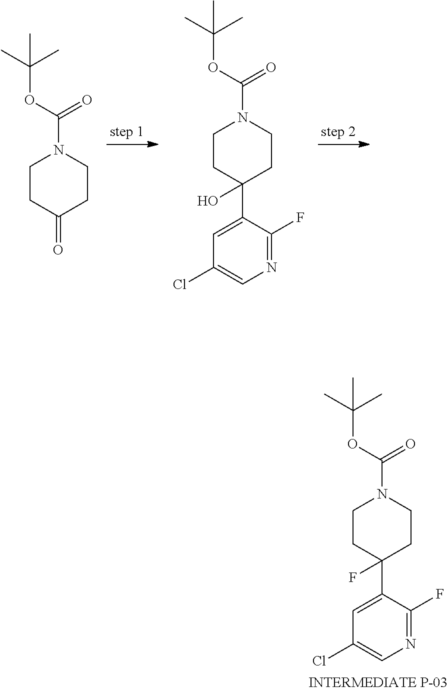 [SMILES: CC(C)(C)OC(=O)N1CCC(=O)CC1 ; CC(C)(C)OC(=O)N1CCC(O)(c2cc(Cl)cnc2F)CC1 ; CC(C)(C)OC(=O)N1CCC(F)(c2cc(Cl)cnc2F)CC1]

This intermediate was prepared according to the synthesis of Intermediate P-01 (steps 1-step 2) starting from tert-butyl 4-oxopiperidine-1-carboxylate.

P-03 step1: tert-butyl 4-(5-chloro-2-fluoropyridin-3-yl)-4-hydroxypiperidine-1-carboxylate

ESI-MS: 275/277 (1Cl) [M+H]⁺; Rₜ (HPLC): 0.61 min (Method A)

P-03 step2: tert-Butyl 4-(5-chloro-2-fluoropyridin-3-yl)-4-fluoropiperidine-1-carboxylate

ESI-MS: 333/335 (1Cl) [M+H]⁺; Rₜ (HPLC): 0.74 min (Method A)

### Intermediate P-04

### tert-Butyl (3S,4R)-4-(5-bromo-2-fluoropyridin-3-yl)-4-fluoro-3-methylpiperidine-1-carboxylat

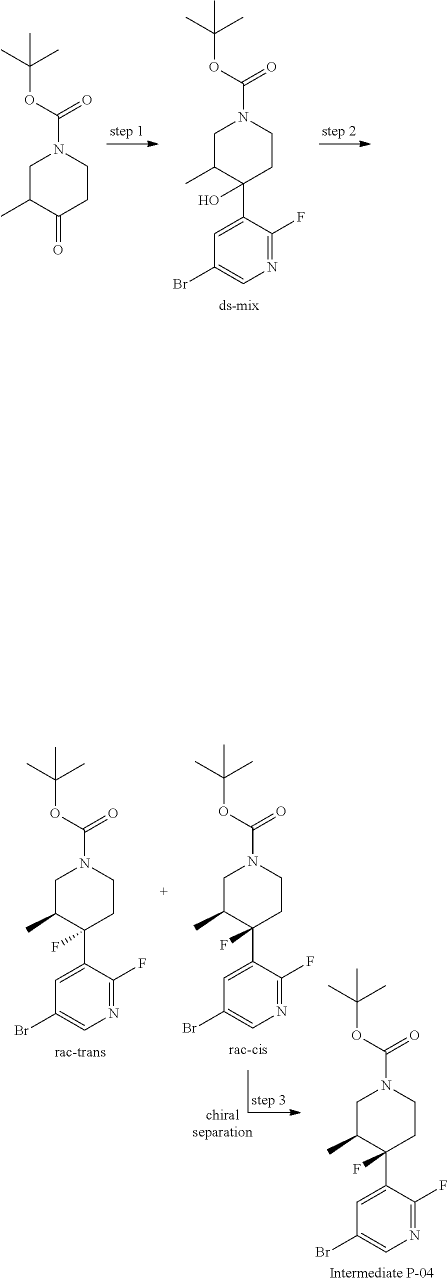

This intermediate was prepared according to the synthesis of Intermediate P-01 (steps 1-3), starting from 3,5-dibromo-2-fluoro-pyridine. Analytical data for step 2: ESI-MS: 335/337 [M-isobutene]+, chlorine isotope pattern, Rt (HPLC): 0.75 min (rac-cis) and 0.80 min (rac-trans) (Method A).

Chiral separation conditions for cis-enantiomers: separated by chiral SFC (column: DAICEL CHIRALPAK IG (250 mm*50 mm, 10 μm); mobile phase: MeOH [0.1% NH3]/CO2; 15/85.

Intermediate P-04 (3S,4R) was the second eluting peak under these conditions.

The final assignment of absolute stereochemistry for these products was done retrospectively based on X-ray of EXAMPLE EX-17.

### INTERMEDIATE P-05 and P-06

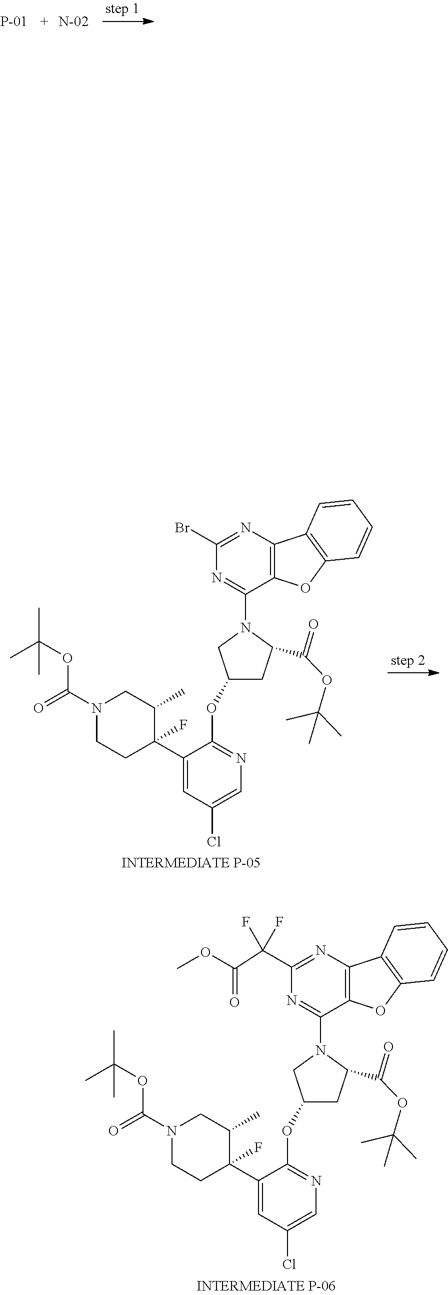 [SMILES: C[C@H]1CN(C(=O)OC(C)(C)C)CC[C@]1(F)c1cc(Cl)cnc1O[C@H]1C[C@@H](C(=O)OC(C)(C)C)N(c2nc(Br)nc3c2oc2ccccc23)C1 ; COC(=O)C(F)(F)c1nc(N2C[C@@H](Oc3ncc(Cl)cc3[C@@]3(F)CCN(C(=O)OC(C)(C)C)C[C@@H]3C)C[C@H]2C(=O)OC(C)(C)C)c2oc3ccccc3c2n1]

P-05: tert-butyl (3S,4R)-4-(2-{[(3S,5S)-1-{4-bromo-8-oxa-3,5-diazatricyclo[7.4.0.0^{2,7}]trideca-1(9),2(7),3,5,10,12-hexaen-6-yl}-5-[(tert-butoxy)carbonyl]pyrrolidin-3-yl]oxy}-5-chloropyridin-3-yl)-4-fluoro-3-methylpiperidine-1-carboxylate

This intermediate was prepared from intermediates P-01 and N-02 according to General procedure Int-D.

ESI-MS: 760/762/764 (Cl, Br) [M+H]⁺; Rₜ (HPLC): 0.99 min (Method A)

P-06: tert-butyl (3S,4R)-4-(2-{[(3S,5S)-5-[(tert-butoxy)carbonyl]-1-[4-(1,1-difluoro-2-methoxy-2-oxoethyl)-8-oxa-3,5-diazatricyclo[7.4.0.0^{2,7}]trideca-1(9),2(7),3,5,10,12-hexaen-6-yl]pyrrolidin-3-yl]oxy}-5-chloropyridin-3-yl)-4-fluoro-3-methylpiperidine-1-carboxylate

Under argon at RT, molecular sieves (3 Å) were added to a degassed solution of INTERMEDIATE P-05 (240 mg, 0.300 mmol) in 4.26 mL DMSO, followed by methylbromodifluoroacetate (89.7 μL, 0.792 mmol) and Copper bronze powder (101 mg, 1.52 mmol). After stirring at RT for 4 days, the reaction mixture was diluted with 10 mL EA and quenched with aq. KH₂PO₄ solution (1.27 M, 5.00 mL). The mixture was filtered through a pad of celite and the solid was washed with EA. The aqueous phase was extracted with EA. The combined organic phase was washed with water, dried with sodium sulfate, filtered and evaporated to afford the crude product. The residue was dissolved in DMF/ACN, acidified with 10% TFA and purified by means of prep. HPLC (XBridge C18; 60-100% ACN/H₂O/TFA).

ESI-MS: 790/792 (1Cl) [M+H]⁺; Rₜ (HPLC): 1.00 min (Method A)

### Intermediate P-07: Pent-4-en-1-yl (3S,4R)-4-(5-chloro-2-fluoropyridin-3-yl)-4-fluoro-3-methylpiperidine-1-carboxylate

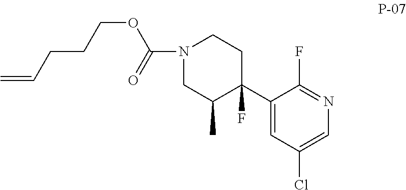 [SMILES: C=CCCCOC(=O)N1CC[C@](F)(c2cc(Cl)cnc2F)[C@@H](C)C1]

A mixture of Intermediate P-02 (100 mg, 0.279 mmol), DIPEA (144 μL, 0.892 mmol) and 4-nitrophenyl pent-4-en-1-yl carbonate (94 mg, 0.200 mmol, prepared as described in WO2014/11769) in 1.00 mL THF was stirred at 80° C. for 2 h. The volatiles were removed in vacuo and the crude mixture purified by means of prep. HPLC (ACN, Sunfire, TFA, Narrow).

ESI-MS: 359 [M+H]⁺; Rₜ (HPLC): 1.11 min (Method B)

### Intermediate P-08: 3-{2-[(3S,4R)-4-(5-chloro-2-fluoropyridin-3-yl)-4-fluoro-3-methylpiperidine-1-carbonyloxy]ethoxy}propanoic acid

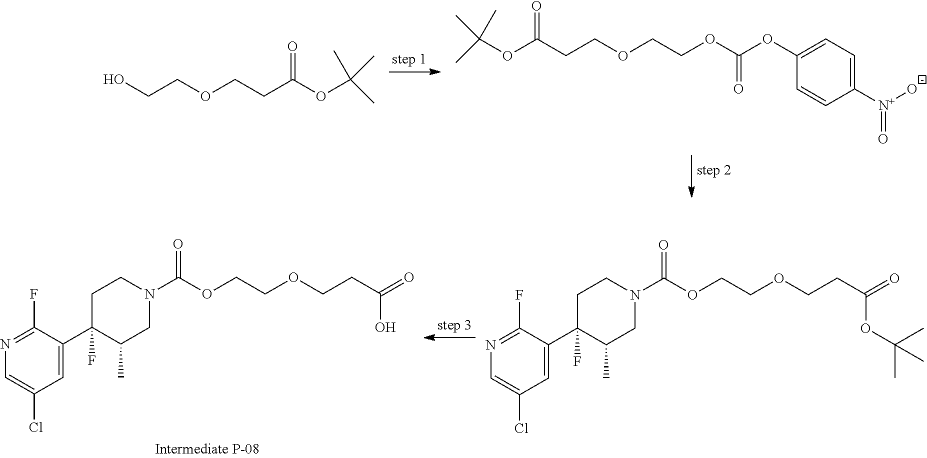

P-08 step 1: tert-butyl 3-(2-{[(4-nitrophenoxy)carbonyl]oxy}ethoxy)propanoate

Tert-butyl 3-(2-hydroxyethoxy)propanoate (5.10 g, 26.8 mmol, preparation described in WO2019/195609) was dissolved in 40.0 mL DCM, pyridine (2.16 mL, 26.9 mmol) was added and the mixture cooled to 0° C. At this temperature, a solution of 4-nitrophenyl chloroformate (5.57 g 26.8 mmol) in 20.0 mL DCM was slowly added. Once, addition was complete, the reaction mixture was allowed to come to RT and stirred for 2 h. Water (150 mL) was added and the layers were separated. The organic layer was washed with water and dried over sodium sulfate, filtered and concentrated in vacuo. The remainder was purified by means of silica gel chromatography (CH/EtOAc=95/5->80/20).

ESI-MS: 378 [M+Na]⁺, Rₜ (HPLC): 0.93 min (Method B)

P-08 step 2: tert-butyl 3-{2-[(3S,4R)-4-(5-chloro-2-fluoropyridin-3-yl)-4-fluoro-3-methylpiperidine-1-carbonyloxy]ethoxy}propanoate

To a mixture of the product obtained in P-08 step 1 (3.24 g, 7.31 mmol) and Intermediate P-02 (2.30 g (8.12 mmol) in 46 mL THF was added DIPEA (4.36 mL) and the mixture was heated to 65° C. for 2 h. The reaction mixture was cooled to RT and diluted with EtOAc. Phases were separated and the organic phase was washed with diluted sodium hydroxide solution. The organic layer was dried over sodium sulfate and evaporated to dryness.

ESI-MS: 463 [M+H]⁺, Rₜ (HPLC): 1.05 min (Method B)

P-08 step 3: 3-{2-[(3S,4R)-4-(5-chloro-2-fluoropyridin-3-yl)-4-fluoro-3-methylpiperidine-1-carbonyloxy]ethoxy}propanoic acid

This step was carried out according to General procedure H.

ESI-MS: 407 [M+H]⁺, Rₜ (HPLC): 0.79 min (Method B)

### Intermediate P-09

### 5-Bromo-2-fluoro-3-[(3S,4R)-4-fluoro-3-methylpiperidin-4-yl]pyridine hydrochloride

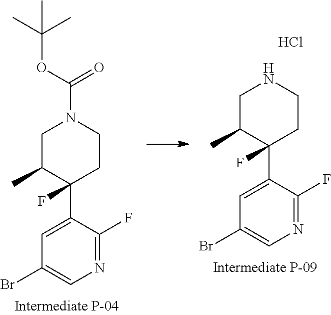 [SMILES: C[C@H]1CN(C(=O)OC(C)(C)C)CC[C@]1(F)c1cc(Br)cnc1F ; C[C@H]1CNCC[C@]1(F)c1cc(Br)cnc1F ; Cl]

This intermediate was prepared according to the synthesis of Intermediate P-02 starting from P-04 ESI-MS: 291/293 (1Br) [M+H]⁺; Rₜ (HPLC): 0.35 min (Method A)

### Syntheses of Examples

The syntheses described hereinafter were in part carried out according to the following general procedures indicated.

General procedure A: Amidation (TBTU/NMP) (see: EX-01 step 1)

General procedure B: S_{N}Ar (NaH/DMA) (see: EX-01-step 2)

General procedure C: ester hydrolysis (LiOH/THF) (see: EX-01 step 4)

General procedure D: Amidation (HATU/DMF) (see: EX-01 step 5)

General procedure E: amidation (HATU/DMA) (see: EX-02 step 5)

General procedure F: BOC deprotection applying PTSA (see: EX-03, step 2)

General procedure G: Macrocyclization (HATU/DMA) (see: EX-03 step 3)

General procedure H: tBu deprotection (TFA/DCM) (see: EX-04 step 6)

General procedure I: cyclization to pyridine (NaH/NMP) (see: EX-05 step 2)

General procedure J: Amidation applying DCC (see: EX-08 step 3)

General procedure K: Olefin metathesis (see: EX-08 step 4)

General procedure L: Hydrogenation using Raney-Ni (see: EX-08 step 5)

General procedure M: Heck coupling (see: EX-22 step 1)

General procedure N: Hydrogenation using Pd/C (see: EX15 step 3)

General procedure O: Suzuki coupling (see: EX-20.01 step 1)

### Example EX-01

### (1R,9S,11S,34S)-4-Chloro-1-fluoro-34-methyl-26,31-dioxo-8,15-dioxa-6,12,23,27,32,37-hexaaza-heptacyclo[30.2.2.1^{9,12}.1^{13,24}.0^{2,7}.0^{14,22}.0^{16,21}]octatriaconta-2,4,6,13(37),14(22),16(21),17,19,23-nonaene-11-carboxylic acid

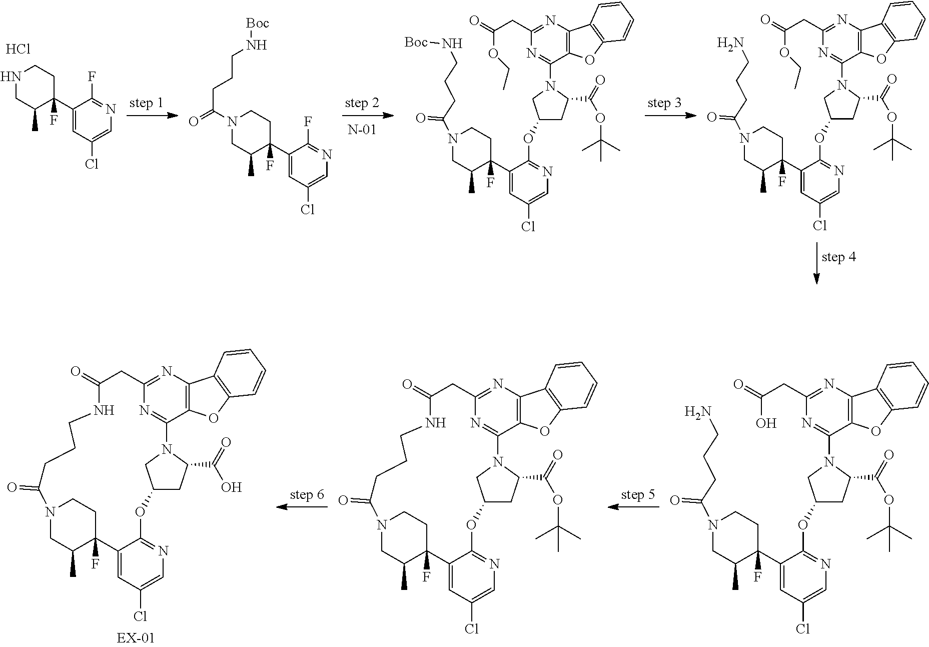

EX-01 Step 1: tert-butyl N-{4-[(3S,4R)-4-(5-chloro-2-fluoropyridin-3-yl)-4-fluoro-3-methylpiperidin-1-yl]-4-oxobutyl}carbamate

General Procedure A: Amidation (TBTU/NMP)

To a solution of 4-{[(tert-Butoxy)carbonyl]amino}butanoic acid (BOC-GABA-OH) (59.8 mg, 0.29 mmol) in 1.5 mL 1-methyl-2-pyrrolidinon was added DIPEA (270 μL, 1.20 mmol) followed by TBTU (94.5 mg, 0.290 mmol) and the mixture was stirred at RT for 5 min. Then, Intermediate P-02 was added and the reaction mixture was stirred at RT overnight. Water and ethyl acetate were added, phases were separated and the aqueous phase was extracted with ethyl acetate. The combined organic phase was washed successively with water, 5% L1Cl solution, sat NaHCO₃ solution and brine, dried over sodium sulfate and evaporated to dryness.

ESI-MS: 432/434 [M+H]⁺ (1Cl); Rₜ (HPLC): 0.70 min (Method A)

EX-01 Step 2:

General Procedure B: S_{N}Ar (NaH/DMA)

tert-Butyl (2S,4S)-4-({3-[(3S,4R)-1-(4-{[(tert-butoxy)carbonyl]amino}butanoyl)-4-fluoro-3-methylpiperidin-4-yl]-5-chloropyridin-2-yl}oxy)-1-[4-(2-ethoxy-2-oxoethyl)-8-oxa-3,5-diazatricyclo[7.4.0.0^{2,7}]trideca-1(9),2(7),3,5,10,12-hexaen-6-yl]pyrrolidine-2-carboxylate

This reaction was carried out under an argon atmosphere. To a stirred, ice/water bath cooled solution of hydroxyproline ester N-01 (100 mg, 0.220 mmol) and the product obtained in step 1 (120 mg, 0.270 mmol) in 2.00 mL DMA was added sodium hydride (60% dispersion in mineral oil, 25.8 mg, 0.65 mmol). Stirring was continued at 0° C. until the hydroxyproline was consumed (15 min). Water was added dropwise under ice cooling, followed by EtOAc and water. The mixture was acidified, and the phases were separated. The organic phase was washed with brine, dried over sodium sulfate, filtered and evaporated to dryness under vacuum.

ESI-MS: 853 [M+H]⁺; Rₜ (HPLC): 1.18 min (Method B)

EX-01 Step 3: tert-Butyl (2S,4S)-4-({3-[(3S,4R)-1-(4-aminobutanoyl)-4-fluoro-3-methylpiperidin-4-yl]-5-chloropyridin-2-yl}oxy)-1-[4-(2-ethoxy-2-oxoethyl)-8-oxa-3,5-diazatricyclo[7.4.0.0^{2,7}]trideca-1(9),2(7),3,5,10,12-hexaen-6-yl]pyrrolidine-2-carboxylate

A mixture of the product obtained in step 2 (158 mg, 0.185 mmol) in 10 mL dioxane was reacted with 4N HCl in dioxane (0.14 mL, 0.555 mmol) at RT overnight. Another 0.05 mL (0.18 mmol) of 4N HCl in dioxane was added, and after 4 h another 0.05 mL (0.18 mmol). Water was added to the reaction mixture, and the volatiles were removed under reduced pressure. The residue was co-evaporated with toluene, and the crude product was further purified by means of prep. HPLC (Sunfire, ACN/TFA, Narrow).

ESI-MS: 753/755 [M+H]⁺ (1Cl); Rₜ (HPLC): 0.83 min (Method B)

EX-01 Step 4: 2-{6-[(2S,4S)-4-({3-[(3S,4R)-1-(4-Aminobutanoyl)-4-fluoro-3-methylpiperidin-4-yl]-5-chloropyridin-2-yl}oxy)-2-[(tert-butoxy)carbonyl]pyrrolidin-1-yl]-8-oxa-3,5-diazatricyclo-[7.4.0.0^{2,7}]trideca-1(9),2(7),3,5,10,12-hexaen-4-yl}acetic acid

General Procedure C: Ester Hydrolysis (LiOH/THF)

To a mixture of the product obtained in step 3 (59 mg, 70.0 mmol) in 0.59 mL THF at RT was added 2M aqueous LiOH solution (0.223 mL, 0.440 mmol) and the reaction mixture was stirred overnight. The reaction mixture was diluted with EA and water, acidified with acetic acid. The phases were separated, the organic phase was washed with water and brine and dried over sodium sulfate, filtered and evaporated to dryness.

ESI-MS: 725/727 [M+H]⁺ (1Cl); Rₜ (HPLC): 0.75 min (Method B)

EX-01 Step 5: tert-Butyl (1R,9S,11S,34S)-4-chloro-1-fluoro-34-methyl-26,31-dioxo-8,15-dioxa-6,12,23,27,32,37-hexaazaheptacyclo[30.2.2.1^{9,12}.1^{13,24}.0^{2,7}.0^{14,22}.0^{16,21}]octatriaconta-2,4,6,13(37),14(22),16(21),17,19,23-nonaene-11-carboxylate

General Procedure D: Amidation (HATU/DMF)

To a mixture of HATU (25 mg, 0.065 mmol) in 2.50 mL DMF at RT was added slowly under stirring a solution of the product obtained in step 4 (39 mg, 0.050 mmol) and DIPEA (19 μL, 0.11 mmol) in 2.50 mL DMF. The mixture was stirred at RT for 1 h, then acidified with acetic acid and diluted with ACN. The crude product was purified using HPLC (Sunfire, ACN, TFA, Narrow).

Rₜ (HPLC): 0.75 min (Method B)

EX-01 Step 6: (1R,9S,11S,34S)-4-Chloro-1-fluoro-34-methyl-26,31-dioxo-8,15-dioxa-6,12,23,27,32,37-hexaaza-heptacyclo[30.2.2.1^{9,12}.1^{13,24}.0^{2,7}.0^{14,22}.0^{16,21}]]octatriaconta-2,4,6,13(37),14(22),16(21),17,19,23-nonaene-11-carboxylic acid

The product obtained in step 5 (15 mg, 0.021 mmol) in 2.00 mL DCM was reacted with TFA (16 μL, 0.21 mmol) at RT and the mixture was stirred overnight. Again, TFA (32 μL, 0.42 mmol) was added, and the mixture stirred for 4 h. Again, TFA (16 μL, 0.21 mmol) was added and the mixture stirred for 3 days. The mixture was diluted with ACN and water, filtered and directly purified by means of prep. HPLC (Sunfire, ACN, TFA, Narrow).

ESI-MS: 649/651 [M+H]⁺ (1Cl); Rₜ (HPLC): 0.85 min (Method B)

### Example 02

### (1R,9S,11S,34S)-4-chloro-1-fluoro-34-methyl-26,31-dioxo-8,15,30-trioxa-6,12,23,27,32,37-hexaazaheptacyclo[30.2.2.1^{9,12}.1^{13,24}.0^{14,22}..0^{16,21}]octatriaconta-2,4,6,13(37),14(22),16(21),17,19,23-nonaene-11-carboxylic acid

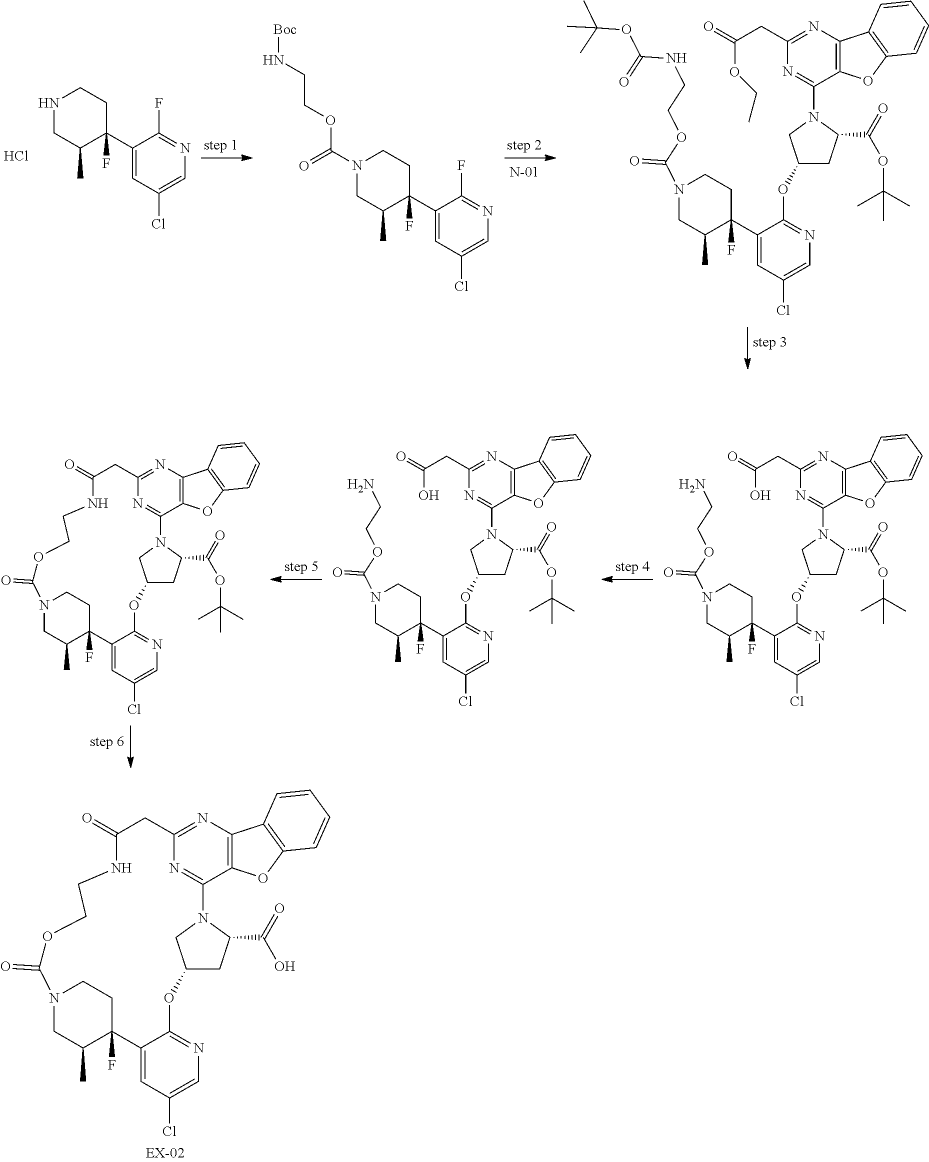

EX-02 Step1: 2-{[(tert-butoxy)carbonyl]amino}ethyl (3S,4R)-4-(5-chloro-2-fluoropyridin-3-yl)-4-fluoro-3-methylpiperidine-1-carboxylate

2.70 m LTH F, P-02 (270 mg, 0.906 mmol), 1-{[(2-{[(tert-butoxy)carbonyl]amino}ethoxy)carbonyl]oxy}-4-nitrobenzene (373 mg, 0.915 mmol, preparation described in US2019/192668) and DIPEA (439 μL, 2.72 mmol) were mixed and heated under stirring at 80° C. for 2 h. After cooling to RT, the reaction mixture was diluted with EA and water. NaHCO₃-sol. was added and the phases were separated. The organic phase was washed twice with water and brine, dried over Na₂SO₄, filtered and evaporated. The crude product was purified by means of prep. HPLC (sunfire, ACN, TFA, Narrow).

ESI-MS: 434 [M+H]⁺; Rₜ (HPLC): 0.99 min (Method B)

EX-02 Step2: 2-{[(tert-butoxy)carbonyl]amino}ethyl (3S,4R)-4-(2-{[(3S,5S)-5-[(tert-butoxy)carbonyl]-1-[4-(2-ethoxy-2-oxoethyl)-8-oxa-3,5-diazatricyclo[7.4.0.0^{2,7}]trideca-1(9),2(7),3,5,10,12-hexaen-6-yl]pyrrolidin-3-yl]oxy}-5-chloropyridin-3-yl)-4-fluoro-3-methylpiperidine-1-carboxylate

To a mixture of Intermediate N-01 (150 mg, 0.340 mmol) and tert-butyl N-{4-[(3S,4R)-4-(5-chloro-2-fluoropyridin-3-yl)-4-fluoro-3-methylpiperidin-1-yl]-4-oxobutyl}-carbamate (145 mg, 0.334 mmol) in 3.00 mL DMA was added sodium hydride (60% dispersion in mineral oil, 25.8 mg, 0.65 mmol) and the mixture was stirred at RT for 1 h. The reaction mixture was quenched by dropwise addition of slightly acidified water (TFA) and directly purified by means of preparative HPLC (Sunfire, ACN/TFA, Narrow).

ESI-MS: 855 [M+H]⁺; Rₜ (HPLC): 1.17 min (Method B)

EX-02 Step 3: 2-aminoethyl (3S,4R)-4-(2-{[(3S,5S)-5-[(tert-butoxy)carbonyl]-1-[4-(2-ethoxy-2-oxoethyl)-8-oxa-3,5-diazatricyclo[7.4.0.0^{2,7}]trideca-1(9),2(7),3,5,10,12-hexaen-6-yl]pyrrolidin-3-yl]oxy}-5-chloropyridin-3-yl)-4-fluoro-3-methylpiperidine-1-carboxylate

Under an argon atmosphere, the product obtained in step 2 in 2.0 mL DCM was cooled to −15° C. (acetone/ice bath) and TMSI (17 μL, 0.12 mmol) was added slowly. The reaction mixture was kept at −15° C. for 45 min, then at 0° C. for 30 min. Again, TMSI (9 μL, 0.060 mmol) was added and stirring continued at 0° C. for 45 min. The reaction was stopped by addition of 2.0 mL methanol. The solvent was removed under reduced pressure, the residue was purified by means of preparative HPLC (Sunfire, ACN/TFA, Narrow).

ESI-MS: 755 [M+H]⁺; Rₜ (HPLC): 0.82 min (Method B)

EX-02 Step 4: 2-{6-[(2S,4S)-4-({3-[(3S,4R)-1-[(2-aminoethoxy)carbonyl]-4-fluoro-3-methylpiperidin-4-yl]-S-chloropyridin-2-yl}oxy)-2-[(tert-butoxy)carbonyl]pyrrolidin-1-yl]-8-oxa-3,5-diazatricyclo-[7.4.0.0^{2,7}]trideca-1(9),2(7),3,5,10,12-hexaen-4-yl}acetic acid

To a solution of the product obtained in step 3 (53 mg, 0.070 mmol) in 0.53 mL THF was added 2N aqueous LiOH solution (100 μL, 0.200 mmol) and water (60 μL, 3.33 mmol). The reaction mixture was stirred at RT for 4 h, then acidified with acetic acid (13 μL, 0.23 mmol) and diluted with ACN and methanol. The precipitate that formed was removed by filtration, and the filtrate was evaporated under reduced pressure to afford the title compound.

ESI-MS: 727 [M+H]⁺; Rₜ (HPLC): 0.75 min (Method B)

EX-02 Step 5: tert-butyl (1R,9S,11S,34S)-4-chloro-1-fluoro-34-methyl-26,31-dioxo-8,15,30-trioxa-6,12,23,27,32,37-hexaazaheptacyclo[30.2.2.1^{9,12}.1^{13,24}.0^{2,7}.0^{14,22}.0^{16,21}]octatriaconta-2,4,6,13(37),14(22),16(21),17,19,23-nonaene-11-carboxylate

General Procedure E

A solution of the product obtained in step 4 (46 mg, 0.063 mmol) and DIPEA (0.030 mL, 0.18 mmol) in 1.00 mL DMA was added slowly over 30 min via syringe pump into a solution of HATU (25 mg, 0.065 mmol) in 3.0 mL DMA, and stirring was continued for 30 min at RT. The reaction mixture was acidified with TFA and purified directly by means of prep. RP-HPLC (Sunfire, ACN/H₂O/TFA, Narrow).

ESI-MS: 769 [M+H]⁺; Rₜ (HPLC): 1.01 min (Method B)

EX-02 Step 6: (1R,9S,11S,34S)-4-chloro-1-fluoro-34-methyl-26,31-dioxo-8,15,30-trioxa-6,12,23,27,32,37-hexaazaheptacyclo[30.2.2.1^{9,12}.1^{13,24}.0^{2,7}.0^{14,22}.0^{16,21}]octatriaconta-2,4,6,13(37),14(22),16(21),17,19,23-nonaene-11-carboxylic acid

To a solution of the product obtained in step 5 (5.0 mg, 0.0071 mmol) in 0.25 mL DCM was added TFA (100 μL, 1.30 mmol) at RT and the reaction mixture was stirred overnight. Again, TFA (100 μL, 1.30 mmol) was added and stirring continued for 6 h. The volatiles were removed under a stream of nitrogen, the residue was taken up in ACN/water and lyophilized.

ESI-MS: 653 [M+H]⁺; Rₜ (HPLC): 0.91 min (Method B)

### Example 03 (1R,9S,11S,34S)-4-Chloro-1,25,25-trifluoro-27,34-dimethyl-26,31-dioxo-8,15-dioxa-6,12,23,27,32,37-hexaazaheptacyclo[30.2.2.1^{9,12}.1^{13,24}.0^{2,7}.0^{14,22}.0^{16,21}]octatriaconta-2,4,6,13,16,18,20,22,24(37)-nonaene-11-carboxylic acid

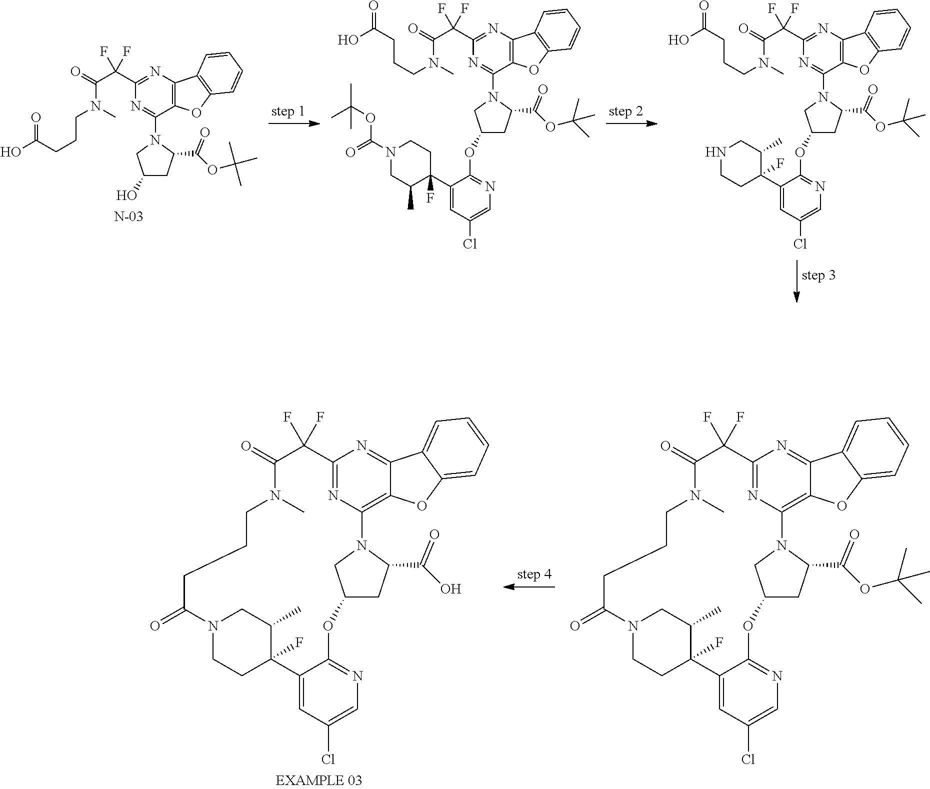

EX-03 Step 1: 4-(2-{6-[(2S,4S)-2-[(tert-butoxy)carbonyl]-4-({3-[(3S,4R)-1-[(tert-butoxy)carbonyl]-4-fluoro-3-methylpiperidin-4-yl]-5-chloropyridin-2-yl}oxy)pyrrolidin-1-yl]-8-oxa-3,5-diazatricyclo-[7.4.0.0^{2,7}]trideca-1(9),2(7),3,5,10,12-hexaen-4-yl}-2,2-difluoro-N-methylacetamido)butanoic acid

Intermediate N-03 was reacted with Intermediate P-01 according to General procedure to yield the title compound.

ESI-MS: 875/877 (1Cl) [M+H]⁺; Rₜ (HPLC): 0.92 min (Method A)

EX-03 Step 2: 4-(2-{6-[(2S,4S)-2-[(tert-butoxy)carbonyl]-4-({5-chloro-3-[(3S,4R)-4-fluoro-3-methylpiperidin-4-yl]pyridin-2-yl}oxy)pyrrolidin-1-yl]-8-oxa-3,5-diazatricyclo[7.4.0.0^{2,7}]trideca-1(9),2(7),3,5,10,12-hexaen-4-yl}-2,2-difluoro-N-methylacetamido)butanoic acid

General Procedure F: BOC Deprotection Applying PTSA

At 0° C., a mixture of the BOC-protected amine obtained in the previous step (72 mg, 0.078 mmol) in 2.50 mL ACN was reacted with PTSA (31.3 mg, 0.156 mmol, 2.00 eq.). The cooling bath was removed and the mixture stirred until complete conversion of the starting material to the free amine was observed (2-24 h). The reaction mixture was quenched by addition of water, diluted with ACN, filtered and purified by means of semi-prep. HPLC (XBridge C18; ACN/H₂O/TFA) to yield the title compound.

ESI-MS: 775/777 (1Cl) [M+H]⁺; Rₜ (HPLC): 0.65 min (Method A)

EX-03 Step 3: tert-butyl (1R,9S,11S,34S)-4-chloro-1,25,25-trifluoro-27,34-dimethyl-26,31-dioxo-8,15-dioxa-6,12,23,27,32,37-hexaazaheptacyclo[30.2.2.1^{9,12}.1^{13,24}.0^{2,7}.0^{14,22}.0^{16,21}]octatriaconta-2,4,6,13,16,18,20,22,24(37)-nonaene-11-carboxylate

General Procedure G: Macrocyclization (HATU/DMA)

A mixture of the product obtained in step 3 (44 mg, 0.054 mmol) and DIPEA (0.030 mL, 0.162 mmol) in 1.10 mL DMA was added slowly over 30 min via syringe pump into a solution of HATU (23 mg, 0.060 mmol) in 2.42 mL DMA, and stirring was continued at RT until complete consumption of the starting material was observed. The reaction mixture was acidified with TFA and purified directly by means of prep. RP-HPLC (Sunfire, ACN/H₂O/TFA, Narrow).

ESI-MS: 757/759 (1Cl) [M+H]⁺; Rₜ (HPLC): 0.86 min (Method A)

EX-03 Step 4: (1R,9S,11S,34S)-4-chloro-1,25,25-trifluoro-27,34-dimethyl-26,31-dioxo-8,15-dioxa-6,12,23,27,32,37-hexaazaheptacyclo[30.2.2.1^{9,12}.1^{13,24}.0^{2,7}.0^{14,22}.0^{16,21}]octatriaconta-2,4,6,13,16,18,20,22,24(37)-nonaene-11-carboxylic acid The product obtained in the previous step was dissolved in 0.50 mL DCM at RT and treated with TFA (38.7 μL, 0.502 mmol, 20 eq.) at 36° C. for 21 h. An additional amount of TFA (19.4 μL; 10.0 eq.) was added and stirring at 36° C. was continued for an additional 6 h. Again, TFA (38.7 μL; 20.0 eq.) was added and stirring at 36° C. was continued for an additional 20 h. The volatiles were removed in vacuum, the residue was dissolved in ACN/H₂O and purified by means of semi-prep. HPLC (XBridge C18; ACN/H₂O/TFA).

ESI-MS: 701/703 (1Cl) [M+H]⁺; Rₜ (HPLC): 0.71 min (Method A)

The absolute configuration of the final compound was confirmed from a co-crystal of the compound with human cGAS protein.

### Example 04

### (1R,9S,11S,34S)-4-chloro-1,25,25-trifluoro-34-methyl-26,31-dioxo-8,15-dioxa-6,12,23,27,32,37-hexaazaheptacyclo[30.2.2.1^{9,12}.1^{13,24}.0^{2,7}.0^{14,22}.0^{16,21}]octatriaconta-2,4,6,13,16,18,20,22,24(37)-nonaene-11-carboxylicacid

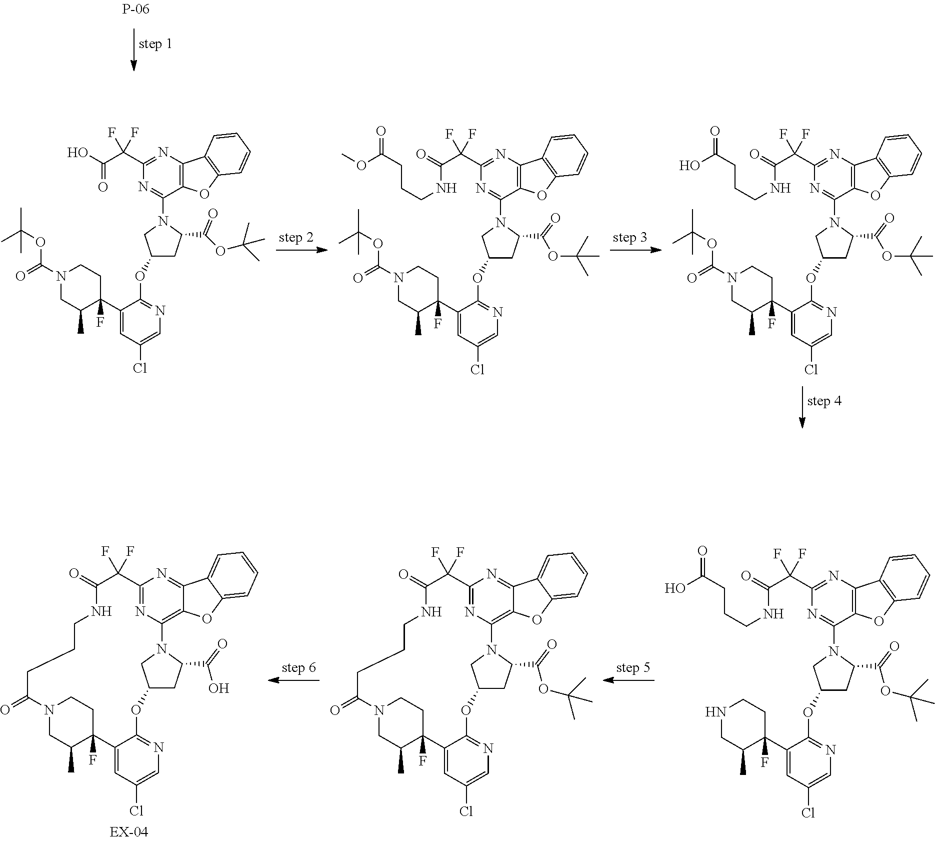

EX-04 Step 1: 2-{6-[(2S,4S)-2-[(tert-butoxy)carbonyl]-4-({3-[(3S,4R)-1-[(tert-butoxy)carbonyl]-4-fluoro-3-methylpiperidin-4-yl]-5-chloropyridin-2-yl}oxy)pyrrolidin-1-yl]-8-oxa-3,5-diazatricyclo-[7.4.0.0^{2,7}]trideca-1(9),2(7),3,5,10,12-hexaen-4-yl}-2,2-difluoroacetic acid

This compound was prepared from P-06 according to General Procedure C.

ESI-MS: 776/778 (1Cl) [M+H]⁺; Rₜ (HPLC): 0.94 min (Method A)

EX-04 Step 2: tert-butyl (3S,4R)-4-(2-{[(3S,5S)-5-[(tert-butoxy)carbonyl]-1-(4-{difluoro[(4-methoxy-4-oxobutyl)carbamoyl]methyl}-8-oxa-3,5-diazatricyclo[7.4.0.0^{2,7}]trideca-(9),2(7),3,5,10,12-hexaen-6-yl)pyrrolidin-3-yl]oxy}-5-chloropyridin-3-yl)-4-fluoro-3-methylpiperidine-1-carboxylate

This compound was prepared from the product obtained in the previous step and methyl-4-aminobutanoate hydrochloride according to General Procedure D.

ESI-MS: 875/877 (1Cl) [M+H]⁺; Rₜ (HPLC): 0.96 min (Method A)

EX-04 Step 3: 4-(2-{6-[(2S,4S)-2-[(tert-butoxy)carbonyl]-4-({3-[(3S,4R)-1-[(tert-butoxy)carbonyl]-4-fluoro-3-methylpiperidin-4-yl]-5-chloropyridin-2-yl}oxy)pyrrolidin-1-yl]-8-oxa-3,5-diazatricyclo-[7.4.0.0^{2,7}]trideca-1(9),2(7),3,5,10,12-hexaen-4-yl}-2,2-difluoroacetamido)butanoic acid

This compound was prepared from the product obtained in the previous step according to General Procedure C.

ESI-MS: 861/863 (1Cl) [M+H]⁺; Rₜ (HPLC): 0.91 min (Method A)

EX-04 Step 4: 4-(2-{6-[(2S,4S)-2-[(tert-butoxy)carbonyl]-4-({5-chloro-3-[(3S,4R)-4-fluoro-3-methylpiperidin-4-yl]pyridin-2-yl}oxy)pyrrolidin-1-yl]-8-oxa-3,5-diazatricyclo[7.4.0.0^{2,7}]trideca-1(9),2(7),3,5,10,12-hexaen-4-yl}-2,2-difluoroacetamido)butanoic acid

This compound was prepared from the product obtained in the previous step according to General Procedure D.

ESI-MS: 761/763 (1Cl) [M+H]⁺; Rₜ (HPLC): 0.64 min (Method A)

EX-04 Step 5: tert-butyl (1R,9S,11S,34S)-4-chloro-1,25,25-trifluoro-34-methyl-26,31-dioxo-8,15-dioxa-6,12,23,27,32,37-hexaazaheptacyclo[30.2.2.1^{9,12}.1^{13,24}.0^{2,7}.0^{14,22}.0^{16,21}]octatriaconta-2,4,6,13,16,18,20,22,24(37)-nonaene-11-carboxylate

This compound was prepared from the product obtained in the previous step according to General Procedure G.

ESI-MS: 743/745 (1Cl) [M+H]⁺; Rₜ (HPLC): 0.93 min (Method A)

EX-04 Step 6: 2-{6-[(2S,4S)-2-[(tert-butoxy)carbonyl]-4-({3-[(3S,4R)-1-[(tert-butoxy)carbonyl]-4-fluoro-3-methylpiperidin-4-yl]-5-chloropyridin-2-yl}oxy)pyrrolidin-1-yl]-8-oxa-3,5-diazatricyclo-[7.4.0.0^{2,7}]trideca-1(9),2(7),3,5,10,12-hexaen-4-yl}-2,2-difluoroacetic acid

General Procedure H: tBu Deprotection (TFA/DCM)

The product obtained in the previous step (33 mg, 0.042 mmol) was dissolved in 0.33 mL DCM at RT and treated with TFA (130 μL, 1.67 mmol, 40 eq.). The mixture was stirred at RT until complete consumption of the starting material was observed (24 h). The volatiles were removed in vacuum, the residue was dissolved in ACN/H₂O and purified by means of semi-prep. HPLC (XBridge C18; ACN/H₂O/TFA).

ESI-MS: 687/689 (1Cl) [M+H]⁺; Rₜ (HPLC): 0.73 min (Method A)

### Example 05

### (1R,9S,11S,34S)-4-chloro-1,25,25-trifluoro-34-methyl-31-oxo-8,15-dioxa-6,12,23,32,37-pentaazaheptacyclo[30.2.2.1^{9,12}.1^{13,24}.0^{2,7}.0^{14,22}.0^{16,21}]octatriaconta-2,4,6,13,16,18,20,22,24(37)-nonaene-11-carboxylicacid

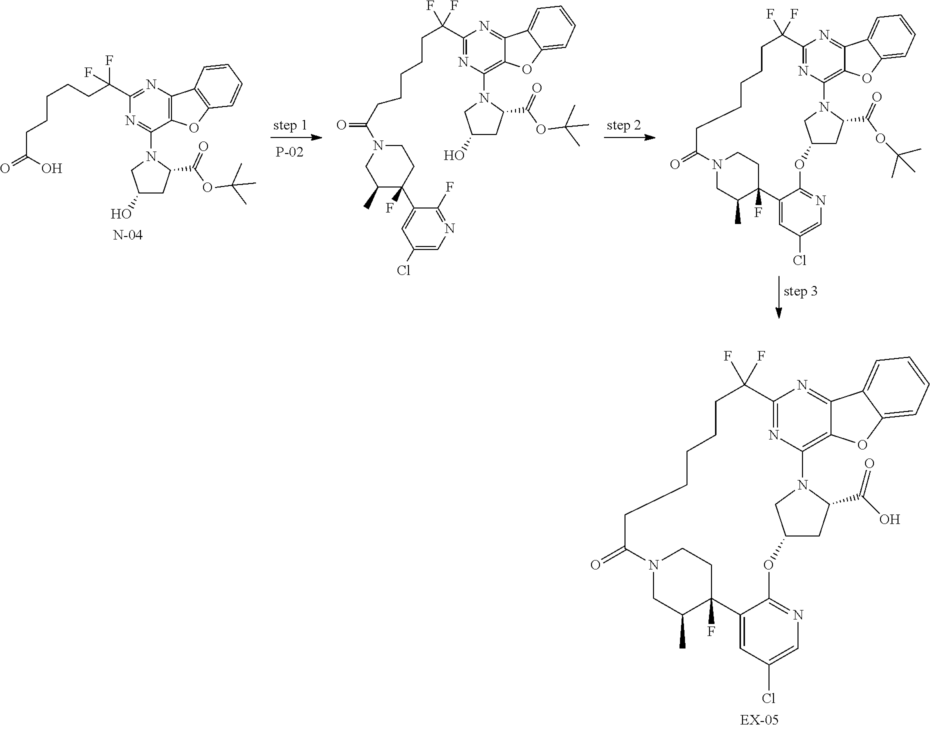 [SMILES: CC(C)(C)OC(=O)[C@@H]1C[C@H](O)CN1c1nc(C(F)(F)CCCCCC(=O)O)nc2c1oc1ccccc12 ; C[C@H]1CN(C(=O)CCCCCC(F)(F)c2nc(N3C[C@@H](O)C[C@H]3C(=O)OC(C)(C)C)c3oc4ccccc4c3n2)CC[C@]1(F)c1cc(Cl)cnc1F ; C[C@H]1CN2CC[C@]1(F)c1cc(Cl)cnc1O[C@H]1C[C@@H](C(=O)OC(C)(C)C)N(C1)c1nc(nc3c1oc1ccccc13)C(F)(F)CCCCCC2=O ; C[C@H]1CN2CC[C@]1(F)c1cc(Cl)cnc1O[C@H]1C[C@@H](C(=O)O)N(C1)c1nc(nc3c1oc1ccccc13)C(F)(F)CCCCCC2=O]

EX-05 Step 1:

This compound was prepared from Intermediate N-04 and Intermediate P-02 according to general procedure A.

ESI-MS: 748/750 [M+H]⁺; Rₜ (HPLC): 0.79 min (Method A)

EX-05 Step 2:

General Procedure I: Cyclization Via Pyridine (NaH/NMP)

A mixture of the product (fluoropyridine) obtained in EX-05 step 1 (75.0 mg; 0.0095 mmol) in 2.00 mL NMP was added dropwise within 2 min to a suspension of NaH (16.6 mg; 0.381 mmol) in 1.00 mL NMP. After completed addition, the reaction mixture was stirred at RT for 1 h. Additional NaH was added (16.6 mg, 0.381 mmol) and stirring at RT was continued for 20 min. To avoid formation of further byproducts, the reaction mixture was quenched with water, acidified with TFA, diluted with ACN/H₂O, filtered and purified by means of semi-prep. HPLC (XBridge C18; ACN/H₂O/TFA) to afford the macrocyclization product.

ESI-MS: 728 [M+H]⁺; Rₜ (HPLC): 0.91 min (Method A)

EX-05 Step 3:

The product obtained in EX-05 step 2 was reacted according to General Procedure H to yield the final product.

ESI-MS: 672/674 (1Cl) [M+H]⁺; Rₜ (HPLC): 0.87 min (Method A)

The following compounds were prepared analogously to EXAMPLE EX-05 described above, following the general procedures A, I, H.

| | | | | Rₜ (HPLC) | |
|---|---|---|---|---|---|
| | Starting | | | [min] | Synthesis |
| Ex. | materials | Structure | ESI-MS | (method) | comment |
| EX-16 | N-09 + P-02 | 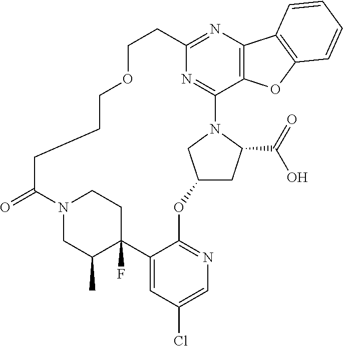 [SMILES: C[C@H]1CN2CC[C@]1(F)c1cc(Cl)cnc1O[C@H]1C[C@@H](C(=O)O)N(C1)c1nc(nc3c1oc1ccccc13)CCOCCCC2=O] | 638 / 640 [M + H]⁺ | 0.60 A | |
| EX-17 | N-09 + P-09 | 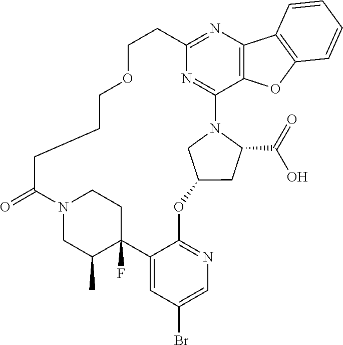 [SMILES: C[C@H]1CN2CC[C@]1(F)c1cc(Br)cnc1O[C@H]1C[C@@H](C(=O)O)N(C1)c1nc(nc3c1oc1ccccc13)CCOCCCC2=O] | 682 [M + H]⁺ | 0.62 A | |

### Example EX-06: (1R,9S,11S,34S)-4-chloro-1,25,25,30,30-pentafluoro-34-methyl-31-oxo-8,15-dioxa-6,12,23,32,37-pentaazaheptacyclo[30.2.2.1^{9,12}.1^{13,24}.0^{2,7}.0^{14,22}.0^{16,21}]-octatriaconta-2,4,6,13(37),14(22),16(21),17,19,23-nonaene-11-carboxylic acid

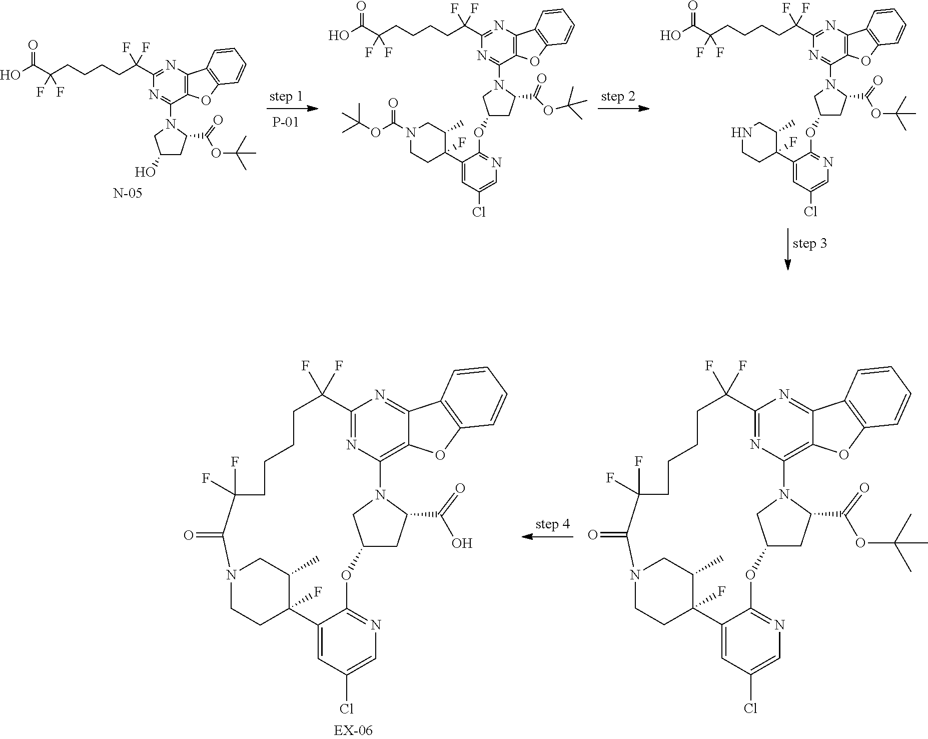

EX-06 Step 1:

Intermediate N-05 was reacted with Intermediate P-01 according to General procedure B using NMP as solvent to yield the title compound.

ESI-MS: 883/885 (1Cl) [M+H]⁺; Rₜ (HPLC): 0.95 min (Method A)

EX-06 Step 2:

This compound was prepared from the product obtained in EX-06 step 1 according to General Procedure F.

ESI-MS: 782/784 (1Cl) [M+H]⁺; Rₜ (HPLC): 0.69 min (Method A)

EX-06 Step 3:

This compound was prepared from the product obtained in EX-06 step 2 according to General Procedure G.

ESI-MS: 765/767 (1Cl) [M+H]⁺; Rₜ (HPLC): 0.96 min (Method A)

EX-06 Step 4:

The product obtained in EX-06 step 3 was reacted according to General Procedure H to yield the final compound.

ESI-MS: 708/710 (1Cl) [M+H]⁺; Rₜ (HPLC): 0.86 min (Method A)

The following compounds can be prepared analogously to EXAMPLE 06 described above, applying the General Procedures B, F, G and H.

| | | | | Rₜ (HPLC) | |
|---|---|---|---|---|---|
| | Starting | | | [min] | Synthesis |
| Ex. | materials | Structure | ESI-MS | (method) | comment |
| EX-07 | N-04 + P-01 | 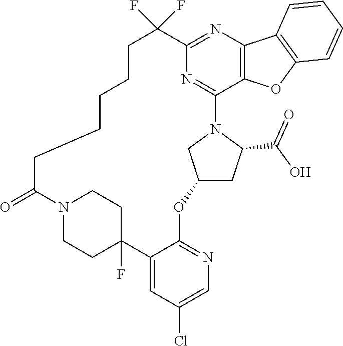 [SMILES: O=C(O)[C@@H]1C[C@H]2CN1c1nc(nc3c1oc1ccccc13)C(F)(F)CCCCCC(=O)N1CCC(F)(CC1)c1cc(Cl)cnc1O2] | 658 / 660 [M + H]⁺ | 0.87 A | |
| EX-14 | N-07 + P-01 | 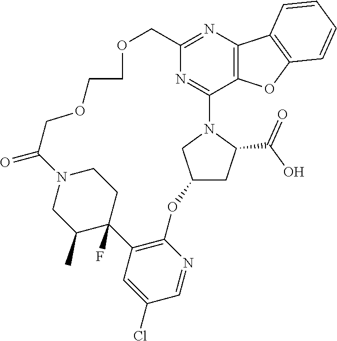 [SMILES: C[C@H]1CN2CC[C@]1(F)c1cc(Cl)cnc1O[C@H]1C[C@@H](C(=O)O)N(C1)c1nc(nc3c1oc1ccccc13)COCCOCC2=O] | 640 / 642 [M + H]⁺ | 0.94 min C | Step1: SNAr & hydrolysis Step 4: EDC in DCM |
| EX-25 | N-10 + P-04 | 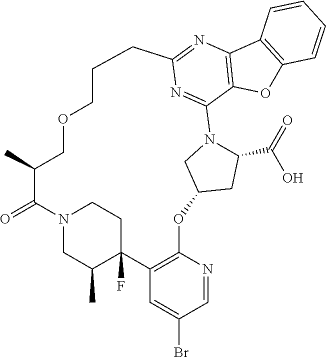 [SMILES: C[C@H]1COCCCc2nc(c3oc4ccccc4c3n2)N2C[C@H](C[C@H]2C(=O)O)Oc2ncc(Br)cc2[C@@]2(F)CCN(C[C@@H]2C)C1=O] | 696 [M + H]⁺ | 0.72 B | |
| EX-26 | N-11 P-04 | 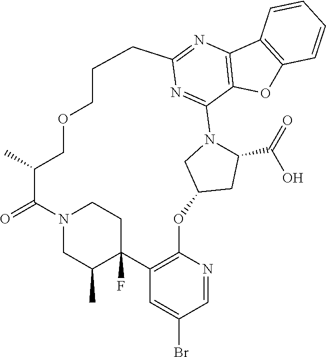 [SMILES: C[C@@H]1COCCCc2nc(c3oc4ccccc4c3n2)N2C[C@H](C[C@H]2C(=O)O)Oc2ncc(Br)cc2[C@@]2(F)CCN(C[C@@H]2C)C1=O] | 696 [M + H]⁺ | 0.62 A | |
| EX-27 | N-12 P-01 | 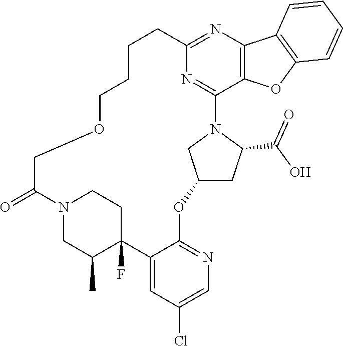 [SMILES: C[C@H]1CN2CC[C@]1(F)c1cc(Cl)cnc1O[C@H]1C[C@@H](C(=O)O)N(C1)c1nc(nc3c1oc1ccccc13)CCCCOCC2=O] | 638/640 [M + H]⁺ | 0.61 A | |
| EX-28 | N-12 P-04 | 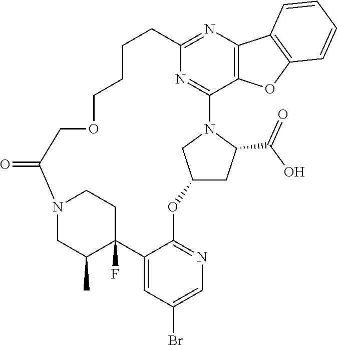 [SMILES: C[C@H]1CN2CC[C@]1(F)c1cc(Br)cnc1O[C@H]1C[C@@H](C(=O)O)N(C1)c1nc(nc3c1oc1ccccc13)CCCCOCC2=O] | 682/684 [M + H]⁺ | 0.61 A | |
| EX-30 | N-13 P-01 | 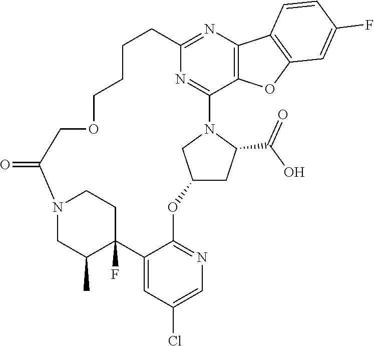 [SMILES: C[C@H]1CN2CC[C@]1(F)c1cc(Cl)cnc1O[C@H]1C[C@@H](C(=O)O)N(C1)c1nc(nc3c1oc1cc(F)ccc13)CCCCOCC2=O] | 656/658 [M + H]⁺ | 0.61 A | |
| EX-31 | N-14 P-01 | 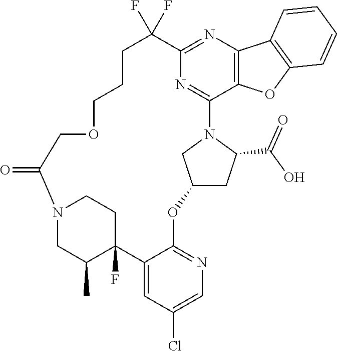 [SMILES: C[C@H]1CN2CC[C@]1(F)c1cc(Cl)cnc1O[C@H]1C[C@@H](C(=O)O)N(C1)c1nc(nc3c1oc1ccccc13)C(F)(F)CCCOCC2=O] | 674/676 [M + H]⁺ | 0.78 A | |
| EX-32 | N-15 P-01 | 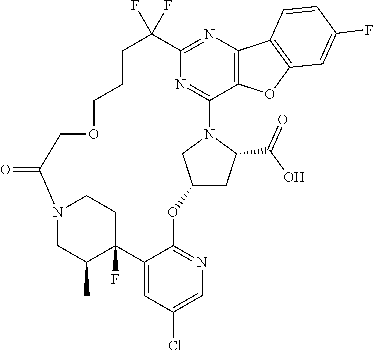 [SMILES: C[C@H]1CN2CC[C@]1(F)c1cc(Cl)cnc1O[C@H]1C[C@@H](C(=O)O)N(C1)c1nc(nc3c1oc1cc(F)ccc13)C(F)(F)CCCOCC2=O] | 692 [M + H]⁺ | 0.79 A | |
| EX-33 | N-16 P-01 | 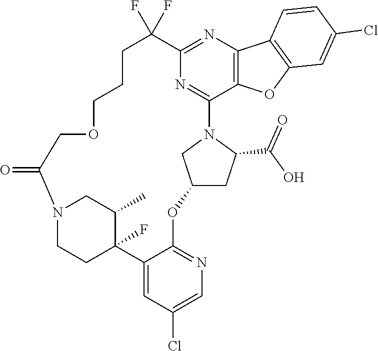 [SMILES: C[C@H]1CN2CC[C@]1(F)c1cc(Cl)cnc1O[C@H]1C[C@@H](C(=O)O)N(C1)c1nc(nc3c1oc1cc(Cl)ccc13)C(F)(F)CCCOCC2=O] | 708 / 710 / 712 [M + H]⁺ | 0.84 A | |
| EX-36 | N-17 P-01 | 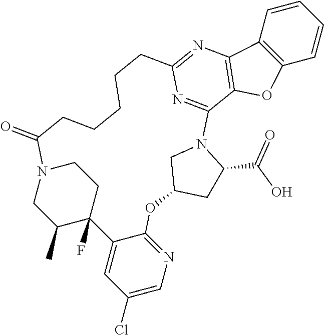 [SMILES: C[C@H]1CN2CC[C@]1(F)c1cc(Cl)cnc1O[C@H]1C[C@@H](C(=O)O)N(C1)c1nc(nc3c1oc1ccccc13)CCCCCC2=O] | 620 / 622 [M + H]⁺ | 0.73 B | |

### Example EX-08: (1R,9S,11S,34S)-4-chloro-1-fluoro-34-methyl-31-oxo-8,15,26-trioxa-6,12,23,32,37-pentaazabestacyclo[30.2.2.1^{9,12}.1^{13,24}.0^{2,7}.0^{14,22}.0^{16,21}]octatriaconta-2,4,6,13(37),14(22),16(21),17,19,23-nonaene-11-carboxylic acid

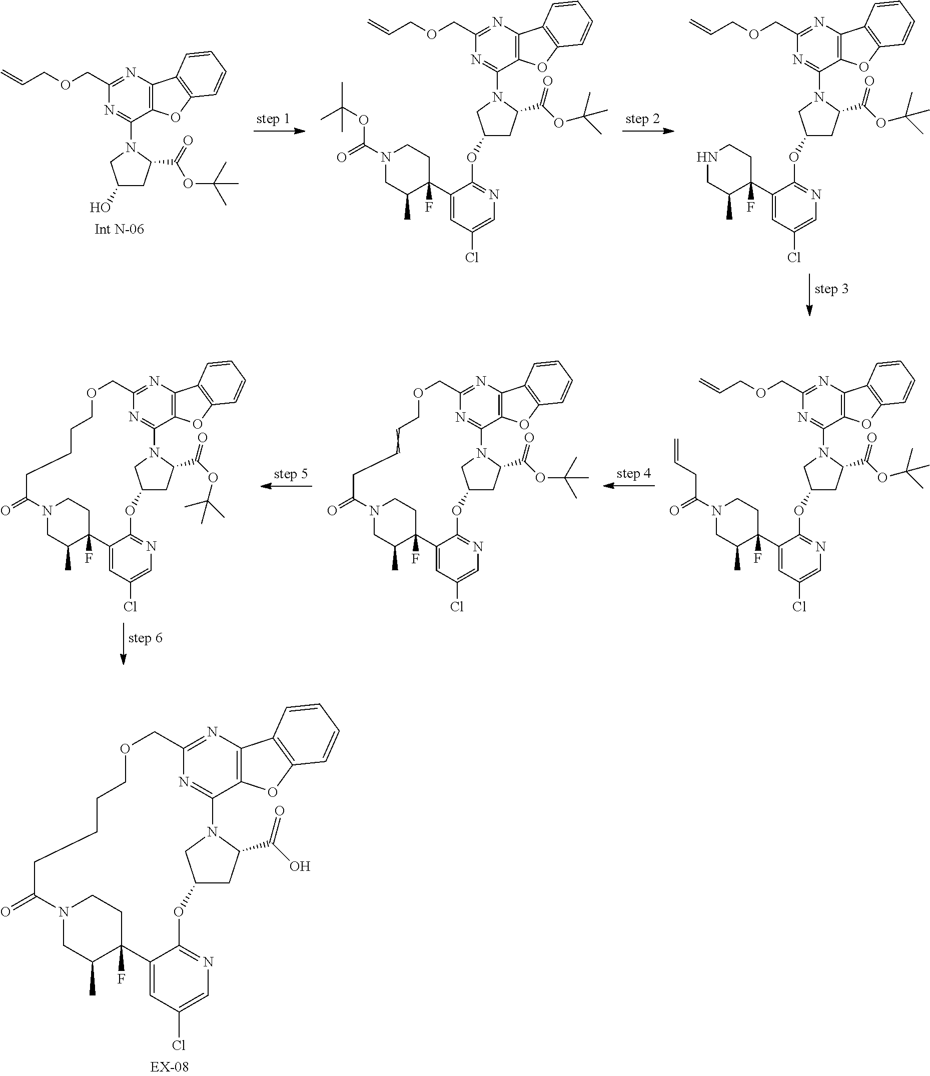

EX-08 Step 1:

Intermediate N-06 was reacted with Intermediate P-01 according to General procedure B using NMP as solvent to yield the title compound.

ESI-MS: 752/754 (1Cl) [M+H]⁺; Rₜ (HPLC): 0.89 min (Method A)

EX-08 Step 2:

The product obtained in EX-08 step 1 was reacted according to General Procedure F.

ESI-MS: 652/654 (1Cl) [M+H]⁺; Rₜ (HPLC): 1.03 min (Method B)

EX-08 Step 3:

General Procedure J: Amidation Applying DCC

At 0° C., a 1 M solution of DCC in DCM (387 μL, 0.387 mmol, 1.30 eq.) was added dropwise under stirring to a mixture of the piperidine obtained from step EX-08 step 2 (200 mg, 0.297 mmol), DMAP (4.7 mg, 0.039 mmol) and vinylacetic acid (34.0 μL, 0.387 mmol) in 2.00 mL DCM. The reaction mixture was allowed to reach RT and stirred at RT until complete conversion of the starting material was observed (30 h). Then, the reaction mixture was cooled to 0° C., diluted with DCM, filtered and the filtrate was concentrated to afford the crude product which was further purified by prep. HPLC (XBridge C18; 30-100% ACN/H₂O/TFA).

ESI-MS: 720/722 (1Cl) [M+H]⁺; Rₜ (HPLC): 0.80 min (Method A)

EX-08 Step 4:

General Procedure K: Olefin Metathesis

Under argon, Grubbs Catalyst® 2^{nd} Generation (25.0 mg, 0.028 mmol, 10 mol %) was added to a degassed solution of the product obtained in EX-08 step 3 (212 mg, 0.280 mmol) in 8.48 mL 1,2-dichloroethane. The vial was sealed, the reaction mixture was heated to 60° C. and stirred at this temperature until the starting material was consumed (here: ˜ 45 min). If necessary, an additional amount of catalyst (25.0 mg, 0.028 mmol, 10 mol %) was added and stirring at 60° C. was continued. Once conversion of starting material was complete (here: ˜ 2 h), the volatiles were removed in vacuo, the residue was dissolved in ACN/H₂O, filtered and purified by means of semi-prep. HPLC (XBridge C18; ACN/H₂O/TFA).

ESI-MS: 692/694 (1Cl) [M+H]⁺; Rₜ (HPLC): 0.73 min (Method A)

EX-08 Step 5:

General Procedure L: Hydrogenation Using Raney-Ni

Raney-Ni (2 mg) was added to a mixture of the product obtained in EX-08 step 5 in 0.10 mL MeOH and 1.00 mL EtOAc in a Parr apparatus. The reaction mixture was placed under 2 bar hydrogen pressure for 2 h at RT. The solids were removed by filtration and the filtrate was concentrated in vacuo to dryness. The residue was taken up in ACN/H₂O and freeze-dried.

ESI-MS: 694/696 (1Cl) [M+H]⁺; Rₜ (HPLC): 0.76 min (Method A)

EX-08 Step 6:

The product obtained in EX-08 step 5 was reacted according to General Procedure H to yield the final compound.

ESI-MS: 638/640 (1Cl) [M+H]⁺; Rₜ (HPLC): 0.63 min (Method A)

### Example EX-09

### (1R,9S,11S,30S,34S)-4-chloro-1-fluoro-30,34-dimethyl-31-oxo-8,15,26-trioxa-6,12,23,32,37-pentaazaheptacyclo[30.2.2.1^{9,12}.1^{13,24}.0^{2,7}.0^{14,22}.0^{16,21}]octatriaconta-2,4,6,13,16,18,20,22,24(37)-nonaene-11-carboxylicacid

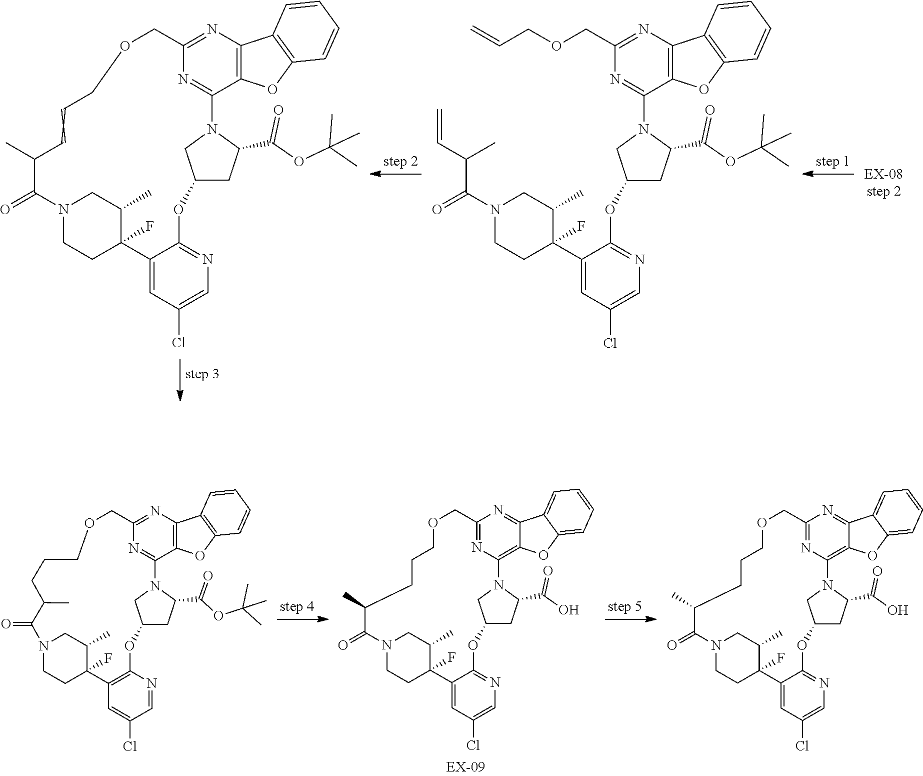

EX-09 step 1:

tert-Butyl (2S,4S)-4-({5-chloro-3-[(3S,4R)-4-fluoro-3-methylpiperidin-4-yl]pyridin-2-yl}oxy)-1-{4-[(prop-2-en-1-yloxy)methyl]-8-oxa-3,5-diazatricyclo[7.4.0.0¹]trideca-1(9),2(7),3,5,10,12-hexaen-6-yl}pyrrolidine-2-carboxylate obtained as product from EX-08 step 2 was reacted with 2-methyl-3-butenoic acid according to General procedure J.

ESI-MS: 734/736 (1Cl) [M+H]⁺; Rₜ (HPLC): 0.83 min (Method A)

EX-09 Step 2:

The product obtained in the previous step was reacted according to General Procedure K and carried on to the next step as a mixture of diastereomers.

ESI-MS: 706/708 (1Cl) [M+H]⁺; Rₜ (HPLC): 0.74/0.76 min (Method A)

EX-09 Step 3:

The product obtained in the previous step was reacted according to General Procedure L and carried on to the next step as a mixture of diastereomers.

ESI-MS: 708/710 (1Cl) [M+H]⁺; Rₜ (HPLC): 0.77 min (Method A)

EX-09 Step 4:

The product obtained in the previous step was reacted according to General Procedure H to afford two diastereomers, which were separated by HPLC (XBridge C18, CAN/H₂O/TFA). EX-09 was the first eluting peak under the applied conditions. The absolute configuration of the alpha methyl substituent was assigned arbitrarily.

ESI-MS: 652/654 (1Cl) [M+H]⁺; Rₜ (HPLC): 0.61 min (Method A)ds of EX-08

ESI-MS: 652/654 (1Cl) [M+H]⁺; Rₜ (HPLC): 0.69 min (Method A)EX-08

### Example EX-10

### (1R,9S,11S,34S)-4-chloro-1,30,30-trifluoro-34-methyl-31-oxo-8,15,26-trioxa-6,12,23,32,37-pentaazaheptacyclo[30.2.2.1^{9,12}.1^{13,24}.0^{2,7}.0^{14,22}.0^{16,21}]octatriaconta-2,4,6,13,16,18,20,22,24(37)-nonaene-11-carboxylic acid

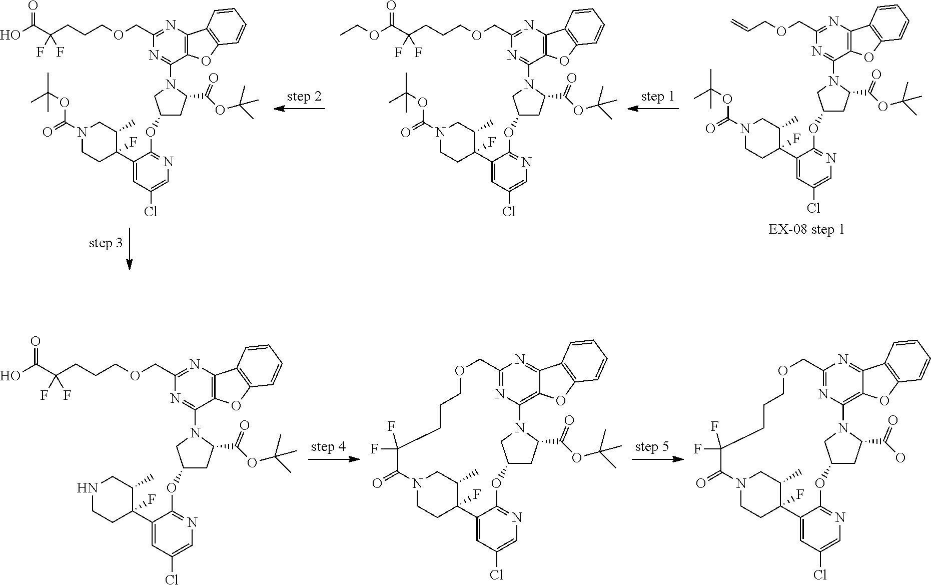

EX-10 Step 1:

Under argon at RT, ethyl bromodifluoroacetate (41.8 mg, 26.7 μL, 0.200 mmol) and phenylsilane (44.6 mg, 50.9 μL, 0.400 mmol) were added to a degassed suspension of tert-butyl (3S,4R)-4-(2-{[(3S,5S)-5-[(tert-butoxy)carbonyl]-1-{4-[(prop-2-en-1-yloxy)methyl]-8-oxa-3,5-diazatricyclo[7.4.0.0^{2,7}]trideca-1(9),2(7),3,5,10,12-hexane-6-yl}pyrrolidin-yl]oxy}5-chloropyridin-3-yl)-4-fluoro-3-methylpiperidine-1-carboxylate (obtained as product from EX-08 step 1) (80.0 mg, 0.100 mmol), Nickel (II) chloride (0.66 mg, 0.0050 mmol) and sodium carbonate (10.6 mg, 0.100 mmol) in 1.00 mL anhydrous DMF. The vial was sealed, and the reaction mixture was stirred for 16 h at 70 T. The reaction mixture was quenched with 10% aq. TFA, diluted with ACN/H₂O, and purified by means of semi-prep. HPLC (XBridge C18; ACN/H₂O/TFA).

ESI-MS: 876/878 (1Cl) [M+H]⁺; Rₜ (HPLC): 0.93 min (Method A)

The product obtained in EX-10 Step 1 was further reacted applying the following reaction sequence:

EX-10 Step 2: Ester hydrolysis applying general procedure C
ESI-MS: 848/850 (1Cl) [M+H]⁺; Rₜ (HPLC): 0.85 min (Method A)

EX-10 Step 3: BOC-deprotection applying General Procedure F
ESI-MS: 748/750 (1Cl) [M+H]⁺; Rₜ (HPLC): 0.64 min (Method A)

EX-10 Step 4: Amidation applying General Procedure D
ESI-MS: 730/732 (1Cl) [M+H]⁺; Rₜ (HPLC): 0.86 min (Method A)

EX-10 Step 5: tert butyl ester deprotection applying General Procedure H.

ESI-MS: 674/676 (1Cl) [M+H]⁺; Rₜ (HPLC): 0.68 min (Method A)

### Example EX-11: (1R,9S,11S,33S)-4-Chloro-1-fluoro-33-methyl-27,30-dioxo-8,15,26-trioxa-6,12,23,31,36-pentaazaheptacyclo[29.2.2.1^{9,12}.1^{13,24}.0^{2,7}.0^{14,22}.0^{16,21}]heptatriaconta-2,4,6,13(36),14(22),16(21),17,19,23-nonaene-11-carboxylic acid

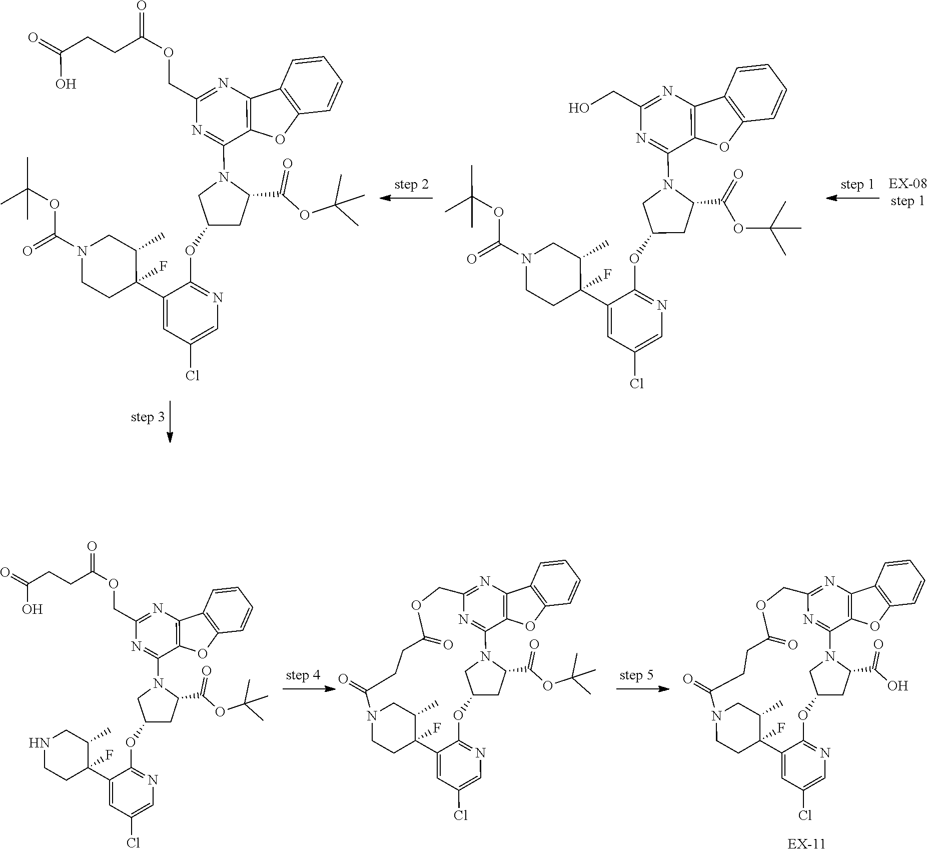

EX-11 step 1:

A mixture of tert-butyl (3S,4R)-4-(2-{[(3S,5S)-5-[(tert-butoxy)carbonyl]-1-{4-[(prop-2-en-1-yloxy)methyl]-8-oxa-3,5-diazatricyclo[1.4.0.0^{2,7}]trideca-1(9),2(7),3,4,10,12-hexan-6-yl}pyrrolidin-3-yl]oxy}-5-chloropyridin-3-yl)-4-fluoro-3-methylpiperidine-1-carboxylate (see EX-08 step 1) (31.6 mg, 0.040 mmol), 1,3-dimethylbarbituric acid (12.6 mg, 0.080 mmol) and Pd(PPh₃)₄ (2.31 mg, 0.0020 mmol) in 0.32 mL methanol was stirred at RT for 5.5 h. The reaction mixture was then heated to 65° C. for 15 h. After cooling to RT, the mixture was diluted with ACN/H₂O, filtered, and purified by means of semi-prep. HPLC (XBridge C18; ACN/H₂O/TFA).

ESI-MS: 712/714 (1Cl) [M+H]⁺; Rₜ (HPLC): 0.81 min (Method A)

EX-11 step 2:

To a mixture of the product obtained in the previous step (60.0 mg, 0.0800 mmol) in 0.60 mL pyridine at RT was added succinic anhydride (25.0 mg, 0.250 mmol) and the mixture was stirred at 50° C. for 5 h. The reaction mixture was allowed to come to RT and stirred at RT for 2 days. Further succinic anhydride (25.0 mg; 0.25 mmol) was added and the mixture was stirred at 50° C. for 6 h. The reaction mixture was acidified by addition of TFA, diluted with ACN/water and purified by means of prep. HPLC (ACN/H₂O/TFA).

ESI-MS: 812 [M+H]⁺; Rₜ (HPLC): 1.20 min (Method B)

The product obtained in EX-11 Step 2 was further reacted applying the following reaction sequence:

EX-11 Step 3: BOC-deprotection applying General Procedure F
ESI-MS: 712 [M+H]⁺; Rₜ (HPLC): 0.77 min (Method B)

EX-11 Step 4: Amidation applying General Procedure D
ESI-MS: 694 [M+H]⁺; Rₜ (HPLC): 0.92 min (Method D)

EX-11 Step 5: tert butyl ester deprotection applying General Procedure H
ESI-MS: 638 [M+H]⁺; Rₜ (HPLC): 0.82 min (Method B)

### Example EX-12

### tert-butyl (2S,4S)-4-({5-Chloro-3-[(3S,4R)-4-fluoro-3-methylpiperidin-4-yl]pyridin-2-yl}oxy)-1-{4-[(prop-2-en-1-yloxy)methyl]-8-oxa-3,5-diazatricyclo[7.4.0.0^{2,7}]trideca-1(9),2(7),3,5,10,12-hexaen-6-yl}pyrrolidine-2-carboxylate

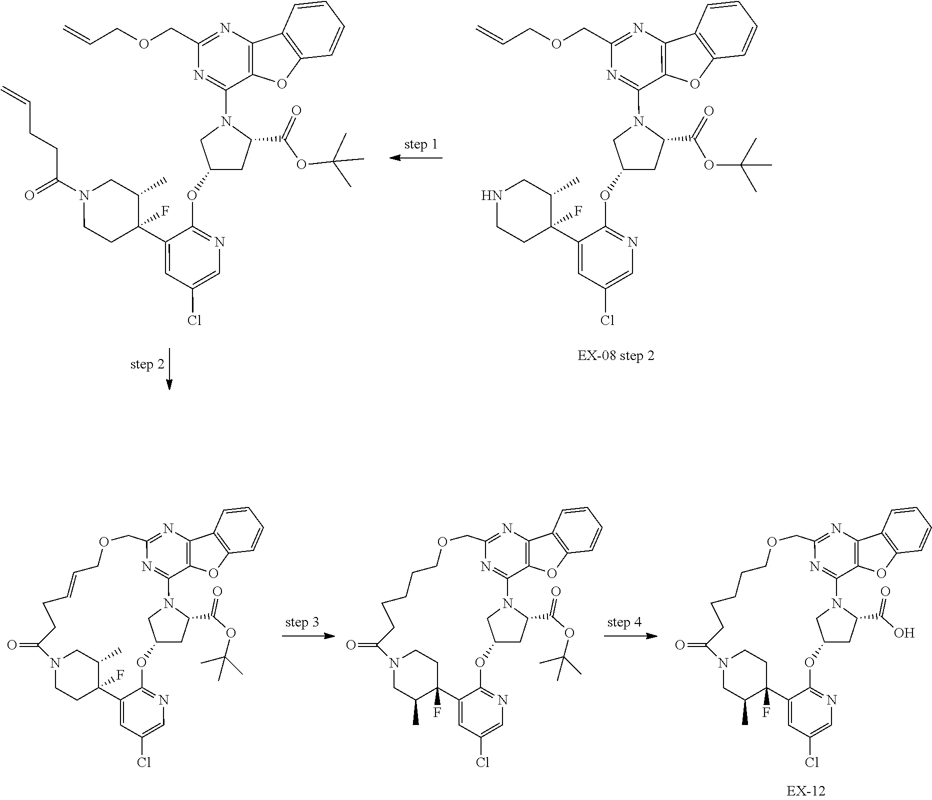

EX-12 Step 1:

To a mixture of pent-4-enoic acid (30.0 mg, 0.303 mmol) in 2.00 mL DCM at RT was added triethylamine (0.084 mL, 0.606 mmol), followed by EDC hydrochloride (70.0 mg, 0.363 mmol). After stirring for 10 min at RT, the product obtained in EX-08 step 2 (290 mg, 0.303 mmol) was added and the mixture was stirred at RT for 4 h. The reaction mixture was poured onto ice water and extracted with EtOAc. The organic phase was combined, washed with water, and dried over sodium sulfate, and evaporated to dryness.

ESI-MS: 734 [M+H]⁺; R₁ (HPLC): 1.059 min (Method C)

The product obtained in EX-11 Step 2 was further reacted applying the following reaction sequence:

EX-12 Step 2: Olefin metathesis according to General Procedure K
ESI-MS: 706 [M+H]⁺; R₁ (HPLC): 0.998 min (Method C)

EX-12 Step 3: Hydrogenation applying General Procedure L
ESI-MS: 708 [M+H]⁺; R₁ (HPLC): 1.063 min (Method C)

EX-12 Step4: tert butyl ester deprotection applying General Procedure H
ESI-MS: 652 [M+H]⁺; R₁ (HPLC): 0.951 mi (Method C)

### Example EX-13

### (1R,9S,11S,34S)-4-chloro-1-fluoro-34-methyl-31-oxo-8,15,26,30-tetraoxa-6,12,23,32,37-pentaaabestacyclo[30.2.2.1^{9,12}.1^{13,24}.0^{14,22}.0^{16,21}]octatriaconta-2(7),3,5,13(37),14(22),16(21),17,19,23-nonaene-11-carboxylic acid

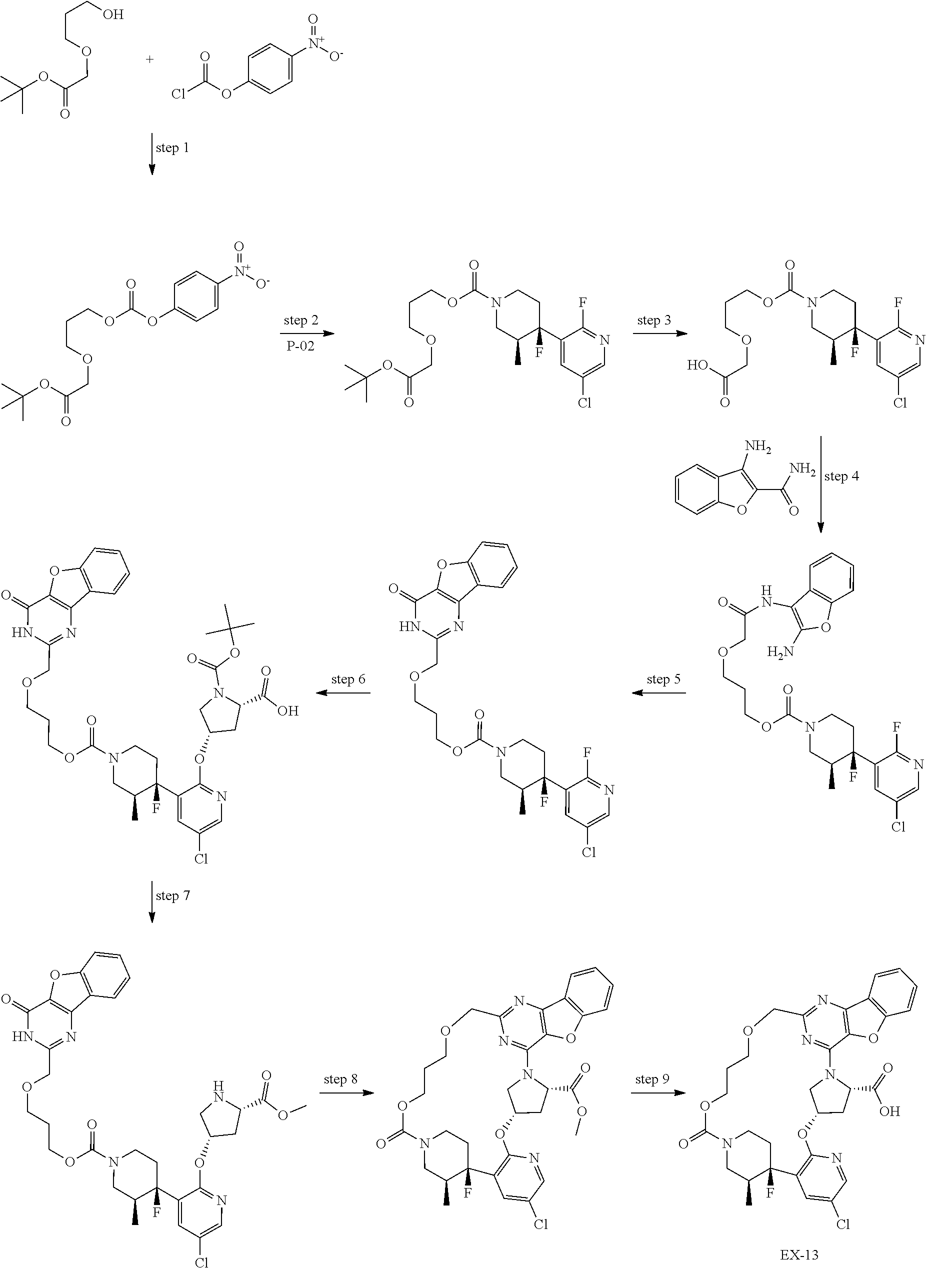

EX-13 Step 1: tert-Butyl 2-(3{[(4-nitrophenoxy)carbonyl]oxy}propoxy)acetate

To a solution of tert-butyl-2-(3-hydroxypropoxy)acetate (1.000 g, 5.10 mmol, preparation described in WO2019/195609) and pyridine (0.441 mL, 5.48 mmol) in 10.0 mL DCM at 0° C. was added dropwise over 30 min a solution of 4-nitrophenyl chloroformate in DCM (10.0 mL). The reaction mixture was allowed to come to RT over night. Water was added, and the phases were separated.

The organic phase was dried and evaporated to dryness.

ESI-MS: 300 [M−tBu+H]⁺; Rₜ (HPLC): 1.00 min (Method C)

EX-13 Step 2: tert-Butyl 2-{3-[(3S,4R)-4-(5-chloro-2-fluoropyridin-3-yl)-4-fluoro-3-methylpiperidine-1-carbonyloxy]propoxy}acetate

A mixture of Intermediate P-02 (250 mg, 0.883 mmol), tert-butyl 2-(3-{[(4-nitrophenoxy)carbonyl]-oxy}propoxy)acetate (396 mg, 0.891 mmol) and DIPEA (0.426 mL, 2.65 mmol) in 4.60 mL THF was heated under reflux for 2 h. After cooling to RT, the reaction mixture was diluted with diethyl ether and extracted with 1M aqueous NaOH solution twice. The organic phase was washed with water and saturated aqueous NaCl solution, dried over sodium sulfate and concentrated under reduced pressure. The crude product was used for the next step without further purification.

ESI-MS: 463 [M+H]⁺; Rₜ (HPLC): 0.76 min (Method A)

EX-13 Step 3 2-{3-[(3S,4R)-4-(5-Chloro-2-fluoropyridin-3-yl)-4-fluoro-3-methylpiperidine-1-carbonyloxy]propoxy}acetic acid

The product obtained from the previous step was reacted according to General Procedure H to yield the anticipated product.

ESI-MS: 407/409 (1Cl) [M+H]⁺; Rₜ (HPLC): 0.58 min (Method A)

EX-13 Step 4: 3-{[(2-Amino-1-benzofuran-3-yl)carbamoyl]methoxy}propyl (3S,4R)-4-(5-chloro-2-fluoropyridin-3-yl)-4-fluoro-3-methylpiperidine-1-carboxylate

At RT, DIPEA (360 μL, 2.08 mmol) was added to a stirred solution of 2-{3-[(3S,4R)-4-(5-chloro-2-fluoropyridin-3-yl)-4-fluoro-3-methylpiperidine-1-carbonyloxy]propoxy}acetic acid (380 mg, 0.934 mmol) and PFTU (440 mg, 1.03 mmol) in 5.50 mL DMF and the mixture was stirred for 5 min at this temperature. Then, 3-amino-1-benzofuran-2-carboxamide (190 mg, 1.035 mmol) was added and the mixture was stirred for 1 h at RT. The reaction mixture was heated to 50° C. and stirred for 36 h at this temperature. It was diluted with diethyl ether and the organic phase was washed with diluted NaOH and brine. The org. layer was dried with sodium sulfate and evaporated to dryness. The residue was dissolved in MeOH acidified with TFA, filtered and purified by means of prep. HPLC (XBridge C18; ACN/H₂O/TFA).

ESI-MS: 565/567 (1Cl) [M+H]⁺; Rₜ (HPLC): 0.68 min (Method A)

EX-13 Step 5: A mixture of 3-{[(2-amino-1-benzofuran-3-yl)carbamoyl]methoxy}propyl (3S,4R)-4-(5-chloro-2-fluoropyridin-3-yl)-4-fluoro-3-methylpiperidine-1-carboxylate (110 mg, 0.195 mmol), chlorotrimethylsilane (0.352 mL, 2.77 mmol) and triethylamine (1.185 mL, 8.44 mmol) in 4.75 mL dichloroethane was heated under reflux for 40 h. After cooling to RT, the reaction mixture was diluted with DCM. Phases were separated and the organic phase was washed with diluted hydrochloric acid, dried over sodium sulfate and concentrated in vacuo to yield the crude product which was used without purification for the next step.

ESI-MS: 447/449 (1Cl) [M+H]⁺; Rₜ (HPLC): 0.68 min (Method A)

EX-13 Step 6: The product obtained from EX-13 step 5 was reacted with (2S,4S)-1-[(tert-butoxy)carbonyl]-4-hydroxypyrrolidine-2-carboxylic acid according to General procedure B.

ESI-MS: 758/760 (1Cl) [M+H]⁺; Rₜ (HPLC): 0.81 min (Method A)

EX-13 Step 7: The product obtained from EX-13 step 5 (140 mg, 0.185 mmol) was dissolved in methanol and thionyl chloride (89 μL, 1.21 mmol) was added. After stirring for 2 h another portion of thionyl chloride (55 μL, 0.754 mmol) was added and stirring was continued over night. The reaction mixture was concentrated in vacuo, and the crude product was purified by means of HPLC (XBridge C18; ACN/H₂O/TFA).

ESI-MS: 672/674 (1Cl) [M+H]⁺; Rₜ (HPLC): 0.58 min (Method A)

EX-13 Step 8: The product from EX-13 step 7 (21 mg, 0.030 mmol) was taken up in 2.00 mL DMF at RT. DBU (22.4 μL, 0.148 mmol) and BOP (35 mg, 0.077 mmol) was added and the mixture was stirred at RT for 2 h. After addition of water, the mixture was extracted with diethyl ether (twice). The combined organic phase was washed with brine, dried over sodium sulfate and concentrated in vacuo.

ESI-MS: 654/656 (1Cl) [M+H]⁺; Rₜ (HPLC): 0.74 min (Method A)

EX-13 Step 9: To a mixture of the product from EX-13 step 8 (25 mg, 0.038 mmol) in 2.00 mL dioxane was added 1M aqueous NaOH solution (450 μL, 0.450 mmol). The reaction mixture was heated at 60° C. for 30 min. At RT, the reaction mixture was acidified, diluted with methanol and subjected to HPLC purification (XBridge C18; ACN/H₂O/TFA).

ESI-MS: 640/642 (1Cl) [M+H]⁺; Rₜ (HPLC): 0.68 min (Method A)

### Example EX-15: (1R,9S,11S,34S)-4-chloro-1,25,25-trifluoro-34-methyl-31-oxo-8,15,26-trioxa-6,12,23,32,37-pentaazaheptacyclo[30.2.2.1^{9,12}.1^{13,24}.0^{2,7}.0^{14,22}.0^{16,21}]octatriaconta-2(7),3,5,13(37),14(22),16(21),17,19,23-nonaene-11-carboxylic acid

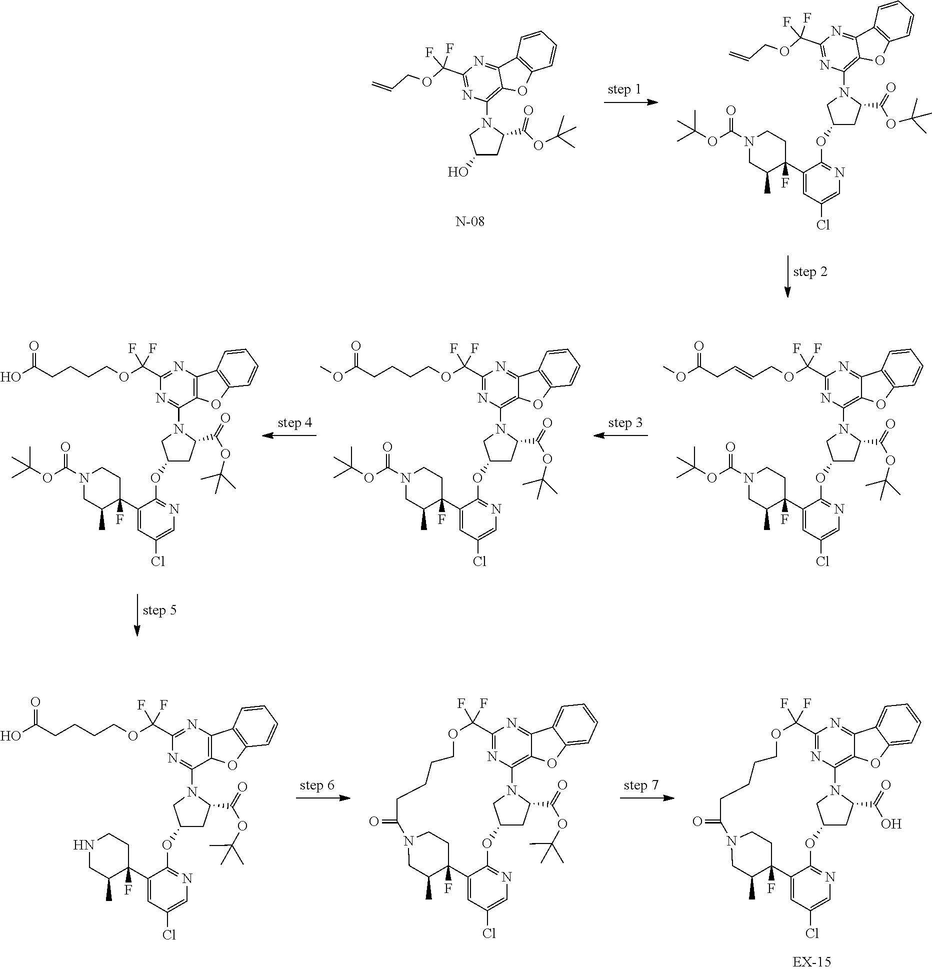

EX-15 Step 1:

NaH (130 mg, 3.25 mmol, 60% in mineral oil) was added to a mixture of N-08 (370 mg, 0.802 mmol) and P-01 (285 mg, 0.812 mmol) in 10.0 mL DMF, and the reaction mixture was stirred at RT for 1 h. The reaction mixture was added dropwise into cold water and was acidified using 1M aq. HCl solution. The precipitate was taken up in diethyl ether. The combined organic layers were washed with brine, dried with sodium sulfate and concentrated in vacuo. The residue was further purified via silica gel chromatography using cyclohexane/EA as eluent.

ESI-MS: 788/790 (1Cl) [M+H]⁺; Rₜ (HPLC): 1.01 min (Method A)

EX-15 Step 2:

Under argon, Grubbs Catalyst® 2^{nd} Generation (4.07 mg, 0.005 mmol, 6 mol %) was added to a degassed solution of the product obtained in EX-15 step 1 (63.0 mg, 0.076 mmol) and methyl-3-butenoate (128 μL, 1.139 mmol) in 0.50 mL DCM. The vial was sealed and the reaction mixture was stirred at RT for 2 h. Additional methyl-3-butenoate (42.7 μL, 5.0 eq.) was added and stirring was continued for 2 h. Then, an additional aliquot of Grubbs Catalyst® 2^{nd} Generation (4.07 mg, 0.005 mmol, 6 mol %) was added and stirring continued for 1.5 h. Again, catalyst (4.07 mg; 6 mol %) and methyl-3-butenoate (42.7 μL; 3.00 eq.) were added and stirring at RT was continued for an additional 18 h. The volatiles were removed in vacuo, the residue was dissolved in EtOAc/cyclohexane and purified directly by silica gel chromatography.

ESI-MS: 860/862 (1Cl) [M+H]⁺; Rₜ (HPLC): 0.75 min (Method G)

EX-15 Step 3:

General Procedure N: Hydrogenation Using Pd/C 10% Pd/C (90.0 mg) was added to a solution of the product obtained in EX-15 step 3 (250 mg, 0.276 mmol) in 40 mL EtOAc in a Parr apparatus and the reaction mixture was placed under 3 bar hydrogen pressure for 18 h at rt. The solids were removed by filtration and the filtrate was concentrated in vacuo to dryness to afford the crude product which was purified by means of semi-prep. HPLC (Sunfire C18; ACN/H₂O/TFA).

ESI-MS: 862/864 (1Cl) [M+H]⁺; Rₜ (HPLC): 1.12 min (Method B)

The product obtained in EX-15 Step 3 was further reacted applying the following reaction sequence:

EX-15 Step 4: Ester hydrolysis applying General procedure C
ESI-MS: 848/850 (1Cl) [M+H]⁺; Rₜ (HPLC): 0.94 min (Method A)

EX-15 Step 5: BOC-deprotection applying General procedure F
ESI-MS: 748/750 (1Cl) [M+H]⁺; Rₜ (HPLC): 0.82 min (Method B)

EX-15 Step 6: Amidation applying General procedure E
ESI-MS: 730/732 (1Cl) [M+H]⁺; Rₜ (HPLC): 0.98 min (Method D)

EX-15 Step 7: tert butyl ester deprotection applying General procedure H
ESI-MS: 674/676 (1Cl) [M+H]⁺; Rₜ (HPLC): 0.68 min (Method A)

### Example EX-18

### (1R,9S,11S,34S)-4-ethynyl-1-fluoro-34-methyl-31-oxo-8,15,27-trioxa-6,12,23,32,37-pentaazaheptacyclo[30.2.2.1^{9,12}.1^{13,24}.0^{2,7}.0^{14,22}.0^{16,21}]octatriaconta-2,4,6,13,16,18,20,22,24(37)-nonaene-11-carboxylic acid

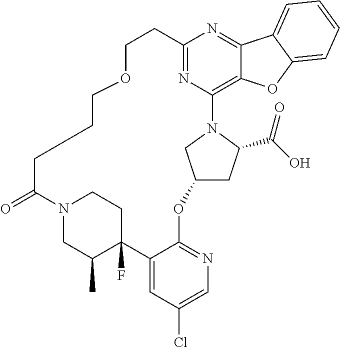 [SMILES: C[C@H]1CN2CC[C@]1(F)c1cc(Cl)cnc1O[C@H]1C[C@@H](C(=O)O)N(C1)c1nc(nc3c1oc1ccccc13)CCOCCCCC2=O]

Under an argon atmosphere, Pd(dppf)Cl₂ (9.0 mg, 0.012 mmol) and Cul (3.0 mg, 0.016 mmol) was added to a mixture of EXAMPLE EX-17 (30 mg, 0.044 mmol), (triisopropylsilyl)acetylene (24 μL, 0.11 mmol) and triethylamine (30 μL, 4.8 eq) in 0.75 mL 2-methyltetrahydrofuran, and the reaction mixture was stirred at 80° C. for 2 h. After cooling to RT, it was filtered and the filtrate concentrated in vacuo. The remainder was taken up in 2.00 mL, TBAF (1M solution in THF, 100 μL, 0.100 mmol) was added, and the mixture was stirred at RT for 30 min. It was then added dropwise to cold water, acidified using 1 M aqueous HCl solution and extracted with diethyl ether twice. The combined organic layers were washed with water and brine and concentrated in vacuo. The product was purified by means of HPLC (XBridge C18; ACN/H₂O/TFA).

ESI-MS 628 [M+H]⁺; Rₜ (HPLC): 0.59 min (Method A)

### Example EX-19

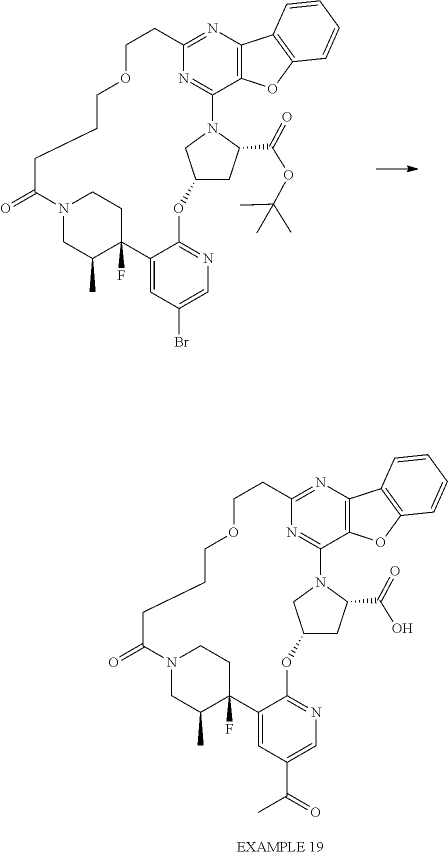 [SMILES: C[C@H]1CN2CC[C@]1(F)c1cc(Br)cnc1O[C@H]1C[C@@H](C(=O)OC(C)(C)C)N(C1)c1nc(nc3c1oc1ccccc13)CCOCCCC2=O ; CC(=O)c1cnc2c(c1)[C@@]1(F)CCN(C[C@@H]1C)C(=O)CCCOCCc1nc(c3oc4ccccc4c3n1)N1C[C@H](C[C@H]1C(=O)O)O2]

The product obtained from the synthesis of EX-17 step 3 was submitted to Sonogashira reaction conditions according to EX-18, and subsequently the t-butyl group was deprotected applying general procedure EX-D to afford the title compound.

ESI-MS 646 [M+H]⁺; Rₜ (HPLC): 0.64 min (Method A)

### Example EX-20.01

### (1R,9S,11S,34S)-1-fluoro-4-(2-methoxypyridin-3-yl)-34-methyl-31-oxo-8,15,27-trioxa-6,12,23,32,37-pentaazaheptacyclo[30.2.2.1^{9,12}.1^{13,24}.0^{2,7}.0^{14,22}.0^{16,21}]octatriaconta-2,4,6,13(37),14(22),16(21),17,19,23-nonaene-11-carboxylic acid

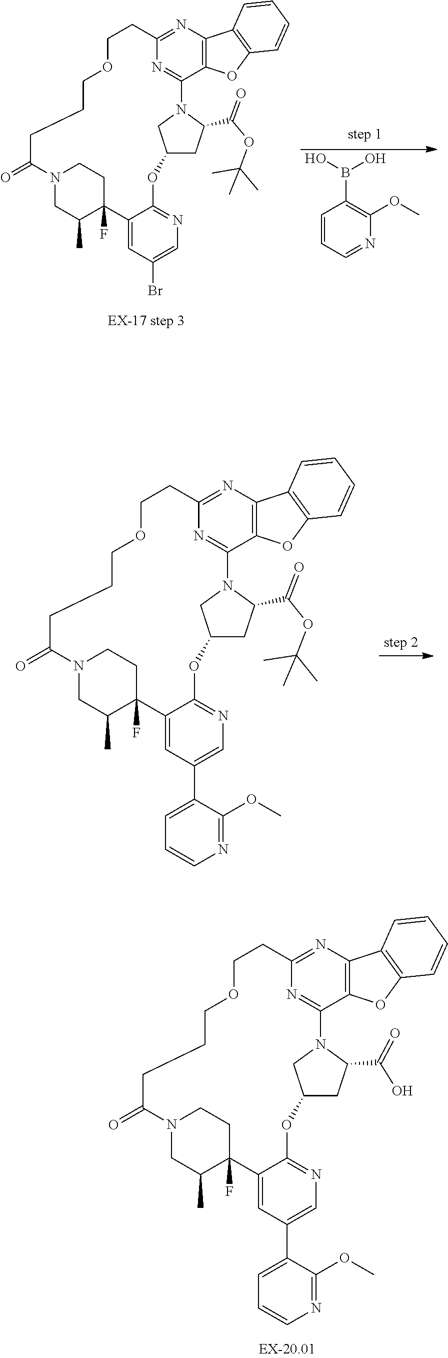 [SMILES: C[C@H]1CN2CC[C@]1(F)c1cc(Br)cnc1O[C@H]1C[C@@H](C(=O)OC(C)(C)C)N(C1)c1nc(nc3c1oc1ccccc13)CCOCCCC2=O ; COc1ncccc1B(O)O ; COc1ncccc1-c1cnc2c(c1)[C@@]1(F)CCN(C[C@@H]1C)C(=O)CCCOCCc1nc(c3oc4ccccc4c3n1)N1C[C@H](C[C@H]1C(=O)OC(C)(C)C)O2 ; COc1ncccc1-c1cnc2c(c1)[C@@]1(F)CCN(C[C@@H]1C)C(=O)CCCOCCc1nc(c3oc4ccccc4c3n1)N1C[C@H](C[C@H]1C(=O)O)O2]

EX-20.01 step 1:

General Procedure O: Suzuki Coupling

Under an argon atmosphere at RT, (2-dicyclohexylphosphino-2′,4′,6′-triisopropyl-1,1′-biphenyl)[2-(2′-amino-1,1′-biphenyl)]palladium(ii) methanesulfonate (XPhos Pd G3) was added to a degassed mixture of aryl bromide (EX-17 step 3, 35 mg, 0.047 mmol), (2-methoxypyridin-3-yl)boronic acid (11 mg, 0.071 mmol) and potassium carbonate (2N aqueous sol., 95 μL, 0.19 mmol). The vial was sealed and heated at 90° C. until full conversion of the starting material was observed (1 h). The heating was removed and the mixture was allowed to come to RT. Water was added and the mixture was extracted with DCM twice. The organic layer was washed with brine, dried with sodium sulfate and evaporated to dryness.

ESI-MS: 767 [M+H]⁺; Rₜ (HPLC): 0.70 min (Method A)

EX-20.01 step 2:

The product of the Suzuki coupling reaction was deprotected applying general procedure General Procedure H to afford the final compound.

ESI-MS 711 [M+H]⁺; Rₜ (HPLC): 0.61 min (Method A)

The following EXAMPLES were prepared according to the reaction sequence described above (General Procedures O and H (t-Bu not applicable if free acid subjected to Suzuki coupling)):

| | | | ESI-MS | |
|---|---|---|---|---|
| | | | Rₜ (HPLC) | |
| | Starting | | [min] | Synthesis |
| Ex. | materials | Structure | (method) | comment |
| EX-20.02 | EX-17 step 3 + 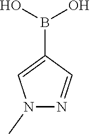 | 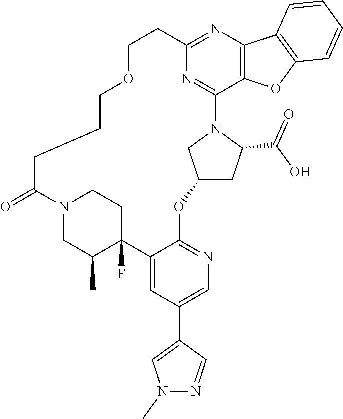 [SMILES: C[C@H]1CN2CC[C@]1(F)c1cc(-c3cnn(C)c3)cnc1O[C@H]1C[C@@H](C(=O)O)N(C1)c1nc(nc3c1oc1ccccc13)CCOCCCC2=O] | 684 [M + H]⁺ method A | — |
| EX-20.03 | EX-17 + 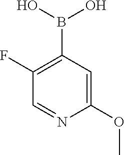 | 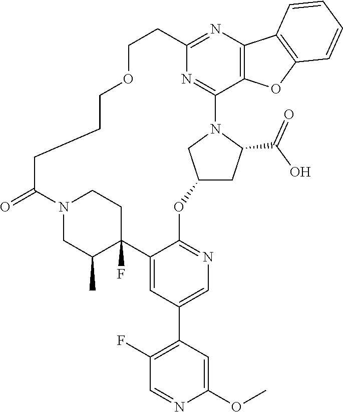 [SMILES: COc1cc(-c2cnc3c(c2)[C@@]2(F)CCN(C[C@@H]2C)C(=O)CCCOCCc2nc(c4oc5ccccc5c4n2)N2C[C@H](C[C@H]2C(=O)O)O3)c(F)cn1] | 729 [M + H]⁺ 0.62 min method A | — |

### Example EX-21: (1R,9S,11S,34S)-4-chloro-1,25,25-trifluoro-34-methyl-31-oxo-8,15,27-trioxa-6,12,23,32,37-penta-azaheptacyclo[30.2.2.1^{9,12}.1^{13,24}.0^{2,7}.0^{14,22}.0^{16,21}]octatriaconta-2,4,6,13(37),14(22),16(21),17,19,23-nonaene-11-carboxylic acid

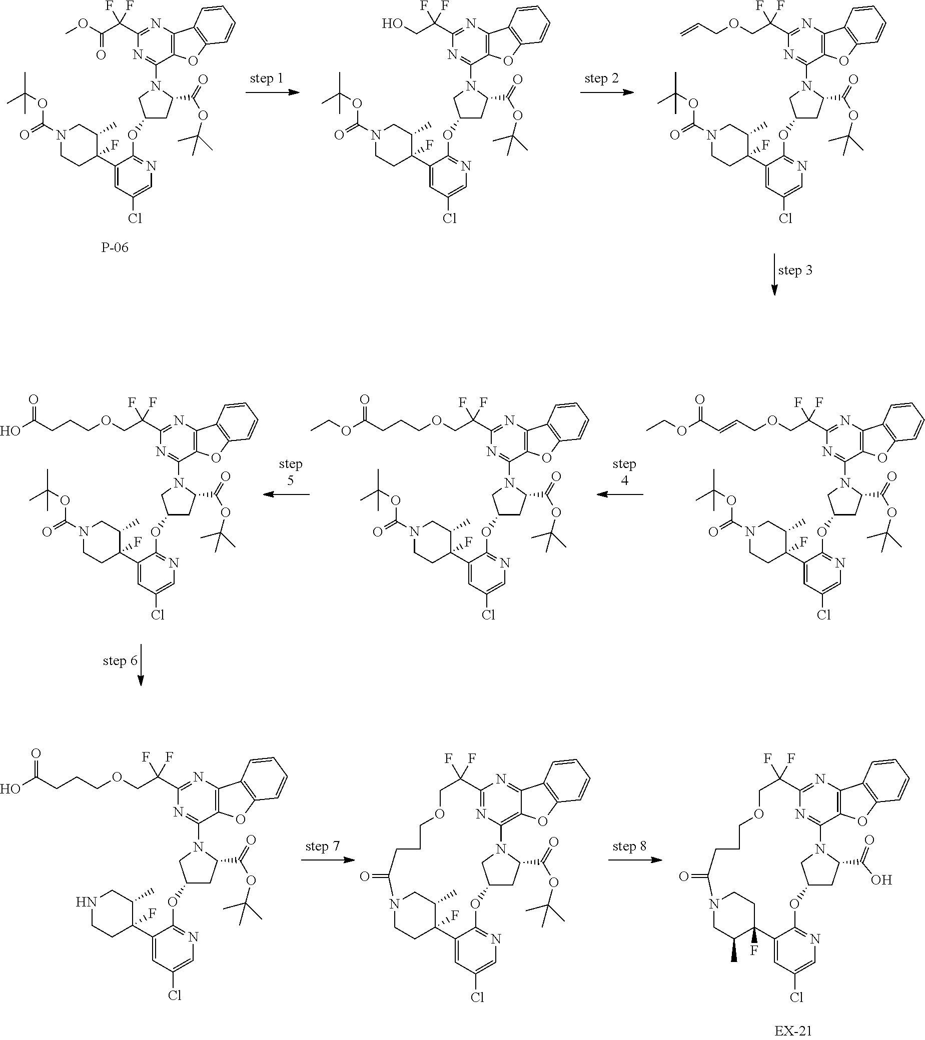

EX-21 step 1: Under an argon atmosphere at 0° C., sodium borohydrate (90 mg, 2.38 mmol) was added to a solution of Intermediate P-06 (730 mg, 794 mmol) in 8.34 mL absolute ethanol. The reaction mixture was stirred at 0° C. for 1 h, and 2 h at RT. Water was added to the mixture, and the product was extracted with EtOAc thrice. The organic phase was washed subsequently with water and brine, dried over MgSO₄, filtered and concentrated under reduced pressure. The crude product was purified by means of silica column chromatography (10%-≥100% EtOAc/CH).

ESI-MS 762/764 (1Cl) [M+H]⁺; Rₜ (HPLC): 0.94 min (Method A)

EX-21 step 2: The material obtained in the previous step was dissolved in 2.44 mL DMA and sodium hydride (50 mg, 1.15 mmol) was added, followed immediately by allyl bromide (250 μL, 2.86 mmol), and the mixture was stirred at RT for 10 min. Ice was added to the reaction mixture, and the product was extracted with EtOAc thrice. The organic phase was washed subsequently with water and brine, dried over MgSO₄, filtered and concentrated under reduced pressure. The crude material was purified by semi prep HPLC (ACN, Xbridge, TFA) using a narrow gradient 80-100% ACN in water.

ESI-MS 803/805 (1Cl) [M+H]⁺; Rₜ (HPLC): 1.03 min (Method A)

EX-21 step 3: The product obtained from the previous step (232 mg, 0.289 mmol) was dissolved in 2.00 mL ethyl acrylate and the mixture was degassed by bubbling with argon for 5 minutes before Grubbs Catalyst® 2^{nd} Generation (24.5 mg, 0.029 mmol) was added to the reaction. The mixture was stirred at RT under an argon atmosphere for 1 h, then directly adsorbed onto extrelute while concentrating under reduced pressure and purified by flash chromatography. 20%->100% EtOAc/CH.

ESI-MS 875/877 (1Cl) [M+H]⁺; Rₜ (HPLC): 1.00 min (Method A)

The product obtained in EX-21 Step 3 was further reacted applying the following reaction sequence:

EX-21 step 4: Hydrogenation applying general Procedure L

EX-21 step 5: Ester hydrolysis applying general procedure C ESI-MS 848/850 (1Cl) [M+H]⁺; Rₜ(HPLC): 0.68 min (Method G)

EX-21 step 6: BOC-deprotection applying general Procedure F ESI-MS: 748/751 (1Cl) [M+H]⁺; Rₜ(HPLC): 0.69 min (Method A)

EX-21 step 7: Amidation applying general Procedure D
ESI-MS: 731/733 (1Cl) [M+H]⁺; Rₜ (HPLC): 0.88 min (Method A)

EX-21 step 8: tert butyl ester deprotection applying general Procedure H
ESI-MS: 675/677 (1Cl) [M+H]⁺; Rₜ (HPLC): 0.79 min (Method A)

### Example EX-22

### (1R,9S,11S,34S)-4-chloro-1-fluoro-34-methyl-31-oxo-8,15,30-trioxa-6,12,23,32,37-pentaaabestacyclo[30.2.2.1^{9,12}.1^{13,24}.0^{2,7}.0^{14,22}.0^{16,21}]octatriaconta-2,4,6,13(37),14(22),16(21),17,19,23-nonaene-11-carboxylic acid

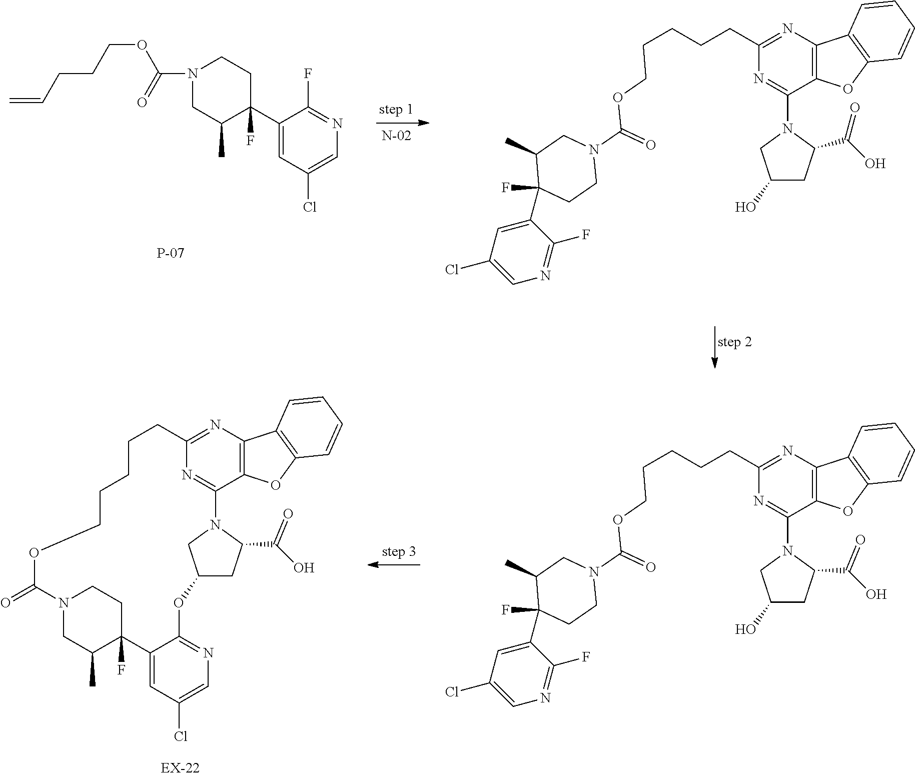 [SMILES: C=CCCCOC(=O)N1CC[C@](F)(c2cc(Cl)cnc2F)[C@@H](C)C1 ; C[C@H]1CN(C(=O)OCCCCCc2nc(N3C[C@@H](O)C[C@H]3C(=O)O)c3oc4ccccc4c3n2)CC[C@]1(F)c1cc(Cl)cnc1F ; C[C@H]1CN2CC[C@]1(F)c1cc(Cl)cnc1O[C@H]1C[C@@H](C(=O)O)N(C1)c1nc(nc3c1oc1ccccc13)CCCCCOC2=O ; C[C@H]1CN(C(=O)OCCCCCc2nc(N3C[C@@H](O)C[C@H]3C(=O)O)c3oc4ccccc4c3n2)CC[C@]1(F)c1cc(Cl)cnc1F]

EX-22 Step 1: General Procedure M: Heck Coupling

A mixture of Intermediate P-07 (85 mg, 0.213 mmol), Intermediate N-02 (70 mg, 0.176 mmol), palladium(II)-acetate (7.9 mg, 0.035 mmol), tri (o-tolyl)phosphine (22 mg, 0.070 mmol) and triethylamine (71 mg, 0.70 mmol) in 2.85 mL DMF was heated in a sealed tube under an argon atmosphere to 95° C. for 6 h. The reaction mixture was allowed to cool to RT and stirred at RT overnight. The volatiles were removed in vacuo and the crude mixture was purified my means of silica column chromatography (20-50% EE in CH). The product was isolated as a mixture of isomers and was used as such for the next reaction step.

ESI-MS 656 [M+H]⁺; Rₜ (HPLC): 0.70/0.71/0.73 min (Method B)

The product obtained in EX-22 Step 1 was further reacted applying the following reaction sequence:

EX-22 step 2: Hydrogenation applying General Procedure L

ESI-MS 658 [M+H]⁺; Rₜ (HPLC): 0.76 min (Method B)

EX-22 step 3: Macrocyclization via S_{N}Ar applying General Procedure I

ESI-MS 638 [M+H]⁺; R (HPLC): 0.85 mm (Method B)

### Example EX-23: (1R,9S,11S,34S)-4-Chloro-1-fluoro-34-methyl-31-oxo-8,15,28-trioxa-6,12,23,32,37-pentaazabestacyclo[30.2.2.1^{9,12}.1^{13,24}.0^{2,7}.0^{14,22}.0^{16,21}]octatriaconta-2,4,6,13(37),14(22),16(21),17,19,23-nonaene-11-carboxylic acid

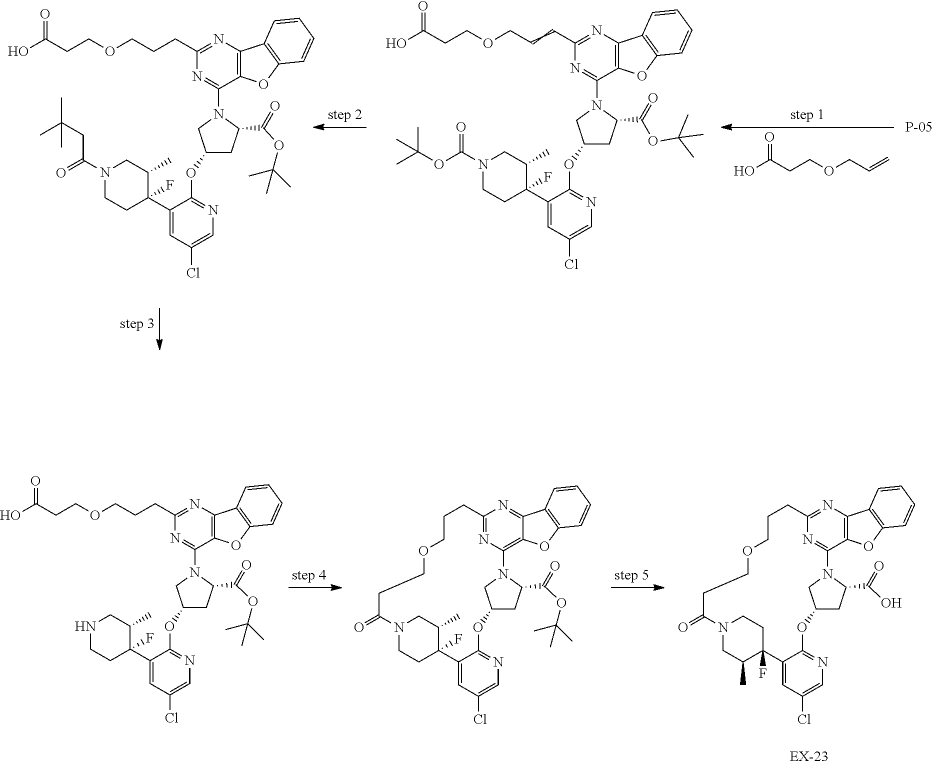

The title compound was prepared starting from Intermediate P-05 applying the following reaction sequence:

EX-23 step 1: Heck coupling with 3-(prop-2-en-1-yloxy)propanoic acid applying General Procedure M

ESI-MS: 810/812 (1Cl); Rₜ (HPLC): 0.81/0.82 min (Method A)

EX-23 step 2: Hydrogenation (Pd/C) according to General Procedure N

ESI-MS: 812/814 (1Cl) [M+H]⁺; Rₜ (HPLC): 0.98 min (Method B)

EX-23 step 3: BOC-deprotection applying General Procedure F

ESI-MS 712/714 (1Cl) [M+H]⁺; Rₜ (HPLC): 0.67 min (Method B)

EX-23 step 4: Amidation applying General Procedure E

ESI-MS 694/696 (1Cl) [M+H]⁺; Rₜ (HPLC): 0.97 min (Method D)

EX-23 step 5: tert butyl ester deprotection applying General Procedure H

ESI-MS 638/640 (1Cl) [M+H]⁺; Rₜ (HPLC): 0.74 min (Method A)

### Example EX-24

### (1R,9S,11S,34S)-1-fluoro-34-methyl-31-oxo-8,15,28-trioxa-6,12,23,32,37-pentaazaheptacyclo-[30.2.2.1^{9,12}.1^{13,24}.0^{2,7}.0^{14,22}.0^{16,21}]octatriaconta-2,4,6,13(37),14(22),16(21),17,19,23-nonaene-11-carboxylic acid

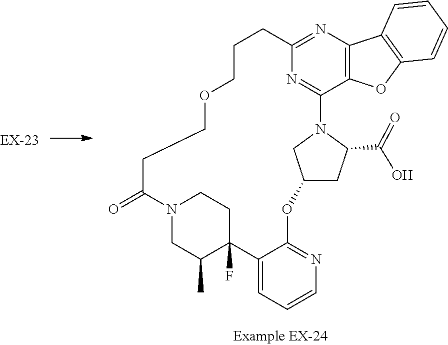 [SMILES: C[C@H]1CN2CC[C@]1(F)c1cccnc1O[C@H]1C[C@@H](C(=O)O)N(C1)c1nc(nc3c1oc1ccccc13)CCCOCCC2=O]

This compound was prepared from EX-24 via hydrogenation applying General Procedure N.

ESI-MS 604 [M+H]⁺; Rₜ (HPLC): 0.65 min (Method B)

### Example EX-29

### (1R,9S,11S,34S)-1-fluoro-34-methyl-31-oxo-4-(prop-1-yn-1-yl)-8,15,29-trioxa-6,12,23,32,37-penta-azaheptacyclo[30.2.2.1^{9,12}.1^{13,24}.0^{2,7}.0^{14,22}.0^{16,21}]octatriaconta-2(7),3,5,13,16(21),17,19,22,24(37)-nonaene-11-carboxylic acid

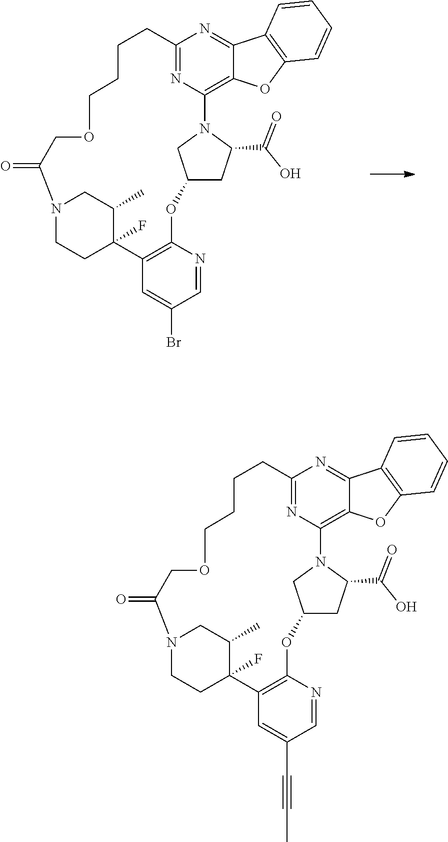 [SMILES: C[C@H]1CN2CC[C@]1(F)c1cc(Br)cnc1O[C@H]1C[C@@H](C(=O)O)N(C1)c1nc(nc3c1oc1ccccc13)CCCCOCC2=O ; CC#Cc1cnc2c(c1)[C@@]1(F)CCN(C[C@@H]1C)C(=O)COCCCCc1nc(c3oc4ccccc4c3n1)N1C[C@H](C[C@H]1C(=O)O)O2]

This compound was prepared from EX-28 via Sonogashira coupling as described for EX-18.

ESI-MS: 642 [M+H]⁺; Rₜ (HPLC): 0.61 min (Method A)

### Example EX-34

### (1R,9S,11S,34S)-4-chloro-1-fluoro-34-methyl-31-oxo-8,15,27,30-tetraoxa-6,12,23,32,37-pentaaza-heptacyclo[30.2.2.1^{9,12}.1^{13,24}.0^{2,7}.0^{14,22}.0^{16,21}]octatriaconta-2,4,6,13(37),14(22),16(21),17,19,23-nonaene-11-carboxylic acid

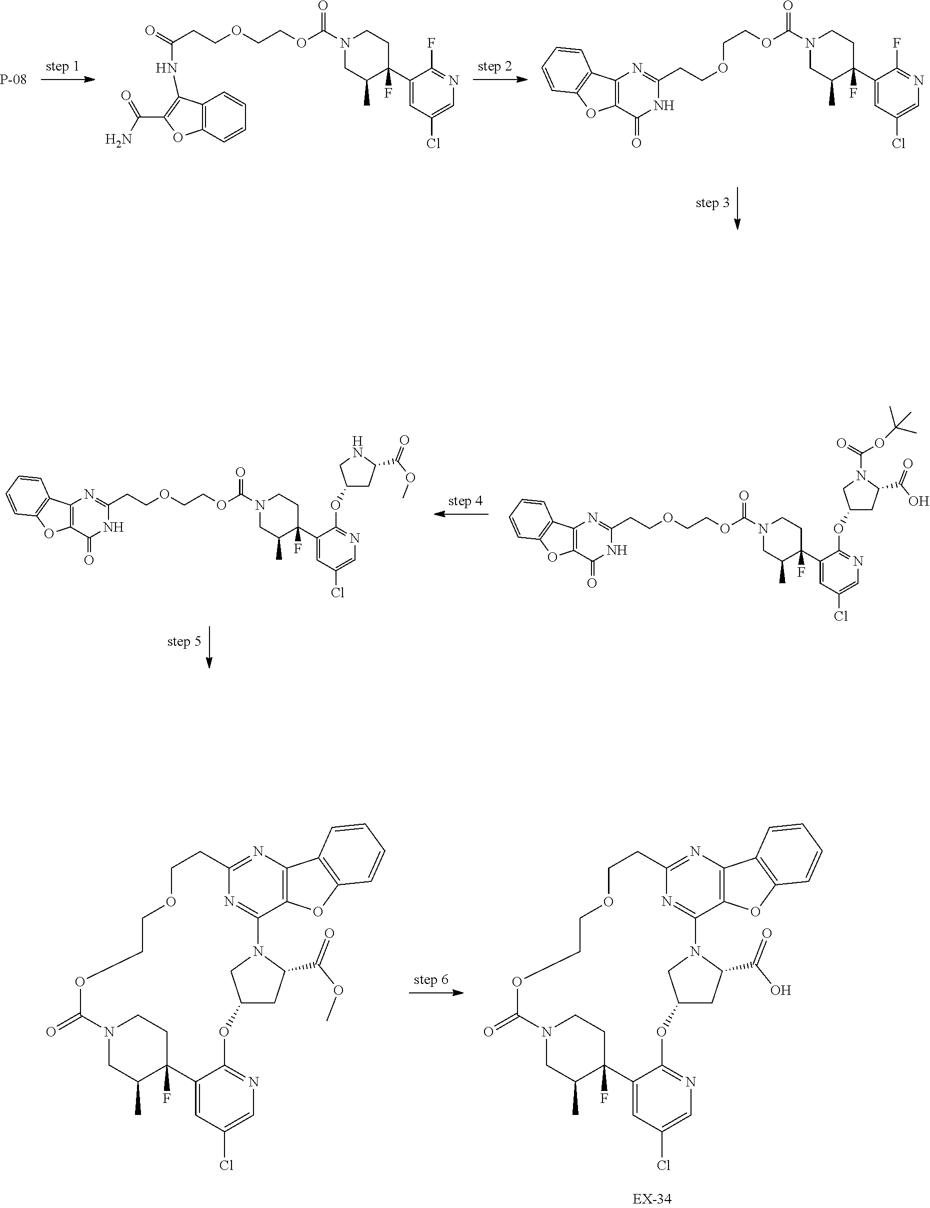

EX-34 Step 1:

To a solution of Intermediate P-08 (115 mg, 0.284 mmol) in 2.00 mL acetonitrile at RT was added 1-chloro-N,N,2-trimethylpropenylamine (50 μL, 0.378 mmol) and the resulting mixture was stirred for 10 min. Then, pyridine (70 μL, 0.856 mmol) was added to the mixture, followed by 3-aminobenzofuran-2-carboxamide (230 mg, 1.31 mmol), and the mixture was stirred at RT for 2 h. The reaction mixture was concentrated to dryness and water and DCM was added. Phases were separated and the organic phase was dried and concentrated to dryness.

ESI-MS: 565/567 (1Cl) [M+H]⁺; Rₜ (HPLC): 0.64 min (Method A)

EX-34 Step 2:

To a solution of the product from step 1 (160 mg, 0.283 mmol) in 7.00 mL 1,2-dichloroethane at RT was added chlorotrimethylsilane (0.512 mL, 4.03 mmol) followed by triethylamine (1.724 mL, 12.3 mmol), and the resulting mixture was stirred under reflux for 40 h. The reaction was cooled to RT and diluted with DCM. The organic phase was washed with aqueous diluted HCl solution, dried over sodium sulfate and concentrated to dryness.

ESI-MS: 547/549 (1Cl) [M+H]⁺; Rₜ (HPLC): 0.64 min (Method A)

EX-34 Step 3:

To a solution of tert-butyl (2S,4S)-4-hydroxypyrrolidine-2-carboxylate hydrochloride (151 mg, 0.635 mmol) in 2.17 mL DMF at RT was added NaH (60% in mineral oil, 50.8 mg, 1.27 mmol). After stirring for 5 min, a solution of product obtained in EX-34 step 2 (217 mg, 0.32 mmol) in 2.17 mL DMF was added slowly, and the mixture was stirred at RT for 1 h. The reaction mixture was added dropwise to water, acidified with acetic acid and extracted with EtOAc twice. The combined organic phase was washed with brine, dried over sodium sulfate and evaporated to dryness. The crude product was purified by means of HPLC (Sunfire, ACN/H₂O/TFA, Narrow).

ESI-MS: 758/760 (1Cl) [M+H]⁺; Rₜ (HPLC): 1.06 min (Method B)

EX-34 Step 4:

The product from EX-34 step 3 (100 mg, 0.130 mmol) was dissolved in 2.00 mL methanol and thionyl chloride (0.060 mL, 0.79 mmol) was added slowly at RT. The reaction mixture was stirred at RT for 48 h, then concentrated under reduced pressure und purified by means of HPLC (Sunfire, ACN/H₂O/TFA, Narrow).

ESI-MS: 672/674 (1Cl) [M+H]⁺; Rₜ (HPLC): 0.70 min (Method B)

EX-34 Step 5:

DBU (54 μL, 0.353 mmol) was added to a mixture of the product obtained in EX-34 step 4 (50 mg, 0.071 mmol) and BOP (84 mg, 0.184 mmol) in 5.00 mL acetonitrile and 5.00 mL DMF. The reaction mixture was stirred at RT overnight. The volatiles were removed under reduced pressure and the crude product was purified by means of HPLC (Sunfire, ACN/H₂O/TFA).

ESI-MS: 654/656 (1Cl) [M+H]⁺; Rₜ (HPLC): 0.84 min (Method B)

EX-34 Step 6:

The product from EX-34 step 6 was deprotected according to general procedure C to afford the title compound.

ESI-MS: 640/642 (1Cl) [M+H]⁺; Rₜ (HPLC): 0.78 min (Method B)

### Example EX-35: (1R,9S,11S,33S)-4-chloro-1-fluoro-33-methyl-30-oxo-8,15,29-trioxa-6,12,23,31,36-pentaazaheptacyclo[29.2.2.1^{9,12}.1^{13,24}.0^{2,7}.0^{14,22}.0^{16,21}]heptatriaconta-2,4,6,13(36),14(22),16(21),17,19,23-nonaene-11-carboxylic acid

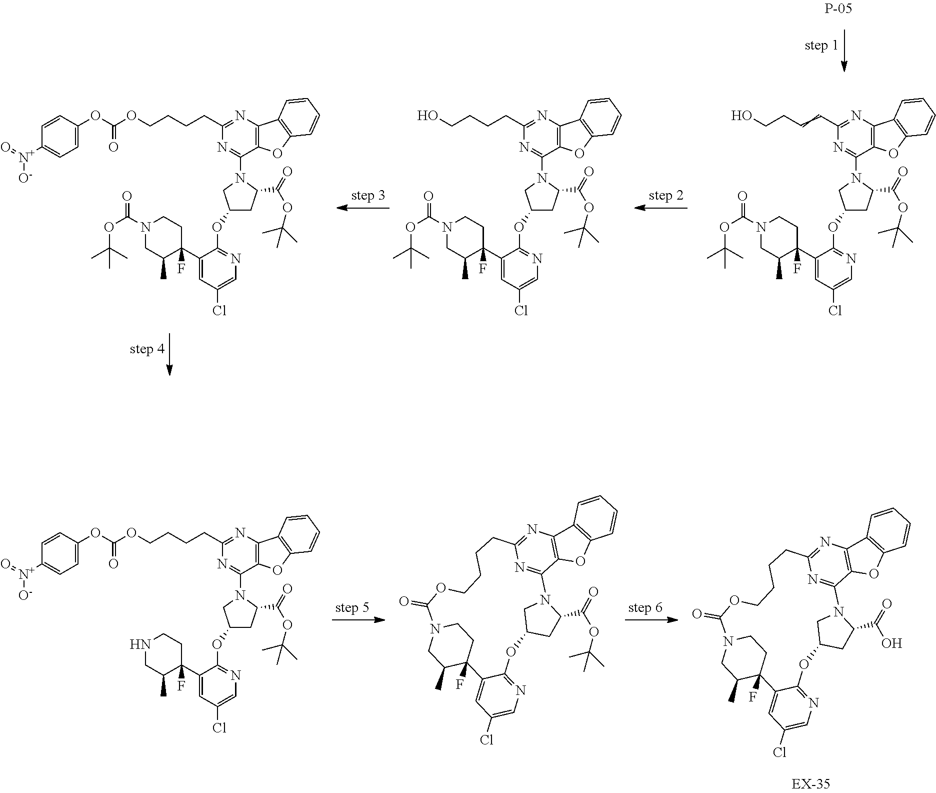

EX-35 Step 1:

Intermediate P-05 (200 mg, 0.250 mmol) and 3-buten-1-ol (86 μL, 1.00 mmol) were added to a microwave vial and the system was flushed with argon. 2.31 mL DMA followed by triethyl amine (696 μL, 4.99 mmol) was added and the tube was degassed for 5 m in under a stream of argon. Then 1,1′-Bis(di-tert-butylphosphino)ferrocene palladium dichloride (24.4 mg, 0.037 mmol) was added, the vial was sealed and the reaction mixture heated at 115° C. for 4 h. After cooling to RT, the reaction mixture was filtered over a catalyst scavenging cartridge, diluted with additional ACN and acidified with 2.5 M acetic acid. The crude product was purified directly by means of HPLC (Sunfire, ACN/H₂O/TFA).

ESI-MS: 753/755 (1Cl) [M+H]⁺; Rₜ (HPLC): 0.79 min (Method A)

EX-35 Step 2:

The product obtained in EX-35 step 2 was hydrogenated according to General Procedure N.

ESI-MS: 755/757 (1Cl) [M+H]⁺; Rₜ (HPLC): 0.77 min (Method A)

EX-35 Step 3:

The product obtained in EX-35 step 2 (21 mg, 0.028 mmol) was dissolved in 0.36 mL DCM and pyridine (2 μL, 0.03 mmol) was added. The mixture was cooled to 0° C. and 4-nitrophenyl chloroformate (6.3 mg, 0.030 mmol) was added. The reaction was allowed to come to RT and stirred for 3 h. The volatiles were removed under reduced pressure and the remaining crude was taken to the next step without further purification.

ESI-MS: 920/922 (1Cl) [M+H]⁺; Rₜ (HPLC): 0.85 min (Method A)

EX-35 Step 4:

The product obtained in EX-35 step 3 was deprotected according to General Procedure F.

ESI-MS: 820/822 (1Cl) [M+H]⁺; Rₜ (HPLC): 0.66 min (Method A)

EX-35 Step 5:

Triethylamine (39 mg, 0.053 mL, 0.384 mmol) was added to a solution of the product obtained in EX-35 step 4 (21 mg, 0.026 mmol) in 3.00 mL THF. The reaction mixture was heated to 70° C. for 3 h, then concentrated under reduced pressure and taken to the next step without further purification.

ESI-MS: 681/683 (1Cl) [M+H]⁺; Rₜ (HPLC): 0.73 min (Method A)

EX-35 Step 6:

The product obtained in EX-35 step 5 was deprotected according to General Procedure H.

ESI-MS: 625 [M+H]⁺; Rₜ (HPLC): 0.60 min (Method A)

### Example EX-37

### (1R,9S,11S,33S)-4-chloro-1-fluoro-33-methyl-30-oxo-8,15,28-trioxa-6,12,23,31,36-pentaaza-heptacyclo[29.2.2.1^{9,12}.1^{13,24}.0^{2,7}.0^{14,22}.0^{16,21}]heptatriaconta-2,4,6,13(36),14(22),16(21),17,19,23-nonaene-11-carboxylic acid

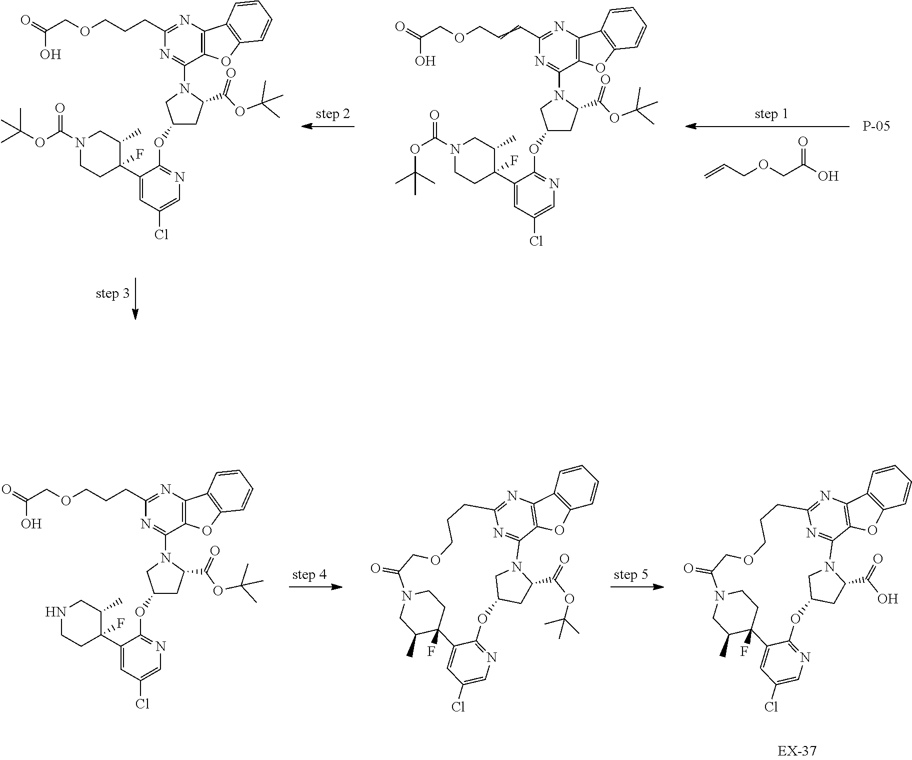

The title compound was prepared starting from Intermediate P-05 applying the following reaction sequence:

EX-37 step 1: Heck coupling with 2-(prop-2-en-1-yloxy)acetic acid applying General Procedure M

ESI-MS: 796/798 (1Cl) [M+H]⁺ Rₜ (HPLC): 0.65/0.66 min (Method D)

EX-37 step 2: Hydrogenation (Pd/C) according to General Procedure N

ESI-MS: 798/800 (1Cl) [M+H]⁺; Rₜ (HPLC): 0.97 min (Method B)

EX-37 step 3: BOC-deprotection applying General Procedure F

ESI-MS 698/700 (1Cl) [M+H]⁺; Rₜ (HPLC): 0.63 min (Method B)

EX-37 step 4: Amidation applying General Procedure E

ESI-MS 680/682 (1Cl) [M+H]⁺; Rₜ (HPLC): 0.93 min (Method D)

EX-37 step 5: tert butyl ester deprotection applying General Procedure H

ESI-MS 624/626 (1Cl) [M+H]⁺; Rₜ (HPLC): 0.48 min (Method D)

### Example EX-38

### (1R,9S,11S,33S)-4-chloro-1-fluoro-33-methyl-30-oxo-8,15,27-trioxa-6,12,23,31,36-pentaaza-heptacyclo[29.2.2.1^{9,12}.1^{13,24}.0^{2,7}.0^{14,22}.0^{16,21}]heptatriaconta-2,4,6,13(36),14(22),16(21),17,19,23-nonaene-11-carboxylic acid

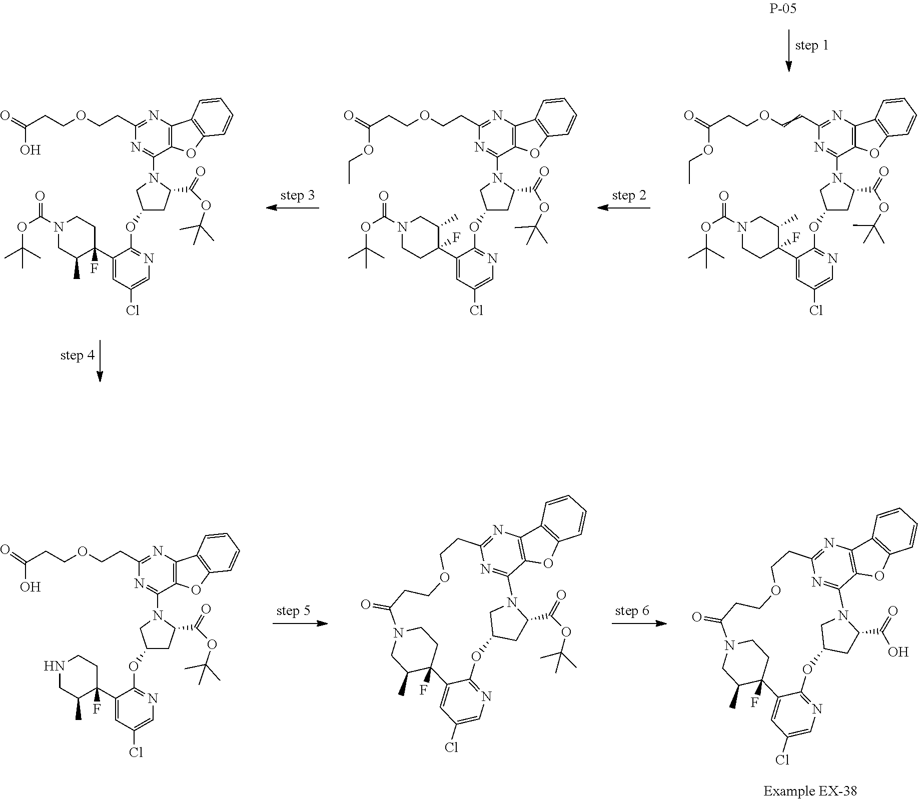

The title compound was prepared starting from Intermediate P-05 applying the following reaction sequence:

EX-38 step 1: Heck coupling with ethyl 3-(ethenyloxy)propanoate applying General Procedure M

ESI-MS: 768/770 (1Cl) [M-tBu+H]⁺ Rₜ (HPLC): 0.83 min (Method A)

EX-38 step 2: Hydrogenation (Pd/C) according to General Procedure N

ESI-MS: 826/828 (1Cl) [M+H]⁺; Rₜ (HPLC): 1.10 min (Method B)

EX-38 step 3: Ester hydrolysis according to general procedure C

ESI-MS: 798/800 (1Cl) [M+H]⁺; Rₜ (HPLC): 0.99 min (Method B)

EX-38 step 4: BOC-deprotection applying General Procedure F

ESI-MS 698/700 (1Cl) [M+H]⁺; Rₜ (HPLC): 0.65 min (Method B)

EX-38 step 5: Amidation applying General Procedure E

ESI-MS 680/682 (1Cl) [M+H]⁺; Rₜ (HPLC): 0.93 min (Method D)

EX-37 step 6: tert butyl ester deprotection applying General Procedure H

ESI-MS 624/626 (1Cl) [M+H]⁺; Rₜ (HPLC): 0.53 min (Method A)

General Technical Remarks

The terms “ambient temperature” and “room temperature” are used interchangeably and designate a temperature of about 20° C., e.g. 15 to 25° C.

As a rule, ¹H NMR spectra and/or mass spectra have been obtained of the compounds prepared. Unless otherwise stated, all chromatographic operations were performed at room temperature.

General Method

NMR spectra were recorded on Bruker Avance 400 MHz for 1H NMR. LCMS were taken on a quadrupole Mass Spectrometer on Shimadzu LCMS 2010 (Column: sepax ODS 50×2.0 mm, 5 um) or Agilent 1200 HPLC, 1956 MSD (Column: Shim-pack XR-ODS 30×3.0 mm, 2.2 um) operating in ES (+) ionization mode. Chromatographic purifications were performed by flash chromatography using 100-200 mesh silica gel. Anhydrous solvents were pre-treated with 3 Å MS column before use. All commercially available reagents were used as received unless otherwise stated.

The absolute configuration of selected EXAMPLES (EX-01, EX-03, EX-11, EX-17, EX-37) was assigned from a co-crystal structure with human cGAS protein according to methods described by D. J. Patel et al., PNAS 2019, 11946-11955 (doi.org/10.1073/pnas.1905013116).

List of Abbreviations
ACN Acetonitrile
aq. Aqueous
BOP ((1H-Benzo[d][1,2,3]triazol-1yl)oxy)tris(dimethylamino)-phosphonium hexafluorophosphate(V)
° C. degree Celsius
CH cyclohexane
DAST diethylaminosulfur trifluoride
DBU diazabicyclo[5.4.0]undec-7-ene
DCM Dichloromethane
DIPEA Diisopropylethylamine
DMA Dimethylacetamide
DMAP Dimethylaminopyridine
DMF N,N-dimethylformamide
ds Diastereoisomer
EDC (3-dimethylamino-propyl)-ethyl-carbodiimide
ESI-MS electrospray ionisation mass spectrometry
EA or EtOAc ethyl acetate
eq Equivalent
h hour(s)
HCl Hydrogenchloride
HATU [dimethylamino-(1,2,3-triazolo[4,5-b]pyridin-3-yloxy)-methylene]-dimethyl-ammonium hexafluorophosphate
HPLC high performance liquid chromatography
Grubbs Catalyst® 2^{nd} Dichlor[1,3-bis(2,4,6-trimethylphenyl)-2-Generation imidazolidinyliden](benzyliden), CAS 246047-72-3
K₂CO₃ potassium carbonate
L liter
MeOH methanol
Min minute
mL milliliter
M molar
NMP N-methyl-2-pyrrolidon
Pd(dppf)Cl₂ (1,1′-bis-(diphenylphosphino)-ferrocene)-dichloropalladium (II)
Pd(PPh₃)₄ tetrakis(triphenylphosphine)palladium(O)
PTSA p-toluenesulfonic acid
RT room temperature (about 20° C.)
TBAF tetrabutylammoniumfluoride
TFA trifluoroacetic acid
THF tetrahydrofurane
TMS trimethylsilyl
TosOH p-toluenesulfonic acid
RP-HPLC reverse phase HPLC
Rₜ retention time in minutes

Analytical Methods (HPLC/SFC):

HPLC method A:

| | Vol % water | | |
|---|---|---|---|
| Time [min] | (incl. 0.1% TFA) | Vol % ACN | Flow [mL/min] |
| 0.0 | 99.0 | 1.0 | 1.6 |
| 0.02 | 99.0 | 1.0 | 1.6 |
| 1.0 | 0.0 | 100.0 | 1.6 |
| 1.1 | 0.0 | 100.0 | 1.6 |

Column: XBridge BEH C18_2.1×30 mm 1.7 μm (Waters); CT: 60° C. HPLC method B:

| | Vol % water | | |
|---|---|---|---|
| Time [min] | (incl. 0.1% TFA) | Vol % ACN | Flow [mL/min] |
| 0.0 | 95.0 | 5.0 | 1.5 |
| 1.3 | 0.0 | 100.0 | 1.5 |
| 1.5 | 0.0 | 100.0 | 1.5 |

Column: Sunfire C18_3.0×30 mm 2.5 μm (Waters); CT: 60° C.

HPLC method C:

| | Vol % water | | |
|---|---|---|---|
| Time [min] | (incl. 0.1% TFA) | Vol % ACN | Flow [mL/min] |
| 0.0 | 97.0 | 3.0 | 2.2 |
| 0.2 | 97.0 | 3.0 | 2.2 |
| 1.2 | 0.0 | 100.0 | 2.2 |
| 1.25 | 0.0 | 100.0 | 3.0 |
| 1.4 | 0.0 | 100.0 | 3.0 |

Column: Sunfire C18_3.0×30 mm 2.5 μm (Waters); Cr: 60° C.

HPLC method D:

| | Vol % water | | |
|---|---|---|---|
| Time [min] | (incl. 0.1% TFA) | Vol % ACN | Flow [mL/min] |
| 0.0 | 95.0 | 5.0 | 1.3 |
| 0.02 | 95.0 | 5.0 | 1.3 |
| 1.0 | 0.0 | 100.0 | 1.3 |
| 1.3 | 0.0 | 100.0 | 1.3 |

Column: XBridge BEH C18_2.1×30 mm 2.5 μm (Waters); Cr: 60° C.

HPLC method E:

| | Vol % water | Vol % ACN | |
|---|---|---|---|
| | (incl. 0.1% | (incl. 0.1% | |
| Time [min] | formic acid) | formic acid) | Flow [mL/min] |
| 0.0 | 80 | 20 | 1 |
| 3.35 | 20 | 80 | 1 |
| 3.75 | 20 | 80 | 1 |
| 3.9 | 5 | 95 | 1 |
| 4.75 | 5 | 95 | 1 |
| 5.0 | 80 | 20 | 1 |
| 6.0 | 80 | 20 | 1 |

Column: Kinetex XB-C18 2.6 m (4.6×50 mm), 100 Å; Cr: 25° C.

HPLC method F:

| | Vol % water | Vol % ACN | |
|---|---|---|---|
| | (incl. 0.1% | (incl. 0.1% | |
| Time [min] | formic acid) | formic acid) | Flow [mL/min] |
| 0.0 | 80 | 20 | 0.5 |
| 0.1 | 80 | 20 | 0.5 |
| 1.1 | 0 | 100 | 0.5 |
| 2.5 | 80 | 20 | 0.5 |
| 3.0 | 80 | 20 | 0.5 |

Column: Acquity UPLC BEH C18 1.7 μm (2.1×100 mm); CT: 40° C.

HPLC method G:

| | Vol % water | | |
|---|---|---|---|
| Time [min] | (incl. 0.1% TFA) | Vol % ACN | Flow [mL/min] |
| 0.0 | 50.0 | 50.0 | 1.6 |
| 0.02 | 50.0 | 50.0 | 1.6 |
| 1.0 | 0.0 | 100.0 | 1.6 |
| 1.1 | 0.0 | 100.0 | 1.6 |

Column: XBridge BEH C18 2.1×30 mm 1.7 μm (Waters); CT: 60° C.

HPLC method H:

| | Vol % water | Vol % ACN | |
|---|---|---|---|
| | (incl. 0.1% | (incl. 0.1% | |
| Time [min] | formic acid) | formic acid) | Flow [mL/min] |
| 0.0 | 90 | 10 | 1 |
| 3.35 | 30 | 70 | 1 |
| 3.9 | 5 | 95 | 1 |
| 4.75 | 5 | 95 | 1 |
| 5.0 | 90 | 10 | 1 |
| 6.0 | 90 | 10 | 1 |

Column: Kinetex XB-C18 2.6 μm (4.6×50 mm), 100 Å; CT: 25° C.

### 4. EXAMPLES

5.1 Example Compounds of Formula I or II of the Invention

The following Example compounds of formula I or II as summarized in Table 1 have been synthesized and tested with respect to their pharmacological properties regarding their potency to inhibit cGAS activity.

In particular the “biochemical (in vitro) IC₅₀-values” with regard to cGAS-inhibition (hcGAS IC₅₀), the “IC₅₀-value with regard to the inhibition of IFN induction in virus-stimulated THP1 cells” (THP₍ᵥᵢᵣ₎ IC₅₀), the “IC₅₀-value with regard to the inhibition of IFN induction in cGAMP-stimulated THP1 cells” (THP_{(cGAMP)} IC₅₀) and the “IC₅₀-value with regard to inhibition of IFN induction in dsDNA-stimulated human whole blood” (hWB IC₅₀) has been experimentally determined according to the assay methods as described in section 6 below. The results are summarized in Table 1.

The Example compounds of formula I or II as summarized in Table 1 show at the same time the following three properties:
a satisfying “biochemical (in vitro) IC₅₀-value with regard to cGAS inhibition” (with a hcGAS IC₅₀ of 5100 nM, preferably of ≤50 nM, in particular of ≤10 nM),
a satisfying “cellular IC₅₀-value regarding cGAS inhibition” (with a THP1₍ᵥᵢᵣ₎ IC₅₀ of 51 μM, preferably of ≤500 nM, more preferably of 5100 nM, in particular of ≤50 nM) and
a satisfying selectivity for cGAS-inhibition
(with a ratio THP1_{(cGAMP)}C₅₀/THP1₍ᵥᵢᵣ₎IC₅₀ of 210, more preferably ≥50, more preferably ≥500, in particular ≥1000).

Additionally, the Example compounds of formula I or II also show acceptable IC₅₀-values with regard to inhibition of IFN induction in dsDNA-stimulated human whole blood (hWB IC₅₀).

TABLE 1
Pharmacological properties of the Example compounds of formula I or II of the invention

| | | | | | Ratio | |
|---|---|---|---|---|---|---|
| | | | | | THP_{(cGAMP)} | |
| Example | | hcGAS | THP₍ᵥᵢᵣ₎ | THP_{(cGAMP)} | IC₅₀/ THP₍ᵥᵢᵣ₎ | hWB |
| No. | Structure | IC₅₀ [nM] | IC₅₀ [nM] | IC₅₀ [nM] | IC₅₀ | IC₅₀ [nM] |
| 01 | 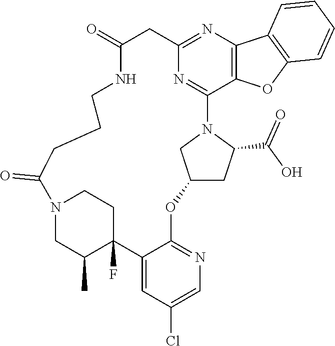 [SMILES: C[C@H]1CN2CC[C@]1(F)c1cc(Cl)cnc1O[C@H]1C[C@@H](C(=O)O)N(C1)c1nc(nc3c1oc1ccccc13)CC(=O)NCCCC2=O] | 25 | 150 | 2900 | 19 | 208 |
| 02 | 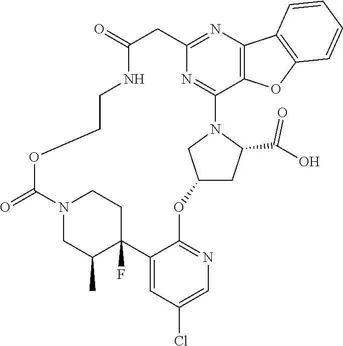 [SMILES: C[C@H]1CN2CC[C@]1(F)c1cc(Cl)cnc1O[C@H]1C[C@@H](C(=O)O)N(C1)c1nc(nc3c1oc1ccccc13)CC(=O)NCCOC2=O] | 17 | 110 | >10000 | 94 | 115 |
| 03 | 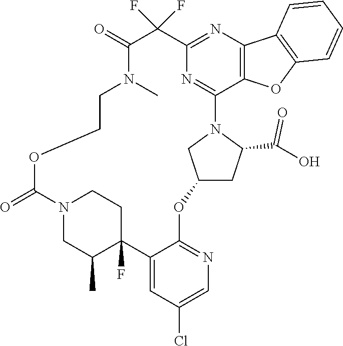 [SMILES: C[C@H]1CN2CC[C@]1(F)c1cc(Cl)cnc1O[C@H]1C[C@@H](C(=O)O)N(C1)c1nc(nc3c1oc1ccccc13)C(F)(F)C(=O)N(C)CCOC2=O] | 7.5 | 60 | >10000 | 165 | 76 |
| 04 | 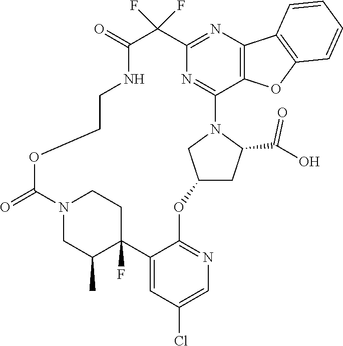 [SMILES: C[C@H]1CN2CC[C@]1(F)c1cc(Cl)cnc1O[C@H]1C[C@@H](C(=O)O)N(C1)c1nc(nc3c1oc1ccccc13)C(F)(F)C(=O)NCCOC2=O] | 3.9 | 46 | 980 | 21 | 49 |
| 05 | 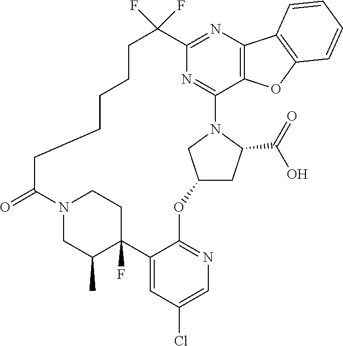 [SMILES: C[C@H]1CN2CC[C@]1(F)c1cc(Cl)cnc1O[C@H]1C[C@@H](C(=O)O)N(C1)c1nc(nc3c1oc1ccccc13)C(F)(F)CCCCCC2=O] | 12 | 59 | 9400 | 158 | 114 |
| 06 | 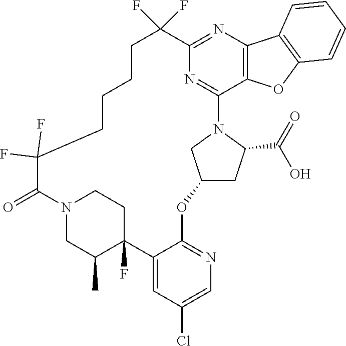 [SMILES: C[C@H]1CN2CC[C@]1(F)c1cc(Cl)cnc1O[C@H]1C[C@@H](C(=O)O)N(C1)c1nc(nc3c1oc1ccccc13)C(F)(F)CCCCC(F)(F)C2=O] | 18 | 150 | >10000 | 65 | 222 |
| 07 | 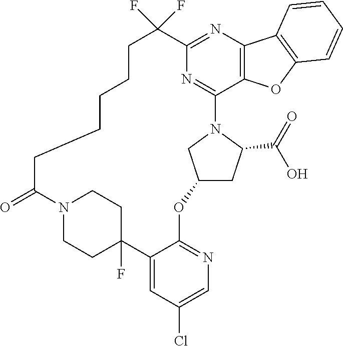 [SMILES: O=C(O)[C@@H]1C[C@H]2CN1c1nc(nc3c1oc1ccccc13)C(F)(F)CCCCCC(=O)N1CCC(F)(CC1)c1cc(Cl)cnc1O2] | 100 | 320 | 12000 | 37 | 750 |
| 08 | 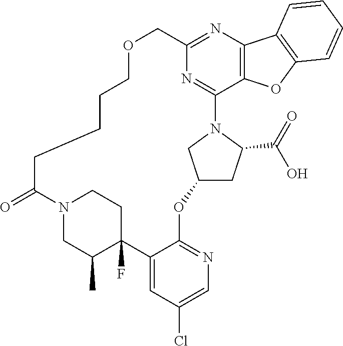 [SMILES: C[C@H]1CN2CC[C@]1(F)c1cc(Cl)cnc1O[C@H]1C[C@@H](C(=O)O)N(C1)c1nc(nc3c1oc1ccccc13)COCCCCC2=O] | 22 | 140 | >10000 | 69 | 44 |
| 09 | 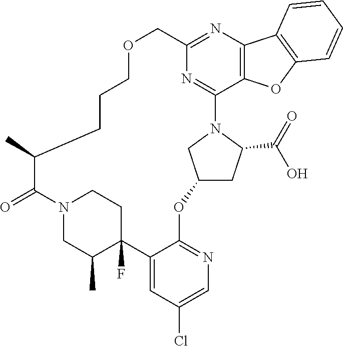 [SMILES: C[C@H]1CCCOCc2nc(c3oc4ccccc4c3n2)N2C[C@H](C[C@H]2C(=O)O)Oc2ncc(Cl)cc2[C@@]2(F)CCN(C[C@@H]2C)C1=O] | 45 | 240 | 3500 | 15 | 161 |
| 10 | 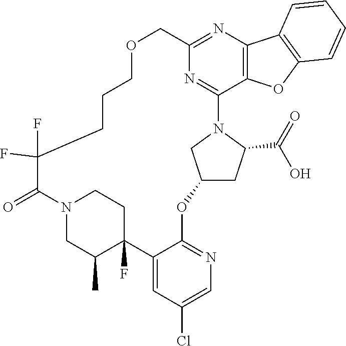 [SMILES: C[C@H]1CN2CC[C@]1(F)c1cc(Cl)cnc1O[C@H]1C[C@@H](C(=O)O)N(C1)c1nc(nc3c1oc1ccccc13)COCCCC(F)(F)C2=O] | 68 | 190 | 12000 | 61 | 339 |
| 11 | 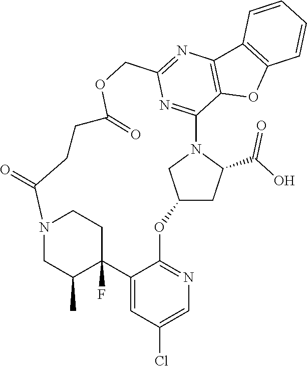 [SMILES: C[C@H]1CN2CC[C@]1(F)c1cc(Cl)cnc1O[C@H]1C[C@@H](C(=O)O)N(C1)c1nc(nc3c1oc1ccccc13)COC(=O)CCC2=O] | 2.8 | 11 | >10000 | 902 | 9 |
| 12 | 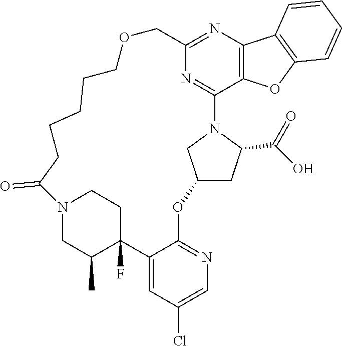 [SMILES: C[C@H]1CN2CC[C@]1(F)c1cc(Cl)cnc1O[C@H]1C[C@@H](C(=O)O)N(C1)c1nc(nc3c1oc1ccccc13)COCCCCCC2=O] | 17 | 610 | 10000 | 17 | 84 |
| 13 | 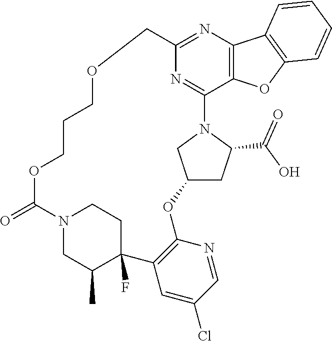 [SMILES: C[C@H]1CN2CC[C@]1(F)c1cc(Cl)cnc1O[C@H]1C[C@@H](C(=O)O)N(C1)c1nc(nc3c1oc1ccccc13)COCCCOC2=O] | 22 | 42 | 16000 | 256 | 149 |
| 14 | 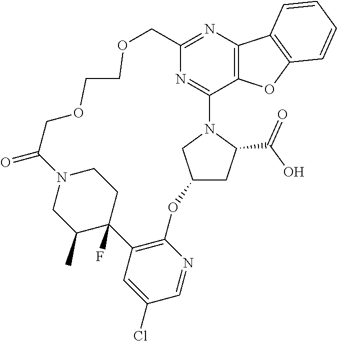 [SMILES: C[C@H]1CN2CC[C@]1(F)c1cc(Cl)cnc1O[C@H]1C[C@@H](C(=O)O)N(C1)c1nc(nc3c1oc1ccccc13)COCCOCC2=O] | 15 | 36 | >10000 | 282 | 136 |
| 15 | 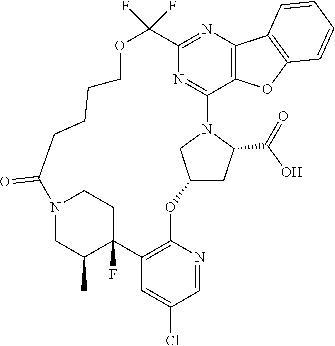 [SMILES: C[C@H]1CN2CC[C@]1(F)c1cc(Cl)cnc1O[C@H]1C[C@@H](C(=O)O)N(C1)c1nc(nc3c1oc1ccccc13)C(F)(F)OCCCCC2=O] | 11 | 70 | 8200 | 117 | 47 |
| 16 | 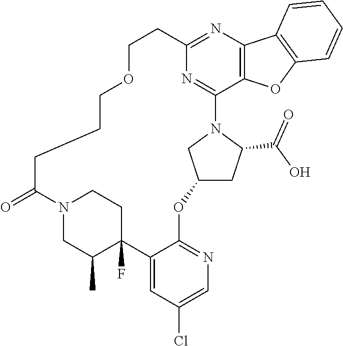 [SMILES: C[C@H]1CN2CC[C@]1(F)c1cc(Cl)cnc1O[C@H]1C[C@@H](C(=O)O)N(C1)c1nc(nc3c1oc1ccccc13)CCOCCCC2=O] | 11 | 36 | 6600 | 184 | 101 |
| 17 | 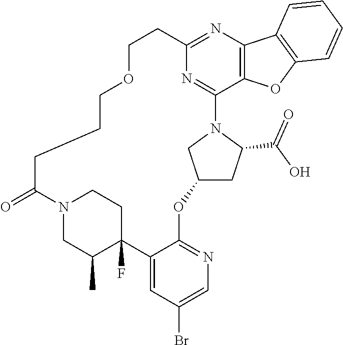 [SMILES: C[C@H]1CN2CC[C@]1(F)c1cc(Br)cnc1O[C@H]1C[C@@H](C(=O)O)N(C1)c1nc(nc3c1oc1ccccc13)CCOCCCC2=O] | 13 | 140 | 5600 | 40 | 96 |
| 18 | 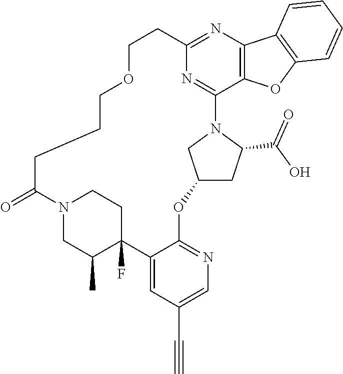 [SMILES: C#Cc1cnc2c(c1)[C@@]1(F)CCN(C[C@@H]1C)C(=O)CCCOCCc1nc(c3oc4ccccc4c3n1)N1C[C@H](C[C@H]1C(=O)O)O2] | 6 | 35 | 4300 | 122 | 18 |
| 19 | 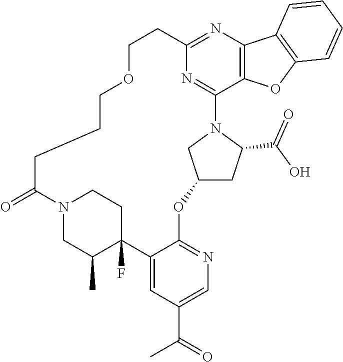 [SMILES: CC(=O)c1cnc2c(c1)[C@@]1(F)CCN(C[C@@H]1C)C(=O)CCCOCCc1nc(c3oc4ccccc4c3n1)N1C[C@H](C[C@H]1C(=O)O)O2] | 8 | 73 | >10000 | 136 | 146 |
| 20.01 | 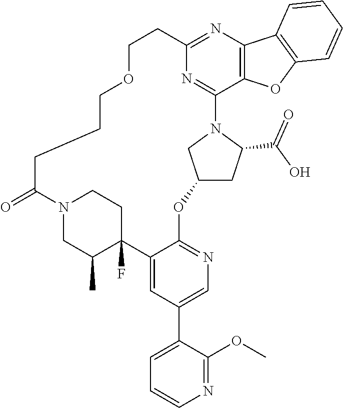 [SMILES: COc1ncccc1-c1cnc2c(c1)[C@@]1(F)CCN(C[C@@H]1C)C(=O)CCCOCCc1nc(c3oc4ccccc4c3n1)N1C[C@H](C[C@H]1C(=O)O)O2] | 18 | 19 | 8100 | 419 | 45 |
| 20.02 | 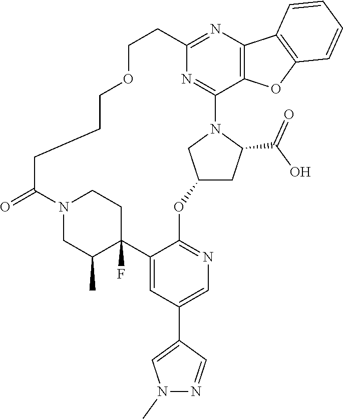 [SMILES: C[C@H]1CN2CC[C@]1(F)c1cc(-c3cnn(C)c3)cnc1O[C@H]1C[C@@H](C(=O)O)N(C1)c1nc(nc3c1oc1ccccc13)CCOCCCC2=O] | 7 | 8 | 7900 | 946 | 10 |
| 20.03 | 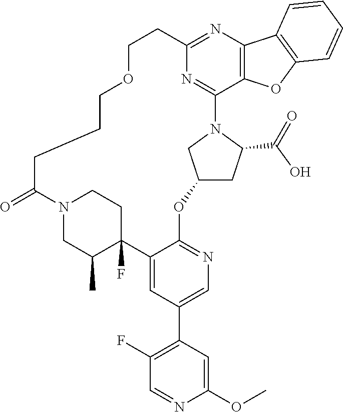 [SMILES: COc1cc(-c2cnc3c(c2)[C@@]2(F)CCN(C[C@@H]2C)C(=O)CCCOCCc2nc(c4oc5ccccc5c4n2)N2C[C@H](C[C@H]2C(=O)O)O3)c(F)cn1] | 18 | 18 | 5900 | 321 | 38 |
| 21 | 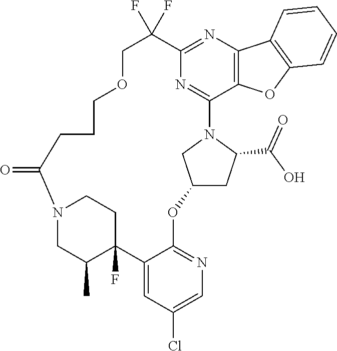 [SMILES: C[C@H]1CN2CC[C@]1(F)c1cc(Cl)cnc1O[C@H]1C[C@@H](C(=O)O)N(C1)c1nc(nc3c1oc1ccccc13)C(F)(F)COCCCC2=O] | 5 | 3 | 10000 | 3281 | 7 |
| 22 | 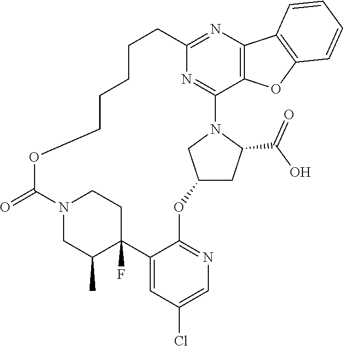 [SMILES: C[C@H]1CN2CC[C@]1(F)c1cc(Cl)cnc1O[C@H]1C[C@@H](C(=O)O)N(C1)c1nc(nc3c1oc1ccccc13)CCCCCOC2=O] | 45 | 490 | >10000 | 20 | 1557 |
| 23 | 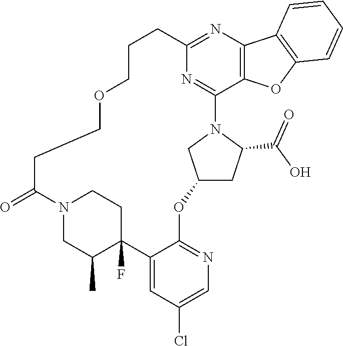 [SMILES: C[C@H]1CN2CC[C@]1(F)c1cc(Cl)cnc1O[C@H]1C[C@@H](C(=O)O)N(C1)c1nc(nc3c1oc1ccccc13)CCCOCCC2=O] | 12 | 69 | 10000 | 148 | 114 |
| 24 | 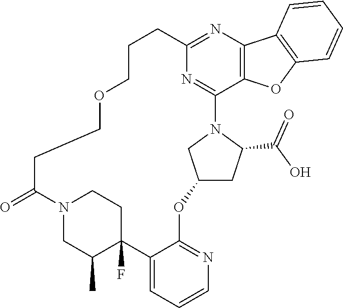 [SMILES: C[C@H]1CN2CC[C@]1(F)c1cccnc1O[C@H]1C[C@@H](C(=O)O)N(C1)c1nc(nc3c1oc1ccccc13)CCCOCCC2=O] | 28 | 640 | >10000 | 16 | 801 |
| 25 | 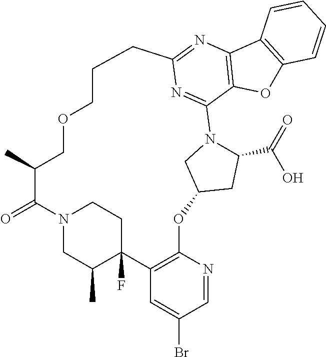 [SMILES: C[C@H]1COCCCc2nc(c3oc4ccccc4c3n2)N2C[C@H](C[C@H]2C(=O)O)Oc2ncc(Br)cc2[C@@]2(F)CCN(C[C@@H]2C)C1=O] | 7 | 110 | 1700 | 16 | 116 |
| 26 | 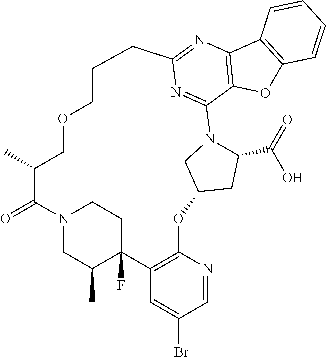 [SMILES: C[C@@H]1COCCCc2nc(c3oc4ccccc4c3n2)N2C[C@H](C[C@H]2C(=O)O)Oc2ncc(Br)cc2[C@@]2(F)CCN(C[C@@H]2C)C1=O] | 18 | 46 | 3200 | 70 | 276 |
| 27 | 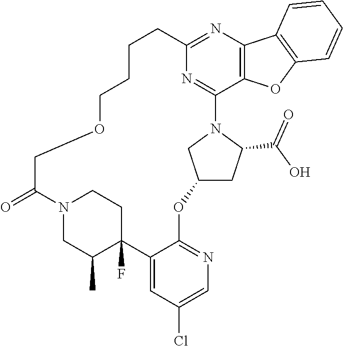 [SMILES: C[C@H]1CN2CC[C@]1(F)c1cc(Cl)cnc1O[C@H]1C[C@@H](C(=O)O)N(C1)c1nc(nc3c1oc1ccccc13)CCCCOCC2=O] | 5 | 83 | 1700 | 20 | 33 |
| 28 | 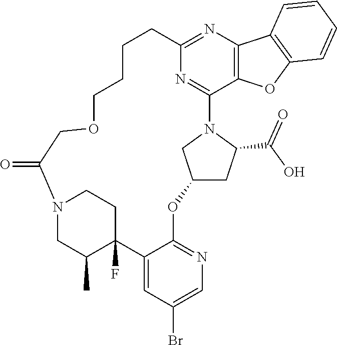 [SMILES: C[C@H]1CN2CC[C@]1(F)c1cc(Br)cnc1O[C@H]1C[C@@H](C(=O)O)N(C1)c1nc(nc3c1oc1ccccc13)CCCCOCC2=O] | 7 | 47 | 1900 | 39 | 15 |
| 29 | 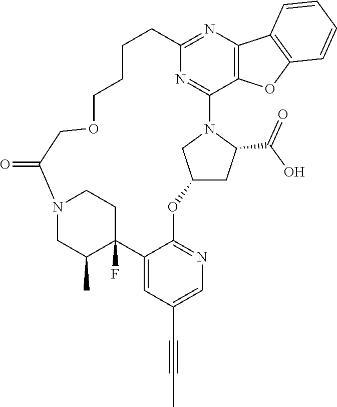 [SMILES: CC#Cc1cnc2c(c1)[C@@]1(F)CCN(C[C@@H]1C)C(=O)COCCCCc1nc(c3oc4ccccc4c3n1)N1C[C@H](C[C@H]1C(=O)O)O2] | 14 | 57 | 2200 | 39 | 22 |
| 30 | 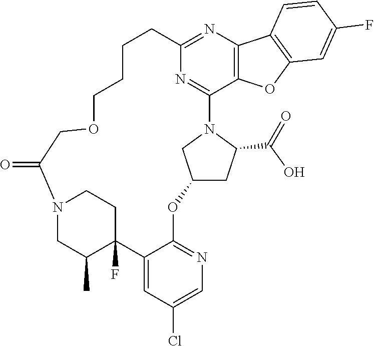 [SMILES: C[C@H]1CN2CC[C@]1(F)c1cc(Cl)cnc1O[C@H]1C[C@@H](C(=O)O)N(C1)c1nc(nc3c1oc1cc(F)ccc13)CCCCOCC2=O] | 7 | 70 | 4700 | 67 | 22 |
| 31 | 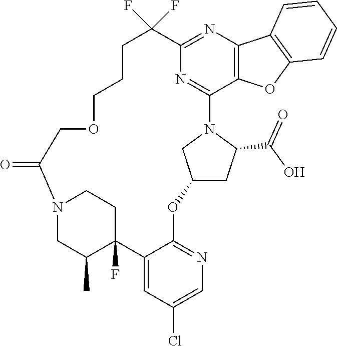 [SMILES: C[C@H]1CN2CC[C@]1(F)c1cc(Cl)cnc1O[C@H]1C[C@@H](C(=O)O)N(C1)c1nc(nc3c1oc1ccccc13)C(F)(F)CCCOCC2=O] | 7 | 3 | 7200 | 3002 | 5 |
| 32 | 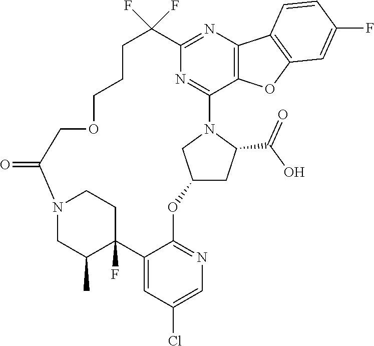 [SMILES: C[C@H]1CN2CC[C@]1(F)c1cc(Cl)cnc1O[C@H]1C[C@@H](C(=O)O)N(C1)c1nc(nc3c1oc1cc(F)ccc13)C(F)(F)CCCOCC2=O] | 5 | 8 | >10000 | 1190 | 9 |
| 33 | 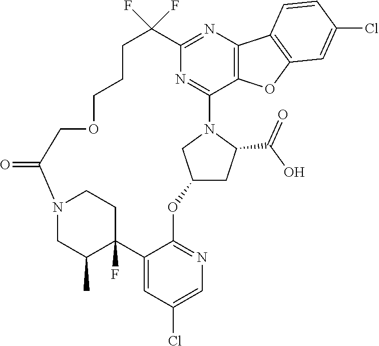 [SMILES: C[C@H]1CN2CC[C@]1(F)c1cc(Cl)cnc1O[C@H]1C[C@@H](C(=O)O)N(C1)c1nc(nc3c1oc1cc(Cl)ccc13)C(F)(F)CCCOCC2=O] | 5 | 3 | 6400 | 2085 | 16 |
| 34 | 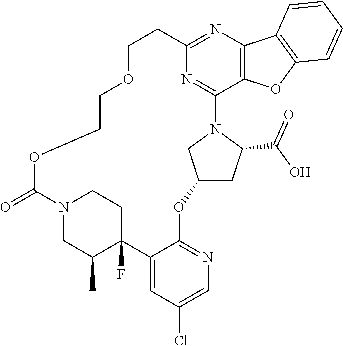 [SMILES: C[C@H]1CN2CC[C@]1(F)c1cc(Cl)cnc1O[C@H]1C[C@@H](C(=O)O)N(C1)c1nc(nc3c1oc1ccccc13)CCOCCOC2=O] | 8 | 36 | 16000 | 450 | 41 |
| 35 | 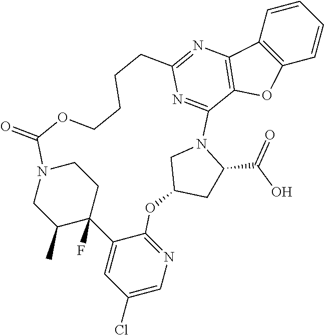 [SMILES: C[C@H]1CN2CC[C@]1(F)c1cc(Cl)cnc1O[C@H]1C[C@@H](C(=O)O)N(C1)c1nc(nc3c1oc1ccccc13)CCCCOC2=O] | 27 | 830 | N/A | N/A | 409 |
| 36 | 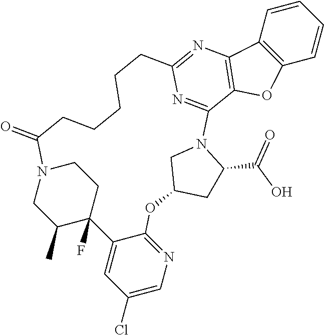 [SMILES: C[C@H]1CN2CC[C@]1(F)c1cc(Cl)cnc1O[C@H]1C[C@@H](C(=O)O)N(C1)c1nc(nc3c1oc1ccccc13)CCCCCC2=O] | 7 | 130 | >10000 | 80 | 79 |
| 37 | 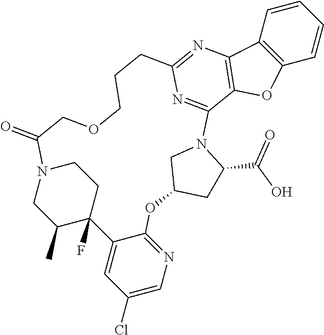 [SMILES: C[C@H]1CN2CC[C@]1(F)c1cc(Cl)cnc1O[C@H]1C[C@@H](C(=O)O)N(C1)c1nc(nc3c1oc1ccccc13)CCCOCC2=O] | 3 | 12 | 11000 | 884 | 11 |
| 38 | 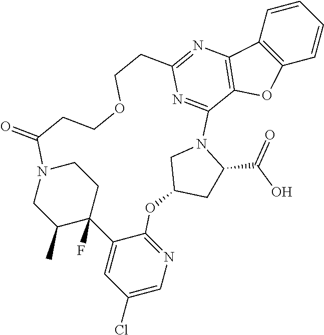 [SMILES: C[C@H]1CN2CC[C@]1(F)c1cc(Cl)cnc1O[C@H]1C[C@@H](C(=O)O)N(C1)c1nc(nc3c1oc1ccccc13)CCOCCC2=O] | 3 | 3 | >10000 | 2964 | 8 |

5.2 Comparison of the Example Compounds of Formula I or II with Prior Art Compounds

5.2.1 Compounds of WO 2020/142729

In WO 2020/142729 cGAS-inhibitors with partially similar structures have been disclosed.

On page 44 and 45 of WO 2020/142729 the “biochemical (in vitro) IC₅₀-values” with regard to cGAS-inhibition (corresponding to “hcGAS IC₅₀”) have been disclosed. Hereby compounds with a “biochemical (in vitro) IC₅₀-value” of less than 100 nM had been designated into “group A”, compounds with a “biochemical (in vitro) IC₅₀-value” of greater than 100 nM and less than 500 nM had been designated into “group B”, compounds with a “biochemical (in vitro) IC₅₀-value” of greater than 500 nM and less than 1 μM had been designated into “group C”, compounds with a “biochemical (in vitro) IC₅₀-value” of greater than 1 μM and less than 10 μM had been designated into “group D” and compounds with a “biochemical (in vitro) IC₅₀-value” of greater than 10 μM had been designated into “group E” (see page 44 of WO 2020/142729).

On page 45 of WO 2020/142729 it is disclosed that only compound No. 25 could be designated to “group A” having a “biochemical (in vitro) IC₅₀-value” of less than 100 nM. All other example compounds of WO 2020/142729 show “biochemical (in vitro) IC₅₀-values” of greater than 100 nM.

Selected prior art compounds of WO 2020/142729 including compound No. 25 have been synthesized and then have been tested with respect to their pharmacological properties regarding their potency to inhibit the cGAS/STING pathway using the same assays as used for testing the compounds of the invention. In particular the “biochemical (in vitro) IC₅₀-values” with regard to cGAS-inhibition (hcGAS IC₅₀), the “cellular IC₅₀-values with regard to inhibition of IFN induction in virus-stimulated THP1 cells” (THP1₍ᵥᵢᵣ₎ IC₅₀), the “cellular IC₅₀-value with regard to inhibition of IFN induction in cGAMP-stimulated THP1 cells” (THP1_{(cGAMP)} IC₅₀) and the “IC₅₀-value with regard to inhibition of IFN induction in human whole blood” (hWB) have been experimentally determined for the structurally closest examples of WO 2020/142729 according to the assay methods as described in section 6 below (see Table 2).

TABLE 2
Pharmacological properties of a selection of Example compounds from WO 2020/142729

| Example No. | | | | | |
|---|---|---|---|---|---|
| (as disclosed | | hcGAS | | | hWB |
| in WO | | IC50 | THP1₍ᵥᵢᵣ₎ | THP1_{(cGAMP)} | IC50 |
| 2020/142729) | Structure | [nM] | IC50 [nM] | IC50 [nM] | [nM] |
| 15 | 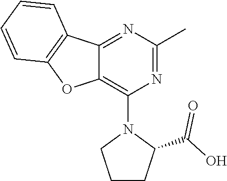 [SMILES: Cc1nc(N2CCC[C@H]2C(=O)O)c2oc3ccccc3c2n1] | 2700 | >17000 | >17000 | — |
| 25 | 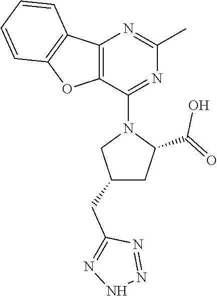 [SMILES: Cc1nc(N2C[C@@H](Cc3nn[nH]n3)C[C@H]2C(=O)O)c2oc3ccccc3c2n1] | 55 | >17000 | >17000 | >9992 |
| 28 | 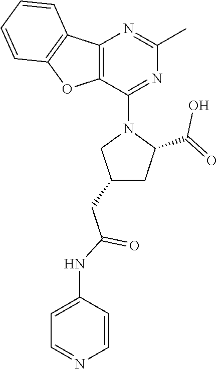 [SMILES: Cc1nc(N2C[C@@H](CC(=O)Nc3ccncc3)C[C@H]2C(=O)O)c2oc3ccccc3c2n1] | 630 | >32000 | >17000 | >9990 |
| 38 | 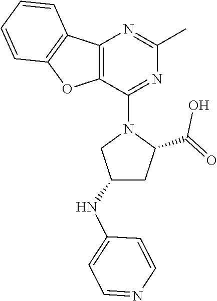 [SMILES: Cc1nc(N2C[C@@H](Nc3ccncc3)C[C@H]2C(=O)O)c2oc3ccccc3c2n1] | 3000 | >17000 | >17000 | >9990 |
| 58 | 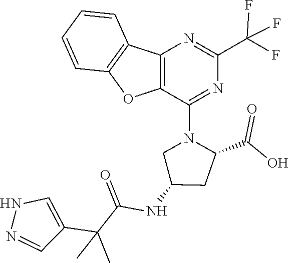 [SMILES: CC(C)(C(=O)N[C@H]1C[C@@H](C(=O)O)N(c2nc(C(F)(F)F)nc3c2oc2ccccc23)C1)c1cn[nH]c1] | 320 | 21000 | 23000 | >9982 |

The pharmacological properties for the Example compounds of the invention as summarized in Table 1 and the respective pharmacological properties for the compounds of WO 2020/142729 as summarized in Table 2 can be compared to each other, since they were experimentally determined according to the identical assay procedures as described in section 6 below.

From data as shown in Table 2 it is clear that all example compounds of WO 2020/142729 show “biochemical (in vitro) IC₅₀-values” (=hcGAS IC₅₀) that are significantly larger than 100 nM—with the only exception of Example No. 25 of WO 2020/142729 (in WO 2020/142729 designated in “Group A” having a “biochemical (in vitro) IC₅₀-value” (=hcGAS IC₅₀) of less than 100 nM). In contrast to that the Example compounds of the invention all have “biochemical (in vitro) IC₅₀-values” (hcGAS IC₅₀) of less than 100 nM. However, Example No. 25 of WO 2020/142729 which has a “biochemical (in vitro) IC₅₀-value” (hcGAS IC₅₀) of 55 nM, does not at all comply with the selection criterium of a “satisfying cellular inhibitory potency” shown by a THP1₍ᵥᵢᵣ₎ IC₅₀ of lower than 1 μM, because THP1₍ᵥᵢᵣ₎ IC₅₀ for Example No. 25 of WO 2020/142729 is 17 μM.

5.2.2 Compounds of WO 2022/174012

In WO 2022/174012 cGAS-inhibitors with partially similar structures have been disclosed.

On page 65 of WO 2022/174012 the “biochemical (in vitro) IC₅₀-values” with regard to cGAS-inhibition and on page 67 of WO 2022/174012 the “cellular IC₅₀-values” (IFNβ ELISA stimulated with THP-1) have been disclosed. Compound 5 (BBL0100455) of WO 2022/174012 seems to be the only compound of WO 2022/174012 that may have the potential to satisfy the selection criteria of the instant invention that means to have
a) an “biochemical (or enzymatic) (in vitro) IC₅₀-value” of smaller than 100 nM (in the “enzymatic assay of WO 2022/174012” compound 5 has been measured to fall into “group B” which represents an “enzymatic IC₅₀-value” of 50 nM to 100 nM”, see page 65, Table 2 of WO 2022/174012)
b) and to have a “cellular IC₅₀-value” (IFNβ ELISA stimulated with THP-1) of smaller than 1 μM (in the “cellular assay” of WO 2022/174012 compound 5 has been measured to fall into “group A” representing an “cellular IC₅₀-value” of <1 μM”, see page 67 and 68, Table 3 of WO 2022/174012).

However both, the biochemical/enzymatic assay and the cellular assays of WO 2022/174012, are not identical to the respective “biochemical/enzymatic assays and cellular assays” of the instant invention and therefore the measured biochemical/enzymatic IC₅₀-values and cellular IC₅₀-values of WO 2022/174012 are not comparable to the respective IC₅₀-values as measured for the compounds of the instant invention. Therefore compound 5 of WO 2022/174012 has been synthesized and then has been tested with respect to its pharmacological properties regarding its potency to inhibit the cGAS/STING pathway using exactly the same assays as used for testing the compounds of the instant invention and as described in Section 6 below.

TABLE 3
Pharmacological properties of Compound No. 5 of WO 2022/174012

| Example No. | | | | | |
|---|---|---|---|---|---|
| (as disclosed | | | | | hWB |
| in WO | | hcGAS | THP1₍ᵥᵢᵣ₎ | THP1_{(cGAMP)} | IC₅₀ |
| 2022/174012) | Structure | IC₅₀ [nM] | IC₅₀ [nM] | IC₅₀ [nM] | [nM] |
| Compound 5 (BBL0100455) | 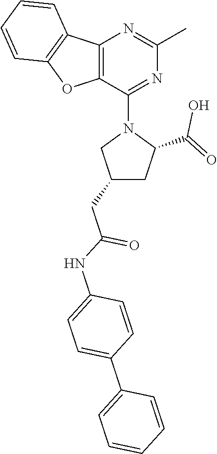 [SMILES: Cc1nc(N2C[C@@H](CC(=O)Nc3ccc(-c4ccccc4)cc3)C[C@H]2C(=O)O)c2oc3ccccc3c2n1] | 55 | >10000 | >10000 | |

As the data from Table 3 shows compound No. 5 (BBL0100455) of WO 2022/174012 has an acceptable biochemical/enzymatic IC₅₀-value of 55 nM (hcGAS IC₅₀=55 nM), but a cellular IC₅₀-value of larger than 10000 nM (THP1₍ᵥᵢᵣ₎ IC₅₀=10000 nM). Consequently the compound of the instant invention all are comparable to the compound No. 5 of WO 2022/174012 with respect to their biochemical/enzymatic IC₅₀-values, but are clearly superior over compound No. 5 of WO 2022/174012 with respect to their cellular IC₅₀-values (which are all smaller than 1000 nM for the compounds of formula I or II of the invention.

5.3 Prodrugs

It is known that esters of active agents with a carboxylic acid group may represent viable prodrugs which may i.e. show an improved oral absorption/bioavailability compared to the respective active agent. Frequently used prodrugs of active agents with a carboxylic acid group are for example methyl esters, ethyl esters, iso-propyl esters etc. (see Beaumont et al., Current Drug Metabolism, 2003, Vol. 4, Issue 6, 461-485).

Further, Nakamura et al., Bioorganic & Medicinal Chem., Vol. 15, Issue 24, p. 7720-7725 (2007), describes that also N-acylsulfonamide derivatives and N-acylsulfonylurea derivatives of a specific active agent with a free carboxylic acid group have the potential of being a viable prodrug.

Additionally, experimental hints have been found that also the methyl esters of the example compounds of formula I or II represent viable prodrugs of the cGAS inhibitors of formula I or II.

PCT/EP2022/062480 and PCT/EP2022/062496 (both so far unpublished) both disclose structurally similar cGAS-inhibitors as the cGAS-inhibitors of the instant invention which all comprise also carboxylic acid group attached to a pyrrolidine moiety. In both, PCT/EP2022/062480 and PCT/EP2022/062496 it has been experimentally shown that methyl esters derivatives of these cGAS-inhibitors carrying a carboxylic acid group attached to the pyrrolidine moiety act as viable prodrugs of the cGAS-inhibitors with the free carboxylic acid group.

Compounds P01, P02, P03 and P04 of PCT/EP2022/062480 were methyl ester derivatives and putative prodrugs of the respective Example compounds 4.04, 1.10, 1.12 and 3.14 of PCT/EP2022/062480 (which all had a free carboxylic group and were active cGAS-inhibitors with low biochemical IC₅₀-values and low cellular IC₅₀.values with regard to cGAS-inhibition).

Compounds P01, P02 and P03 of PCT/EP2022/062496 were methyl ester derivatives and putative prodrugs of the respective Example compounds 2.12, 1.13 and 1.05 of PCT/EP2022/062496 (which all had a free carboxylic group and were active cGAS-inhibitors with low biochemical IC₅₀-values and low cellular IC₅₀-values with regard to cGAS-inhibition).

In both, PCT/EP2022/062480 and PCT/EP2022/062496, the “active cGAS-inhibitors/Example compounds with their free carboxylic acid” and their “respective methyl ester derivatives/putative prodrugs” have been synthesized and have been tested for their pharmacological properties with respect to their potency to inhibit the cGAS/STING pathway.

This comparison of the properties of the Example compounds of PCT/EP2022/062480 and of PCT/EP2022/062496 with their free carboxylic acid on the one hand and the properties of their corresponding methyl ester derivatives/putative prodrugs shows that the “biochemical IC₅₀-values (hcGAS IC₅₀-values)” for the Example compounds are always around or even smaller than 10 nM, whereas the “biochemical IC₅₀-values (hcGAS IC₅₀-values)” for the corresponding methyl ester derivatives/prodrugs are always extremely large, that means generally larger than 7000 nM. That large difference between the IC₅₀-values of the Example compounds on the one hand and the IC₅₀-values of their corresponding methyl ester derivatives/prodrugs on the other hand has never been observed for the respective cellular IC₅₀-values (THP1₍ᵥᵢᵣ₎IC₅₀-values) which always stay more or less in the same range between example compounds and their corresponding prodrugs (see Table 4 below).

One possible explanation for that observation is that the Example compounds all have a free carboxylic group which seems to be crucial for inhibition of cGAS activity, whereas in all “methyl ester derivatives/prodrugs” the carboxyl group is masked by a carboxy-methyl ester group. Consequently, the methyl ester derivatives/prodrugs lose their inhibitory potency in the “in vitro human cGAS enzyme assay” (see section 6.1 below), because in this assay intracellular enzymes that cleave the carboxy-methyl ester group are absent and therefore the crucial free carboxylic acid group can not be restored in the biochemical assay. Therefore the prodrugs show extremely large “biochemical (in vitro) IC₅₀-values” (=hcGAS IC₅₀) in this “in vitro human cGAS enzyme assay”, whereas the corresponding Example compounds (which have a free carboxylic acid group from the beginning on) show small “biochemical (in vitro) IC₅₀-values” (=hcGAS IC₅₀).

In the cellular assay (=“human cGAS cell and the counter cell assay”, see section 6.2 below) endogenous cellular enzymes that cleave the carboxy-methyl ester group are present. Consequently not only the Example compounds of PCT/EP2022/062480 and of PCT/EP2022/062496 themselves (that already carry a free carboxylic acid group) show small THP1₍ᵥᵢᵣ₎IC₅₀-values, but also the corresponding methyl ester derivatives/prodrugs show relatively small “THP1₍ᵥᵢᵣ₎)IC₅₀-values”, because in this “human cGAS cell assay” the carboxy-methyl ester group of the prodrugs can be cleaved by the endogenous intracellular enzymes and thereby will release the “active Example Compounds with the free carboxylic acid group” that shows cGAS-inhibitory potency again.

This explanation together with the measurements as shown in Table 4 imply that carboxy-methyl ester derivatives of the structurally similar Example compounds of PCT/EP2022/062480 and of PCT/EP2022/062496 really seem to represent viable prodrugs of the respective Example compound with the free carboxylic acid group (which themselves have no inhibitory potency regarding the in vitro human biochemical cGAS inhibition). However, upon cleavage of the carboxy-methyl ester by endogenous intracellular enzymes present in the cellular assays the “active Example Compounds” are restored, that exhibit again an inhibitory potency regarding the cGAS/STING pathway.

Since the Example Compounds of formula I or II of the present invention have the very same free carboxylic acid attached to the pyrrolidinyl moiety as the Example Compounds of PCT/EP2022/062480 or of PCT/EP2022/062496, it can be expected that carboxy-methyl ester derivatives of these Compounds of formula I or II will also act as prodrugs.

TABLE 4
Comparison between selected cGAS-inhibitor compounds as disclosed in
PCT/EP2022/062480 and PCT/EP2022/062496 and their respective methyl ester prodrugs:

| | | hcGAS | THP1₍ᵥᵢᵣ₎ | THP1_{(cGAMP)} | |
|---|---|---|---|---|---|
| Example No./ | | IC₅₀ | IC₅₀ | IC₅₀ | hWB |
| Prodrug No. | Structure | [nM] | [nM] | [nM] | IC₅₀ [nM] |
| P01 of PCT/EP2022/062 480) (Prodrug of Ex. 4.04 of PCT/EP 2022/062480) | 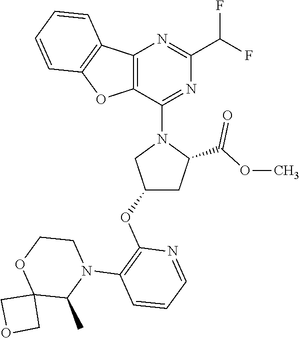 [SMILES: COC(=O)[C@@H]1C[C@H](Oc2ncccc2N2CCOC3(COC3)[C@@H]2C)CN1c1nc(C(F)F)nc2c1oc1ccccc12] | >9956 | 358 | 26058 | 1313 |
| Ex. 4.04 of PCT/EP2022/ 062480) | 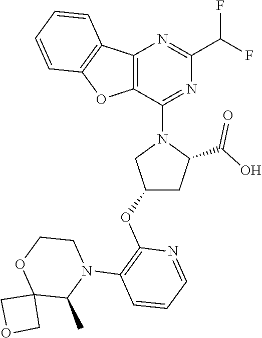 [SMILES: C[C@@H]1N(c2cccnc2O[C@H]2C[C@@H](C(=O)O)N(c3nc(C(F)F)nc4c3oc3ccccc34)C2)CCOC12COC2] | 2 | 386 | >16611 | 77 |
| P02 of PCT/EP2022/ 062480) (Prodrug of Ex. 1.10 of PCT/ EP2022/062480) | 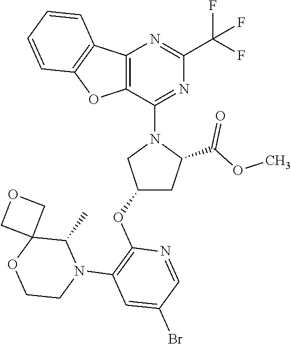 [SMILES: COC(=O)[C@@H]1C[C@H](Oc2ncc(Br)cc2N2CCOC3(COC3)[C@@H]2C)CN1c1nc(C(F)(F)F)nc2c1oc1ccccc12] | >9952 | 444 | 17109 | 5327 |
| Ex. 1.10 of PCT/EP2022/ 062480) | 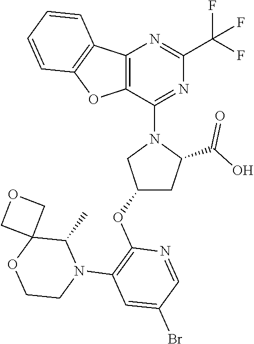 [SMILES: C[C@@H]1N(c2cc(Br)cnc2O[C@H]2C[C@@H](C(=O)O)N(c3nc(C(F)(F)F)nc4c3oc3ccccc34)C2)CCOC12COC2] | 5 | 91 | 16130 | 33 |
| P03 of PCT/EP2022/ 062480) (Prodrug of Ex. 1.12 of PCT/ EP2022/062480) | 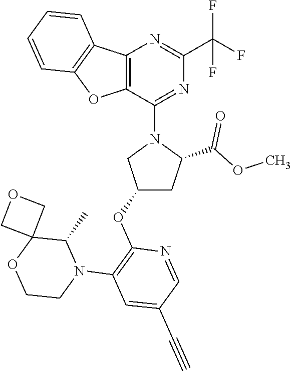 [SMILES: C#Cc1cnc(O[C@H]2C[C@@H](C(=O)OC)N(c3nc(C(F)(F)F)nc4c3oc3ccccc34)C2)c(N2CCOC3(COC3)[C@@H]2C)c1] | 9617 | 188 | 22845 | 1088 |
| Ex. 1.12 of PCT/EP2022/ 062480) | 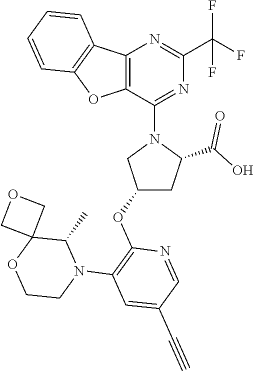 [SMILES: C#Cc1cnc(O[C@H]2C[C@@H](C(=O)O)N(c3nc(C(F)(F)F)nc4c3oc3ccccc34)C2)c(N2CCOC3(COC3)[C@@H]2C)c1] | 3 | 7 | 11892 | 15 |
| P04 of PCT/EP2022/ 062480) (Prodrug of Ex. 3.14 of PCT/ EP2022/062480) | 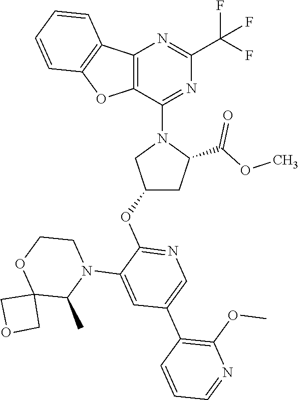 [SMILES: COC(=O)[C@@H]1C[C@H](Oc2ncc(-c3cccnc3OC)cc2N2CCOC3(COC3)[C@@H]2C)CN1c1nc(C(F)(F)F)nc2c1oc1ccccc12] | >9954 | 67 | >16621 | 2747 |
| Ex. 3.14 of PCT/EP2022/ 062480) | 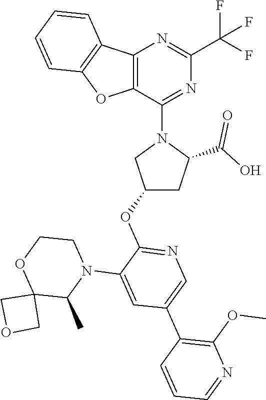 [SMILES: COc1ncccc1-c1cnc(O[C@H]2C[C@@H](C(=O)O)N(c3nc(C(F)(F)F)nc4c3oc3ccccc34)C2)c(N2CCOC3(COC3)[C@@H]2C)c1] | 11 | 10 | 7832 | 26 |
| P01 of PCT/EP2022/ 062496) (Prodrug of Ex. 2.12 of PCT/ EP2022/062496) | 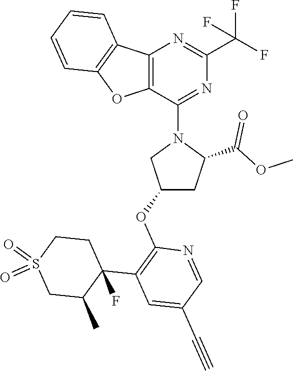 [SMILES: C#Cc1cnc(O[C@H]2C[C@@H](C(=O)OC)N(c3nc(C(F)(F)F)nc4c3oc3ccccc34)C2)c([C@@]2(F)CCS(=O)(=O)C[C@@H]2C)c1] | 19147 | 11 | >16612 | 67 |
| Ex. 2.12 of PCT/ EP2022/062496 | 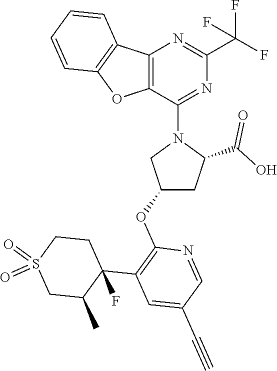 [SMILES: C#Cc1cnc(O[C@H]2C[C@@H](C(=O)O)N(c3nc(C(F)(F)F)nc4c3oc3ccccc34)C2)c([C@@]2(F)CCS(=O)(=O)C[C@@H]2C)c1] | 3 | 8 | 21781 | 8 |
| P02 of PCT/ EP2022/062496) (prodrug of Ex. 1.13 of PCT/ EP2022/062496) | 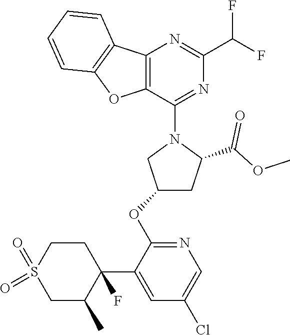 [SMILES: COC(=O)[C@@H]1C[C@H](Oc2ncc(Cl)cc2[C@@]2(F)CCS(=O)(=O)C[C@@H]2C)CN1c1nc(C(F)F)nc2c1oc1ccccc12] | >9954 | 29 | >16620 | 140 |
| Ex. 1.13 of PCT/ EP2022/062496 | 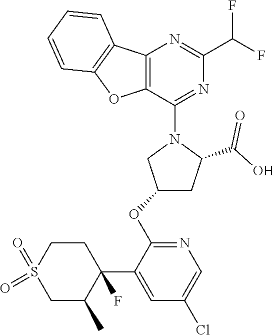 [SMILES: C[C@H]1CS(=O)(=O)CC[C@]1(F)c1cc(Cl)cnc1O[C@H]1C[C@@H](C(=O)O)N(c2nc(C(F)F)nc3c2oc2ccccc23)C1] | 2 | 6 | 22502 | 6 |
| P03 of PCT/ EP2022/062496) (prodrug of Ex. 1.05 of PCT/ EP2022/062496) | 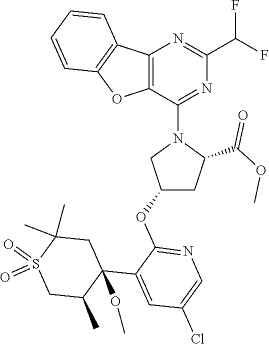 [SMILES: COC(=O)[C@@H]1C[C@H](Oc2ncc(Cl)cc2[C@@]2(OC)CC(C)(C)S(=O)(=O)C[C@@H]2C)CN1c1nc(C(F)F)nc2c1oc1ccccc12] | 7253 | 202 | 20592 | 1994 |
| Ex. 1.05 of PCT/ EP2022/062496 | 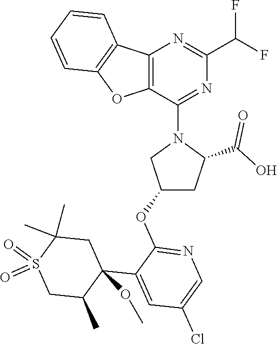 [SMILES: CO[C@]1(c2cc(Cl)cnc2O[C@H]2C[C@@H](C(=O)O)N(c3nc(C(F)F)nc4c3oc3ccccc34)C2)CC(C)(C)S(=O)(=O)C[C@@H]1C] | 2 | 44 | 13188 | 135 |

### 5. BIOLOGICAL EXPERIMENTS

The activity of the compounds of the invention may be demonstrated using the following in vitro cGAS enzyme and cell assays:

6.1 Method: Human cGAS Enzyme Assay (hcGAS ICQ (In Vitro))

Human cGAS enzyme was incubated in the presence of a 45 base pair double stranded DNA to activate the enzyme and GTP and ATP as substrates. Compound activity was determined by measuring the effect of compounds on the formation of the product of the enzyme reaction, cGAMP, which is measured by a mass spectrometry method.

Enzyme Preparation:

HumancGAS (amino acid 1-522) with an N-terminal 6×-His-tag and SUMO-tag was expressed in E. coli BL21(DE3) pLysS (Novagen) cells for 16 h at 18° C. Cells were lysed in buffer containing 25 mM Tris (pH 8), 300 mM NaCl, 10 mM imidazole, 10% glycerol, protease inhibitor cocktail (cOmplete™, EDTA-free, Roche) and DNase (5 μg/mL). The cGAS protein was isolated by affinity chromatography on Ni-NTA agarose resin and further purified by size exclusion chromatography using a Superdex 200 column (GE Healthcare) equilibrated in 20 mM Tris (pH 7.5), 500 mM KCl, and 1 mM TCEP. Purified protein was concentrated to 1.7 mg/mL and stored at −80° C.

Assay Method

Compounds were delivered in 10 mM DMSO solution, serially diluted and transferred to the 384 well assay plate (Greiner #781201) using an Echo acoustic dispenser. Typically, 8 concentrations were used with the highest concentration at 10 μM in the final assay volume followed by ˜1:5 dilution steps. DMSO concentration was set to 1% in the final assay volume. The 384 well assay plate contained 22 test compounds (column 1-22), and DMSO in column 23 and 24.

After the compound transfer, 15 μL of the enzyme-DNA-working solution (12 nM cGAS, 0.32 μM 45base pair DNA in assay buffer, 10 mM Tris pH 7.5/10 mM KCl/5 mM MgCl₂/1 mM DTT) were added to each well from column 1-23 via a MultiDrop Combi dispenser. In column 24, 15 μl of assay buffer without enzyme/DNA were added as a low control.

The plates were then pre-incubated for 60 min at room temperature.

Following that, 10 μL of GTP (ThermoFisher #R0461)-ATP (Promega #V915B) mix in assay buffer were added to the assay plate (columns 1-24, 30 μM final concentration each) using a Multidrop Combi.

The plates were incubated again for 90 min at room temperature.

Following the incubation, the reaction was stopped by 80 μL of 0,1% formic acid in assay buffer containing 5 nM cyclic-di-GMP (Sigma #SML1228) used as internal standard for the mass spectrometry. The total volume/well was 105 μL.

Rapidfire MS Detection

The plates were centrifuged at 4000 rpm, 4° C., for 5 min.

The RapidFire autosampler was coupled to a binary pump (Agilent 1290) and a Triple Quad 6500 (ABSciex, Toronto, Canada). This system was equipped with a 10 μL loop, C_{18 [12} μL bed volume] cartridge (Agilent, Part No. G9210A) containing 10 mM NH4Ac (aq) water (pH7.4) as eluent A (pump 1 at 1.5 mL/min, pump 2 at 1.25 ml/min) and 10 mM NH4Ac in v/v/v 47.5/47.5/5 ACN/MeOH/H₂O (pH7.4) as eluent B (pump 3 at 1.25 ml/min). Aspiration time: 250 ms; Load time: 3000 ms; Elute time: 3000 ms; Wash volume: 500 μL.

The MS was operated in positive ion mode with HESI ion source, with a source temperature of 550° C., curtain gas=35, gas 1=65, and gas 2=80. Unit mass resolution in SRM mode. The following transitions and MS parameters (DP: declustering potential and CE: collision energy) for cGAMP and DicGMP were determined:
Analyte: cGAMP at 675.1/524, DP=130, CE=30 and
Internal standard: cyclic-di-GMP at 690.1/540, DP=130, CE=30.

The formation of cGAMP was monitored and evaluated as ratio to cyclic-di-GMP.

Data Evaluation and Calculation:

For data evaluation and calculation, the measurement of the low control was set as 0% control and the measurement of the high control was set as 100% control. The IC₅₀ values were calculated using the standard 4 parameter logistic regression formula. Calculation: [y=(a−d)/(1+(x/c)\hat{ }b)+d], a=low value, d=high value; x=conc M; c=IC₅₀ M; b=slope

6.2 Method: Human cGAS Cell Assay and cGAMP Stimulated Counter Cell Assay (THP1₍ᵥᵢᵣ₎ IC₅₀ and THP1_{(cGAMP)} IC₅₀)

THP1-Dual™ cells (InvivoGen #thpd-nfis) expressing IRF dependent Lucia luciferase reporter were used as basis for both assays. For the detection of cellular cGAS activity cells were stimulated by a baculovirus (pFastbac-1, Invitrogen, no coding insert) infection that delivers the cGAS enzyme stimulating double-stranded DNA (measurement of THP1₍ᵥᵢᵣ₎ IC₅₀).

For the counter assay, cells were stimulated by cGAMP (SigmaAldrich #SML1232) to activate the identical pathway independent and directly downstream of cGAS (measurement of THP1_{(cGAMP)} IC₅₀). Pathway activity was monitored by measuring the Lucia luciferase activity induced by either DNA stimulated cGAS enzyme activity (measurement of THP1₍ᵥᵢᵣ₎ IC₅₀) or by cGAMP directly (measurement of THP1_{(cGAMP)} IC₅₀, counter assay).

Assay Method

Compounds were delivered in 10 mM DMSO solution, serially diluted and transferred to the 384 well assay plate (Greiner #781201) using an Echo acoustic dispenser. Typically, 8 concentrations were used with the highest concentration at 10 μM in the final assay volume followed by ˜1:5 dilution steps. DMSO concentration was set to 1% in the final assay volume. The 384 well assay plate contained 21 test compounds (column 1-22), and DMSO in column 23 and 24.

Cells, cultivated according to manufacturer conditions, were harvested by centrifugation at 300 g/10 min and were then resuspended and diluted to 1.66E5 cells/ml in fresh cell culture medium (RPMI 1640 (Gibco #A10491-01), 10% FCS (Gibco #10500), 1× GlutaMax (Gibco #35050-061), 1× Pen/Strep solution (Gibco #15140-122), 100 μg/ml Normocin (InvivoGen #ant-nr), 100 μg/ml Zeocin (InvivoGen #ant-zn), 10 μg/ml Blasticidin S (Life Technologies #A11139-03)). The baculovirus solution was then added 1:200 (have varied according to virus batch) to the cells (measurement of THP1₍ᵥᵢᵣ₎ IC₅₀). Alternatively, for the counter assay cGAMP was added to the cells at a final concentration of 10 μM (measurement of THP1_{(cGAMP)} IC₅₀). 30 μL of the cell/virus-mix were added to each well of the compound plate from column 1-23 via MultiDrop Combi dispenser (5000 cells/well). In column 24, 30 μl/5000 cells/well without virus were added as a low control.

The plates were then incubated for 18 h at 37° C. in a humidified incubator.

Following that, 15 μL of QuantiLuc detection reagent (InvivoGen #rep-qlcg5) were added to each well using a MultiDrop Combi. Measurement was done immediately after the addition using an EnVision reader (US-luminescence read-mode).

Data Evaluation and Calculation:

For data evaluation and calculation, the measurement of the low control was set as 0% control and the measurement of the high control was set as 100% control. The IC₅₀ values were calculated using the standard 4 parameter logistic regression formula. Calculation: [y=(a−d)/(1+(x/c)\hat{ }b)+d], a=low value, d=high value; x=conc M; c=IC50 M; b=slope

6.3 Method: Human Whole Blood Assay (Human WB IC₅₀) For the detection of cellular cGAS activity human whole blood was stimulated by transfection with double stranded DNA. Pathway activity was monitored by measuring the IFNα2α production.

Assay Method

Compounds were delivered as 10 mM DMSO solution and serially diluted and transferred to the 96-well cell culture plate (Corning #3595), prefilled with 20 μl OptiMEM (Gibco, #11058-021) in each well, using an Echo acoustic dispenser. Typically, 8 concentrations were used with the highest concentration at 10 μM in the final assay volume followed by ˜1:5 dilution steps. DMSO concentration was set to 0.1% in the final assay volume. The 96-well assay plate contained 10 test compounds, and DMSO in control wells.

Collection of human whole blood from 3 or more healthy donors (male or female, no medication for 7 days except contraceptive and thyroxine) as Na-Citrate blood (e.g. 3.8% in Monovettes from Sarstedt) was conducted in parallel. Whole blood was kept at room temperature for a maximum of 3 hours after collection until use in the assay.

160 μl of the whole blood samples was transferred to each well of the 96-well assay plates filled with compound/OptiMEM. All assay plates were prepared as duplicates with blood from different donors. Blood plates were kept at room temperature for 60 minutes and continuous shaking with 450 rpm, covered with the lid, but not sealed.

DNA-Fugene mix (Herring DNA, Sigma Aldrich #D6898-1G, Fugene (5×1 mL), Promega #E2312) was prepared in OptiMEM and incubated for 10 min at RT (125 ng DNA/20 μl and Fugene ratio 9.6:1). 20 μl of the DNA Fugene mix was added to each well, resulting in 125 ng DNA/well/200 μl, and Fugene Ratio 9.6:1. 20 μl OptiMEM and 9.6:1 Fugene was added to all low control wells.

After covering assay plates with area seals and the lid, blood plates were kept at room temperature for 30 minutes and continuous shaking with 450 rpm, followed by an overnight incubation of 22 h at 37° C. in the incubator, without shaking.

For the detection of IFNα-2a in human plasma, the biotinylated capture antibody (Antibody set IFNA2, Meso Scale Diagnostics #B21VH-3, including coating and capture antibody) was diluted 1:17.5 in Diluent 100 (Meso Scale Diagnostics #R50AA-4), according to the manufacturer's directions. U-Plex MSD GOLD 96-well Small Spot Strepavidin SECTOR Plates (Meso Scale Diagnostics #L45SA-5) were coated with 25 μl diluted capture antibody. Coated plates were incubated for 60 min at room temperature under continuous shaking at 700 rpm. MSD IFNα-2a plates were washed three times with 150 μl wash buffer (1×HBSS, 0.05% Tween).

After blocking the plates with 100 μl block solution/well (1×HBSS with 0.2% Tween, 2% BSA) for 60 min at room temperature and continuous shaking at 700 rpm, plates were emptied as dry as possible by dumping just before continuing with the human plasma.

Whole Blood assay plates were centrifuged at 1600 rpm for 10 minutes. 25 μl of supernatant was transferred with a pipetting robot from each whole blood plate to the corresponding IFNα-2a plate.

Plates were sealed with microplate seals and kept at room temperature again under continuous shaking at 700 rpm for two hours.

Next MSD IFNα-2a plates were washed three times with 150 μl wash buffer (1×HBSS, 0.05% Tween), before adding 25 μl MSD SULFO-TAG IFNα-2a Antibody solution (1:100 diluted in Diluent 3 (Meso Scale Diagnostics #R50AP-2) to each well of the plates.

Afterwards plates were sealed with microplate seals and kept at room temperature again under continuous shaking at 700 rpm for two hours. Finally, MSD IFNα-2a plates were washed three times with 150 μl wash buffer (1×HBSS, 0.05% Tween). 150 μl 2× Read buffer was added to each well and plates were immediately measured with the MSD Sector S600 Reader using the vendor barcode.

Data Evaluation and Calculation:

For data evaluation and calculation,% control calculation of each well was based on the mean of high (DNA stimulated control) and mean of low (unstimulated control) controls by using the following formula:

[counts(sample)−counts(low))/(counts(high)−counts(low))]*100

The IC₅₀-values were calculated using the standard 4 parameter logistic regression formula. Calculation: [y=(a−d)/(1+(x/c)\hat{ }b)+d], a=low value, d=high value; x=conc M; c=IC₅₀ M; b=slope

### 6. INDICATIONS

As has been found, the compounds of formula I or II are characterized by their range of applications in the therapeutic field. Particular mention should be made of those applications for which the compounds of formula I or II according to the invention are preferably used on the basis of their pharmaceutical activity as cGAS inhibitors. While the cGAS pathway is important for host defense against invading pathogens, such as viral infection and invasion by some intracellular bacteria, cellular stress and genetic factors may also cause production of aberrant cellular dsDNA, e.g. by nuclear or mitochondrial leakage, and thereby trigger autoinflammatory responses. Consequently, cGAS inhibitors have a strong therapeutic potential to be used in the treatment of diverse autoinflammatory and autoimmune diseases.

An et al., Arthritis Rheumatol. 2017 April; 69(4):800-807, disclosed that cGAS expression in peripheral blood mononuclear cells (PBMCs) was significantly higher in patients with the autoimmune disease systemic lupus erythematosus (SLE) than in normal controls. Targeted measurement of cGAMP by tandem mass spectrometry detected cGAMP in 15% of the tested SLE patients, but none of the normal or rheumatoid arthritis controls. Disease activity was higher in SLE patients with cGAMP versus those without cGAMP. Whereas higher cGAS expression may be a consequence of exposure to type I interferon (IFN), detection of cGAMP in SLE patients with increased disease activity indicates potential involvement of the cGAS pathway in disease expression.

Park et al., Ann Rheum Dis. 2018 October; 77(10):1507-1515, also discloses the involvement of the cGAS pathway in the development of SLE.

Thim-Uam et al., iScience 2020 Sep. 4; 23(9), 101530 (doi: 10.1016/j.isci.2020.101530), discloses that the STING pathway mediates lupus via the activation of conventional dendritic cell maturation and plasmacytoid dendritic cell differentiation.

Gao et al., Proc. Natl. Acad. Sci. USA. 2015 Oct. 20; 112 (42):E5699-705, describes that the activation of cGAS by self-DNA leads to certain autoimmune diseases such as interferonopathies.

Tonduti et al., Expert Rev. Clin. Immunol. 2020 February; 16(2):189-198 discloses that cGAS inhibitors have particular therapeutic potential in Aicardi-Goutières syndrome and familial chilblain lupus, which are lupus-like severe autoinflammatory immune-mediated disorders.

Steiner et al., Nat Commun. 2022 Apr. 28; 13(1):232; doi: 10.1038, shows that deficiency in coatomer complex I causes aberrant activation of STING signalling and COPA syndrome, and that cGAS is required to drive type I IFN signalling in a COPA syndrome cell model.

Li et al show that plasma-derived DNA containing-extracellular vesicles induce STING-mediated proinflammatory responses in dermatomyositis (Theranostics. 2021; 11(15): 7144-7158). Zhou et al (J Clin Lab Anal. 2022 October; 36(10): e24631) describes a correlation between activation of cGAS-STING pathway and myofiber atrophy/necrosis in dermatomyositis.

In Yu et al., Cell 2020 Oct. 29; 183(3):636-649, the link between TDP-43 triggered mitochondrial DNA and the activation of the cGAS/STING pathway in amyotrophic lateral sclerosis (ALS) is described.

Ryu et al., Arthritis Rheumatol. 2020 November; 72(11):1905-1915, also shows that bioactive plasma mitochondrial DNA is associated with disease progression in specific fibrosing diseases such as systemic sclerosis (SSc) or interstitial lung diseases (ILDs), progressive fibrosing interstitial lung diseases (PF-ILDs), and idiopathic pulmonary fibrosis (IPF).

In Schuliga et al., Clin. Sci. (Lond). 2020 Apr. 17; 134(7):889-905, it is described that self-DNA perpetuates IPF lung fibroblast senescence in a cGAS-dependent manner.

Additional scientific hints linking the cause for other fibrosing diseases such as non-alcoholic steatohepatitis (NASH) with the cGAS/STING pathway have been described in Yu et al., J. Clin. Invest. 2019 Feb. 1; 129(2):546-555, and in Cho et al., Hepatology. 2018 October; 68(4): 1331-1346.

Nascimento et al., Sci. Rep. 2019 Oct. 16; 9(1):14848, discloses that self-DNA release and STING-dependent sensing drives inflammation due to cigarette smoke in mice hinting at a link between the cGAS-STING pathway and chronic obstructive pulmonary disease (COPD).

Ma et al., Sci. Adv. 2020 May 20; 6 (21):eaaz6717, discloses that ulcerative colitis and inflammatory bowel disease (IBD) may be restrained by controlling cGAS-mediated inflammation.

Gratia et al., J. Exp. Med. 2019 May 6; 216(5):1199-1213, shows that Bloom syndrome protein restrains innate immune sensing of micronuclei by cGAS. Consequently cGAS-inhibitors have a therapeutic potential in treating Bloom's syndrome.

Kerur et al., Nat. Med. 2018 January; 24(1):50-61, describes that cGAS plays a significant role in noncanonical-inflammasome activation in age-related macular degeneration (AMD).

Visitchanakun et al., Int J Mol Sci. 2021 Oct. 23; 22(21):11450, shows that GAS deficient mice were less severe than the wildtype mice in the cecal ligation and puncture (CLP) and lipopolysaccharide (LPS) injection sepsis models.

Wang et al., Mediators Inflamm. 2015; 2015:192329, describes that cGAS Is required for cell proliferation and Inflammatory cytokine production in rheumatoid arthritis synoviocytes. It has also bee reported that in an inflammatory arthritis mouse model, cGAS deficiency suppressed interferon responses, inflammatory cell infiltration and joint swelling (Willemsen et al., Cell Rep. 2021 Nov. 9; 37(6):109977).

Guo et al., Osteoarthritis Cartilage. 2021 August; 29(8):1213-1224, described that damaged DNA is a key pathologic factor for osteoarthritis (OA) and this is likely mediated by the cGAS/STING pathway, since STING knockdown alleviated destabilization of the medial meniscus-induced OA development in mice.

Mao et al., Arterioscler Thromb Vasc Biol (2017) 37(5):920-929, shows that the cGAS/STING pathway mediates endothelial inflammation in response to free fatty acid-Induced Mitochondrial damage in diet-Induced obesity, indicating that cGAS inhibitors have also the potential in the treatment of obesity and diabetes.

Kerur et al, Nat Med. 2018 January; 24(1):50-61, describes that cGAS levels were elevated in the retinal pigmented epithelium in human eyes with geographic atrophy, and cGAS drives activation of noncanonical-inflammasome activation in age-related macular degeneration.

cGAS promotes cellular senescence and senescence-associated secretory phenotype (Yang et al, Proc Natl Acad Sci USA 2017 Jun. 6; 114:E4612-E4620). Cytoplasmic chromatin triggers inflammation in senescence through cGAS/STING, and STING null-mice have reduced tissue inflammation and aging (Dou et al, Nature. 2017 550: 402-406). Furthermore, in humans a variation within the STING gene is associated with healthy aging, most likely due to a decreased inflammaging (Hamann et al, Gerontology 2019; 65:145-154). Taken together, a STING inhibitor will reduce senescence associated inflammation and senescent cell accumulation and will leads improvement in senescence associated diseases such as aging, muscle disorders and fibrosis.

Further, the cGAS inhibitors of formula I or II have a therapeutic potential in the treatment of cancer (see Hoong et al., Oncotarget. 2020 Jul. 28; 11(30):2930-2955, and Chen et al., Sci. Adv. 2020 Oct. 14; 6 (42):eabb8941).

Additionally, the cGAS inhibitors of formula I or II have also a therapeutic potential in the treatment of heart failure (Hu et al., Am. J. Physiol. Heart Circ. Physiol. 2020 Jun. 1; 318 (6):H1525-H1537).

Further scientific hints at a correlation between Parkinsons disease and the cGAS/STING pathway (Sliter et al., Nature. 2018 September; 561(7722):258-262) and between Sjogren's syndrome and the cGAS/STING pathway (Papinska et al., J. Dent. Res. 2018 July; 97(8):893-900) exist.

Furthermore, cGAS inhibitors of formula I or II have also a therapeutic potential in the treatment of COVID-19/SARS-CoV-2 infections as shown in Di Domizio et al., Nature. 2022 Jan. 19. doi: 10.1038/s41586-022-04421-w: “The cGAS-STING pathway drives type I IFN immunopathology in COVID-19”, and in Neufeldt et al., Commun Biol. 2022 Jan. 12; 5(1):45. doi: 10.1038/s42003-021-02983-5: “SARS-CoV-2 infection induces a pro-inflammatory cytokine response through cGAS-STING and NF-kappaB”.

Additionally, cGAS inhibitors of formula I or N have a therapeutic potential in the treatment of renal inflammation and renal fibrosis as shown in Chung et al., Cell Metab. 2019 30:784-799: “Mitochondrial Damage and Activation of the STING Pathway Lead to Renal Inflammation and Fibrosis”, and in Maekawa et al., Cell Rep. 2019 29:1261-1273: “Mitochondrial Damage Causes Inflammation via cGAS-STING Signaling in Acute Kidney Injury”.

Furthermore, cGAS inhibitors of formula I or II have a therapeutic potential in the treatment of cancer as shown in Bakhoum et al., Nature. 2018 Jan. 25; 553(7689):467-472: “Chromosomal instability drives metastasis through a cytosolic DNA response”, and in Liu et al., Nature. 2018 November; 563(7729):131-136: “Nuclear cGAS suppresses DNA repair and promotes tumorigenesis”.

Additionally, cGAS inhibitors of formula I or N have a therapeutic potential in the treatment of dysmetabolism, because STING^{gt} animals show reduced macrophage infiltration in adipose tissue upon subchronic high caloric intake (HFD) and STING^{gt} and IRF3-deficiency leads to a decrease in blood glucose and insulin and reduced body weight (Mao et al, Arterioscler Thromb Vasc Biol, 2017; 37 (5): 920-929).

Furthermore, cGAS inhibitors of formula I or II have a therapeutic potential in the treatment of vascular diseases and leads to vascular repair/regeneration, because the release of mitochondrial DNA into the cytosol of endothelial cells results in cGAS/STING pathway activation and suppression of endothelial proliferation. Further, knockout of the cGAS gene restores endothelial repair/regeneration in a mouse model of inflammatory lung injury (Huang et al, Immunity, 2020, March 2017; 52 (3): 475-486.e5. doi: 10.1016/j.immuni.2020,02.002).

Additionally, cGAS inhibitors of formula I or N have a therapeutic potential in the treatment of age-related and obesity-related cardiovascular diseases (Hamann et al, Immun Ageing, 2020, Mar. 14; 17: 7; doi: 10.1186/s12979-020-00176-y.eCollection 2020).

Consequently the compounds of formula I or II as cGAS inhibitors can be used in the therapy of autoinflammatory and autoimmune diseases such as systemic lupus erythematosus (SLE), interferonopathies, Aicardi-Goutières syndrome (AGS), COPA syndrome, familial chilblain lupus, age-related macular degeneration (AMD), amyotrophic lateral sclerosis (ALS), inflammatory bowel disease (IBD), chronic obstructive pulmonary disease (COPD), Bloom's syndrome, Sjogren's syndrome, rheumatoid arthritis and Parkinson disease.

Additionally the compounds of formula I or II as cGAS inhibitors can be used in the therapy of fibrosing disease such as systemic sclerosis (SSc), interferonopathies, non-alcoholic steatotic hepatitis (NASH), interstitial lung disease (ILD), preferably progressive fibrosing interstitial lung disease (PF-ILD), in particular idiopathic pulmonary fibrosis (IPF).

Further, the compounds of formula I or II as cGAS inhibitors can be used in the therapy of age-related macular degeneration (AMD), retinopathy, glaucoma, diabetes, obesity, aging, muscle disorders, sepsis, osteoarthritis, heart failure, COVID-19/SARS-CoV-2 infection, renal inflammation, renal fibrosis, dysmetabolism, vascular diseases, cardiovascular diseases and cancer.

### 7. COMBINATIONS

The compounds of formula I or II may be administered to the patient alone or in combination with one or more other pharmacologically active agents.

In a preferred embodiment of the invention the compounds of formula I or II may be combined with one or more pharmacologically active agents selected from the group of anti-inflammatory agents, anti-fibrotic agents, anti-allergic agents/anti-histamines, bronchodilators, beta 2 agonists/betamimetics, adrenergic agonists, anticholinergic agents, methotrexate, mycophenolate mofetil, leukotriene modulators, JAK inhibitors, anti-interleukin antibodies, non-specific immunotherapeutics such as interferons or other cytokines/chemokines, cytokine/chemokine receptor modulators (i.e. cytokine receptor agonists or antagonists), Toll-like receptor agonists (=TLR agonists), immune checkpoint regulators, anti-TNF antibodies (Humira™), and anti-BAFF agents (Belimumab and Etanercept).

Anti-fibrotic agents are preferably selected from Pirfenidone and tyrosine kinase inhibitors such as Nintedanib, wherein Nintedanib is preferred in particular.

Preferred examples of anti-inflammatory agents are NSAIDs and corticosteroids.

NSAIDs are preferably selected from ibuprofen, naproxen, diclofenac, meloxicam, celecoxib, acetylsalicylic acid (Aspirin™), indomethacin, mefenamic acid and etoricoxib.

Corticosteroids are preferably selected from Flunisolide, Beclomethasone, Triamcinolone, Budesonide, Fluticasone, Mometasone, Ciclesonide, Rofleponide and Dexametasone.

Antiallergic agents/anti-histamines are preferably selected from Epinastine, Cetirizine, Azelastine, Fexofenadine, Levocabastine, Loratadine, Ebastine, Desloratidine and Mizolastine.

Beta 2 agonists/betamimetics may be either long acting beta 2 Agonists (LABAs) or short acting beta agonists (SABAs). Particularly preferred beta 2 agonists/betamimetics are selected from Bambuterol, Bitolterol, Carbuterol, Clenbuterol, Fenoterol, Formoterol, Hexoprenalin, Ibuterol, Pirbuterol, Procaterol, Reproterol, Salmeterol, Sulfonterol, Terbutalin, Tolubuterol, Olodaterol, and Salbutamol, in particular Olodaterol.

Anticholinergic agents are preferably selected from ipratropium salts, tiotropium salts, glycopyrronium salts, and theophylline, wherein tiotropium bromide is preferred in particular.

Leukotriene modulators are preferably selected from Montelukast, Pranlukast, Zafirlukast, Ibudilast and Zileuton.

JAK inhibitors are preferably selected from Baricitinib, Cerdulatinib, Fedratinib, Filgotinib, Gandotinib, Lestaurtinib, Momelotinib, Pacritinib, Peficitinib, Ruxolitinib, Tofacitinib, and Upadacitinib.

Anti-interleukin antibodies are preferably selected from anti-IL23 antibodies such as Risankizumab, anti-IL17 antibodies, anti-IL1 antibodies, anti-IL4 antibodies, anti-IL13 antibodies, anti-IL-5 antibodies, anti-IL-6 antibodies such as Actemra™, anti-IL-12 antibodies, anti-IL-15 antibodies.

### 8. FORMULATIONS

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, intrasternal, and subcutaneous injection or infusion. Inhalation refers to administration into the patient's lungs whether inhaled through the mouth or through the nasal passages. Topical administration includes application to the skin. The compounds of the invention may be administered via eye drops to treat Sjogren's syndrome.

Suitable forms for administration are for example tablets, capsules, solutions, syrups, emulsions or inhalable powders or aerosols. The content of the pharmaceutically effective compound(s) in each case should be in the range from 0.1 to 90 wt. %, preferably 0.5 to 50 wt. % of the total composition, i.e. in amounts which are sufficient to achieve the dosage range specified hereinafter.

The preparations may be administered orally in the form of a tablet, as a powder, as a powder in a capsule (e.g. a hard gelatin capsule), as a solution or suspension. When administered by inhalation the active substance combination may be given as a powder, as an aqueous or aqueous-ethanolic solution or using a propellant gas formulation.

Preferably, therefore, pharmaceutical formulations are characterized by the content of one or more compounds of formula I or II according to the preferred embodiments above.

It is particularly preferable if the compounds of formula I or II are administered orally, and it is also particularly preferable if they are administered once or twice a day. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example kollidone or shellac, gum arabic, talc, titanium dioxide or sugar. To achieve delayed release or prevent incompatibilities the core may also consist of a number of layers. Similarly, the tablet coating may consist of a number of layers to achieve delayed release, possibly using the excipients mentioned above for the tablets.

Syrups containing the active substances or combinations thereof according to the invention may additionally contain a sweetener such as saccharine, cyclamate, glycerol or sugar and a flavor enhancer, e.g. a flavoring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Capsules containing one or more active substances or combinations of active substances may for example be prepared by mixing the active substances with inert carriers such as lactose or sorbitol and packing them into gelatin capsules. Suitable suppositories may be made for example by mixing with carriers provided for this purpose, such as neutral fats or polyethylene glycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral administration the tablets may, of course, contain, apart from the abovementioned carriers, additives such as sodium citrate, calcium carbonate and dicalcium phosphate together with various additives such as starch, preferably potato starch, gelatin and the like. Moreover, lubricants such as magnesium stearate, sodium lauryl sulphate and talc may be used at the same time for the tableting process. In the case of aqueous suspensions, the active substances may be combined with various flavor enhancers or colorings in addition to the excipients mentioned above.

1. A compound of formula I 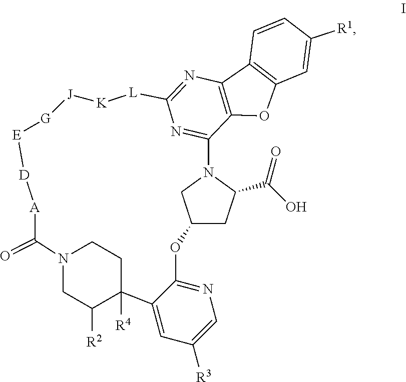
wherein
R¹ is selected from the group consisting of hydrogen, halogen, methyl, ethyl, —CF₃, —CHF₂, —CFH₂ and methoxy,
R² is selected from the group consisting of hydrogen and methyl;
R³ is selected from the group consisting of hydrogen, methyl, halogen, ethinyl, propinyl, —CO—(C₁₋₃-alkyl), —CO—NH₂, —CO—NHCH₃, —CO—N(CH₃)₂ and a 5- or 6-membered heteroaryl ring with 1 or 2 heteroatoms each independently selected from N, S or O, whereby this heteroaryl ring may optionally be further substituted by one or two further substituents each independently selected from the group consisting of F, Cl, Br, —O—CH₃, methyl, —CF₃, —CHF₂, CH₂F;
R⁴ is selected from the group consisting of hydrogen, —OH and F;
and wherein
A is selected from the group consisting of —CH₂—, —O—, —CF₂— and —CHCH₃—;
D is selected from the group consisting of —CH₂—, —O—, —CF₂— and —CHCH₃—;
E is selected from the group consisting of —CH₂—, —CO—, —O—, —CF₂— and —CHCH₃—;
G is selected from the group consisting of —NH—, —NCH₃—, —CH₂—, —O—, —CF₂— and —CHCH₃—;
J is selected from the group consisting of —CO—, —CH₂—, —O—, —CF₂— and —CHCH₃—;
K is selected either from the group consisting of —CH₂—, —CF₂— and —O— or is absent;
L is selected either from the group consisting of —CH₂—, —CHCH₃— and —CF₂— or is absent;
and prodrugs, deuterated analogues and pharmaceutical acceptable salts thereof.

2. The compound of formula II according to claim 1, wherein 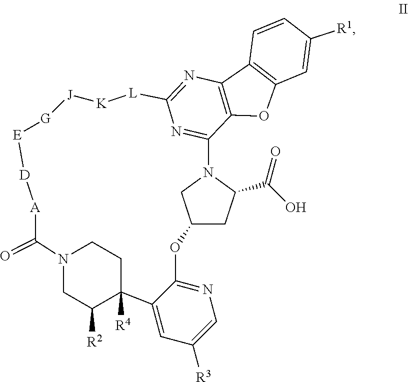
R¹ is selected from the group consisting of hydrogen and or wherein the halogen is selected from the group consisting of F and Cl;
R² is selected from the group consisting of hydrogen and methyl;
R³ is selected from the group consisting of hydrogen, Cl, Br, ethinyl, propinyl, —CO—(CH₃) and a 5- or 6-membered heteroaryl ring with 1 or 2 heteroatoms each independently selected from N, S or O, whereby this heteroaryl ring may optionally be further substituted by one or two further substituents each independently selected from the group consisting of F, —O—CH₃ and methyl;
R⁴ is F;
and wherein
A is selected from the group consisting of —CH₂—, —O—, —CF₂—, and —CHCH₃—;
D is selected from the group consisting of —CH₂— and —O—;
E is selected from the group consisting of —CH₂—, —CO— and —O—;
G is selected from the group consisting of —NH—, —NCH₃—, —CH₂— and —O—;
J is selected from the group consisting of —CO—, —CH₂— and —O—;
K is either selected from the group consisting of —CH₂—, —CF₂— and —O— or is absent;
L is either —CH₂— or is absent;
and prodrugs, deuterated analogues and pharmaceutical acceptable salts thereof.

3. The compound of formula I according to claim 1, wherein L is absent,
and prodrugs, deuterated analogues and pharmaceutical acceptable salts thereof.

4. The compound of formula I according to claim 1, wherein L and K are absent,
and prodrugs, deuterated analogues and pharmaceutical acceptable salts thereof.

5. The compound of formula I according to claim 1, wherein L is absent and wherein K is —CF₂—,
and prodrugs, deuterated analogues and pharmaceutical acceptable salts thereof.

6. The compound of formula I according to claim 1, wherein L is absent and wherein A is selected from the group consisting of —CH₂— and —CF₂—,
and prodrugs, deuterated analogues and pharmaceutical acceptable salts thereof.

7. The compound of formula I according to claim 1, wherein L is absent and whereby A is —O—,
and prodrugs, deuterated analogues and pharmaceutical acceptable salts thereof.

8. The compound of formula I according to claim 1, wherein L is absent and wherein D is —O—,
and prodrugs, deuterated analogues and pharmaceutical acceptable salts thereof.

9. The compound of formula I according to claim 1, wherein L is absent and wherein R³ is selected from the group consisting of Cl, Br, ethinyl, propinyl and a 5- or 6-membered heteroaryl ring selected from the group consisting of pyridinyl and pyrazolyl, whereby this heteroaryl ring may optionally be further substituted by one or two further substituents each independently selected from the group consisting of F, —O—CH₃ and methyl;
and prodrugs, deuterated analogues and pharmaceutical acceptable salts thereof.

10. The compound of formula I according to claim 1, wherein R¹ is selected from the group consisting of F and Cl,
and prodrugs, deuterated analogues and pharmaceutical acceptable salts thereof.

11. The compound of formula I according to claim 1, wherein R¹ is hydrogen,
and prodrugs, deuterated analogues and pharmaceutical acceptable salts thereof.

12. The compound of formula I according to claim 1, which is selected from the group consisting of 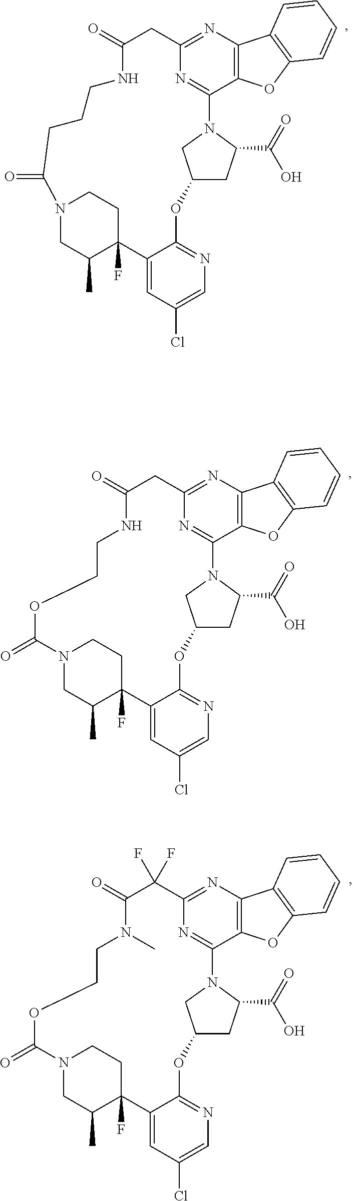 [SMILES: C[C@H]1CN2CC[C@]1(F)c1cc(Cl)cnc1O[C@H]1C[C@@H](C(=O)O)N(C1)c1nc(nc3c1oc1ccccc13)CC(=O)NCCCC2=O ; C[C@H]1CN2CC[C@]1(F)c1cc(Cl)cnc1O[C@H]1C[C@@H](C(=O)O)N(C1)c1nc(nc3c1oc1ccccc13)CC(=O)NCCCOC2=O ; C[C@H]1CN2CC[C@]1(F)c1cc(Cl)cnc1O[C@H]1C[C@@H](C(=O)O)N(C1)c1nc(nc3c1oc1ccccc13)C(F)(F)C(=O)N(C)CCCOC2=O] 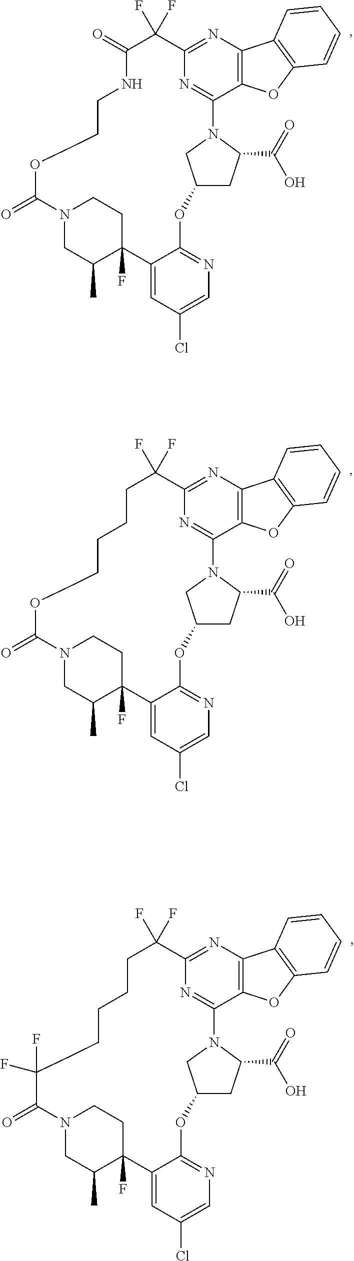 [SMILES: C[C@H]1CN2CC[C@]1(F)c1cc(Cl)cnc1O[C@H]1C[C@@H](C(=O)O)N(C1)c1nc(nc3c1oc1ccccc13)C(F)(F)C(=O)NCCCOC2=O ; C[C@H]1CN2CC[C@]1(F)c1cc(Cl)cnc1O[C@H]1C[C@@H](C(=O)O)N(C1)c1nc(nc3c1oc1ccccc13)C(F)(F)CCCCCOC2=O ; C[C@H]1CN2CC[C@]1(F)c1cc(Cl)cnc1O[C@H]1C[C@@H](C(=O)O)N(C1)c1nc(nc3c1oc1ccccc13)C(F)(F)CCCCCC(F)(F)C2=O] 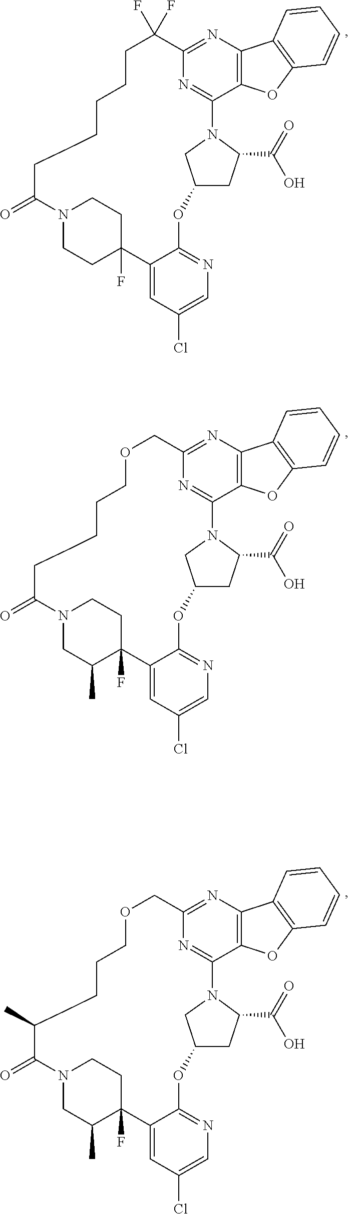 [SMILES: O=C(O)[C@@H]1C[C@H]2CN1c1nc(nc3c1oc1ccccc13)C(F)(F)CCCCCCC(=O)N1CCC(F)(CC1)c1cc(Cl)cnc1O2 ; C[C@H]1CN2CC[C@]1(F)c1cc(Cl)cnc1O[C@H]1C[C@@H](C(=O)O)N(C1)c1nc(nc3c1oc1ccccc13)COCCCCCC2=O ; C[C@H]1CCCCOCc2nc(c3oc4ccccc4c3n2)N2C[C@H](C[C@H]2C(=O)O)Oc2ncc(Cl)cc2[C@@]2(F)CCN(C[C@@H]2C)C1=O] 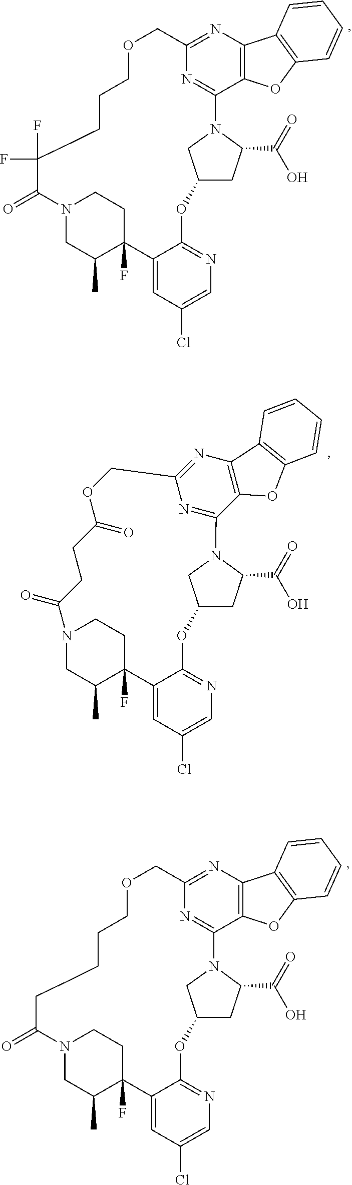 [SMILES: C[C@H]1CN2CC[C@]1(F)c1cc(Cl)cnc1O[C@H]1C[C@@H](C(=O)O)N(C1)c1nc(nc3c1oc1ccccc13)COCCCCC(F)(F)C2=O ; C[C@H]1CN2CC[C@]1(F)c1cc(Cl)cnc1O[C@H]1C[C@@H](C(=O)O)N(C1)c1nc(nc3c1oc1ccccc13)COC(=O)CCC2=O ; C[C@H]1CN2CC[C@]1(F)c1cc(Cl)cnc1O[C@H]1C[C@@H](C(=O)O)N(C1)c1nc(nc3c1oc1ccccc13)COCCCCCC2=O] 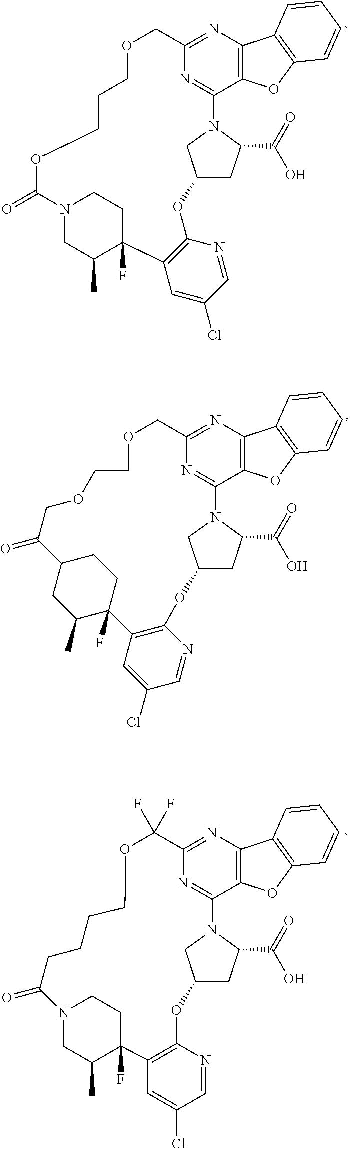 [SMILES: C[C@H]1CN2CC[C@]1(F)c1cc(Cl)cnc1O[C@H]1C[C@@H](C(=O)O)N(C1)c1nc(nc3c1oc1ccccc13)COCCCCOC2=O ; C[C@H]1CC2CC[C@]1(F)c1cc(Cl)cnc1O[C@H]1C[C@@H](C(=O)O)N(C1)c1nc(nc3c1oc1ccccc13)COCCOCC2=O ; C[C@H]1CN2CC[C@]1(F)c1cc(Cl)cnc1O[C@H]1C[C@@H](C(=O)O)N(C1)c1nc(nc3c1oc1ccccc13)C(F)(F)OCCCCC2=O] 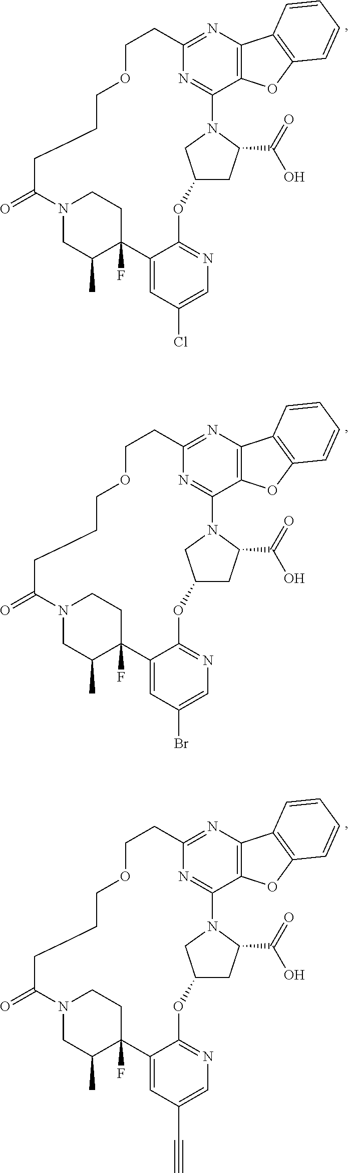 [SMILES: C[C@H]1CN2CC[C@]1(F)c1cc(Cl)cnc1O[C@H]1C[C@@H](C(=O)O)N(C1)c1nc(nc3c1oc1ccccc13)CCOCCCCC2=O ; C[C@H]1CN2CC[C@]1(F)c1cc(Br)cnc1O[C@H]1C[C@@H](C(=O)O)N(C1)c1nc(nc3c1oc1ccccc13)CCOCCCCC2=O ; C#Cc1cnc2c(c1)[C@@]1(F)CCN(C[C@@H]1C)C(=O)CCCCOCCc1nc(c3oc4ccccc4c3n1)N1C[C@H](C[C@H]1C(=O)O)O2] 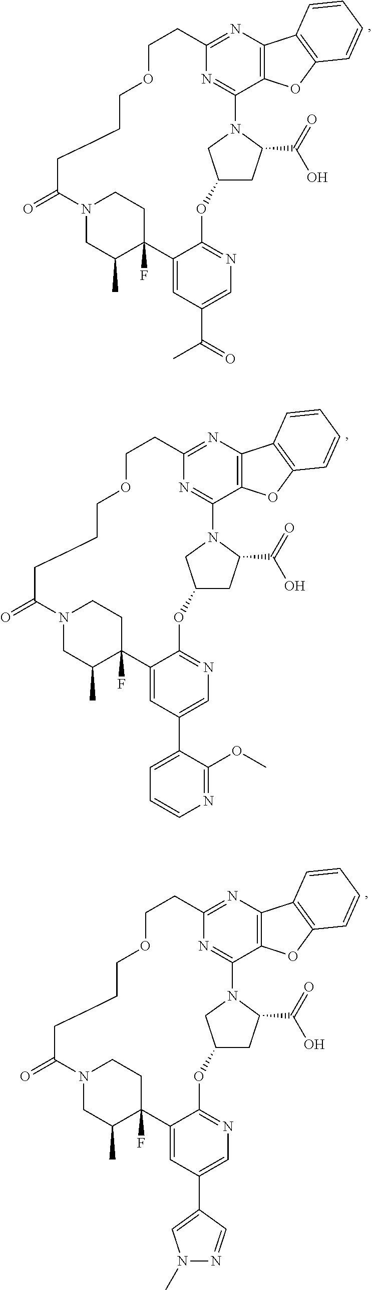 [SMILES: CC(=O)c1cnc2c(c1)[C@@]1(F)CCN(C[C@@H]1C)C(=O)CCCCOCCc1nc(c3oc4ccccc4c3n1)N1C[C@H](C[C@H]1C(=O)O)O2 ; COc1ncccc1-c1cnc2c(c1)[C@@]1(F)CCN(C[C@@H]1C)C(=O)CCCCOCCc1nc(c3oc4ccccc4c3n1)N1C[C@H](C[C@H]1C(=O)O)O2 ; C[C@H]1CN2CC[C@]1(F)c1cc(-c3cnn(C)c3)cnc1O[C@H]1C[C@@H](C(=O)O)N(C1)c1nc(nc3c1oc1ccccc13)CCOCCCCC2=O] 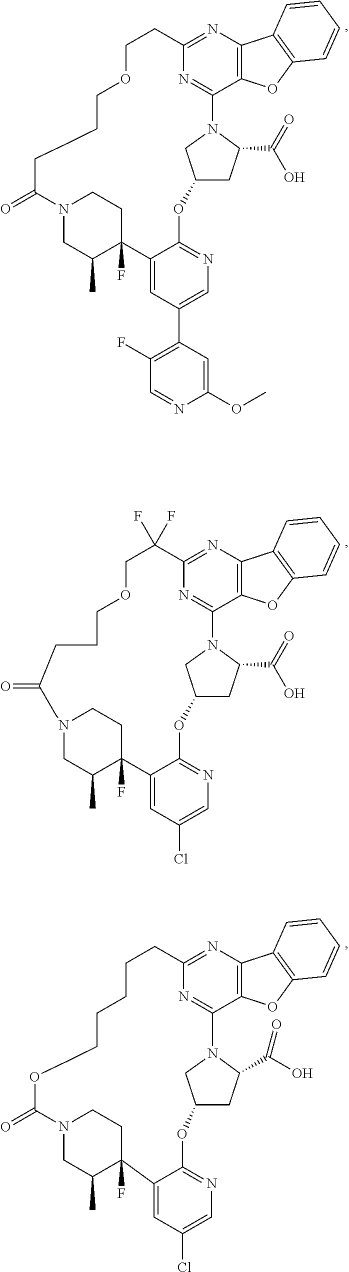 [SMILES: COc1cc(-c2cnc3c(c2)[C@@]2(F)CCN(C[C@@H]2C)C(=O)CCCCOCCc2nc(c4oc5ccccc5c4n2)N2C[C@H](C[C@H]2C(=O)O)O3)c(F)cn1 ; C[C@H]1CN2CC[C@]1(F)c1cc(Cl)cnc1O[C@H]1C[C@@H](C(=O)O)N(C1)c1nc(nc3c1oc1ccccc13)C(F)(F)COCCCC(=O)C2 ; C[C@H]1CN2CC[C@]1(F)c1cc(Cl)cnc1O[C@H]1C[C@@H](C(=O)O)N(C1)c1nc(nc3c1oc1ccccc13)CCCCCCOC2=O] 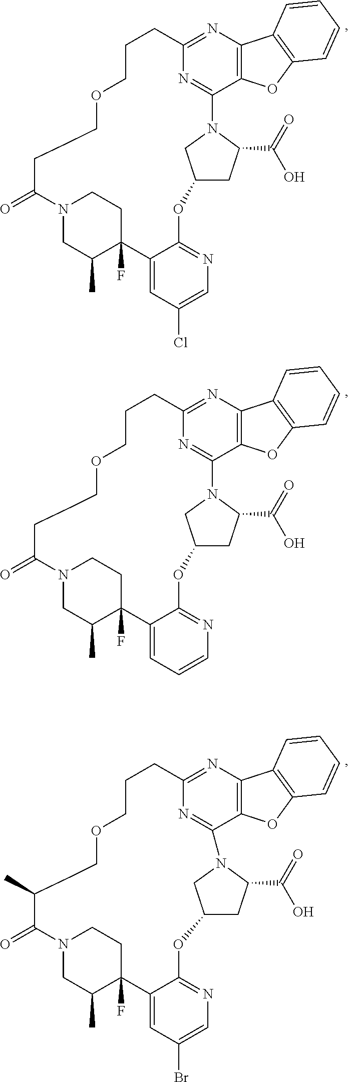 [SMILES: C[C@H]1CN2CC[C@]1(F)c1cc(Cl)cnc1O[C@H]1C[C@@H](C(=O)O)N(C1)c1nc(nc3c1oc1ccccc13)CCCOCCCC2=O ; C[C@H]1CN2CC[C@]1(F)c1cccnc1O[C@H]1C[C@@H](C(=O)O)N(C1)c1nc(nc3c1oc1ccccc13)CCCOCCCC2=O ; C[C@H]1CCOCCCc2nc(c3oc4ccccc4c3n2)N2C[C@H](C[C@H]2C(=O)O)Oc2ncc(Br)cc2[C@@]2(F)CCN(C[C@@H]2C)C1=O] 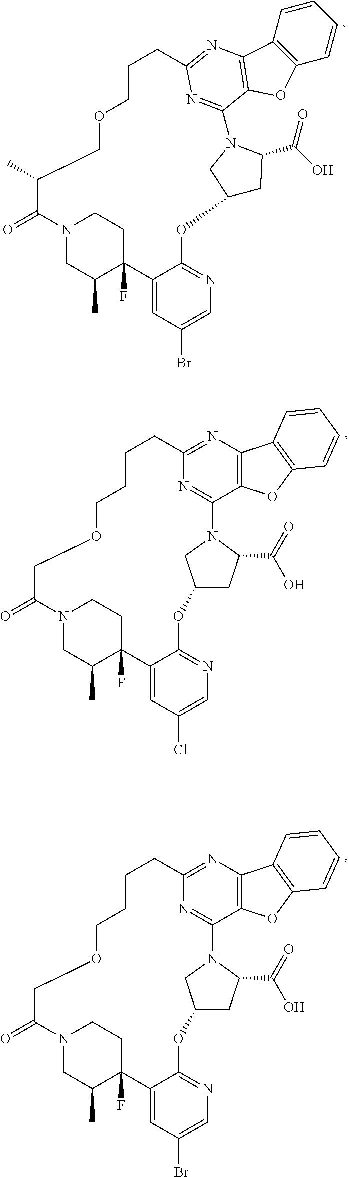 [SMILES: C[C@@H]1CCOCCCc2nc(c3oc4ccccc4c3n2)N2C[C@H](C[C@H]2C(=O)O)Oc2ncc(Br)cc2[C@@]2(F)CCN(C[C@@H]2C)C1=O ; C[C@H]1CN2CC[C@]1(F)c1cc(Cl)cnc1O[C@H]1C[C@@H](C(=O)O)N(C1)c1nc(nc3c1oc1ccccc13)CCCCOCCC2=O ; C[C@H]1CN2CC[C@]1(F)c1cc(Br)cnc1O[C@H]1C[C@@H](C(=O)O)N(C1)c1nc(nc3c1oc1ccccc13)CCCCOCCC2=O] 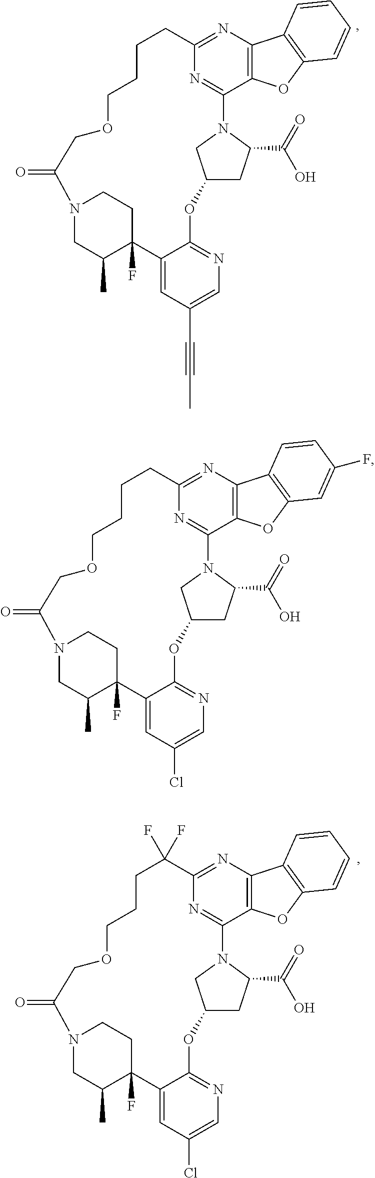 [SMILES: CC#Cc1cnc2c(c1)[C@@]1(F)CCN(C[C@@H]1C)C(=O)COCCCCc1nc(c3oc4ccccc4c3n1)N1C[C@H](C[C@H]1C(=O)O)O2 ; C[C@H]1CN2CC[C@]1(F)c1cc(Cl)cnc1O[C@H]1C[C@@H](C(=O)O)N(C1)c1nc(nc3c1oc1cc(F)ccc13)CCCCOCC2=O ; C[C@H]1CN2CC[C@]1(F)c1cc(Cl)cnc1O[C@H]1C[C@@H](C(=O)O)N(C1)c1nc(nc3c1oc1ccccc13)C(F)(F)CCCOCC2=O] 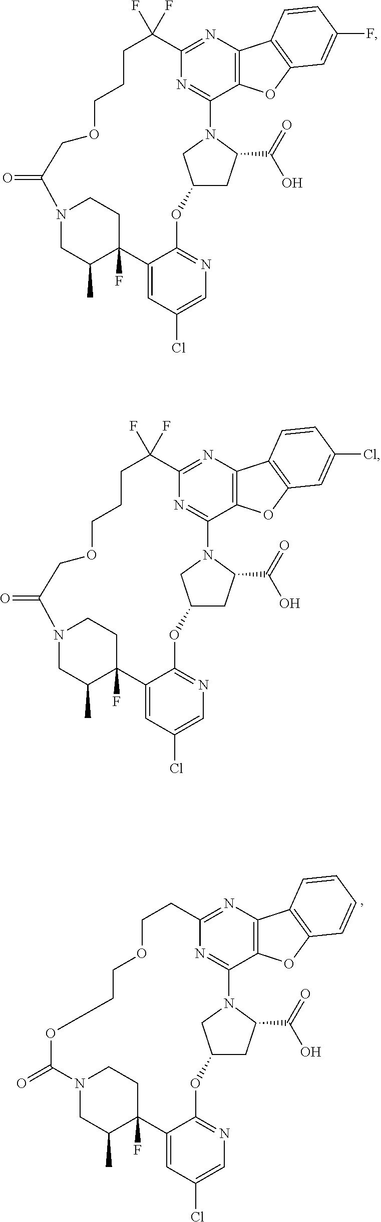 [SMILES: C[C@H]1CN2CC[C@]1(F)c1cc(Cl)cnc1O[C@H]1C[C@@H](C(=O)O)N(C1)c1nc(nc3c1oc1cc(F)ccc13)C(F)(F)CCCOCC2=O ; C[C@H]1CN2CC[C@]1(F)c1cc(Cl)cnc1O[C@H]1C[C@@H](C(=O)O)N(C1)c1nc(nc3c1oc1cc(Cl)ccc13)C(F)(F)CCCOCC2=O ; C[C@H]1CN2CC[C@]1(F)c1cc(Cl)cnc1O[C@H]1C[C@@H](C(=O)O)N(C1)c1nc(nc3c1oc1ccccc13)CCOCCOC2=O] 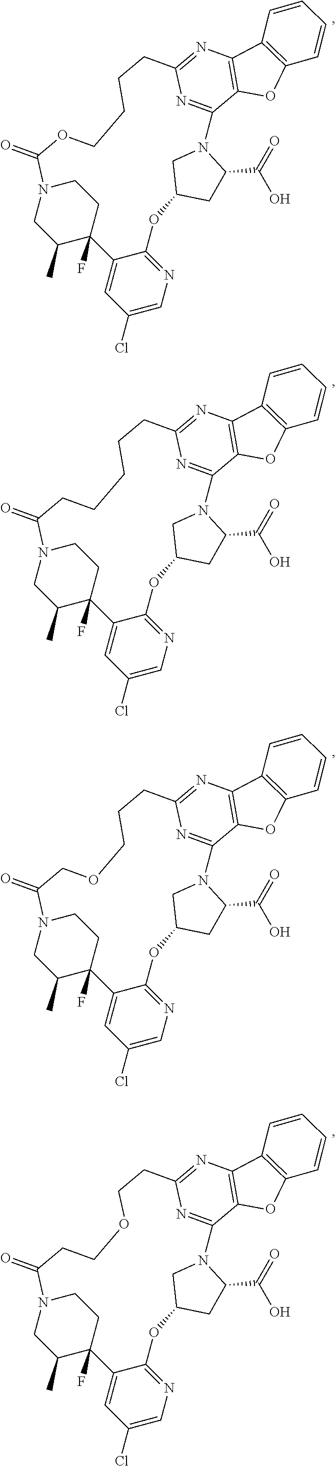 [SMILES: C[C@H]1CN2CC[C@]1(F)c1cc(Cl)cnc1O[C@H]1C[C@@H](C(=O)O)N(C1)c1nc(nc3c1oc1ccccc13)CCCCOC2=O ; C[C@H]1CN2CC[C@]1(F)c1cc(Cl)cnc1O[C@H]1C[C@@H](C(=O)O)N(C1)c1nc(nc3c1oc1ccccc13)CCCCCC2=O ; C[C@H]1CN2CC[C@]1(F)c1cc(Cl)cnc1O[C@H]1C[C@@H](C(=O)O)N(C1)c1nc(nc3c1oc1ccccc13)CCCOCC2=O ; C[C@H]1CN2CC[C@]1(F)c1cc(Cl)cnc1O[C@H]1C[C@@H](C(=O)O)N(C1)c1nc(nc3c1oc1ccccc13)CCOCCC2=O]
and prodrugs, deuterated analogues and pharmaceutical acceptable salts thereof.

13. The compound of formula I according to claim 1, wherein
A is selected from the group consisting of —CH₂— and —O—;
D is selected from the group consisting of —CH— and —O—;
E is selected from the group consisting of-CH₂— and —O—;
G is selected from the group consisting of —CH— and —O—;
J is selected from the group consisting of —CH₂— and —O—;
K is either selected from the group consisting of —CH₂— and —CF₂—;
L is absent;
and prodrugs, deuterated analogues and pharmaceutical acceptable salts thereof.

14. The compound of formula I according to claim 1, which is selected from the group consisting of 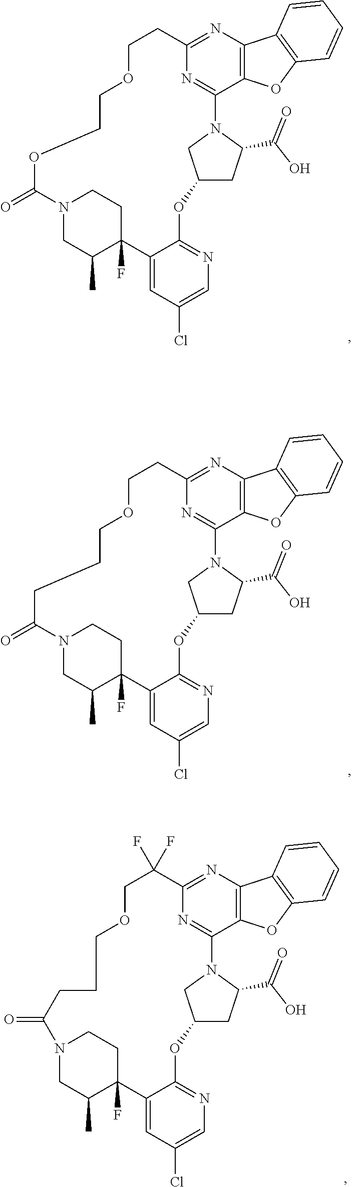 [SMILES: C[C@H]1CN2CC[C@]1(F)c1cc(Cl)cnc1O[C@H]1C[C@@H](C(=O)O)N(C1)c1nc(nc3c1oc1ccccc13)CCOCCOC2=O ; C[C@H]1CN2CC[C@]1(F)c1cc(Cl)cnc1O[C@H]1C[C@@H](C(=O)O)N(C1)c1nc(nc3c1oc1ccccc13)CCOCCCC2=O ; C[C@H]1CN2CC[C@]1(F)c1cc(Cl)cnc1O[C@H]1C[C@@H](C(=O)O)N(C1)c1nc(nc3c1oc1ccccc13)C(F)(F)COCCCC2=O] 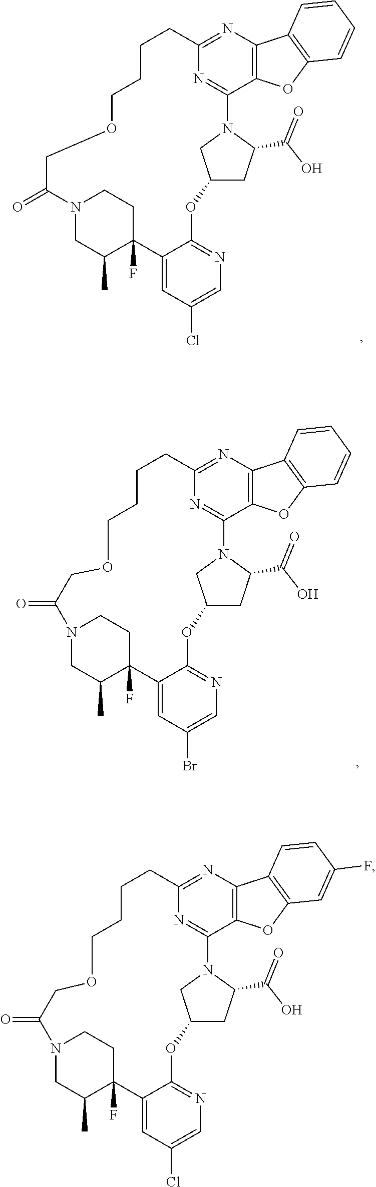 [SMILES: C[C@H]1CN2CC[C@]1(F)c1cc(Cl)cnc1O[C@H]1C[C@@H](C(=O)O)N(C1)c1nc(nc3c1oc1ccccc13)CCCCOCC2=O ; C[C@H]1CN2CC[C@]1(F)c1cc(Br)cnc1O[C@H]1C[C@@H](C(=O)O)N(C1)c1nc(nc3c1oc1ccccc13)CCCCOCC2=O ; C[C@H]1CN2CC[C@]1(F)c1cc(Cl)cnc1O[C@H]1C[C@@H](C(=O)O)N(C1)c1nc(nc3c1oc1cc(F)ccc13)CCCCOCC2=O] 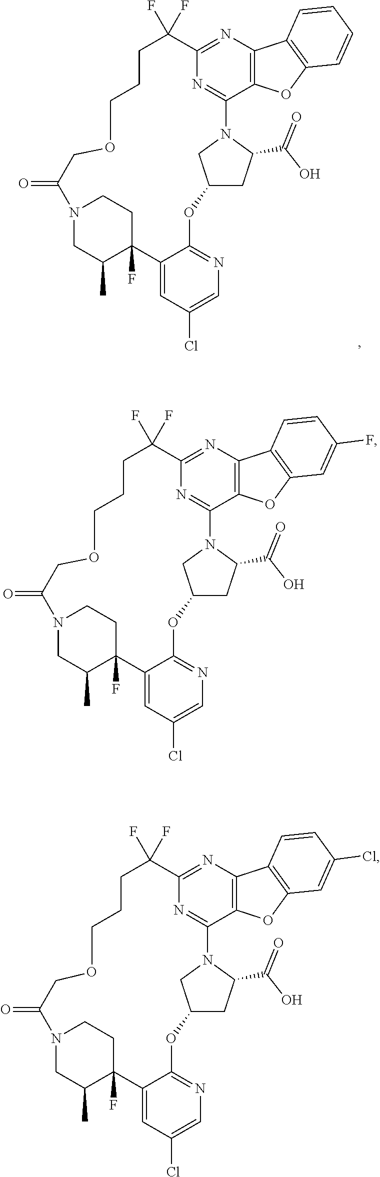 [SMILES: C[C@H]1CN2CC[C@]1(F)c1cc(Cl)cnc1O[C@H]1C[C@@H](C(=O)O)N(C1)c1nc(nc3c1oc1ccccc13)C(F)(F)CCCOCC2=O ; C[C@H]1CN2CC[C@]1(F)c1cc(Cl)cnc1O[C@H]1C[C@@H](C(=O)O)N(C1)c1nc(nc3c1oc1cc(F)ccc13)C(F)(F)CCCOCC2=O ; C[C@H]1CN2CC[C@]1(F)c1cc(Cl)cnc1O[C@H]1C[C@@H](C(=O)O)N(C1)c1nc(nc3c1oc1cc(Cl)ccc13)C(F)(F)CCCOCC2=O] 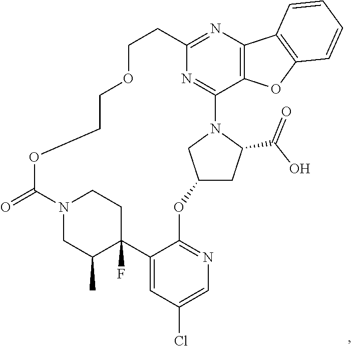 [SMILES: C[C@H]1CN2CC[C@]1(F)c1cc(Cl)cnc1O[C@H]1C[C@@H](C(=O)O)N(C1)c1nc(nc3c1oc1ccccc13)CCOCCOC2=O]
and prodrugs, deuterated analogues and pharmaceutical acceptable salts thereof.

15. The compound of formula I according to claim 1, wherein
A is selected from the group consisting of —CH₂-and —O—;
D is selected from the group consisting of —CH₂-and —O—;
E is —CH₂—;
G is selected from the group consisting of —CH₂-and —O—;
J is —CH₂—;
K is either selected from the group consisting of —CH₂— and —CF₂—;
L is absent;
and prodrugs, deuterated analogues and pharmaceutical acceptable salts thereof.

16. The compound of formula I according to claim 1, which is selected from the group consisting of 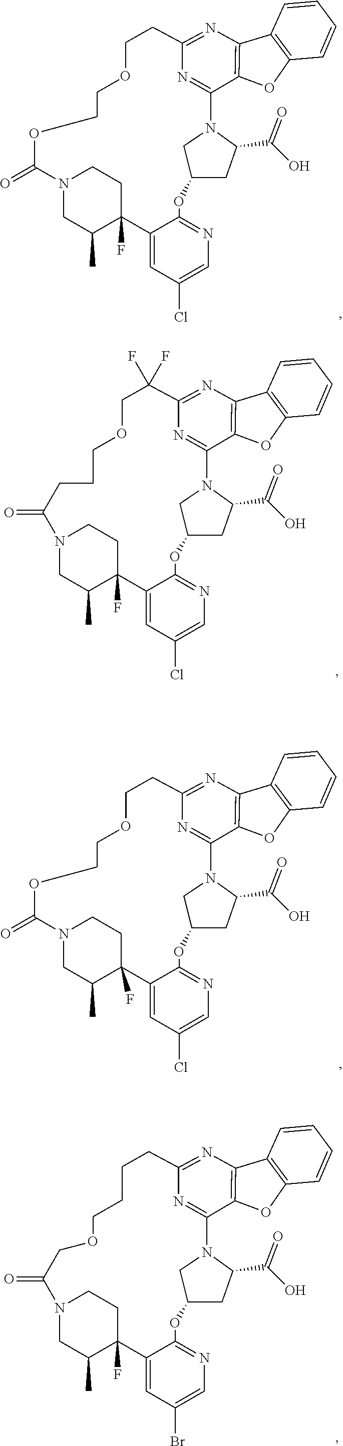 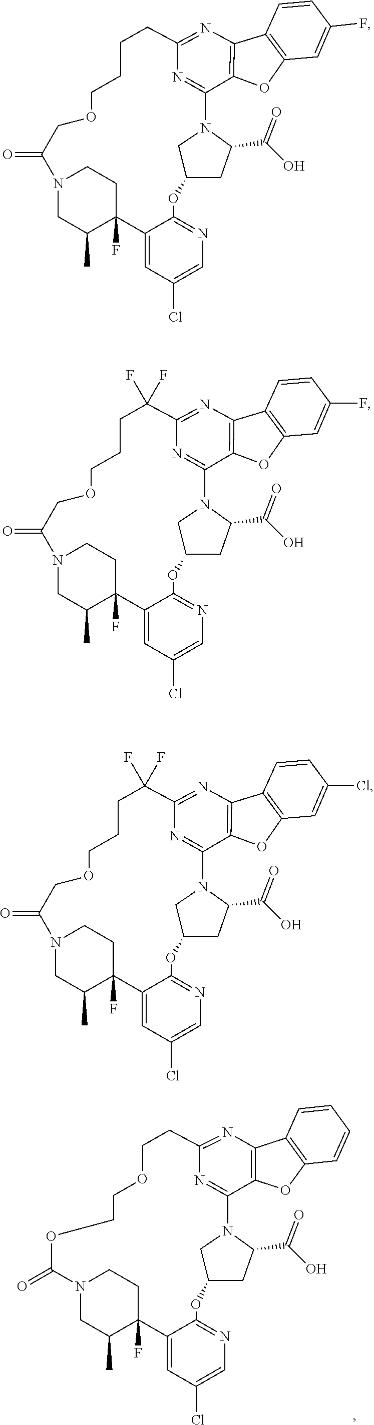 [SMILES: C[C@H]1CN2CC[C@]1(F)c1cc(Cl)cnc1O[C@H]1C[C@@H](C(=O)O)N(C1)c1nc(nc3c1oc1cc(F)ccc13)CCCCOCC2=O ; C[C@H]1CN2CC[C@]1(F)c1cc(Cl)cnc1O[C@H]1C[C@@H](C(=O)O)N(C1)c1nc(nc3c1oc1cc(F)ccc13)C(F)(F)CCCOCC2=O ; C[C@H]1CN2CC[C@]1(F)c1cc(Cl)cnc1O[C@H]1C[C@@H](C(=O)O)N(C1)c1nc(nc3c1oc1cc(Cl)ccc13)C(F)(F)CCCOCC2=O ; C[C@H]1CN2CC[C@]1(F)c1cc(Cl)cnc1O[C@H]1C[C@@H](C(=O)O)N(C1)c1nc(nc3c1oc1ccccc13)CCOCCOC2=O]
and prodrugs, deuterated analogues and pharmaceutical acceptable salts thereof.

17. An intermediate compound of
a) formula (A-I) 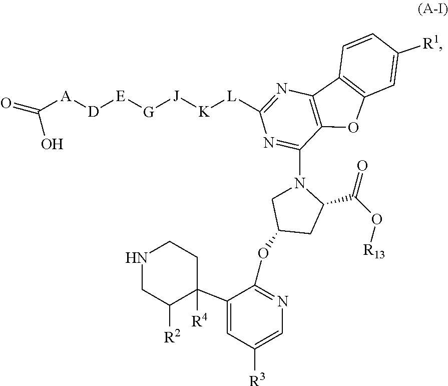
wherein R¹, R², R³, R⁴, A, D, E, G, J, K and L are defined as in claim 1 and wherein R₁₃ is selected from the group consisting of hydrogen, methyl, ethyl and tert-butyl,
b) formula A-II 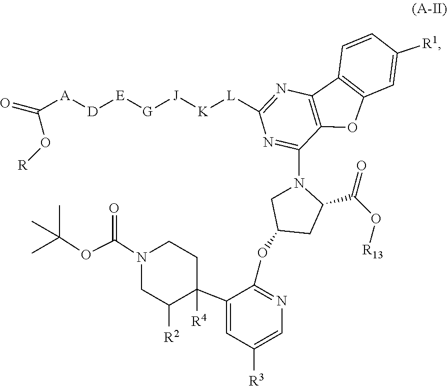
wherein R¹, R², R³, R⁴, A, D, E, G, J, K and L are defined as in claim 1, wherein R₁₃ is selected from the group consisting of hydrogen, methyl, ethyl and tert-butyl, and wherein R is either hydrogen or a protecting group selected from the group consisting of tert-butyl, methyl, ethyl and benzyl,
c) formula (B-I) 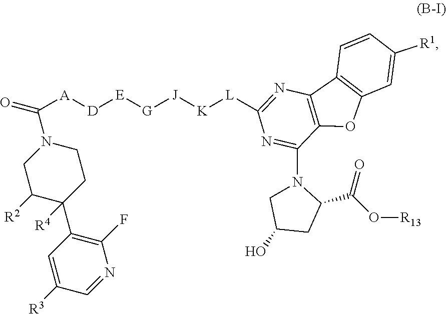
wherein R¹, R², R³, R⁴, A, D, E, G, J, K and L are defined as in claim 1 and wherein R¹³ is selected from the group consisting of hydrogen, methyl, ethyl and tert-butyl,
d) formula (C-I) 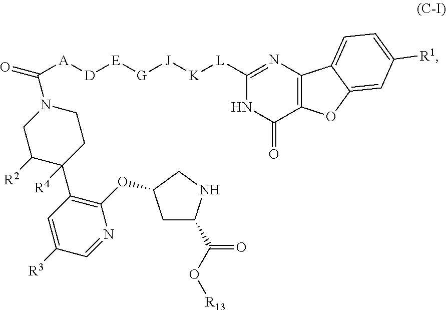
wherein R¹, R², R³, R⁴, A, D, E, G, J, K and L are defined as in claim 1 and wherein R₁₃ is selected from the group consisting of hydrogen, methyl, ethyl and tert-butyl,
or of
e) formula (C-II) 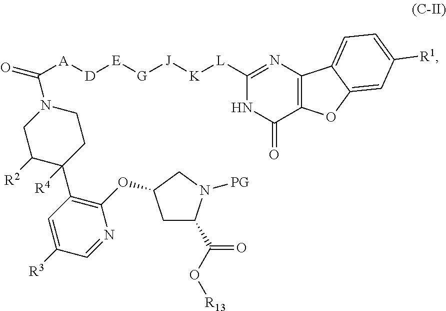
wherein R¹, R², R³, R⁴, A, D, E, G, J, K and L are defined as in claim 1 and wherein R₁₃ is selected from the group consisting of hydrogen, methyl, ethyl and tert-butyl,
wherein PG is selected from the group consisting of tert-butoxycarbonyl (Boc), allyloxycarbonyl (Alloc), benzyloxycarbonyl (Cbz) and fluorenylmethoxycarbonyl (Fmoc).

18. A method of treating in a patient a disease that can be treated by the inhibition of cGAS, said method comprising administering to the patient a compound of formula I according to claim 1.

19. A method of treating a disease in a patient, said method comprising administering to the patient a compound of formula I according to claim 1, wherein the disease is selected from the group consisting of systemic lupus erythematosus (SLE), interferonopathies, Aicardi-Goutières syndrome (AGS), COPA syndrome, familial chilblain lupus, age-related macular degeneration (AMD), amyotrophic lateral sclerosis (ALS), retinopathy, glaucoma, diabetes, obesity, inflammatory bowel disease (IBD), chronic obstructive pulmonary disease (COPD), Bloom's syndrome, dermatomyositis, Sjogren's syndrome, Parkinsons disease, heart failure, cancer, aging, muscle disorders, sepsis, rheumatoid arthritis, osteoarthritis, COVID-19, systemic sclerosis (SSc), non-alcoholic steatotic hepatitis (NASH), interstitial lung disease (ILD), progressive fibrosing interstitial lung disease (PF-ILD), and idiopathic pulmonary fibrosis (IPF).

20. The method according to claim 19, wherein the disease is selected from the group consisting of systemic lupus erythematosus (SLE), interferonopathies, Aicardi-Goutières syndrome (AGS), COPA syndrome, familial chilblain lupus, dermatomyositis, age-related macular degeneration (AMD), amyotrophic lateral sclerosis (ALS), inflammatory bowel disease (IBD), chronic obstructive pulmonary disease (COPD), Bloom's syndrome, Sjogren's syndrome, rheumatoid arthritis and Parkinsons disease.

21. The method according to claim 19, wherein the disease is selected from the group consisting of systemic sclerosis (SSc), non-alcoholic steatohepatitis (NASH), interferonopathies, interstitial lung disease (ILD), progressive fibrosing interstitial lung disease (PF-ILD), and idiopathic pulmonary fibrosis (IPF).

22. The method according to claim 19, wherein the disease is selected from the group consisting of age-related macular degeneration (AMD), retinopathy, glaucoma, diabetes, obesity, aging, muscle disorders, sepsis, osteoarthritis, heart failure, COVID-19/SARS-CoV-2 infection, renal inflammation, renal fibrosis, dysmetabolism, vascular diseases, cardiovascular diseases and cancer.

23. A pharmaceutical composition comprising the compound of formula I according to claim 1 and optionally one or more pharmaceutically acceptable carriers and/or excipients.

24. The pharmaceutical composition according to claim 23 in combination with one or more active agents selected from the group consisting of anti-inflammatory agents, anti-fibrotic agents, anti-allergic agents/anti-histamines, bronchodilators, beta 2 agonists/betamimetics, adrenergic agonists, anticholinergic agents, methotrexate, mycophenolate mofetil, leukotriene modulators, JAK inhibitors, anti-interleukin antibodies, non-specific immunotherapeutics, interferons or other cytokines/chemokines, cytokine/chemokine receptor modulators, toll-like receptor agonists, immune checkpoint regulators, an anti-TNF antibody, Adalimumab, an anti-BAFF antibody, Belimumab and Etanercept,
and optionally one or more pharmaceutically acceptable carriers and/or excipients.

25. The pharmaceutical composition according to claim 23 in combination with one or more anti-fibrotic agents selected from the group consisting of Pirfenidon and Nintedanib, and optionally one or more pharmaceutically acceptable carriers and/or excipients.

26. The pharmaceutical composition according to claim 23 in combination with one or more anti-inflammatory agents selected from the group consisting of NSAIDs and corticosteroids, and optionally one or more pharmaceutically acceptable carriers and/or excipients.

27. The pharmaceutical composition according to claim 23 in combination with one or more active agents selected from the group consisting of bronchodilators, beta 2 agonists/betamimetics, adrenergic agonists and anticholinergic agents, and optionally one or more pharmaceutically acceptable carriers and/or excipients.

28. The pharmaceutical composition according to claim 23 and one or more anti-interleukin antibodies selected from the group consisting of anti-IL-23 antibodies, Risankizumab, anti-IL-17 antibodies, anti-IL-1 antibodies, anti-IL-4 antibodies, 13 antibodies, anti-IL-5 antibodies, anti-IL-6 antibodies, Tocilizumab, anti-IL-12 antibodies and anti-IL-15 antibodies.
